(19) United States
(12) Patent Application Publication
(10) Pub. No.: US 2022/0315968 A1
(43) Pub. Date: Oct. 6, 2022

(54) PEPTIDES AND METHODS FOR THE CARBON-CARBON BOND FORMATION

(71) Applicant: Max-Planck-Gesellschaft zur Förderung der Wissenschaften e.V., Munich (DE)

(72) Inventors: Tobias Erb, Marburg (DE); Simon Burgener, Bern (CH)

(21) Appl. No.: 17/638,863

(22) PCT Filed: Aug. 29, 2020
(86) PCT No.: PCT/EP2020/074168

§ 371 Date: Feb. 28, 2022

(30) Foreign Application Priority Data
Aug. 31, 2019 (EP) EP 19194807.4
Dec. 20, 2019 (EP) EP 19218953.8

(51) Int. Cl. C12P 17/04; C12P 13/00; C12P 7/42; C12P 17/12

(52) U.S. Cl. CPC C12P 17/04; C12P 13/008; C12Y 401/01008; C12P 7/42; C12P 17/12

(57) ABSTRACT

The present invention relates to methods for the preparation of α-hydroxyacyl compounds, and peptides for the catalyzed formation of α-hydroxyacyl compounds as well as their use in the preparation of α-hydroxyacyl compounds.

The present invention relates to methods for the preparation of α-hydroxyacyl compounds, and peptides for the catalyzed formation of α-hydroxyacyl compounds as well as their use in the preparation of α-hydroxyacyl compounds.

### BACKGROUND OF THE INVENTION

One of biotechnology's central goals is the synthesis of multicarbon compounds under mild and sustainable conditions from renewable resources. This requires biocatalysts and methods that enable selective C—C bond formation (carboligation) between two carbon units.

Moreover, optically active compounds are useful for optical resolving agents. For example, optically active compounds can be used for optically resolving agents of medical or agriculture supplies such as 2-amino-1-butanol which is a starting material of the antituberculous drug ethambutol, diltiazem hydrochloride which is a coronary vasodilator and tetramizol which is effective as an anthelmintic. Further, optically active compounds can also be used as starting materials or intermediates for the synthesis of optically pure therapeutic agents.

In the international patent application WO 2016/069929 A1, the formation of α-hydroxyacyl-CoA thioester using α-hydroxyacyl-CoA lyase (HACL1) is disclosed, and is thus not directed to aromatic α-hydroxyacyl-CoA or even less to aromatic α-hydroxy carboxylic acids such as mandelic acid or its derivatives. The conversion and even less the yield of the product is not taught. Furthermore, the enantioselectivity of the reaction and the stereochemical information of the stereocenter of the product are not described.

The US patent application US 2003/0119173 A1 refers to the synthesis of mandelic acid derivatives by enzymatic reduction of α-keto acids. It is not described that the reaction can be performed enantioselective.

In the scientific publication Metabolic Engineering Communications 2018, 1-9, the production of hydroxycarboxylic acids, especially mandelic acid starting from a phenylpyruvate is disclosed. For the production, yeast, especially S. cerevisae, is modified with the help of so-called metabolic engineering.

Albeit enantioselective synthesis of (R)-mandelic acid derivatives is well known in the state of the art, the enzymatic enantioselective synthesis of (S)-mandelic acid derivatives is very limited. In the US patent application US 2010/0036150 the synthesis of (S)-mandelic acid derivatives are described, wherein the reaction starts from acetylated α-hydroxy carboxylic acid ester.

Therefore, there is an interest in further synthesis procedure for the preparation of aromatic α-hydroxy carboxylic acids, salts thereof or their esters in particular enantioselective and especially selective for the (S)-configuration.

It is the objective of the present invention to provide methods and peptides for the preparation of aromatic α-hydroxy carboxylic acids, salts thereof or their esters in particular enantioselective, and especially selective for the (S)-enantiomer.

The objective of the present invention is solved by the teaching of the independent claims. Further advantageous features, aspects and details of the invention are evident from the dependent claims, the description, the figures, and the examples of the present application.

### BRIEF DESCRIPTION OF THE INVENTION

The objective of the present invention can be solved by a method for the preparation of an α-hydroxyacyl compound of the formula (I′)

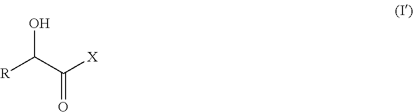

wherein R represents

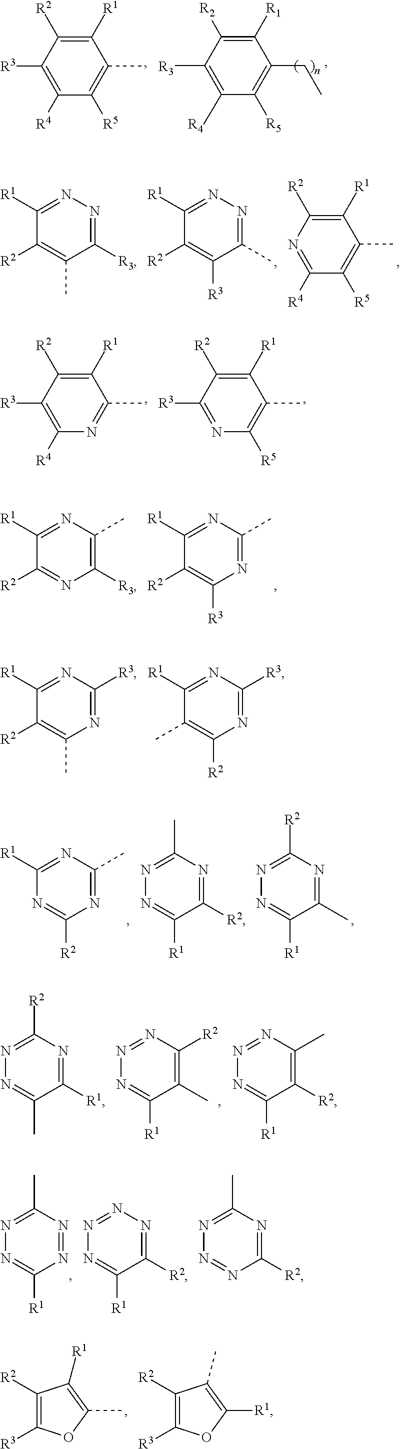

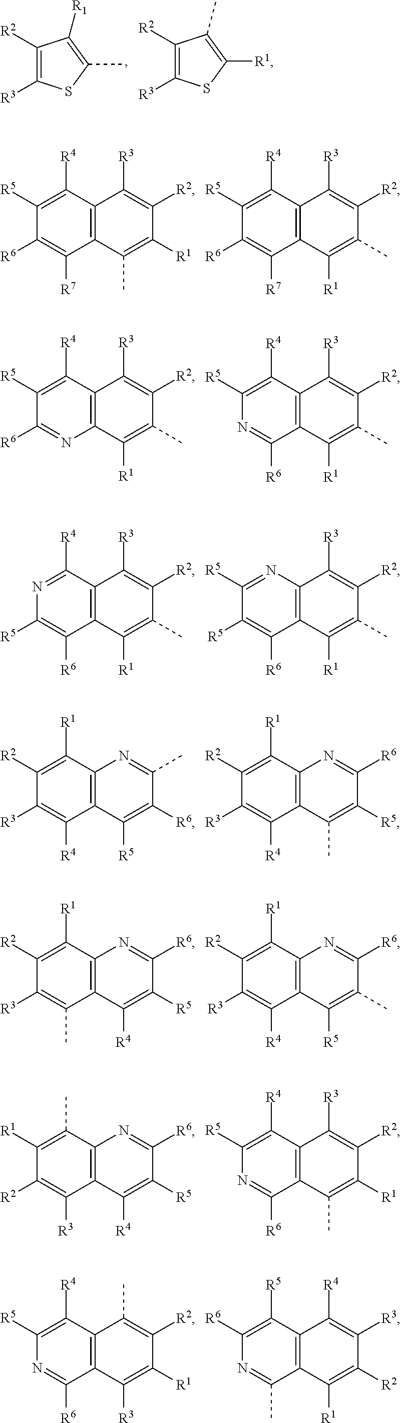

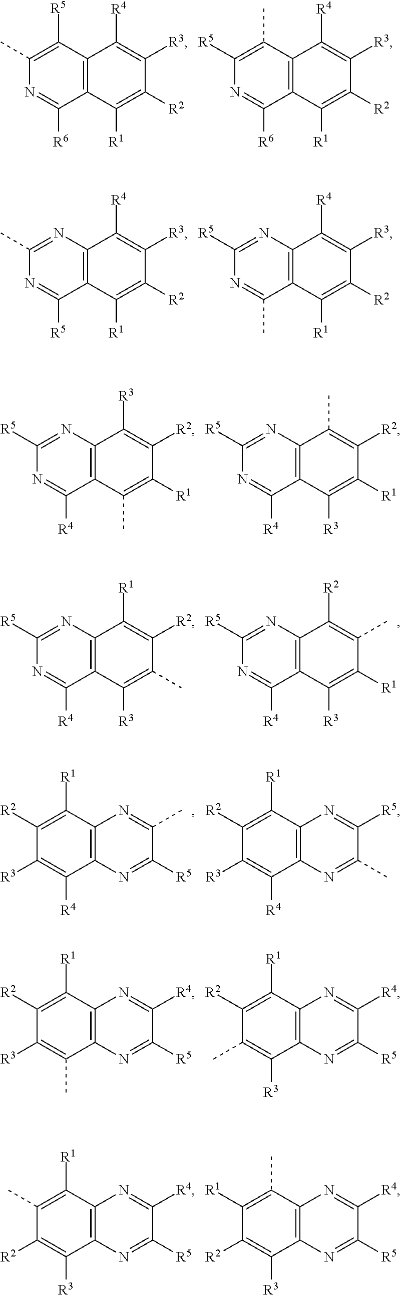

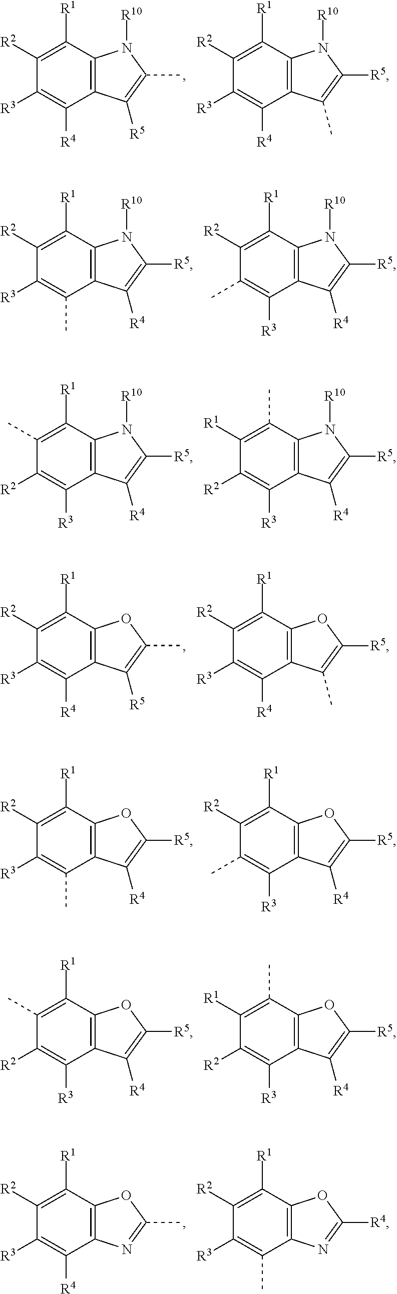

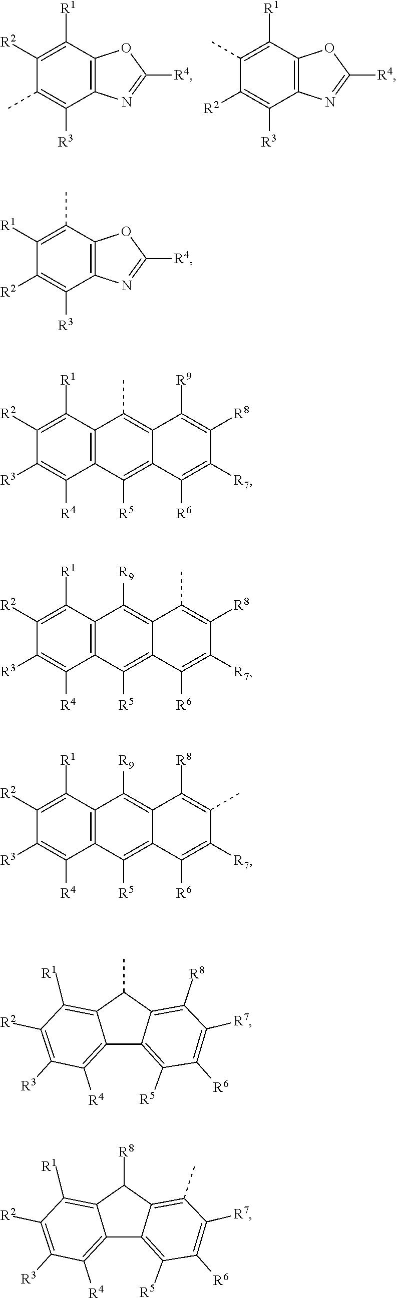

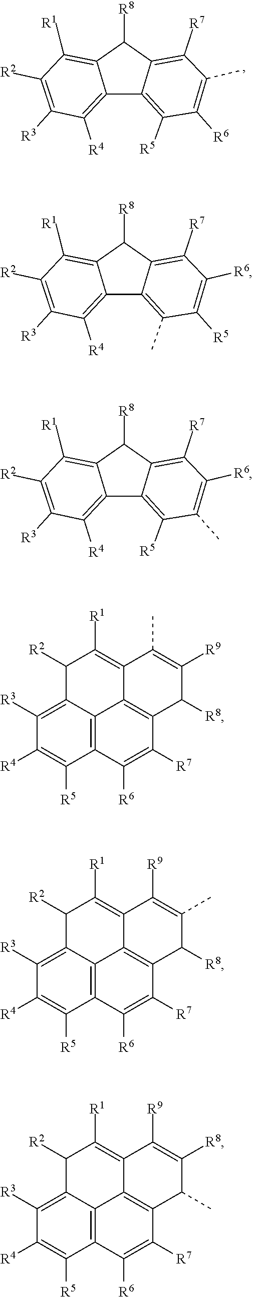

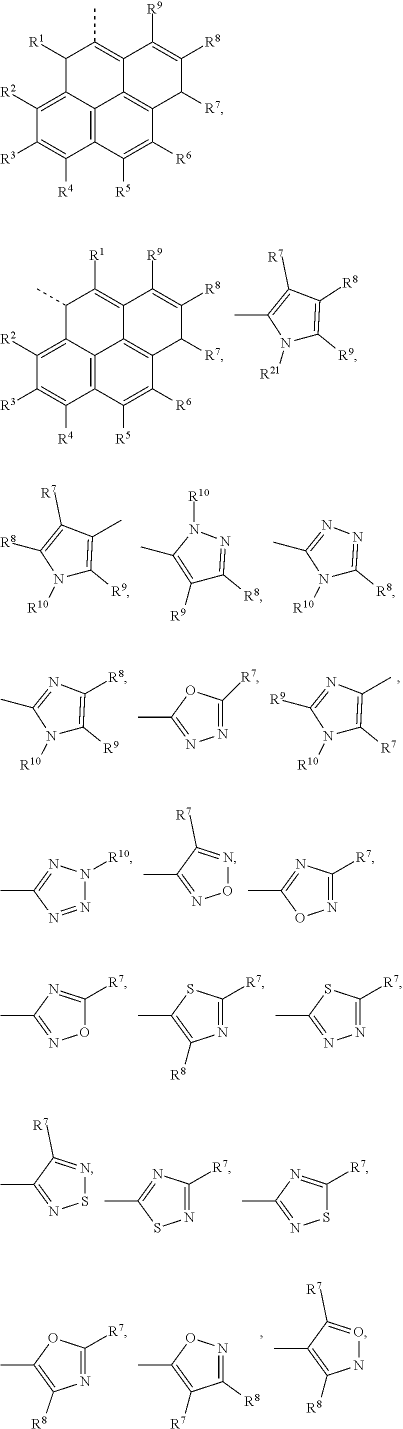

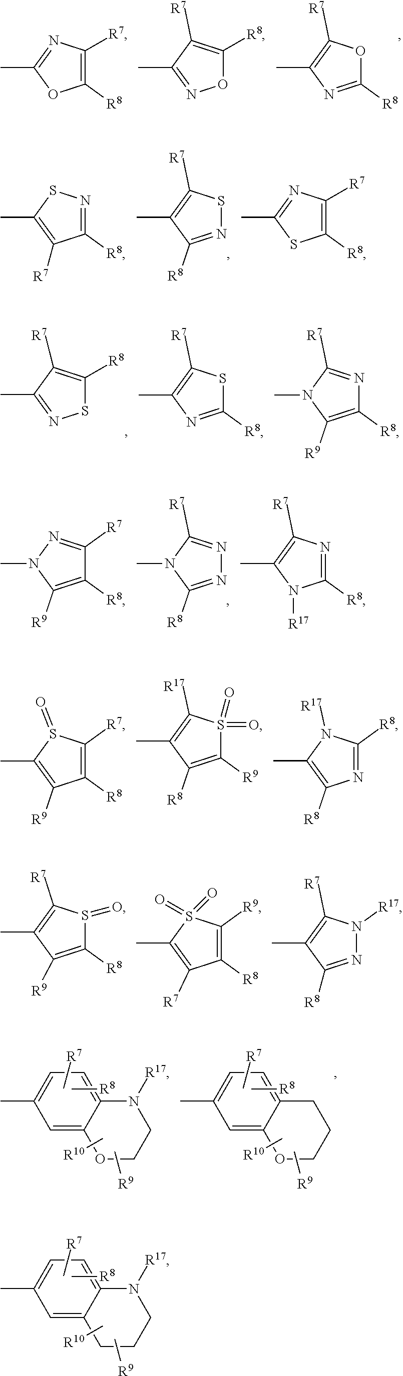

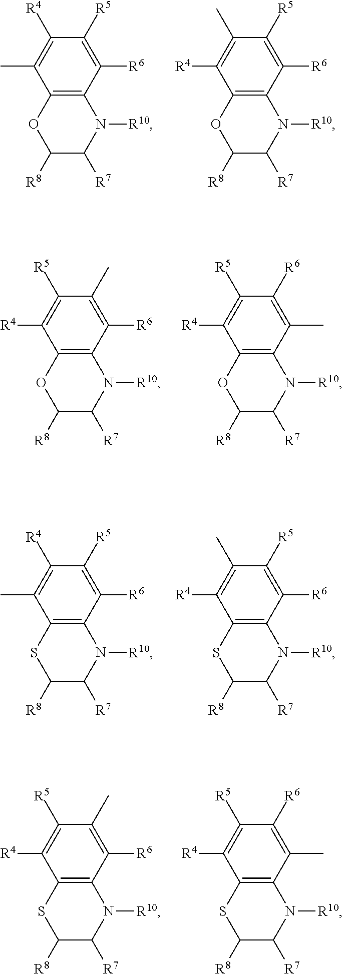

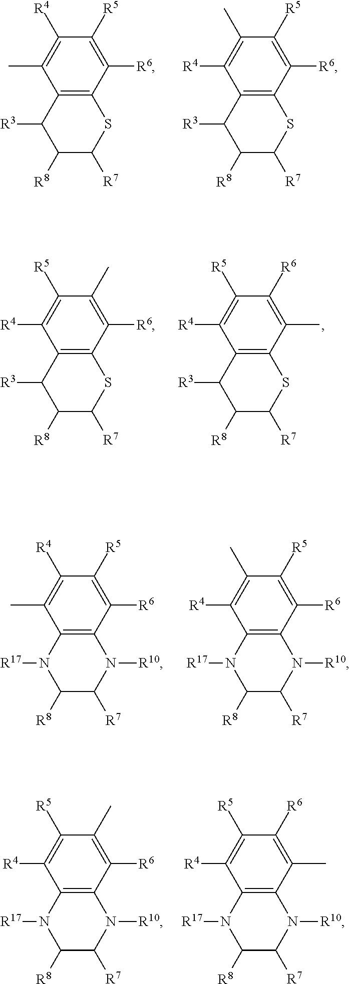

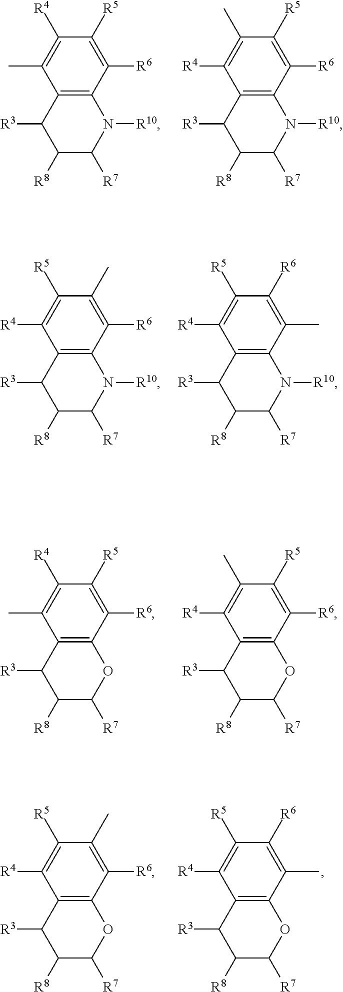

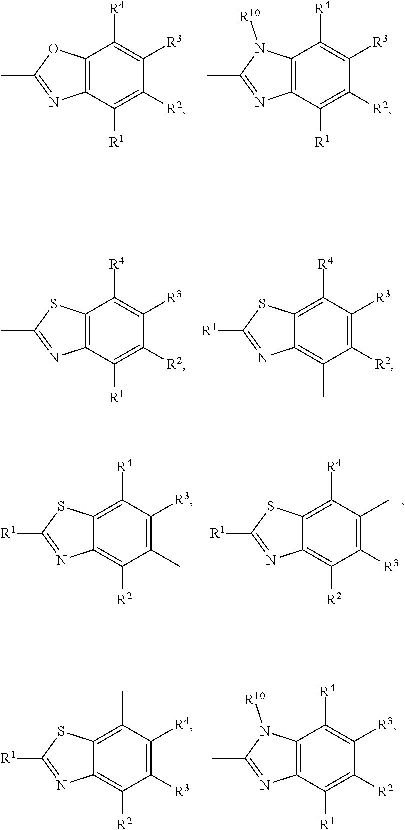

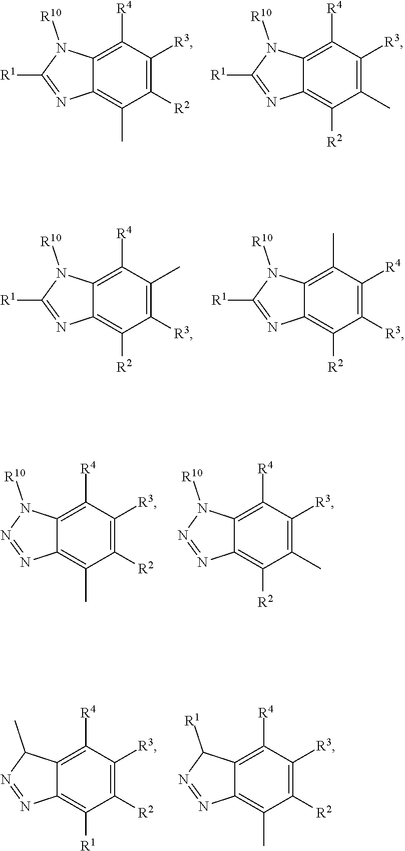

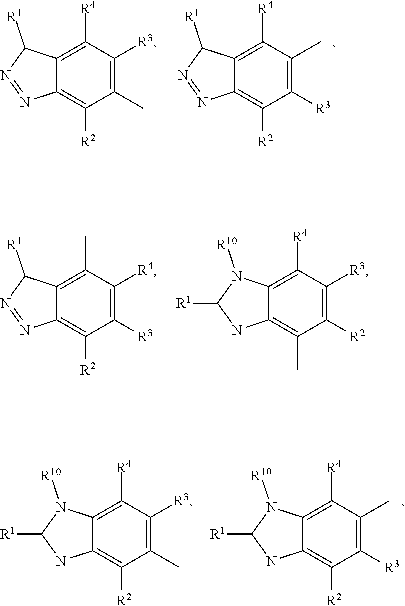

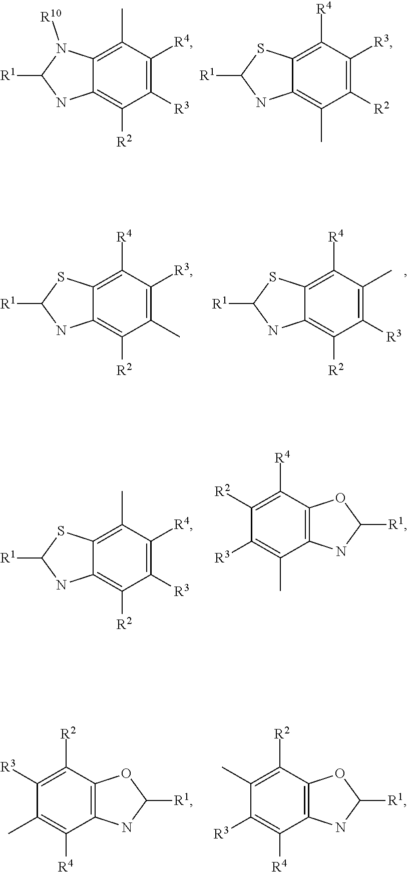

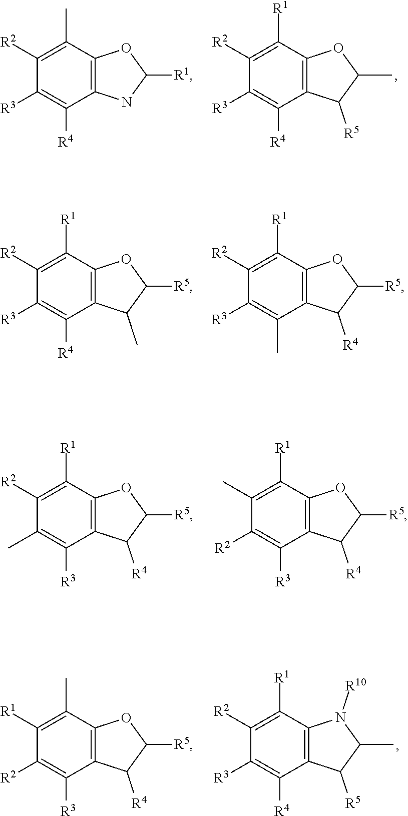

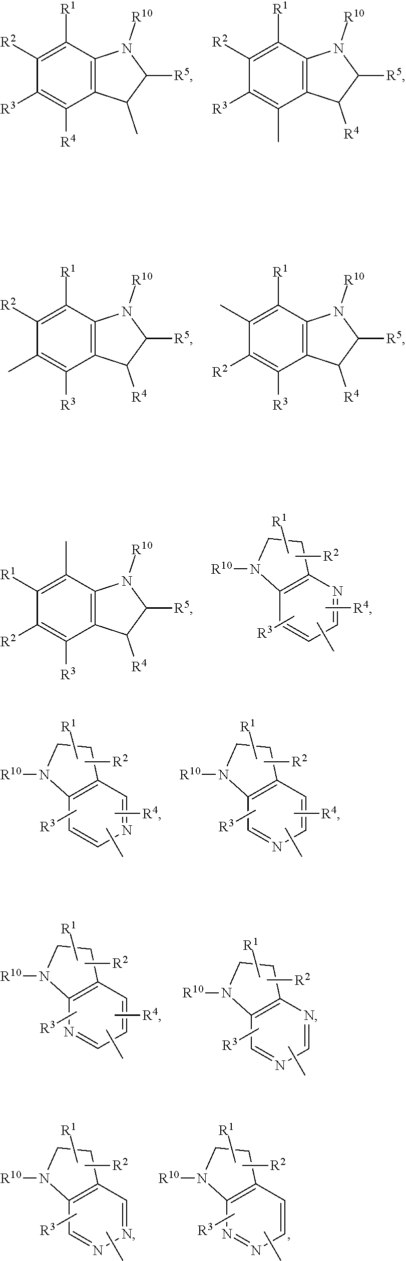

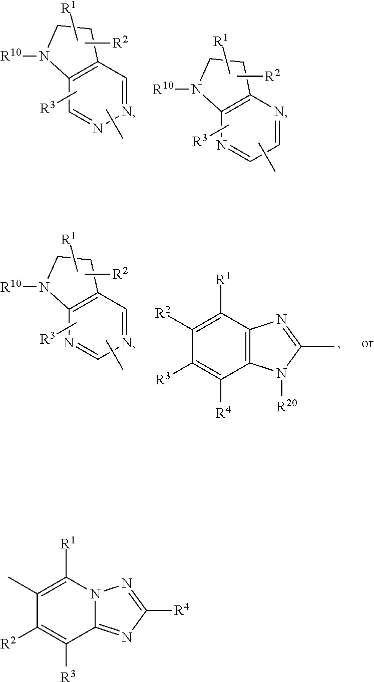

wherein R¹, R², R³, R⁴, R⁵, R⁶, R⁷, R⁸, and R⁹ represent independently of each other —H, —F, —Cl, —Br, —I, -Ph, —OH, —OCH₃, —OC₂H₅, —OC₃H₇, —OCH(CH₃)₂, —OC₄H₉, —OCH(CH₃)CH₂CH₃, —OCH₂CH(CH₃)₂, —OC(CH₃)₃, —O-cyclo-C₃H₅, —O-cyclo-C₄H₇, —O-cyclo-C₅H₉, —O-cyclo-C₆H₁₁, —OPh, —OCH₂-Ph, —OCH₂CH₂-Ph —OCH═CH₂, —OCH₂—CH═CH₂, —OCH₂CH₂—CH═CH₂, —OCF₃, —OC₂F₅, —SCH₃, —SC₂H₅, —SC₃H₇, —SCH(CH₃)₂, —SC₄H₉, —SCH(CH₃)CH₂CH₃, —SCH₂CH(CH₃)₂, —SC(CH₃)₃, —NO₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(C₄H₉)₂, —N(cyclo-C₃H₅)₂, —N[CH(CH₃)₂]₂, —N[C(CH₃)₃]₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH₂—CH₂—CH(CH₃)₂, —CH(CH₃)—CH(CH₃)—CH₃, —C(CH₃)₂—C₂H₅, —C₆H₁₃, —CH(CH₃)—C₄H₉, —CH₂—CH(CH₃)—C₃H₇, —CH₂—CH₂—CH₂—CH(CH₃)₂, —CH(CH₃)—CH(CH₃)—C₂H₅, —C(CH₃)₂—C₃H₇, —C₇H₁₅, —CH(CH₃)—C₅H₁₁, —CH₂—CH(CH₃)—C₄H₉, —CH₂—CH₂—CH₂—CH₂—CH(CH₃)₂, —C₈H₁₇, —C₉H₁₉, —C₁₀H₂₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —CH₂F, —CF₂I, —CHF₂, —CF₃, —CH₂I, —CH₂Br, —CH₂I, —CH₂—CH₂F, —CH₂—CHF₂, —CH₂—CF₃, —CH₂—CH₂I, —CH₂—CH₂Br, —CH₂—CH₂I, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COO-cyclo-C₄H₇, —COO-cyclo-C₅H₉, —COO-cyclo-C₆H₁₁, —COOCH(CH₃)₂, —COOC(CH₃)₃, —O—COOCH₃, —O—COOC₂H₅, —O—COOC₃H₇, —O—COO-cyclo-C₃H₅, —O—COOCH(CH₃)₂, —O—COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON [CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃Na, —SO₃K, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—N H-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —B(OH)₂, —B(OR²²),

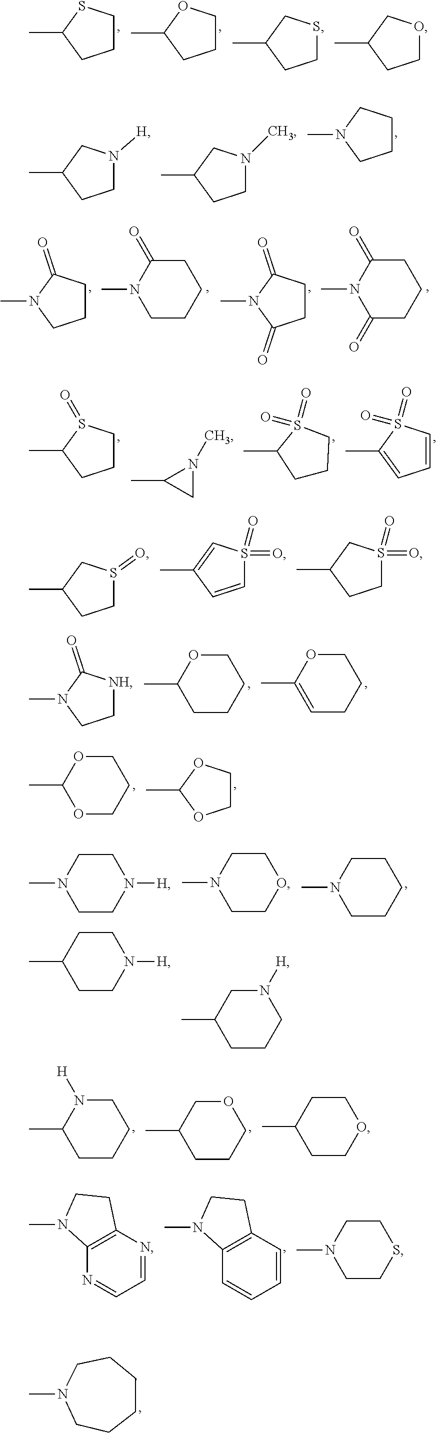

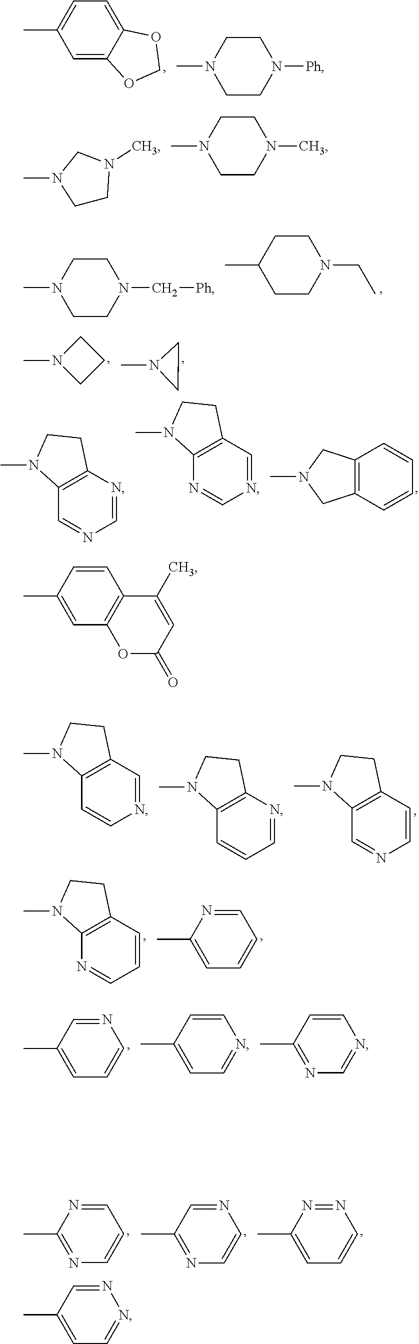

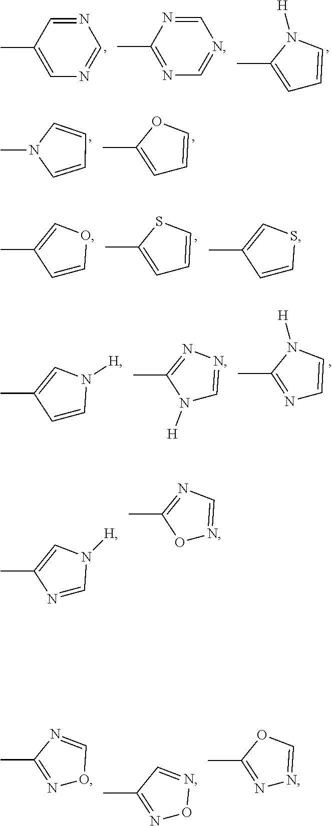

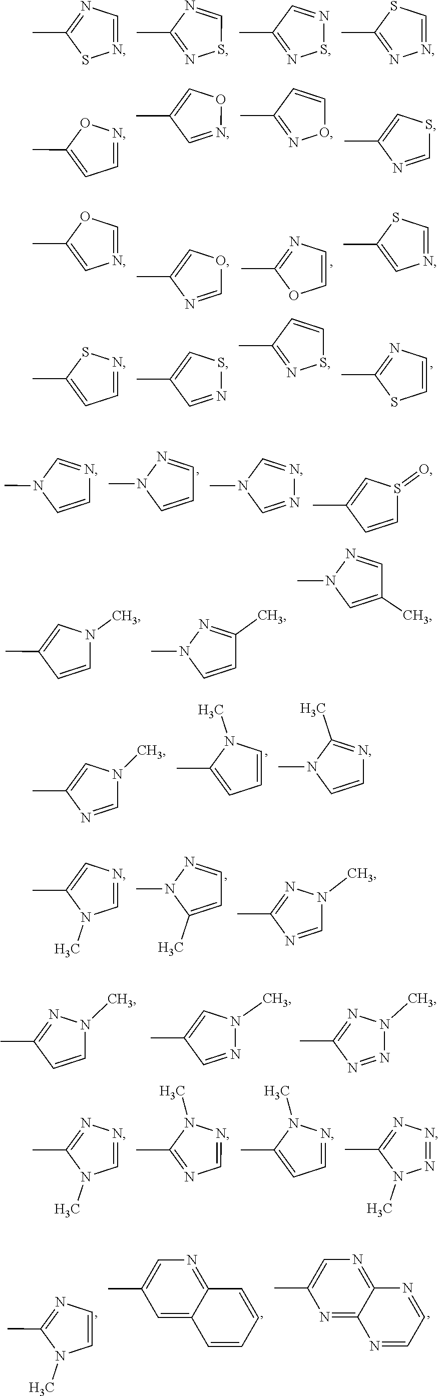

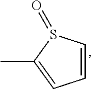

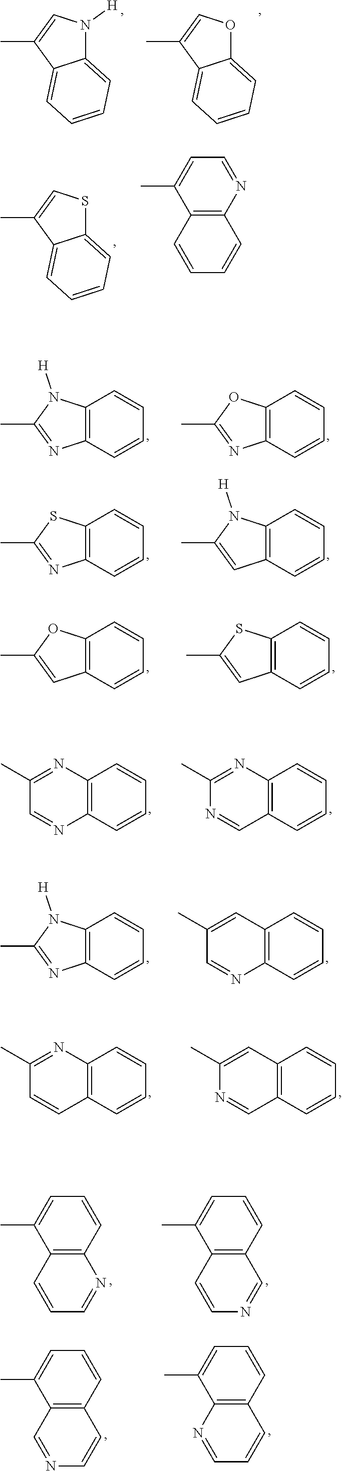

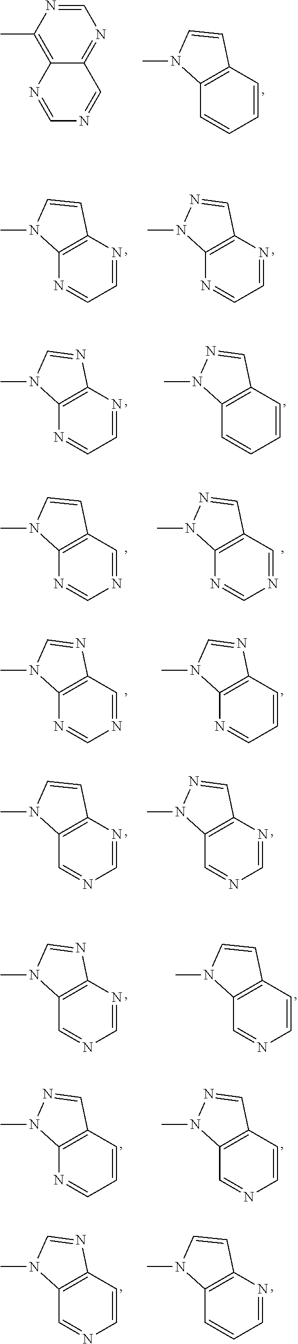

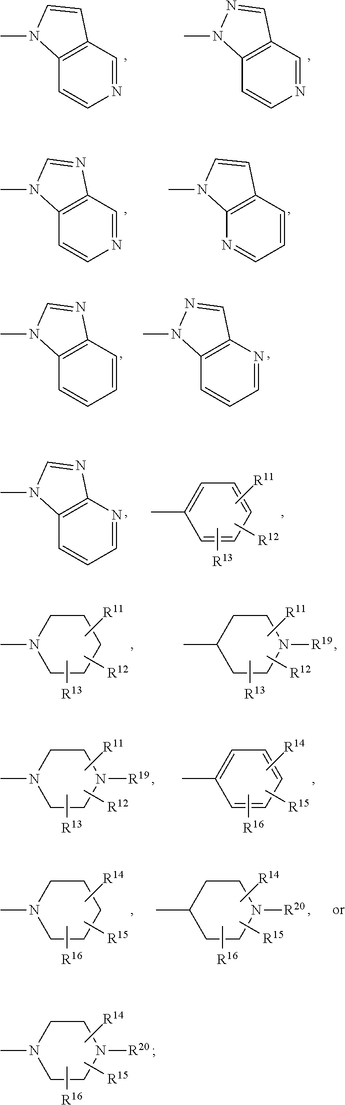

R¹¹ to R¹⁶ represents independently of each other:

—H, —NH₂, —OH, —OCH₃, —OC₂H₅, —OC₃H₇, —OCF₃, —CF₃, —F, —Cl, —Br, —I, —CH₃, —C₂H₅, —C₃H₇, —CH(CH₃)₂, -Ph, or —CN;

R¹⁰, R¹⁷, R¹⁸, R¹⁹, R²⁰, and R²¹ represents independently of each other —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, CH₂—CH₂F, —CH₂—CHF₂, —CH₂—CF₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, -cyclo-C₃H₅, -cyclo-C₄H₇, -cyclo-C₅H₉, -cyclo-C₆H₁₁, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, preferably R²¹ cannot be —H;

R²² represents —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, CH₂—CH₂F, —CH₂—CHF₂, —CH₂—CF₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, -cyclo-C₃H₅,

X represents —OH, —O⁻ or —SCoA,

wherein n is an integer 1, 2, or 3; or a salt thereof,

comprising or consisting of:
A) providing a compound R—CHO (1) and an acyl-CoA (2)

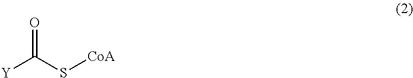

wherein Y represents —H, or —COO⁻,
in a buffer solution with a pH-value in the range of 5.0 to 7.5, optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the α-hydroxyacyl-CoA thioester (I′)

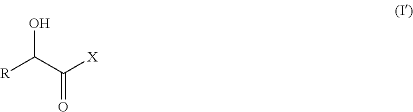

wherein X represents —S—CoA,
and the oxalyl-CoA decarboxylase (3a) is a peptide encoded by a gene selected from Methylobacterium extorquens or a mutant thereof; and optionally comprising the additional step C)
C) cleaving the coenzyme A moiety of said α-hydroxyacyl-CoA thioester (I′) by a hydrolase, α-hydroxyacyl-CoA:oxalate CoA-transferase and/or α-hydroxyacyl-CoA:formate CoA-transferase to obtain the compound of the formula (I′) or a salt thereof

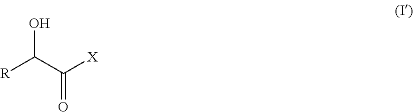

wherein X represents —OH or O⁻,
and the hydrolase is acyl-CoA thioester hydrolase YciA (4) encoded by a gene selected from E. coli.

An embodiment of the present invention is directed to a method for the preparation of an α-hydroxyacyl compound of the formula (I′)

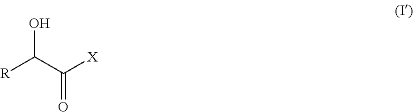

wherein R represents

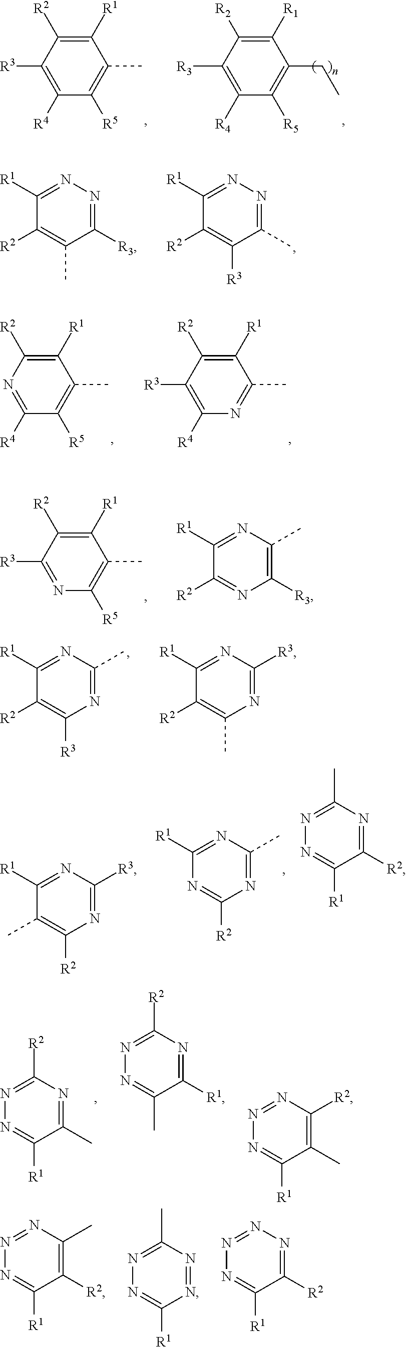

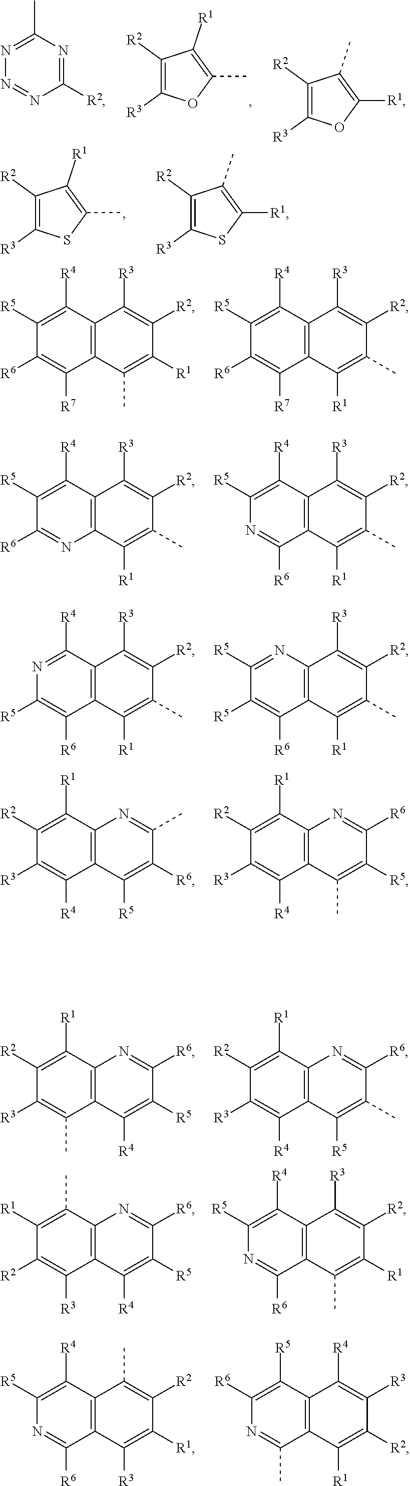

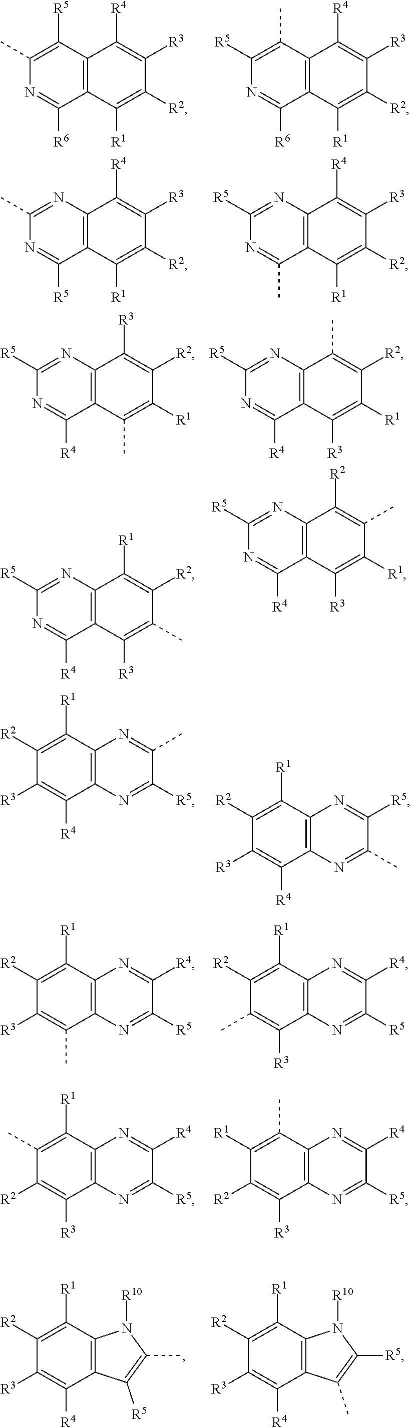

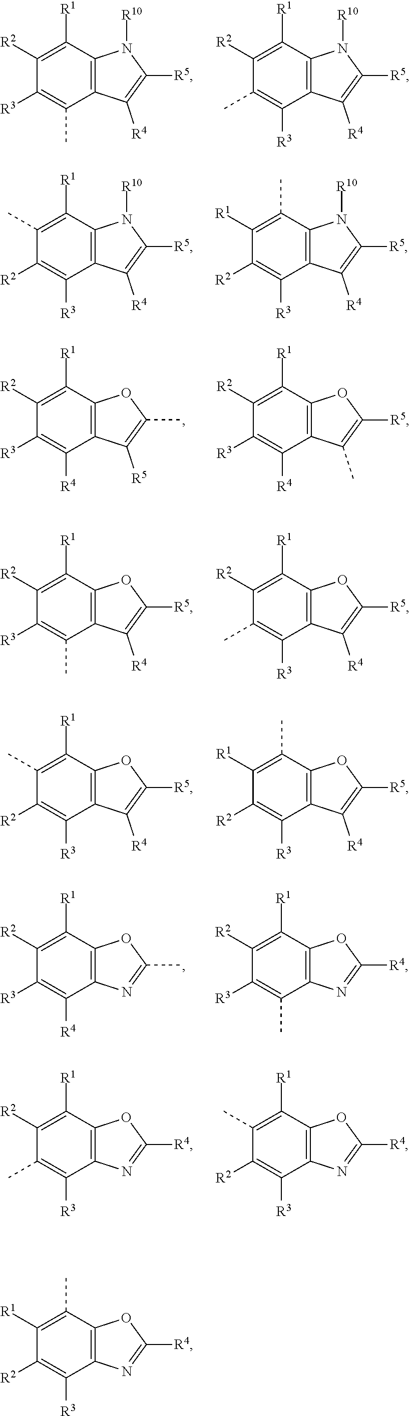

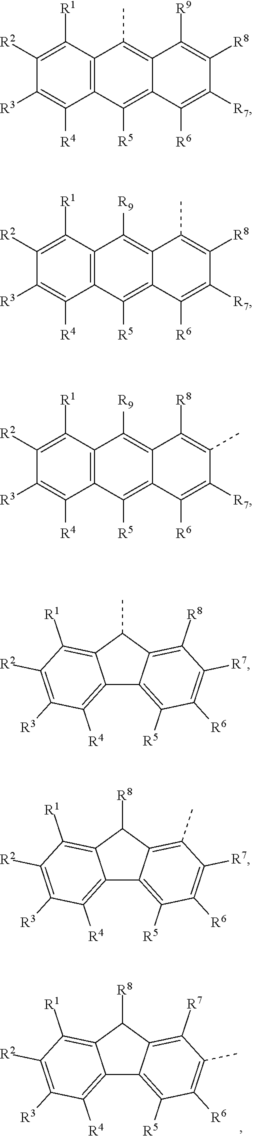

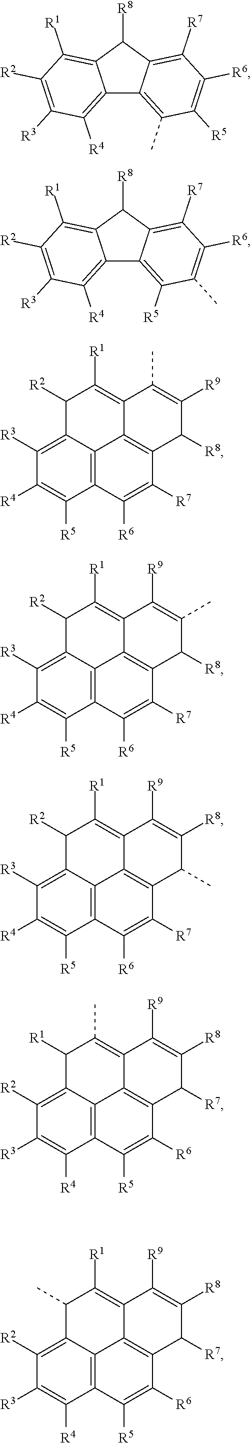

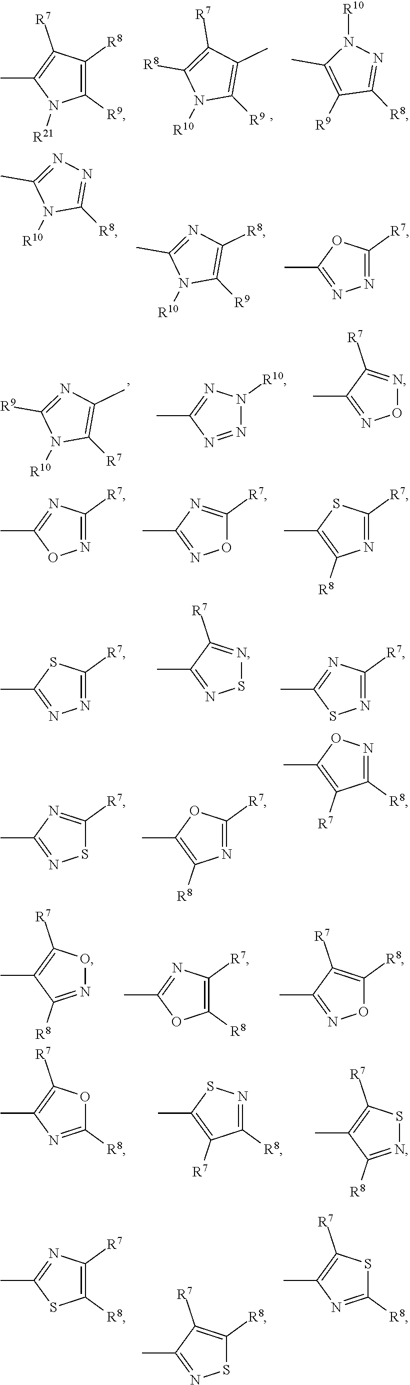

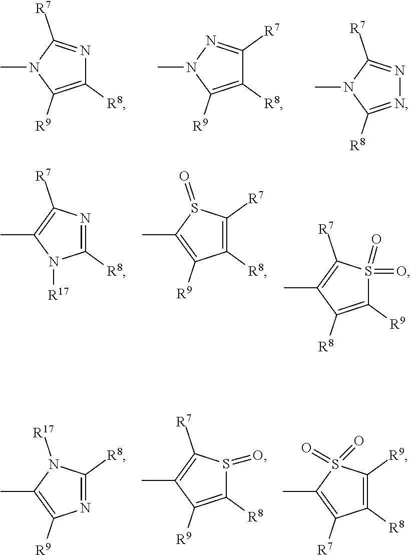

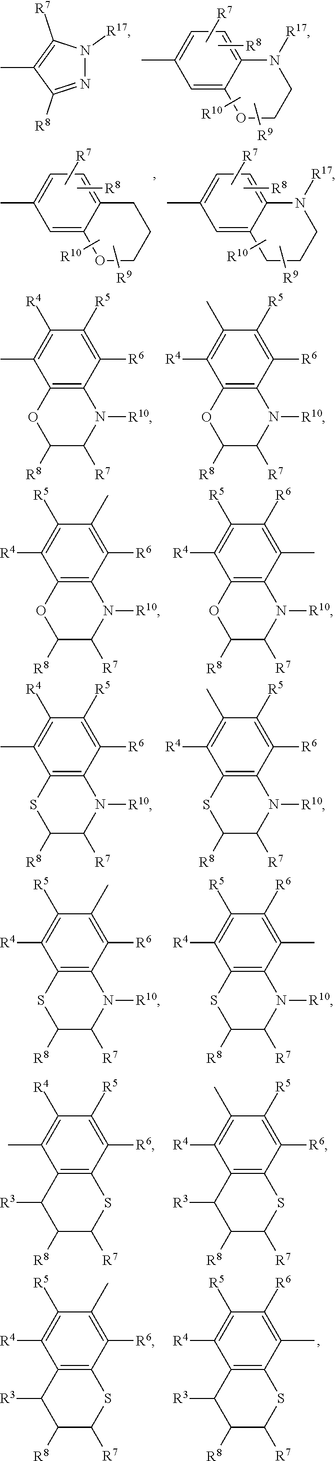

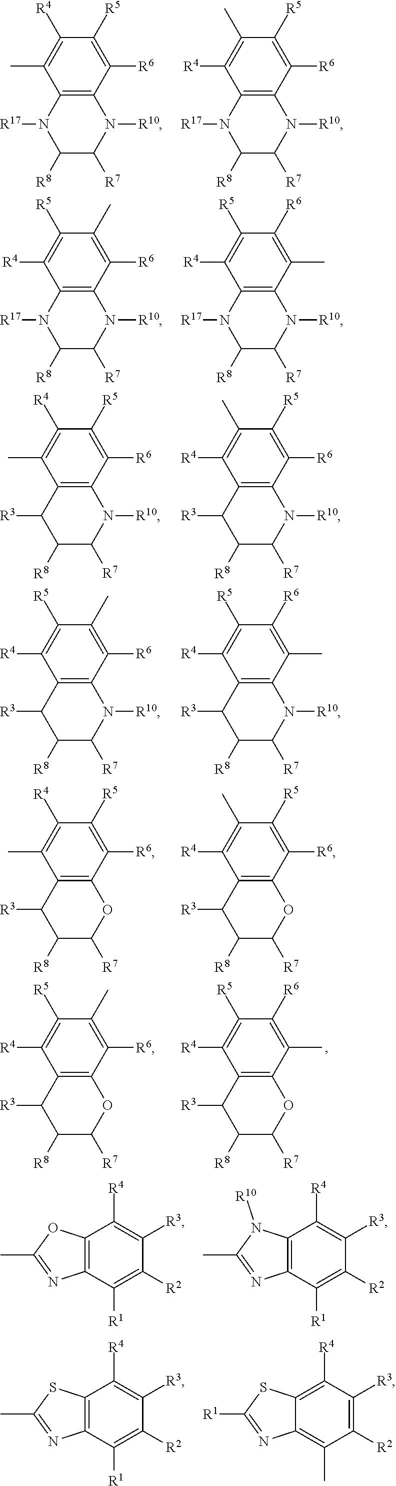

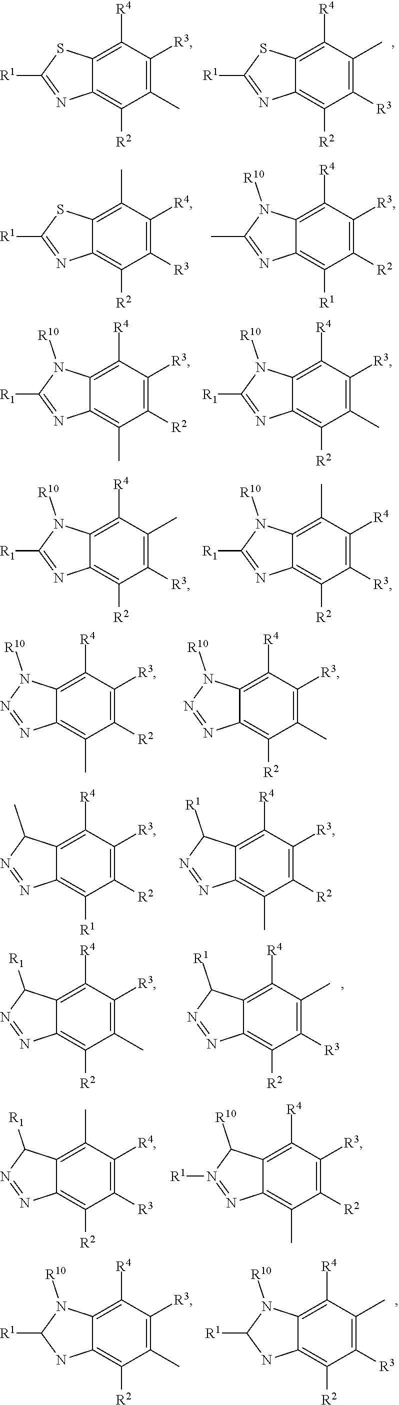

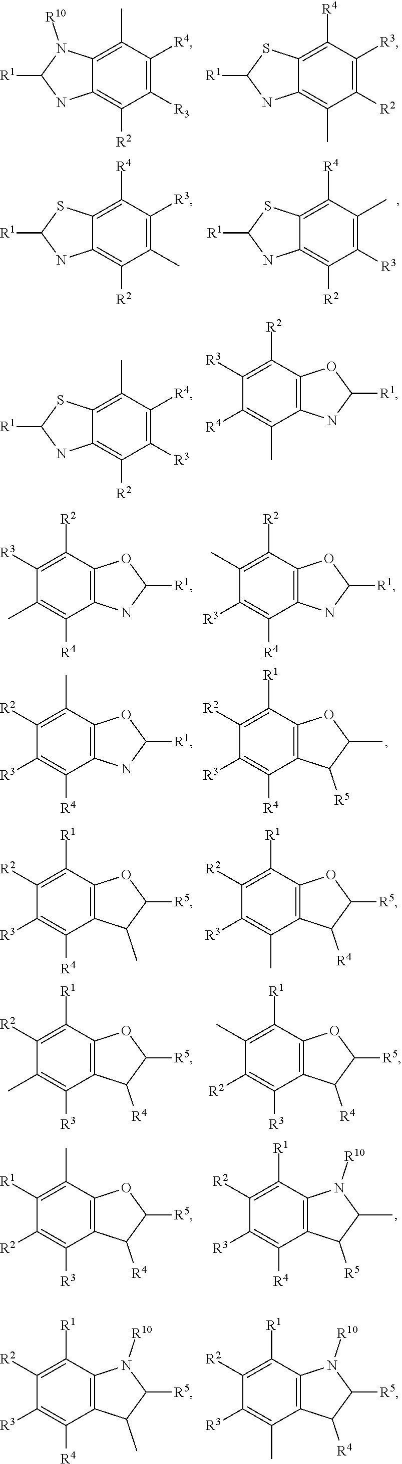

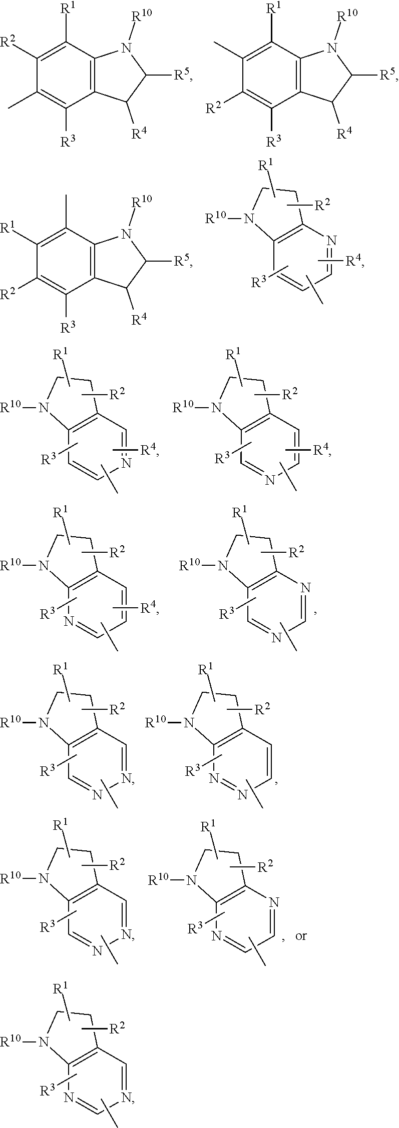

wherein R¹, R², R³, R⁴, R⁵, R⁶, R⁷, R⁸, and R⁹ represent independently of each other —H, —F, —Cl, —Br, —I, -Ph, —OH, —OCH₃, —OC₂H₅, —OC₃H₇, —OCH(CH₃)₂, —OC₄H₉, —OCH(CH₃)CH₂CH₃, —OCH₂CH(CH₃)₂, —OC(CH₃)₃, —O-cyclo-C₃H₅, —O-cyclo-C₄H₇, —O-cyclo-C₅H₉, —O-cyclo-C₆H₁₁, —OPh, —OCH₂-Ph, —OCH₂CH₂-Ph —OCH═CH₂, —OCH₂—CH═CH₂, —OCH₂CH₂—CH═CH₂, —OCF₃, —OC₂F₅, —SCH₃, —SC₂H₅, —SC₃H₇, —SCH(CH₃)₂, —SC₄H₉, —SCH(CH₃)CH₂CH₃, —SCH₂CH(CH₃)₂, —SC(CH₃)₃, —NO₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(C₄H₉)₂, —N(cyclo-C₃H₅)₂, —N[CH(CH₃)₂]₂, —N[C(CH₃)₃]₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH₂—CH₂—CH(CH₃)₂, —CH(CH₃)—CH(CH₃)—CH₃, —C(CH₃)₂—C₂H₅, —C₆H₁₃, —CH(CH₃)—C₄H₉, —CH₂—CH(CH₃)—C₃H₇, —CH₂—CH₂—CH₂—CH(CH₃)₂, —CH(CH₃)—CH(CH₃)—C₂H₅, —C(CH₃)₂—C₃H₇, —C₇H₁₅, —CH(CH₃)—C₅H₁₁, —CH₂—CH(CH₃)—C₄H₉, —CH₂—CH₂—CH₂—CH₂—CH(CH₃)₂, —C₈H₁₇, —C₉H₁₉, —C₁₀H₂₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —CH₂F, —CF₂I, —CHF₂, —CF₃, —CH₂I, —CH₂Br, —CH₂I, —CH₂—CH₂F, —CH₂—CHF₂, —CH₂—CF₃, —CH₂—CH₂I, —CH₂—CH₂Br, —CH₂—CH₂I, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COO-cyclo-C₄H₇, —COO-cyclo-C₅H₉, —COO-cyclo-C₆H₁₁, —COOCH(CH₃)₂, —COOC(CH₃)₃, —O—COOCH₃, —O—COOC₂H₅, —O—COOC₃H₇, —O—COO-cyclo-C₃H₅, —O—COOCH(CH₃)₂, —O—COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂,

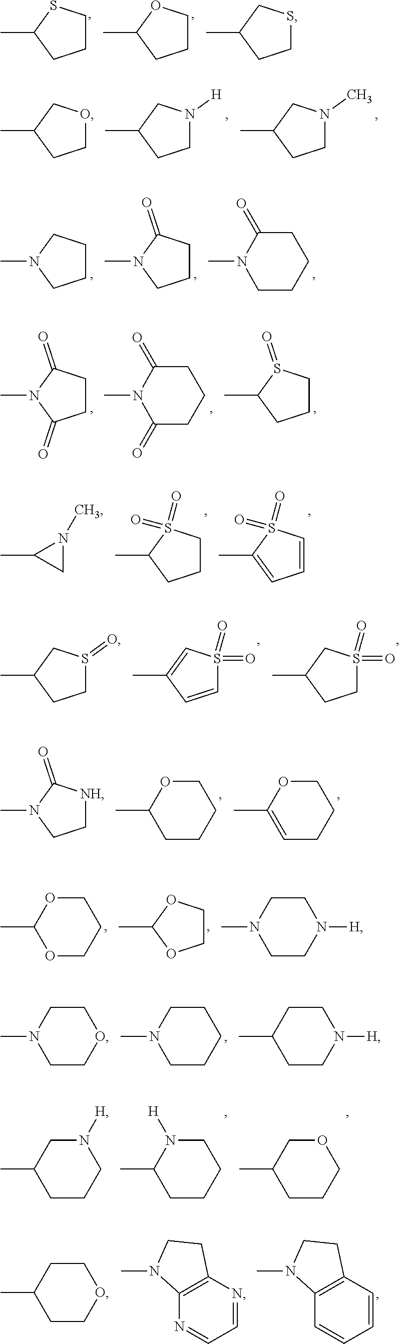

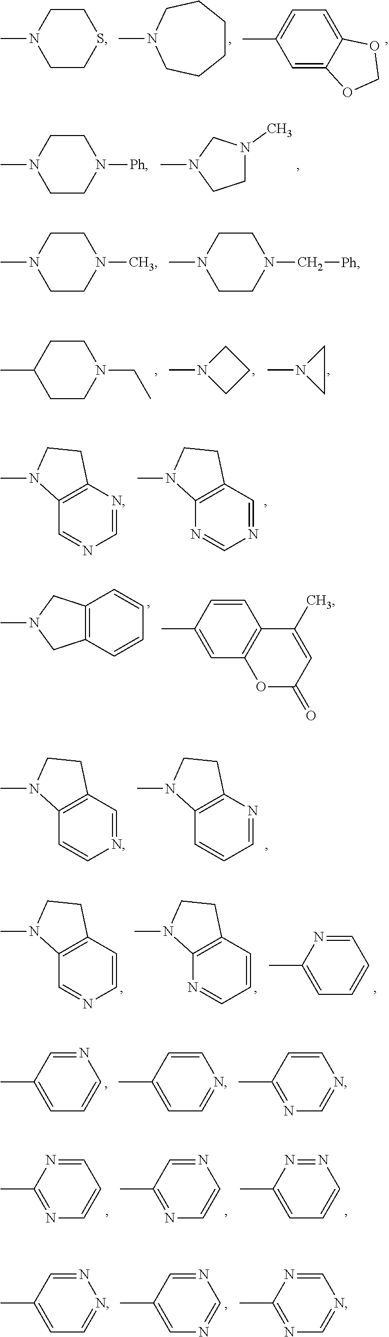

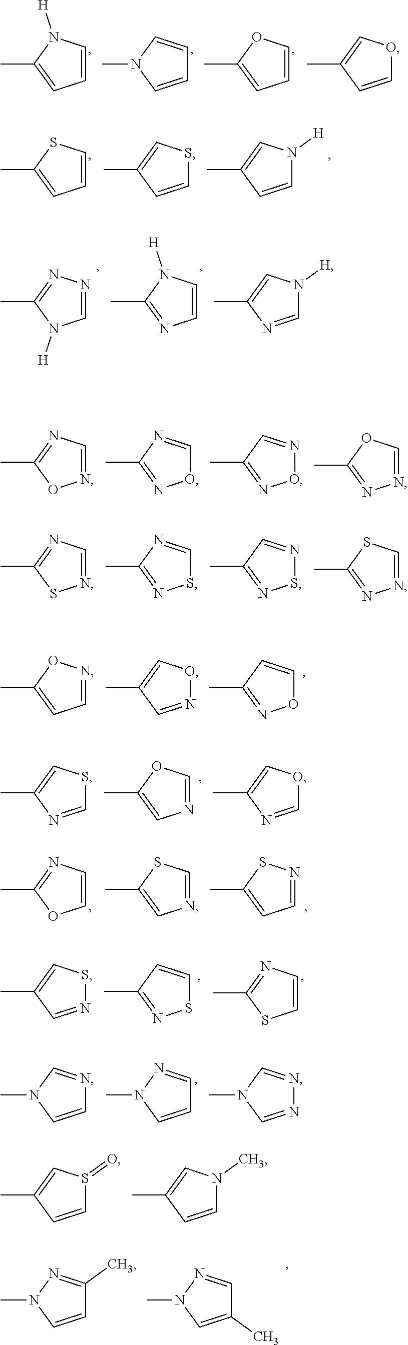

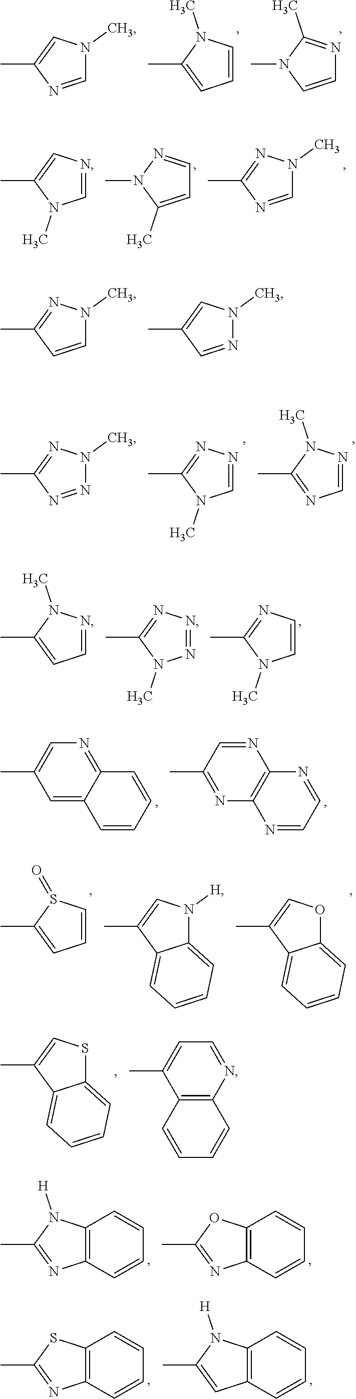

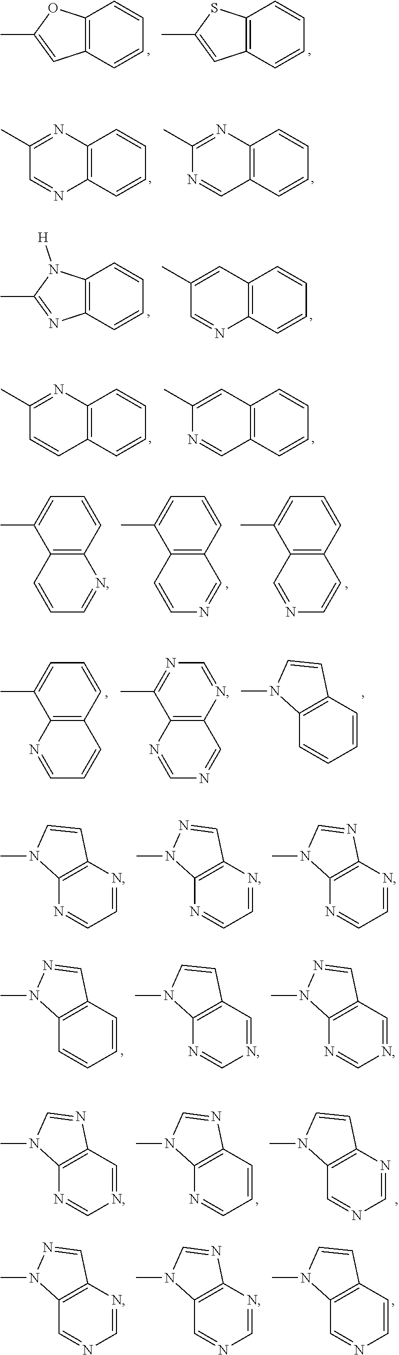

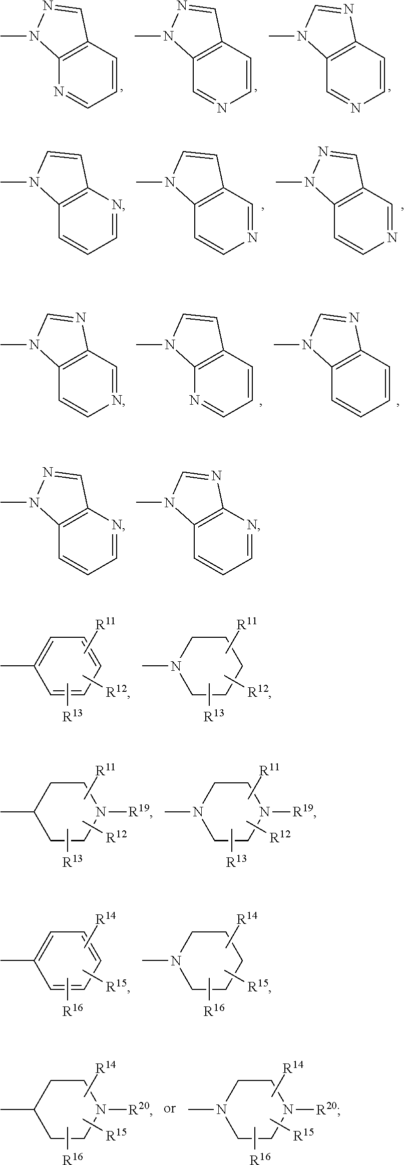

R¹¹ to R¹⁶ represents independently of each other:

—H, —NH₂, —OH, —OCH₃, —OC₂H₅, —OC₃H₇, —OCF₃, —CF₃, —F, —Cl, —Br, —I, —CH₃, —C₂H₅, —C₃H₇, —CH(CH₃)₂, -Ph, or —CN;

R¹⁰, R¹⁷, R¹⁸, R¹⁹, R²⁰, and R²¹ represents independently of each other —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, CH₂—CH₂F, —CH₂—CHF₂, —CH₂—CF₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, preferably R²¹ cannot be —H,

X represents —OH, —O⁻ or —SCoA,

wherein n is an integer 1, 2, or 3; or a salt thereof,

comprising or consisting of:
A) providing a compound R—CHO (1) and an acyl-CoA (2)

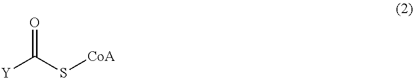

wherein Y represents —H, or —COO⁻,
in a buffer solution with a pH-value in the range of 5.0 to 7.5, optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the α-hydroxyacyl-CoA thioester (I′)

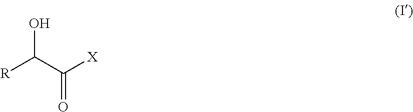

wherein X represents —S—CoA,
and the oxalyl-CoA decarboxylase (3a) is a peptide encoded by a gene selected from Methylobacterium extorquens or a mutant thereof; and optionally comprising the additional step C)
C) cleaving the coenzyme A moiety of said α-hydroxyacyl-CoA thioester (I′) by a hydrolase, α-hydroxyacyl-CoA:oxalate CoA-transferase and/or α-hydroxyacyl-CoA:formate CoA-transferase to obtain the compound of the formula (I′) or a salt thereof

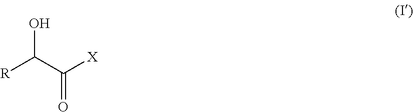

wherein X represents —OH or O⁻,
and the hydrolase is acyl-CoA thioester hydrolase YciA (4) encoded by a gene selected from E. coli.

An example for the carbon-carbon-bond formation is depicted in FIGS. 1 and 2.

The use of oxalyl-CoA decarboxylase from Methylobacterium extorquens or a mutant thereof (3a) were not known in the state of the art. Remarkably, the oxalyl-CoA decarboxylase is normally used for the carbon-carbon-bond cleavage. In particular, the oxalyl-CoA decarboxylase from Methylobacterium extorquens exhibits a high activity towards aromatic aldehydes in comparison to aliphatic aldehydes (example 7, and table 4).

Methylobacterium extorquens is also known as Methylorubrum extorquens.

A further aspect of the present invention is related to the use of peptide OXCMe encoded by a gene from Methylobacterium extorquens or mutant thereof for the carbon-carbon-bond formation in particular for the synthesis of an α-hydroxy carboxylic acid, salts thereof or an α-hydroxyacyl-CoA thioester.

In addition, it could be shown that the α-hydroxyacyl-CoA thioester formed can be hydrolyzed by a hydrolase YciA from E. coli (4) to yield an α-hydroxy carboxylic acid, or its salts. The process can be carried out either in subsequent manner or in one pot.

Surprisingly, the hydrolase YciA from E. coli is selective for aromatic α-hydroxyacyl-CoA thioester, i.e. the hydrolysis of oxalyl-CoA or formyl-CoA is slower than for α-hydroxyacyl-CoA thioester.

A further aspect of the present invention is related to the use of a composition comprising a peptide oxalyl-CoA decarboxylase OXC_{Me} encoded by a gene from Methylobacterium extorquens or a mutant thereof for the preparation of an α-hydroxy carboxylic acid, salt thereof or α-hydroxyacyl-CoA thioester starting from oxalyl-CoA and an aldehyde.

### DESCRIPTION OF THE INVENTION

“Aromatic aldehydes”, as used herein, refer to a compound of the formula (I), (I′), (II), (II″), (3), (3′), wherein the substituent R is an aromatic substituent including benzyl, homo-benzyl and bis-homo-benzyl substituents.

“Aliphatic aldehydes”, as used herein, refer to a compound of (I), (I′), (II), (II″), (3), (3′), wherein the substituent R is an aliphatic substituent.

The substituent —SCoA as used herein refers to the coenzyme, and not to the disulfide. The sulphur is depicted in order to emphasize the position where it bonds.

The stereochemistry of formula (I), formula (II) and formula (3) as depicted in the following refers to an enantiomeric excess of the depicted enantiomer.

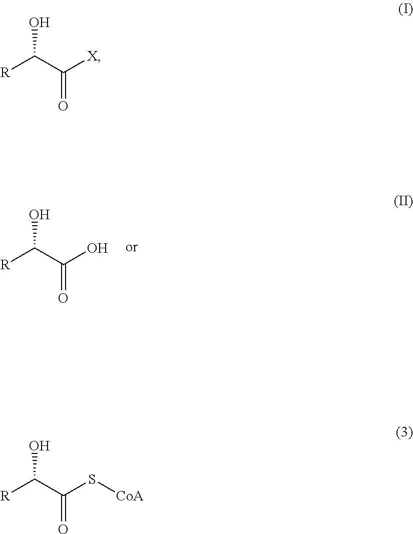

Thus, the compounds can also be a mixture of enantiomers but with an excess of the depicted enantiomer.

It could be shown that the enantiomer having the (S)-configuration can be prepared in an enantiomeric excess. Thus, the methods according to the invention preferably lead to α-hydroxyacyl compound of the formula (I)

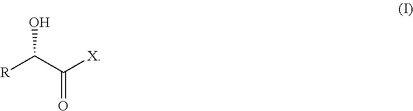

Thus, an embodiment of the invention is related to a method for preparation of the compounds according to a method described herein represented by formula (I)

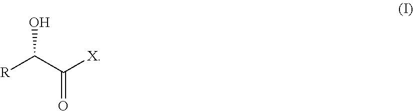

The methods according to the invention are preferably used to prepare an α-hydroxyacyl compound of the formula (I) with an enantiomeric excess of the (S)-enantiomer.

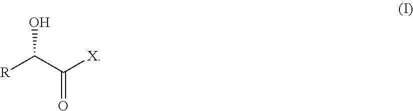

More preferably the method according to the invention is used to prepare a compound of the formula (I), wherein the compound can be mixture of the enantiomers, and wherein the (S)-enantiomer is present in an enantiomeric excess. Preferably, the enantiomeric excess (ee) is at least 5%, preferably 10%, more preferably 15%, more preferably 20%, more preferably 25%, more preferably 30%, more preferably 35%, more preferably 40%, more preferably 45%, more preferably 50%, more preferably 55%, more preferably 60%, more preferably 65%, more preferably 65%, more preferably 70%, more preferably 75%, more preferably 80%, more preferably 85%, more preferably 90%, more preferably 91%, more preferably 92%, more preferably 93%, more preferably 94%, more preferably % 95%, more preferably 96%, more preferably 97%, more preferably 98%, and most preferably 99%.

Moreover, the methods according to the invention preferably lead to α-hydroxyacyl compound of the formula (3)

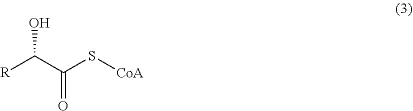

The methods according to the invention are preferably used to prepare an α-hydroxyacyl compound of the formula (I) with an enantiomeric excess of the (S)-enantiomer.

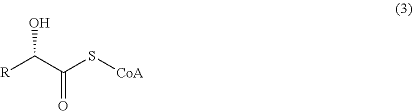

More preferably the method according to the invention is used to prepare a compound of the formula (3), wherein the compound can be mixture of the enantiomers, and wherein the (S)-enantiomer is present in an enantiomeric excess. Preferably, the enantiomeric excess (ee) is at least 5%, preferably 10%, more preferably 15%, more preferably 20%, more preferably 25%, more preferably 30%, more preferably 35%, more preferably 40%, more preferably 45%, more preferably 50%, more preferably 55%, more preferably 60%, more preferably 65%, more preferably 65%, more preferably 70%, more preferably 75%, more preferably 80%, more preferably 85%, more preferably 90%, more preferably 91%, more preferably 92%, more preferably 93%, more preferably 94%, more preferably % 95%, more preferably 96%, more preferably 97%, more preferably 98%, and most preferably 99%.

Moreover, the methods according to the invention preferably lead to α-hydroxyacyl compound of the formula (II)

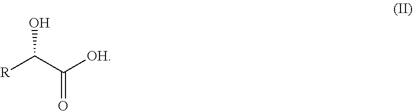

The methods according to the invention are preferably used to prepare an α-hydroxyacyl compound of the formula (II) with an enantiomeric excess of the (S)-enantiomer

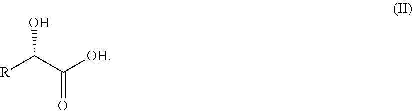

More preferably the method according to the invention is used to prepare a compound of the formula (II), wherein the compound can be mixture of the enantiomers, and wherein the (S)-enantiomer is present in an enantiomeric excess. Preferably, the enantiomeric excess (ee) is at least 5%, preferably 10%, more preferably 15%, more preferably 20%, more preferably 25%, more preferably 30%, more preferably 35%, more preferably 40%, more preferably 45%, more preferably 50%, more preferably 55%, more preferably 60%, more preferably 65%, more preferably 65%, more preferably 70%, more preferably 75%, more preferably 80%, more preferably 85%, more preferably 90%, more preferably 91%, more preferably 92%, more preferably 93%, more preferably 94%, more preferably % 95%, more preferably 96%, more preferably 97%, more preferably 98%, and most preferably 99%.

Therefore, an embodiment according to the invention is related to a method for the preparation of an α-hydroxyacyl compound of the formula (I)

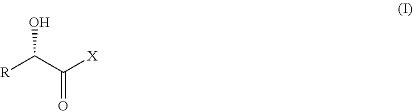

comprising:
A) providing a compound R—CHO (1) and an acyl-CoA (2)

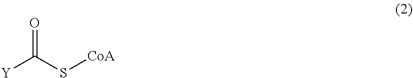

wherein Y represents —H, or —COO⁻,
in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (I)

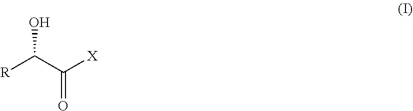

wherein X represents —S—CoA,
and the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (I) by a hydrolase, α-hydroxyacyl-CoA:oxalate CoA-transferase and/or α-hydroxyacyl-CoA:formate CoA-transferase to obtain the compound of the formula (I) or a salt thereof

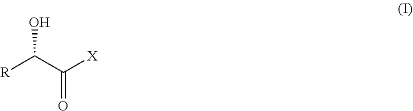

wherein X represents —OH or O⁻,
and the hydrolase is acyl-CoA thioester hydrolase YciA (4) encoded by a gene selected from E. coli.

The peptide in a method according to the invention is preferably used in its isolated form, i.e. the peptide is isolated from the microbe or it is not contained in a microbe.

Herein, the first time the enantioselective carbon-carbon-formation of an α-hydroxyacyl compound according to formula (I), formula (II) or formula (3) by means of an oxalyl-CoA decarboxylase could be shown. In particular, the conditions are suitable for the synthesis of the (S)-enantiomer (example 14, table 5).

It could be shown that the synthesis of α-hydroxyacyl-CoA thioester is faster when oxalyl-CoA thioester is provided as a C₁-unit source (see FIG. 14). Thus, in step A) of a method according to the invention acyl-CoA (2) being provided is preferably an oxalyl-CoA (2′).

Thus, an embodiment of the invention is related to a method for the preparation of the compounds according to a method described herein represented by formula (I′)

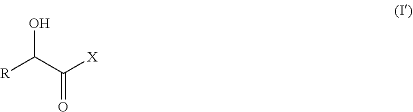

and wherein in step A) an acyl-CoA (2) is provided

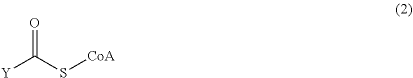

wherein Y represents —COO⁻.

A further embodiment of the invention is related to a method for the preparation of the compounds according to a method described herein represented by formula (I)

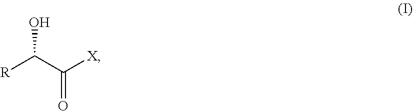

and wherein in step A) an acyl-CoA (2) is provided

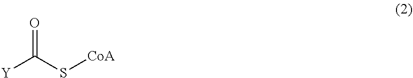

wherein Y represents —COO⁻.

The oxally-CoA according to formula (2)

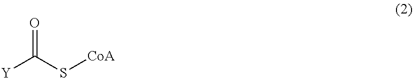

wherein Y represents —COO⁻ is also represented by formula (2′)

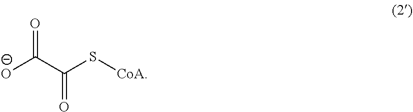

The α-hydroxyacyl-CoA thioester according to formula (I′)

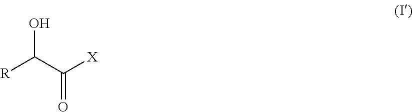

wherein X represents —S—CoA is also represented by formula (3′)

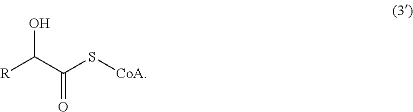

A compound according to formula (I′)

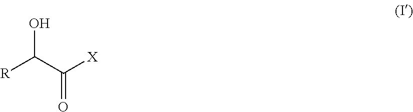

wherein X represents —OH or O⁻ is also represented by formula (II′)

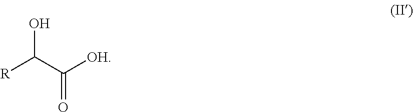

The (S)-α-hydroxyacyl-CoA thioester according to formula (I)

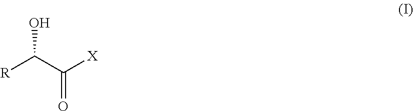

wherein X represents —S—CoA is also represented by formula (3)

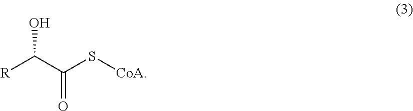

A compound according to formula (I)

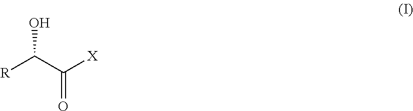

wherein X represents —OH or O⁻ is also represented by formula (II)

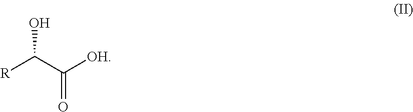

Thus, an embodiment of the invention is directed to a method for preparation of a compound of the formula (I′)

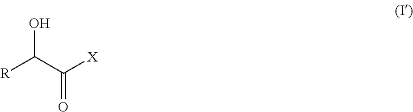

comprising:
A) providing a compound R—CHO (1) and an oxalyl-CoA (2′)

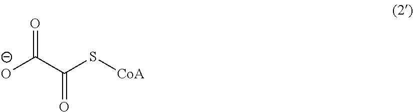

in a buffer solution with a pH-value in the range of 5.0 to 7.5, optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the α-hydroxyacyl-CoA thioester (3′)

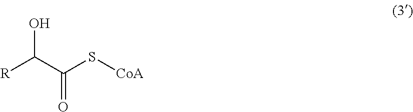

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said α-hydroxyacyl-CoA thioester (3′) by a hydrolase, α-hydroxyacyl-CoA:oxalate CoA-transferase, and/or α-hydroxyacyl-CoA:formate CoA-transferase to obtain the compound of the formula (II′) or a salt thereof

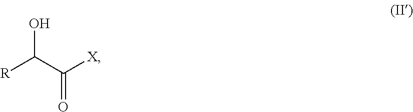

wherein the hydrolase is acyl-CoA thioester hydrolase YciA (4) encoded by a gene selected from E. coli.

Further, an embodiment of the invention is directed to a method for preparation of a compound of the formula (I)

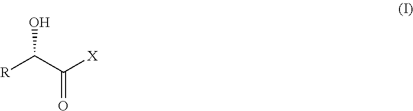

comprising:
A) providing a compound R—CHO (1) and an oxalyl-CoA (2)

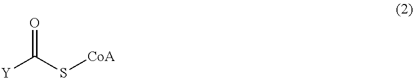

wherein Y represents —COO⁻;
in a buffer solution with a pH-value in the range of 5.0 to 7.5, and optionally together with at least one co-solvent;
B) performing a coupling reaction of R—CHO (1) and oxalyl-CoA (2) by an oxalyl-CoA decarboxylase (3a) in the presence of thiamine diphosphate (3b) and a magnesium (II) cation to obtain the (S)-α-hydroxyacyl-CoA thioester (I)

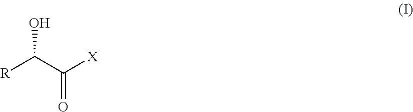

wherein X represents —S—CoA, and the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (I) or a salt thereof

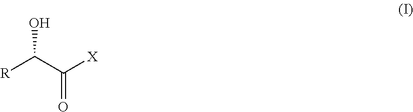

wherein X represents —OH or O⁻, and the hydrolase is acyl-CoA thioester hydrolase YciA (4) encoded by a gene selected from E. coli.

The method according to the invention is preferably used to synthesis a compound of the formula (II′)

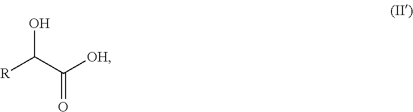

and preferably a compound according to formula (II)

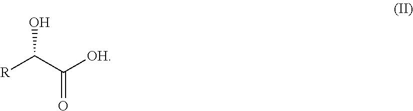

Thus, a preferred embodiment of the present invention is related to a method for preparation of a compound of the formula (II′)

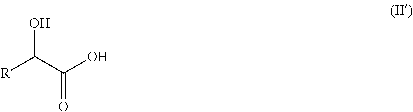

comprising:
A) providing a compound R—CHO (1) and an oxalyl-CoA (2′)

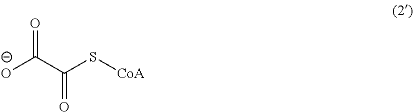

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the α-hydroxyacyl-CoA thioester (3′)

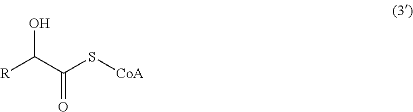

wherein the oxalyl-CoA decarboxylase (3a) is a peptide encoded by a gene selected from Methylobacterium extorquens or a mutant thereof; and
C) cleaving the coenzyme A moiety of said α-hydroxyacyl-CoA thioester (3′) by a hydrolase, α-hydroxyacyl-CoA:oxalate CoA-transferase and/or α-hydroxyacyl-CoA:formate CoA-transferase to obtain the compound of the formula (II′) or a salt thereof

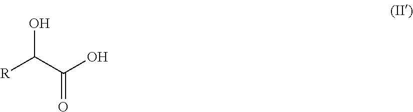

wherein the hydrolase is acyl-CoA thioester hydrolase YciA (4) encoded by a gene selected from E. coli.

A more preferred embodiment of the present invention is related to a method for preparation of a compound of the formula (II)

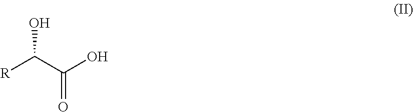

wherein R represents

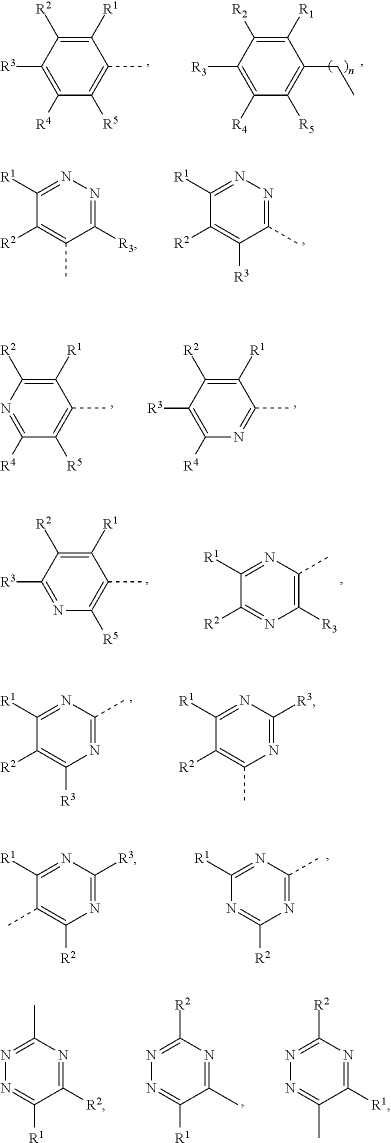

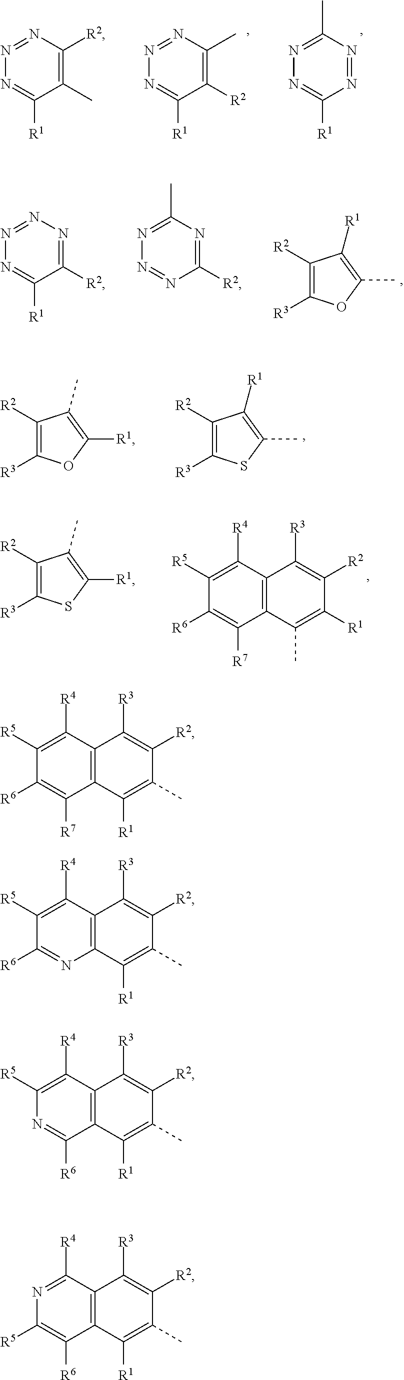

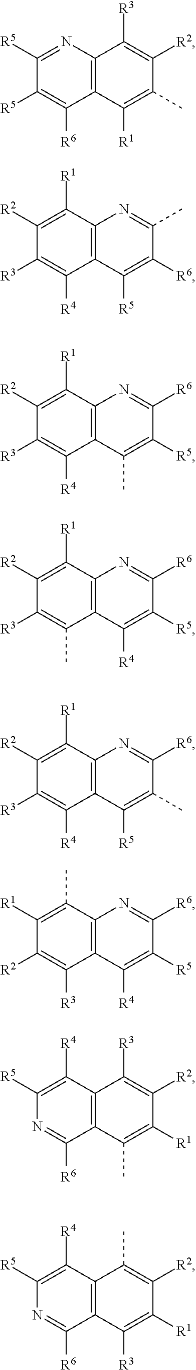

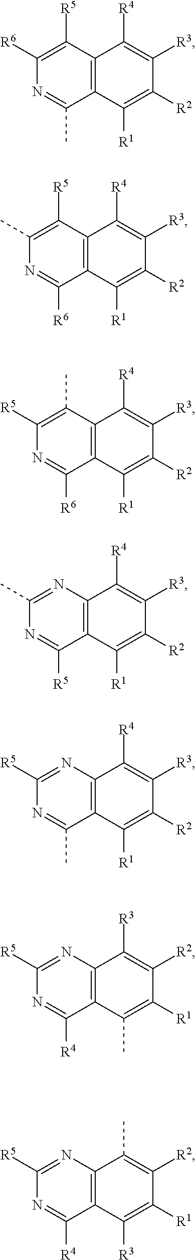

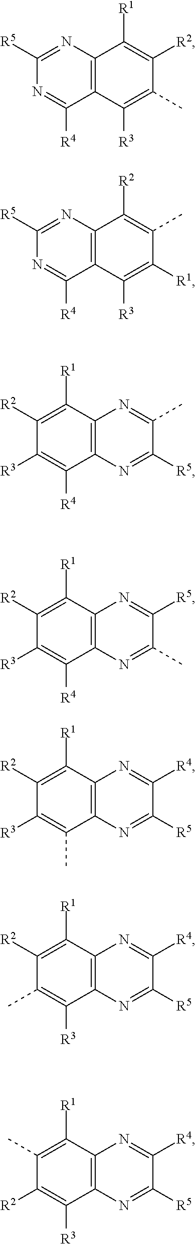

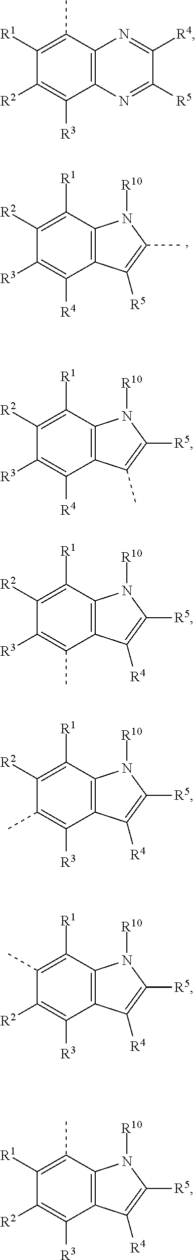

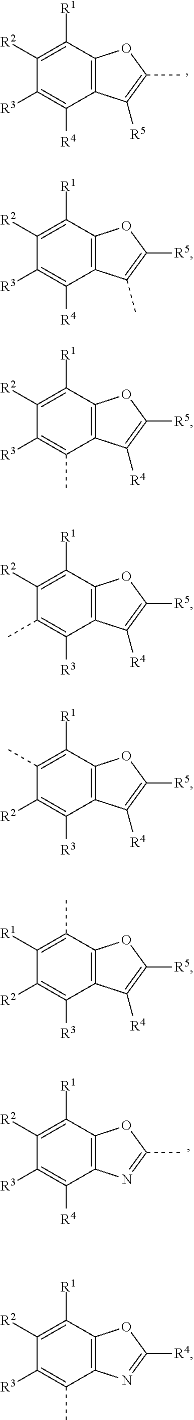

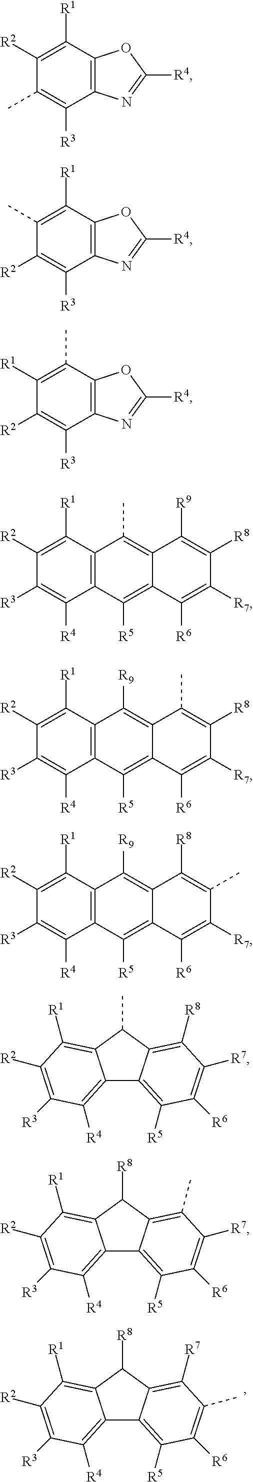

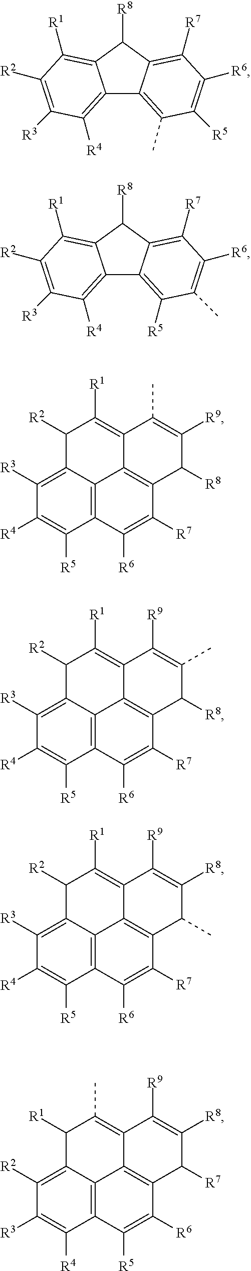

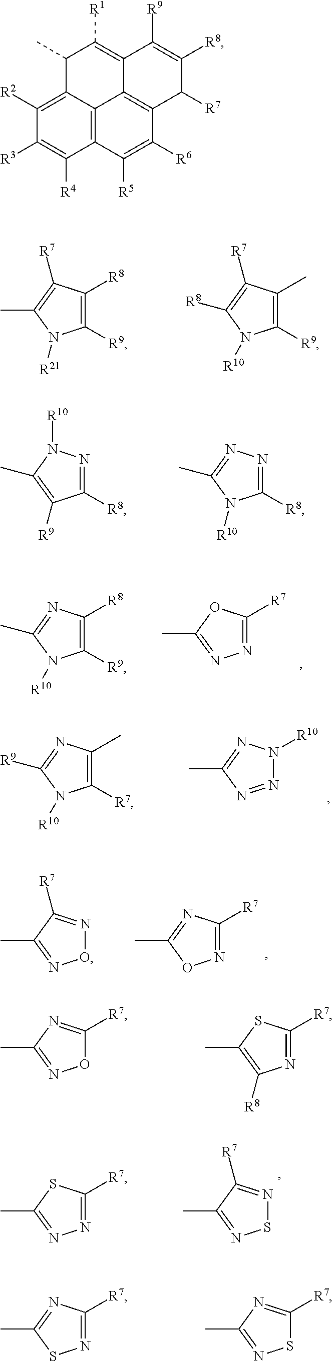

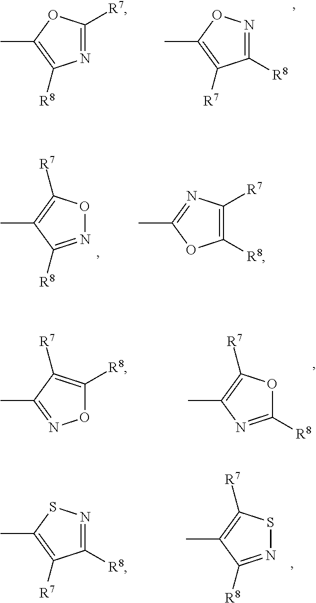

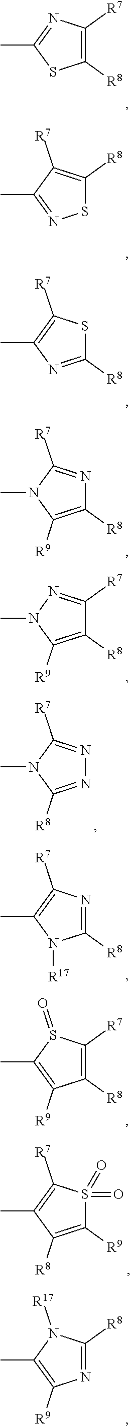

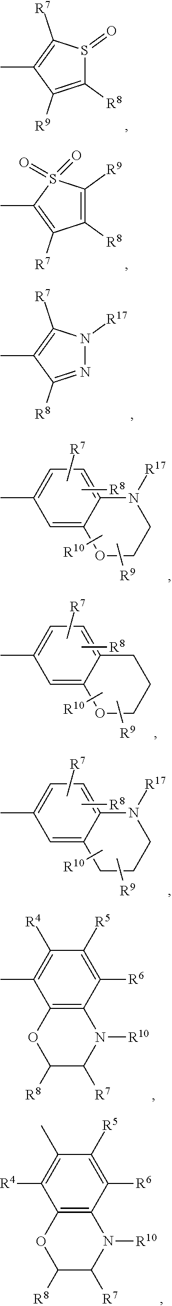

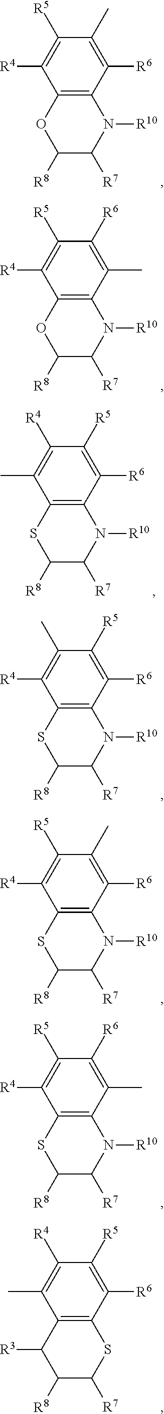

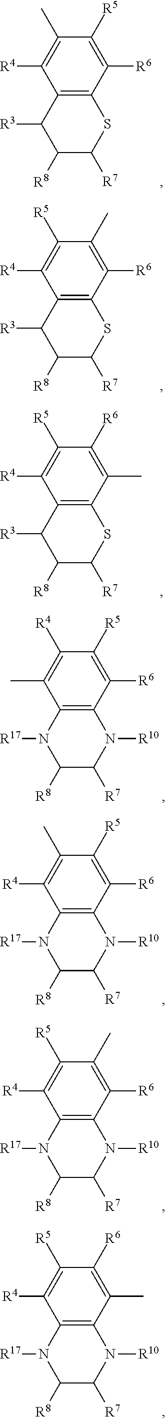

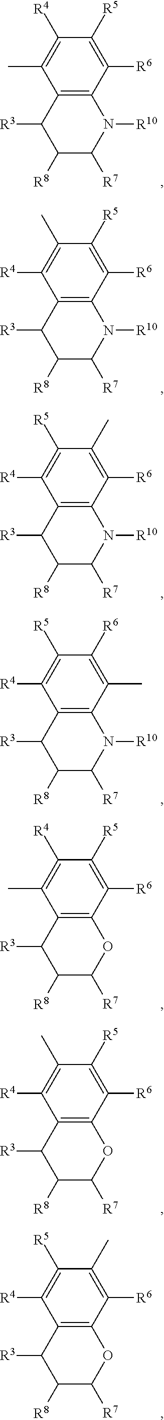

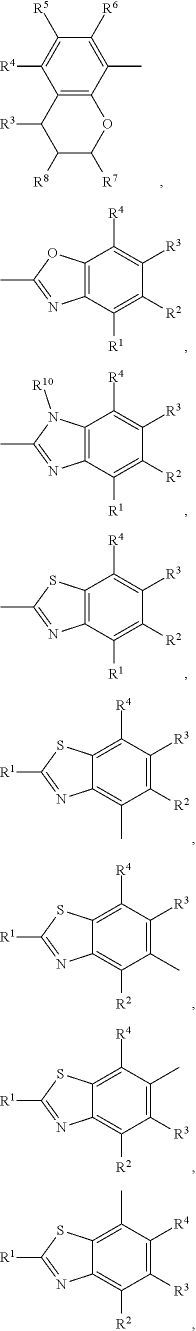

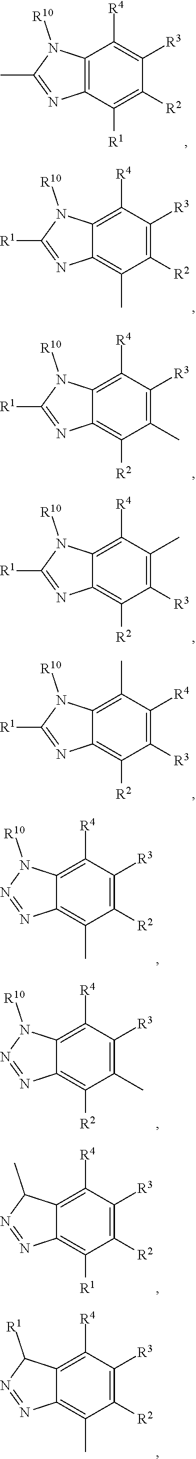

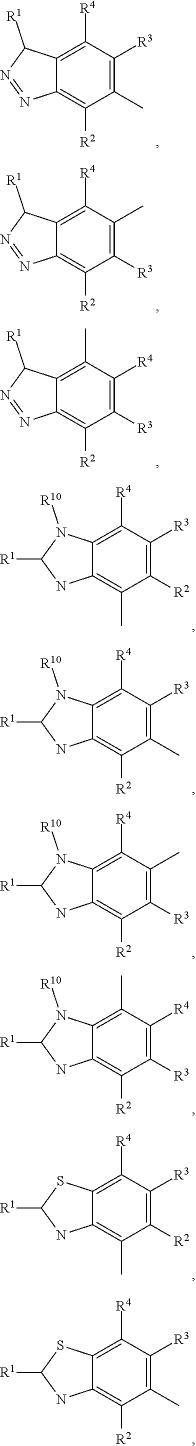

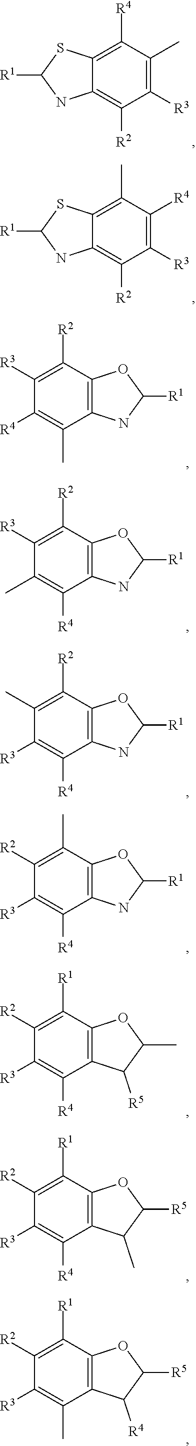

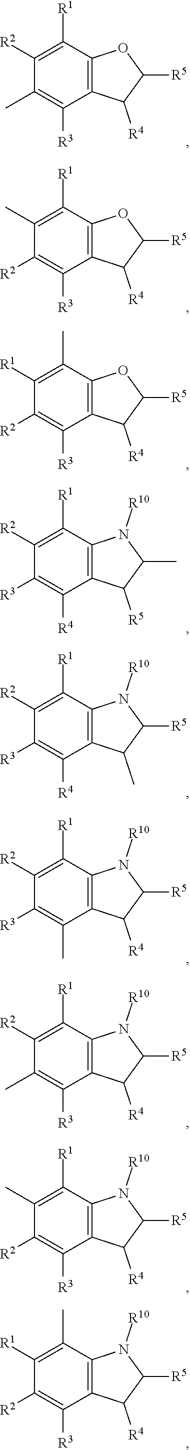

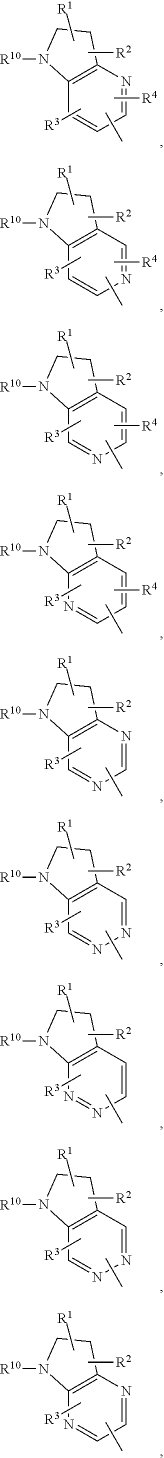

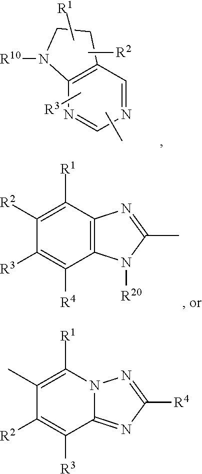

wherein R¹, R², R³, R⁴, R⁵, R⁶, R⁷, R⁸, and R⁹ represent independently of each other —H, —F, —Cl, —Br, —I, -Ph, —OH, —OCH₃, —OC₂H₅, —OC₃H₇, —OCH(CH₃)₂, —OC₄H₉, —OCH(CH₃)CH₂CH₃, —OCH₂CH(CH₃)₂, —OC(CH₃)₃, —O-cyclo-C₃H₅, —O-cyclo-C₄H₇, —O-cyclo-C₅H₉, —O-cyclo-C₆H₁₁, —OPh, —OCH₂-Ph, —OCH₂CH₂-Ph —OCH═CH₂, —OCH₂—CH═CH₂, —OCH₂CH₂—CH═CH₂, —OCF₃, —OC₂F₅, —SCH₃, —SC₂H₅, —SC₃H₇, —SCH(CH₃)₂, —SC₄H₉, —SCH(CH₃)CH₂CH₃, —SCH₂CH(CH₃)₂, —SC(CH₃)₃, —NO₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(C₄H₉)₂, —N(cyclo-C₃H₅)₂, —N[CH(CH₃)₂]₂, —N[C(CH₃)₃]₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH₂—CH₂—CH(CH₃)₂, —CH(CH₃)—CH(CH₃)—CH₃, —C(CH₃)₂—C₂H₅, —C₆H₁₃, —CH(CH₃)—C₄H₉, —CH₂—CH(CH₃)—C₃H₇, —CH₂—CH₂—CH₂—CH(CH₃)₂, —CH(CH₃)—CH(CH₃)—C₂H₅, —C(CH₃)₂—C₃H₇, —C₇H₁₅, —CH(CH₃)—C₅H₁₁, —CH₂—CH(CH₃)—C₄H₉, —CH₂—CH₂—CH₂—CH₂—CH(CH₃)₂, —C₈H₁₇, —C₉H₁₉, —C₁₀H₂₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —CH₂F, —CF₂I, —CHF₂, —CF₃, —CH₂I, —CH₂Br, —CH₂I, —CH₂—CH₂F, —CH₂—CHF₂, —CH₂—CF₃, —CH₂—CH₂I, —CH₂—CH₂Br, —CH₂—CH₂I, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COO-cyclo-C₄H₇, —COO-cyclo-C₅H₉, —COO-cyclo-C₆H₁₁, —COOCH(CH₃)₂, —COOC(CH₃)₃, —O—COOCH₃, —O—COOC₂H₅, —O—COOC₃H₇, —O—COO-cyclo-C₃H₅, —O—COOCH(CH₃)₂, —O—COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃Na, —SO₃K, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —B(OH)₂, —B(OR²²),

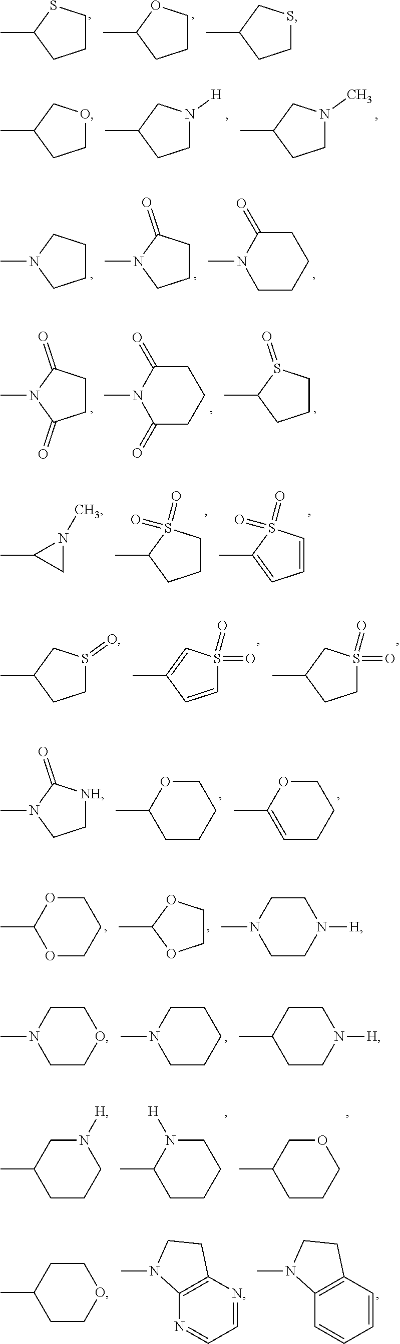

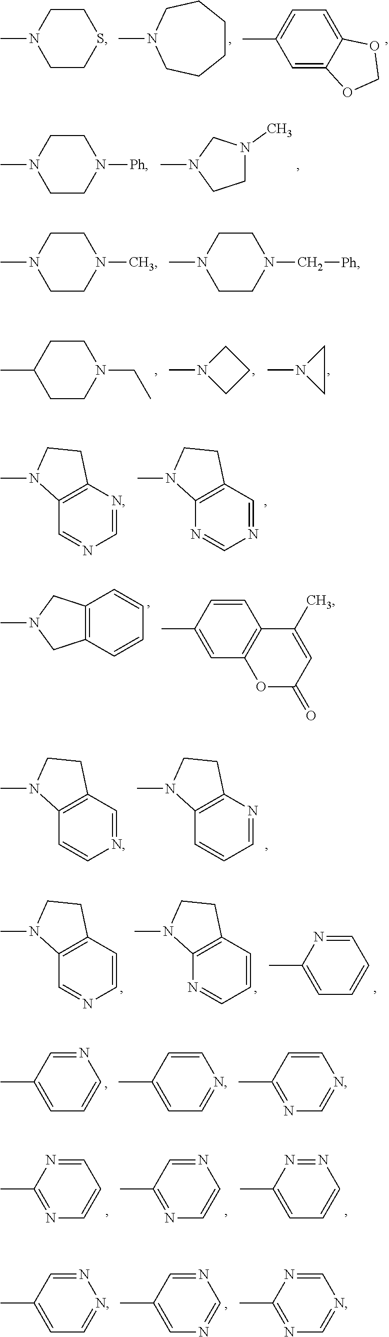

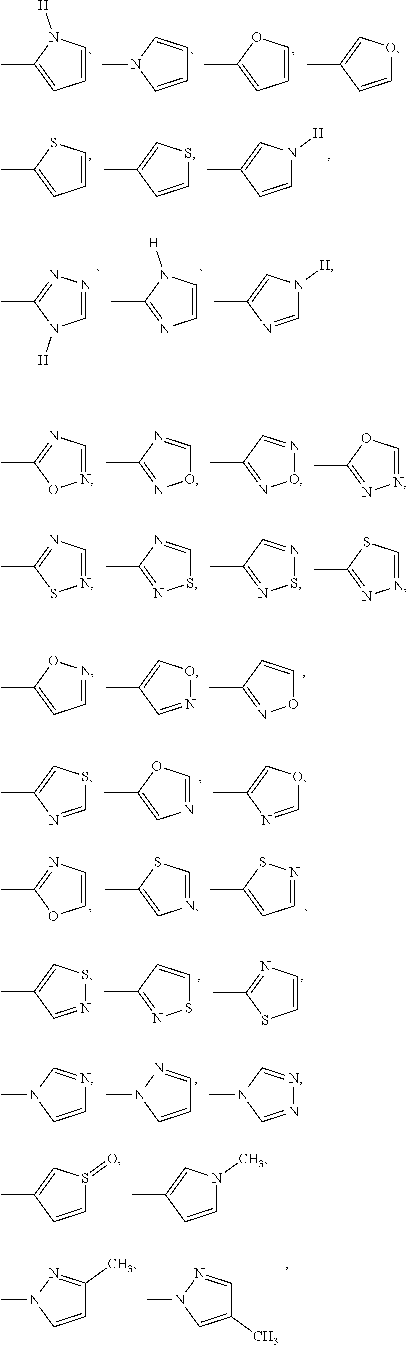

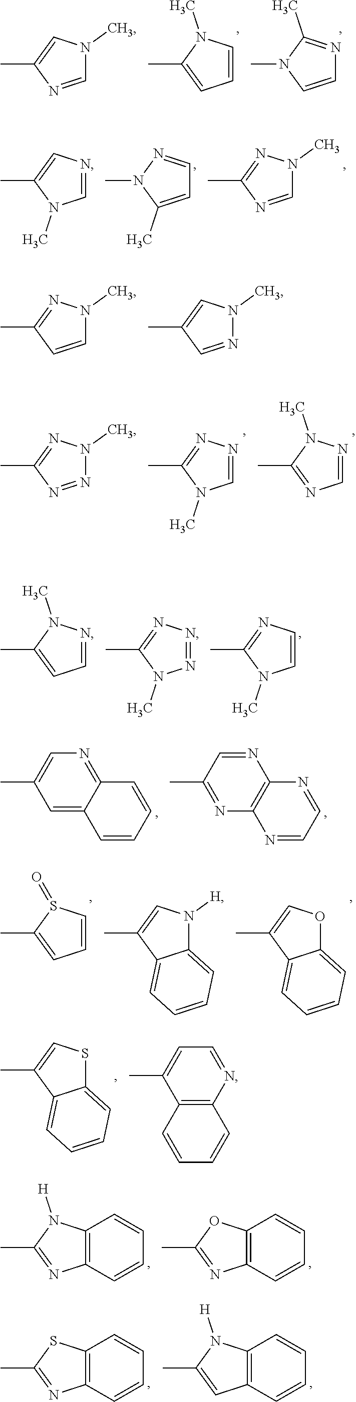

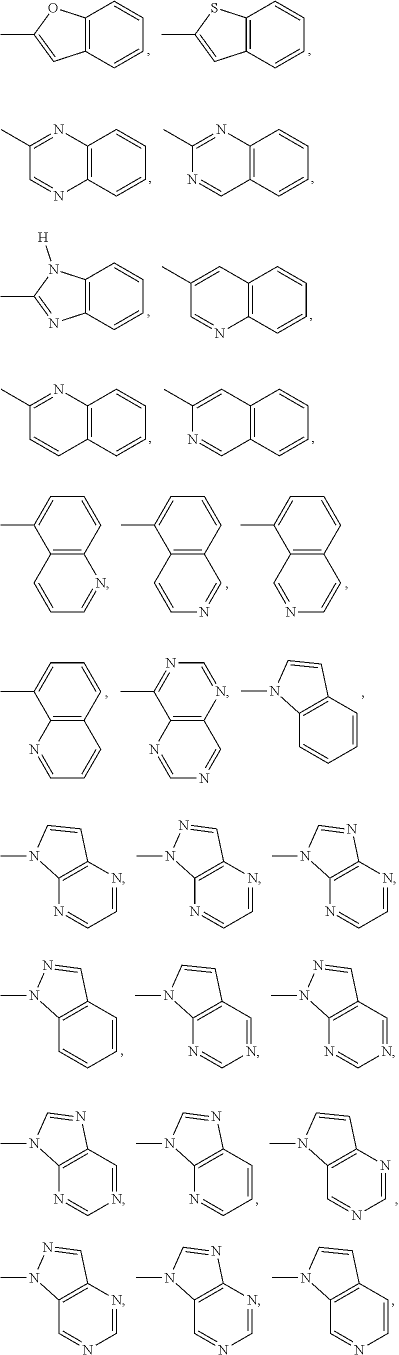

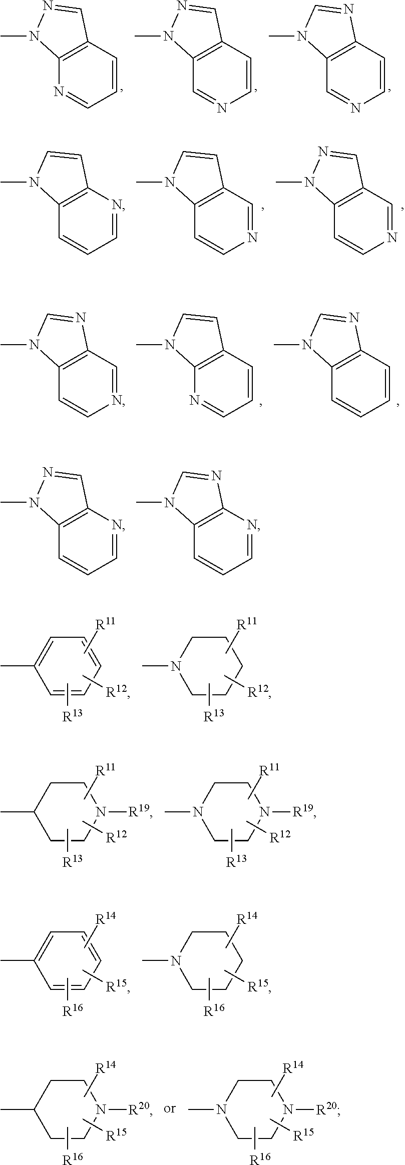

R¹¹ to R¹⁶ represents independently of each other:

—H, —NH₂, —OH, —OCH₃, —OC₂H₅, —OC₃H₇, —OCF₃, —CF₃, —F, —Cl, —Br, —I, —CH₃, —C₂H₅, —C₃H₇, —CH(CH₃)₂, -Ph, or —CN;

R¹⁰, R¹⁷, R¹⁸, R¹⁹, R²⁰, and R²¹ represents independently of each other —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, CH₂—CH₂F, —CH₂—CHF₂, —CH₂—CF₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, -cyclo-C₃H₅, -cyclo-C₄H₇, -cyclo-C₅H₉, -cyclo-C₆H₁₁, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, preferably R²¹ cannot be —H;

R²² represents —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, CH₂—CH₂F, —CH₂—CHF₂, —CH₂—CF₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, -cyclo-C₃H₅,

wherein n is an integer 1, 2, or 3; or a salt thereof, comprising:
A) providing a compound R—CHO (1) and an oxalyl-CoA (2)

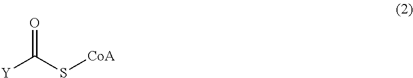

wherein Y represents —COO⁻;
in a buffer solution with a pH-value in the range of 5.0 to 7.5 optionally together with at least one co-solvent;
B) performing a coupling reaction of R—CHO (1) and oxalyl-CoA (2) by an oxalyl-CoA decarboxylase (3a) in the presence of thiamine diphosphate (3b) and a magnesium (II) cation to obtain the (S)-α-hydroxyacyl-CoA thioester (I)

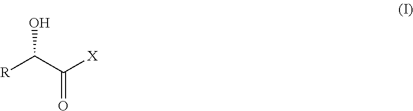

wherein X represents —S—CoA,
and the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (I) by a hydrolase, α-hydroxyacyl-CoA:oxalate CoA-transferase and/or α-hydroxyacyl-CoA:formate CoA-transferase to obtain the compound of the formula (I) or a salt thereof

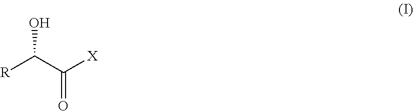

wherein X represents —OH or O⁻,
and the hydrolase is acyl-CoA thioester hydrolase YciA (4) encoded by a gene selected from E. coli.

A more preferred embodiment of the present invention is related to a method for preparation of a compound of the formula (II)

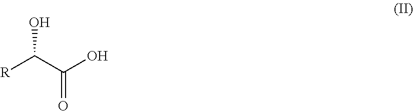

wherein R represents

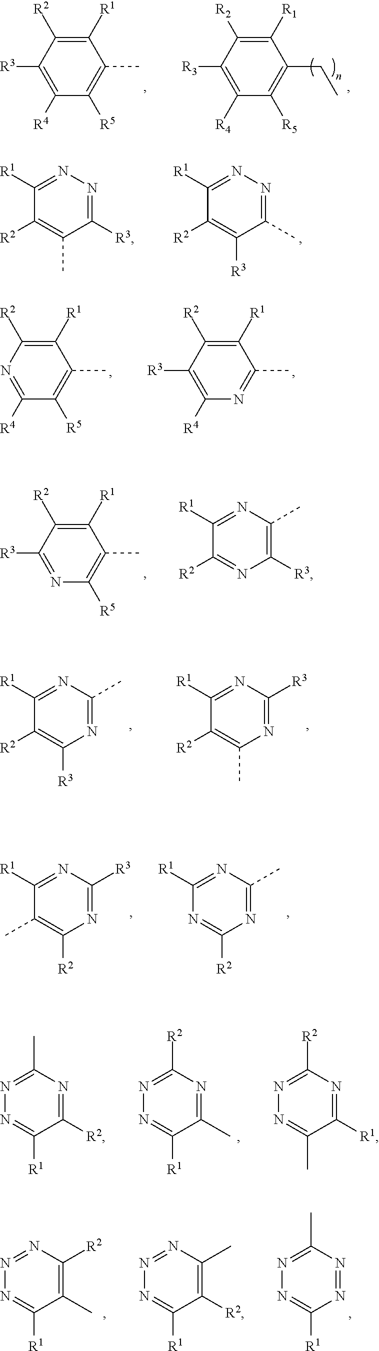

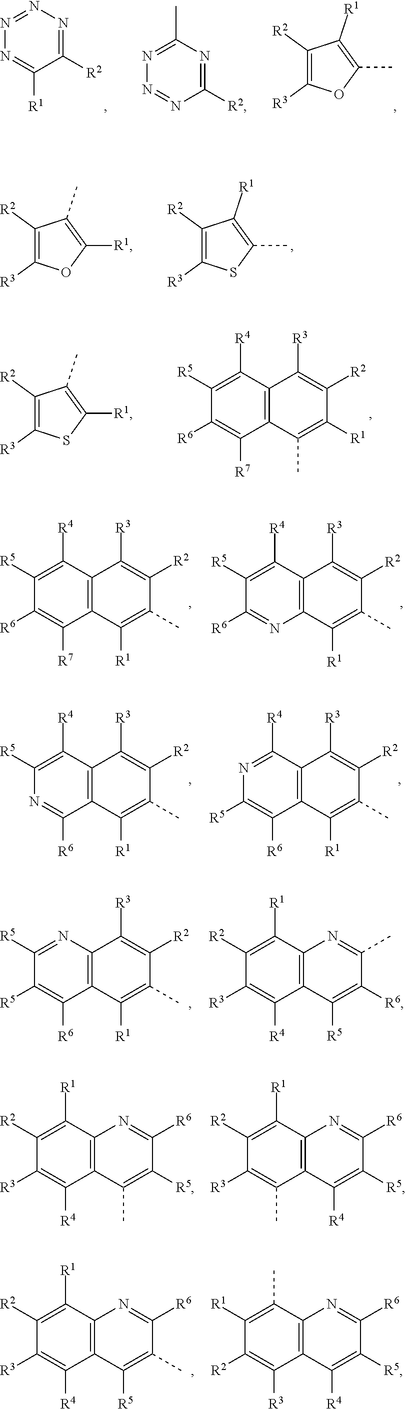

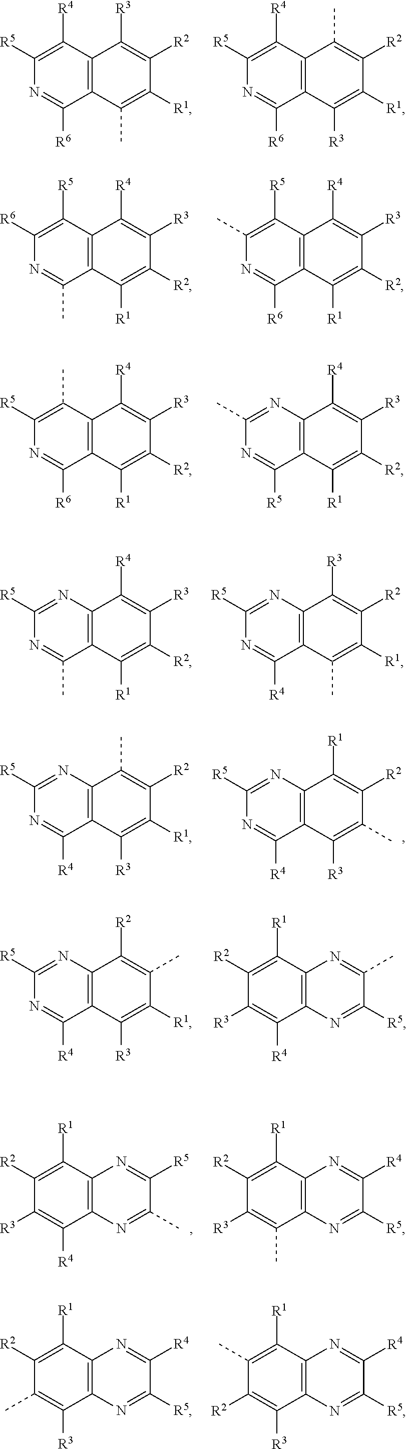

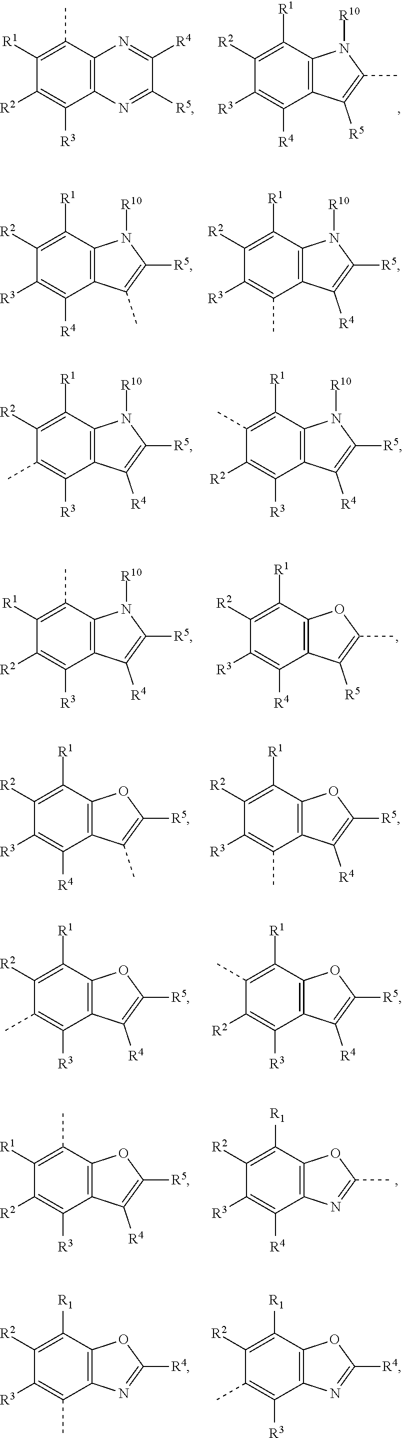

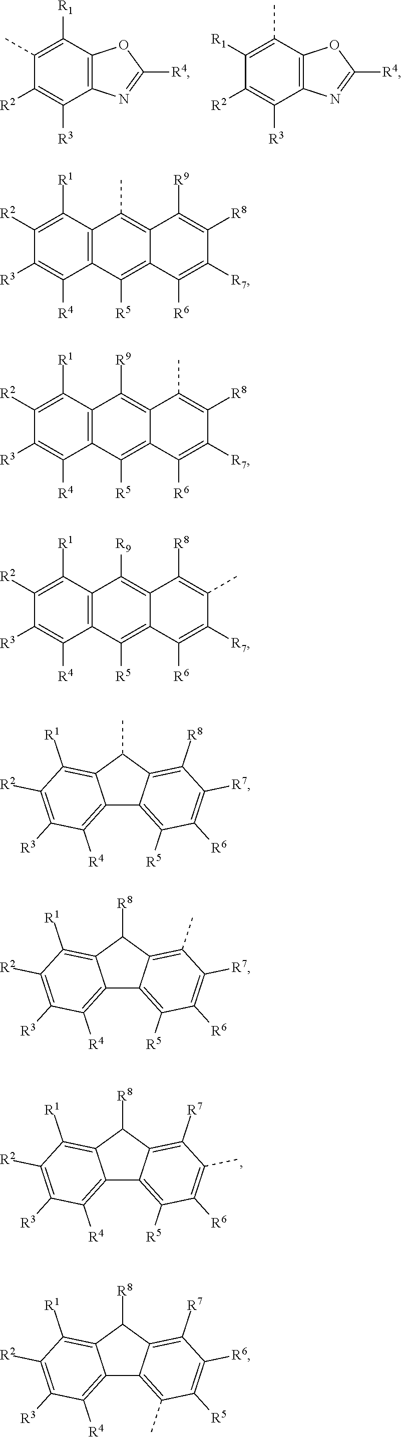

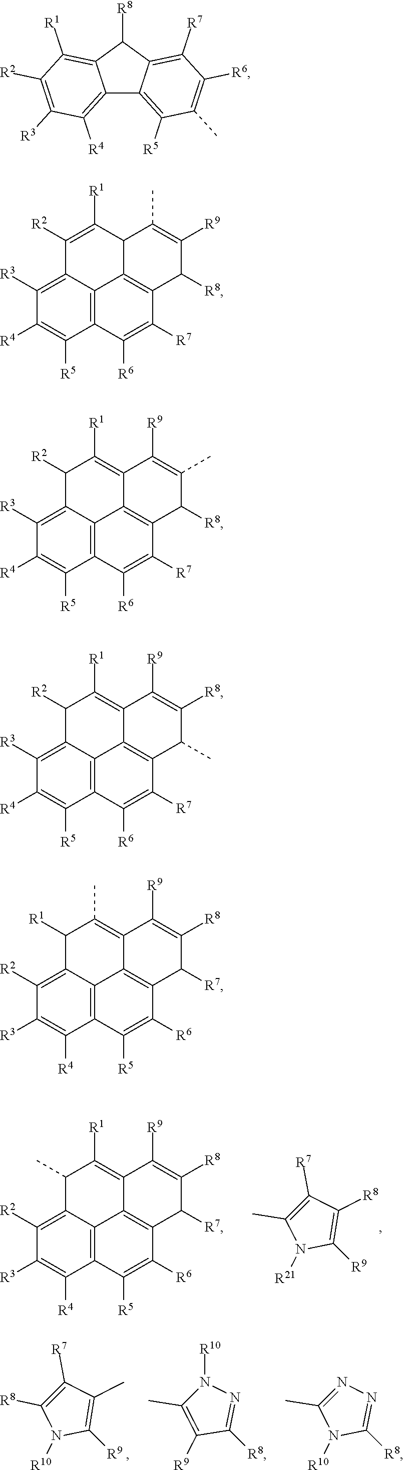

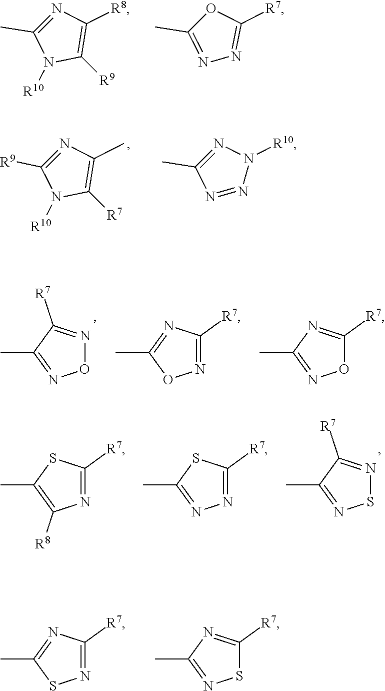

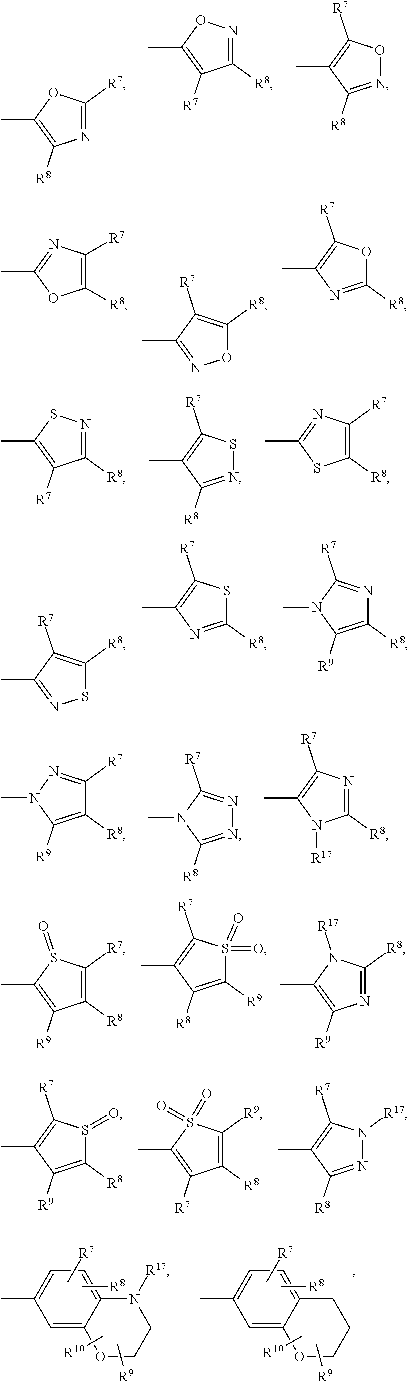

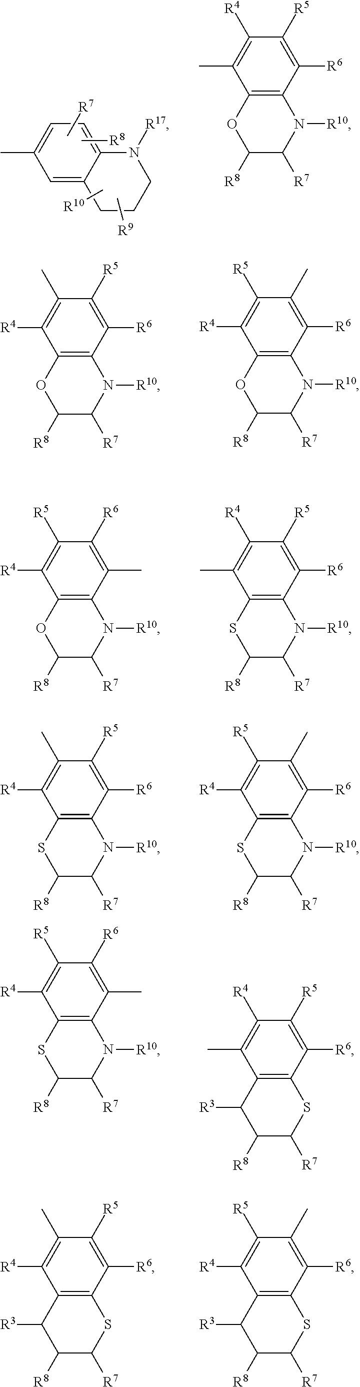

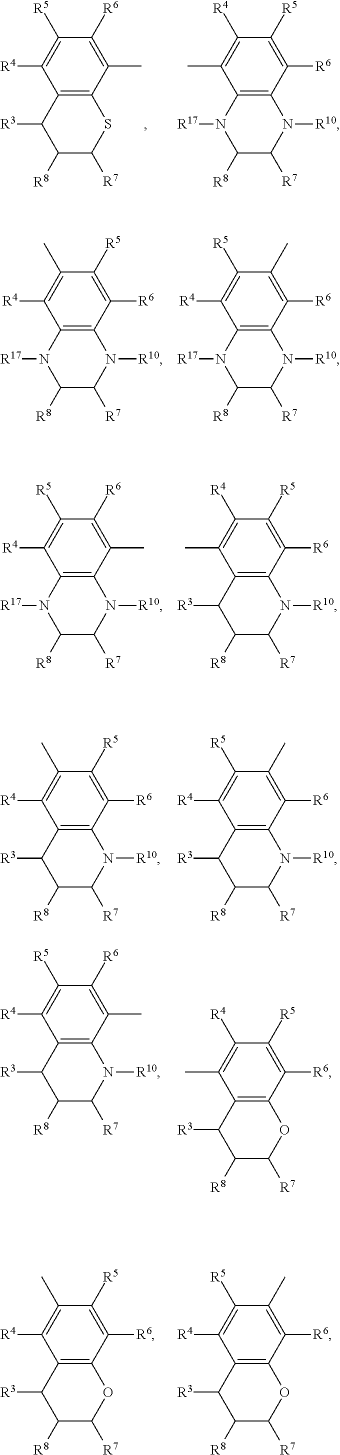

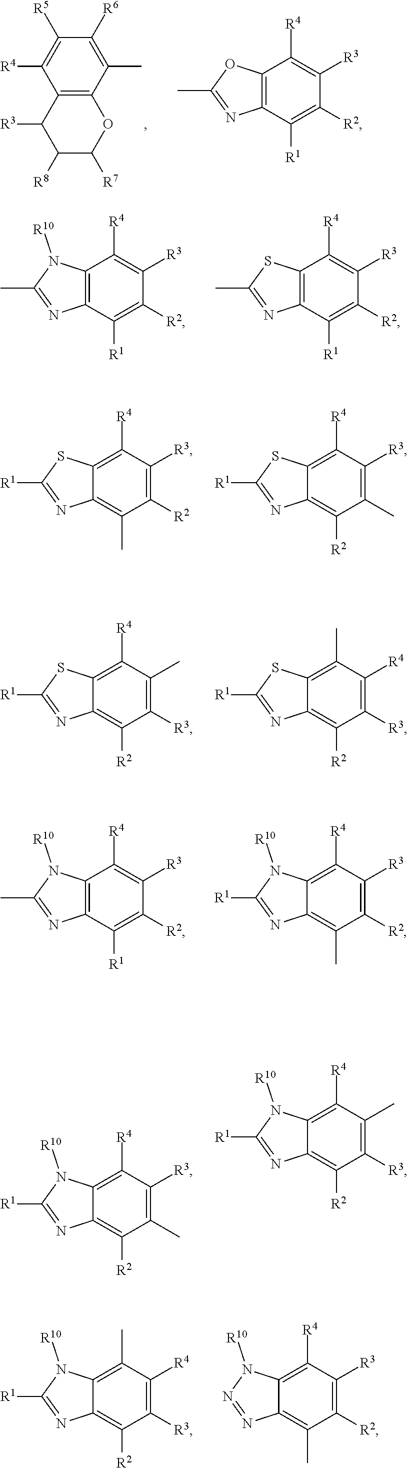

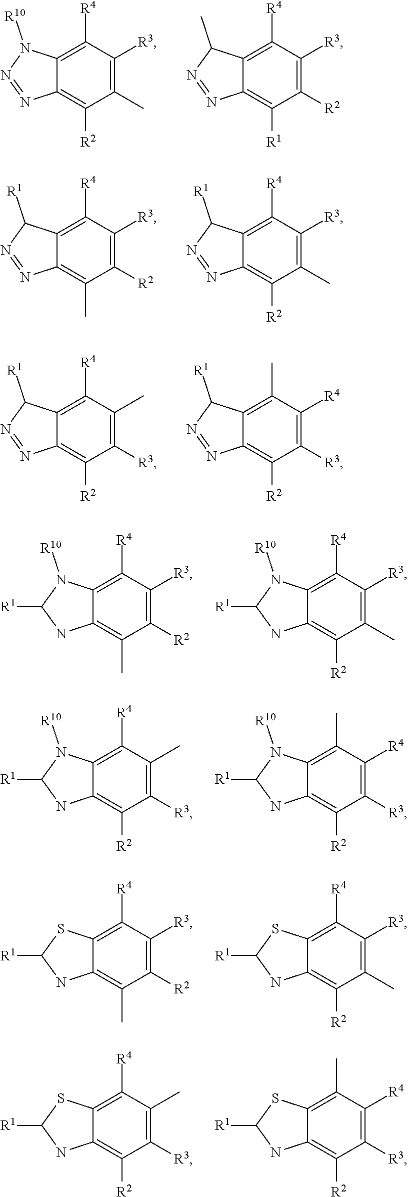

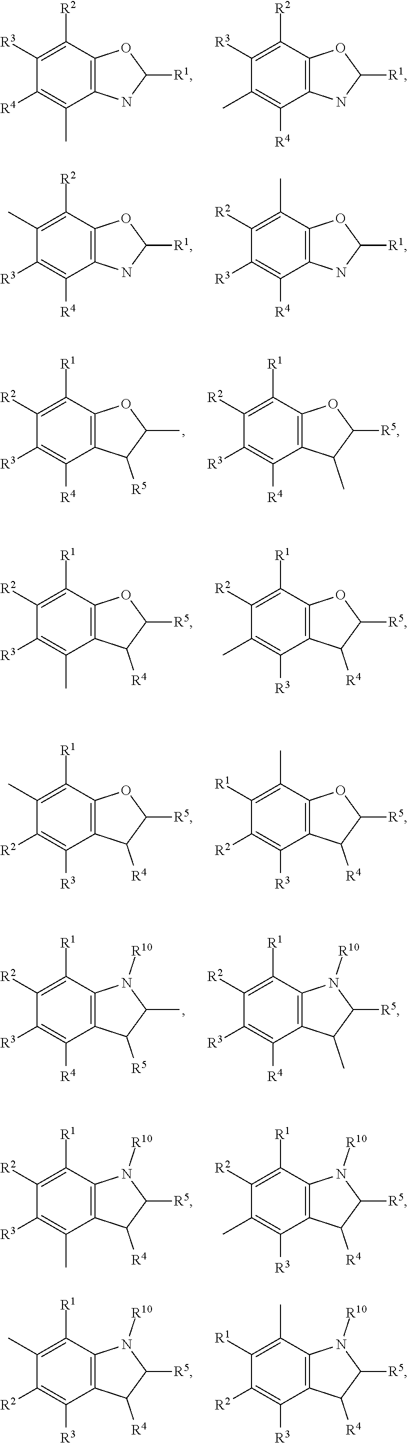

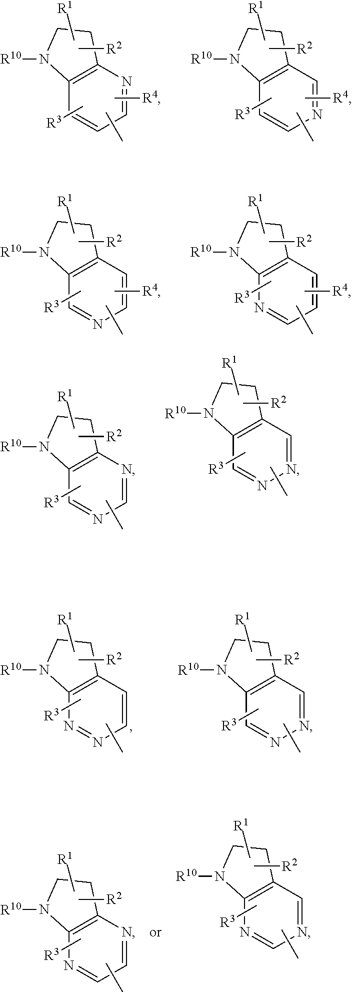

wherein R¹, R², R³, R⁴, R⁵, R⁶, R⁷, R⁸, and R⁹ represent independently of each other —H, —F, —Cl, —Br, —I, -Ph, —OH, —OCH₃, —OC₂H₅, —OC₃H₇, —OCH(CH₃)₂, —OC₄H₉, —OCH(CH₃)CH₂CH₃, —OCH₂CH(CH₃)₂, —OC(CH₃)₃, —O-cyclo-C₃H₅, —O-cyclo-C₄H₇, —O-cyclo-C₅H₉, —O-cyclo-C₆H₁₁, —OPh, —OCH₂-Ph, —OCH₂CH₂-Ph —OCH═CH₂, —OCH₂—CH═CH₂, —OCH₂CH₂—CH═CH₂, —OCF₃, —OC₂F₅, —SCH₃, —SC₂H₅, —SC₃H₇, —SCH(CH₃)₂, —SC₄H₉, —SCH(CH₃)CH₂CH₃, —SCH₂CH(CH₃)₂, —SC(CH₃)₃, —NO₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(C₄H₉)₂, —N(cyclo-C₃H₅)₂, —N[CH(CH₃)₂]₂, —N[C(CH₃)₃]₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH₂—CH₂—CH(CH₃)₂, —CH(CH₃)—CH(CH₃)—CH₃, —C(CH₃)₂—C₂H₅, —C₆H₁₃, —CH(CH₃)—C₄H₉, —CH₂—CH(CH₃)—C₃H₇, —CH₂—CH₂—CH₂—CH(CH₃)₂, —CH(CH₃)—CH(CH₃)—C₂H₅, —C(CH₃)₂—C₃H₇, —C₇H₁₅, —CH(CH₃)—C₅H₁₁, —CH₂—CH(CH₃)—C₄H₉, —CH₂—CH₂—CH₂—CH₂—CH(CH₃)₂, —C₈H₁₇, —C₉H₁₉, —C₁₀H₂₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —CH₂F, —CF₂I, —CHF₂, —CF₃, —CH₂I, —CH₂Br, —CH₂I, —CH₂—CH₂F, —CH₂—CHF₂, —CH₂—CF₃, —CH₂—CH₂I, —CH₂—CH₂Br, —CH₂—CH₂I, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COO-cyclo-C₄H₇, —COO-cyclo-C₅H₉, —COO-cyclo-C₆H₁₁, —COOCH(CH₃)₂, —COOC(CH₃)₃, —O—COOCH₃, —O—COOC₂H₅, —O—COOC₃H₇, —O—COO-cyclo-C₃H₅, —O—COOCH(CH₃)₂, —O—COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂,

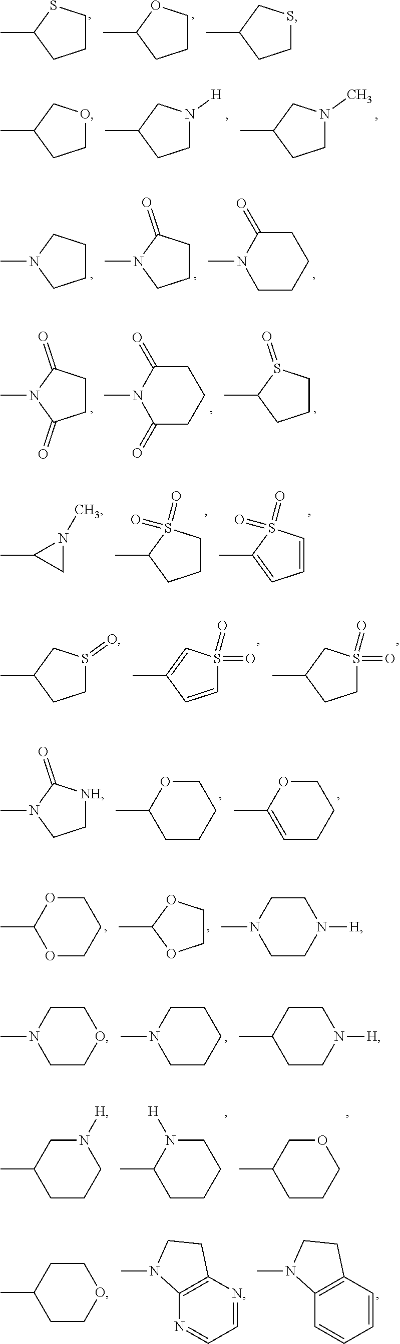

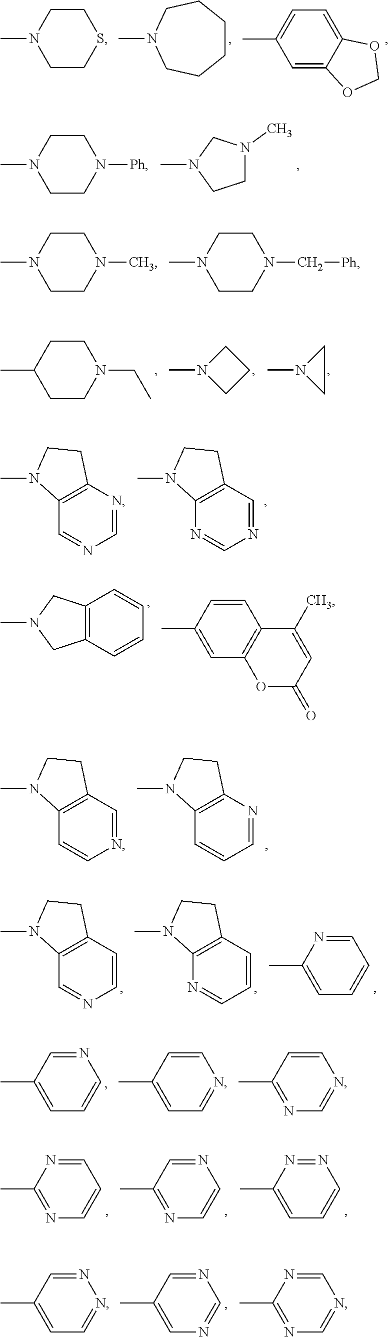

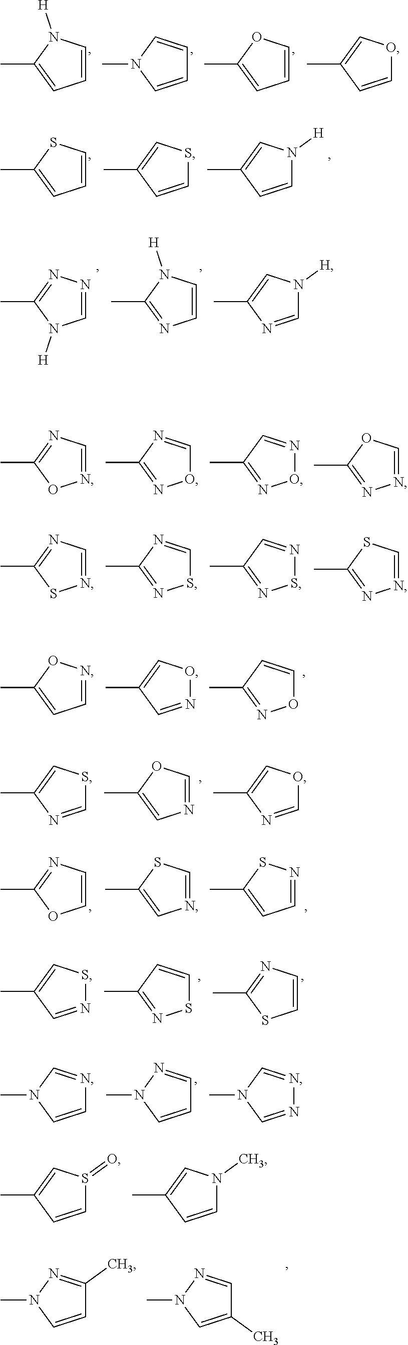

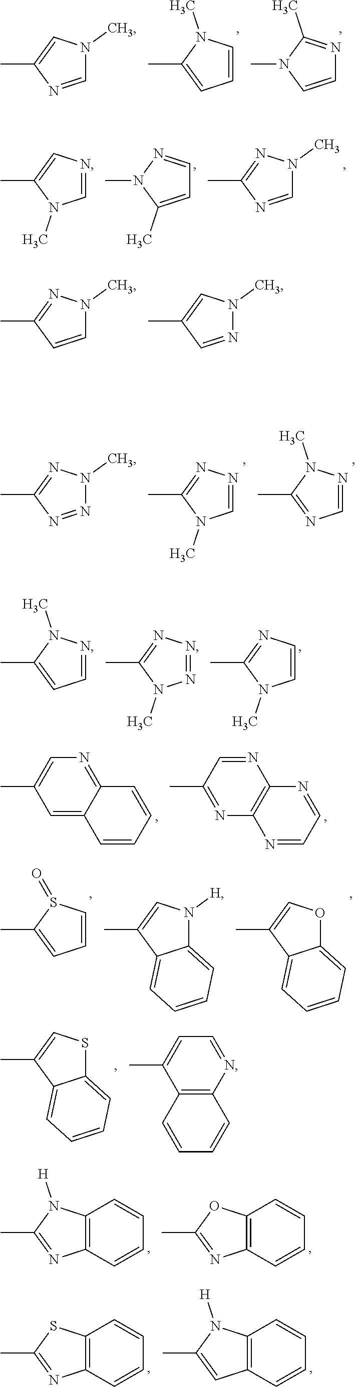

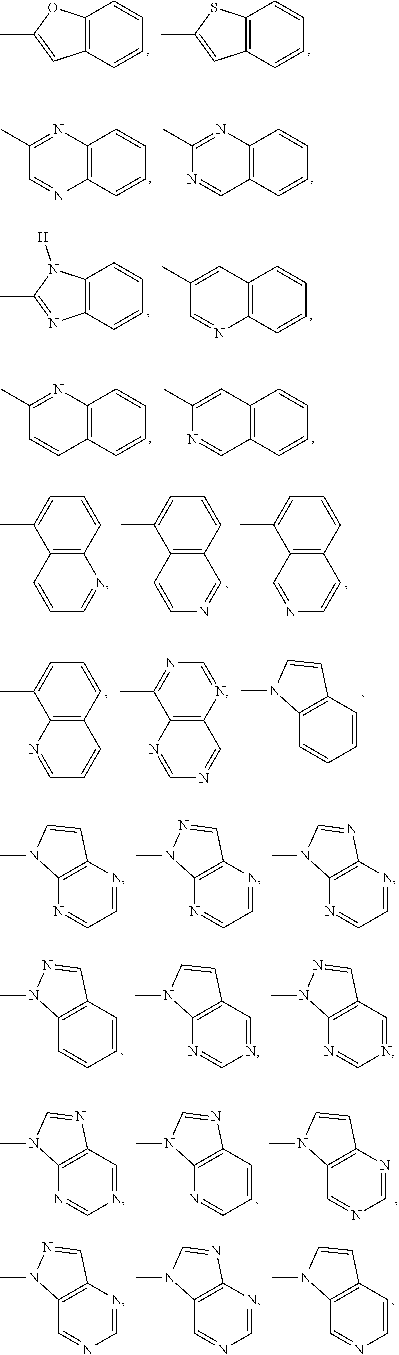

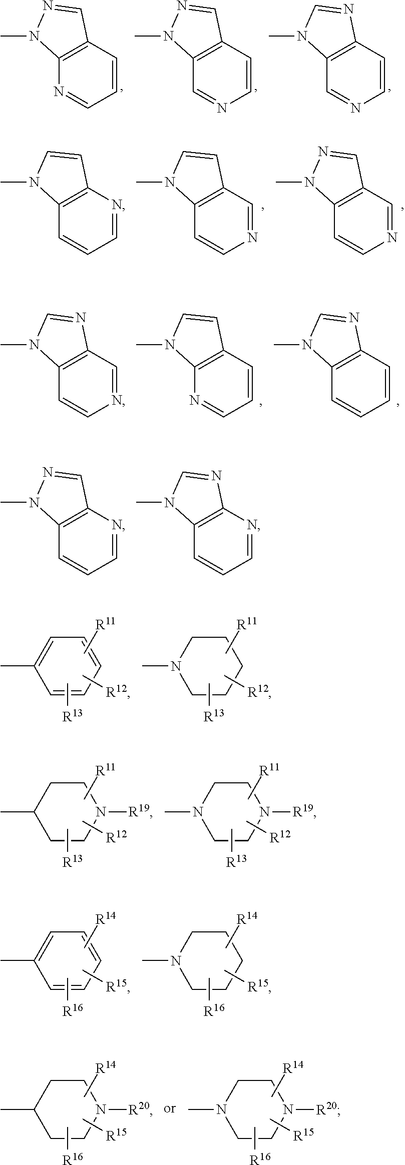

R¹¹ to R¹⁶ represents independently of each other:

—H, —NH₂, —OH, —OCH₃, —OC₂H₅, —OC₃H₇, —OCF₃, —CF₃, —F, —Cl, —Br, —I, —CH₃, —C₂H₅, —C₃H₇, —CH(CH₃)₂, -Ph, or —CN;

R¹⁰, R¹⁷, R¹⁸, R¹⁹, R²⁰, and R²¹ represents independently of each other —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, CH₂—CH₂F, —CH₂—CHF₂, —CH₂—CF₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, preferably R²¹ cannot be —H,

wherein n is an integer 1, 2, or 3; or a salt thereof,

comprising:
A) providing a compound R—CHO (1) and an oxalyl-CoA (2)

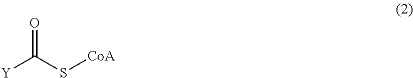

wherein Y represents —COO⁻;
in a buffer solution with a pH-value in the range of 5.0 to 7.5 optionally together with at least one co-solvent;
B) performing a coupling reaction of R—CHO (1) and oxalyl-CoA (2) by an oxalyl-CoA decarboxylase (3a) in the presence of thiamine diphosphate (3b) and a magnesium (II) cation to obtain the (S)-α-hydroxyacyl-CoA thioester (I)

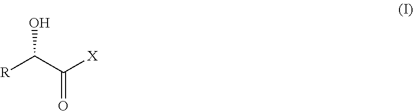

wherein X represents —S—CoA,
and the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (I) by a hydrolase, α-hydroxyacyl-CoA:oxalate CoA-transferase and/or α-hydroxyacyl-CoA:formate CoA-transferase to obtain the compound of the formula (I) or a salt thereof

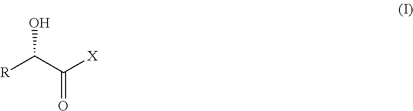

wherein X represents —OH or O⁻,
and the hydrolase is acyl-CoA thioester hydrolase YciA (4) encoded by a gene selected from E. coli.

Step B) can be performed for at least 0.5 hour, preferably 1 hour, more preferably 2 hours, more preferably 3 hours, more preferably 4 hours, more preferably 5 hours, more preferably 6 hours, more preferably 7 hours, more preferably 8 hours, more preferably 9 hours, more preferably 10 hours, more preferably 11 hours, more preferably 12 hours, more preferably 13 hours, more preferably 14 hours, more preferably 15 hours, more preferably 16 hours, more preferably 17 hours, more preferably 18 hours, more preferably 19 hours, more preferably 20 hours, more preferably 21 hours, more preferably 22 hours, more preferably 23 hours, more preferably 24 hours, more preferably 25 hours, more preferably 26 hours, more preferably 27 hours, and more preferably 28 hours.

Step B) can be performed from 0.5 hour to 48 hours, preferably from 1 hour to 40 hours, more preferably from 2 hours to 35 hours, more preferably from 3 hours to 30 hours, more preferably from 4 hours to 28 hours, more preferably from 5 hours to 26 hours, more preferably from 6 hours to 24 hours, more preferably from 8 hours to 24 hours, more preferably from 10 hours to 24 hours, more preferably form 12 hours to 24 hours, more preferably from 14 hours to 24 hours, more preferably from 16 hours to 24 hours, and most preferably form 18 hours to 24 hours.

In general, after performing step B), i.e. the carbon-carbon-bond forming step, the formed α-hydroxyacyl-CoA thioester can be isolated, and hydrolyzed in a subsequent step C) to obtain the α-hydroxy carboxylic acid, or salts thereof.

In order to increase the efficiency of the method, the steps in particular the steps A), B) and C) of a method according to invention described herein can be carried out as cascade reaction in one-pot system. If in a method according to the invention the steps A), B) and C) is performed as cascade reaction in one-pot system preferably ATP, coenzyme A and/or carboxylate such as oxalate and formate is added last. More preferably oxalate is added last.

For this, an enzyme such as thioesterase capable of hydrolyzing the non-natural metabolite aromatic α-hydroxyacyl-CoA thioester in particular mandelic acid and its derivatives, and it particular capable of selectively hydrolyzing said compound would be necessary since the acyl-CoA (2) as well as the α-hydroxyacyl-CoA thioester encompass a thioester moiety.

Surprisingly, it could be found that YciA from E. coli has shown a relatively high α-hydroxyacyl-CoA in particular mandelyl-CoA thioesterase activity, low activity with formyl-CoA and no activity with oxalyl-CoA (example 11, FIG. 15).

A further aspect of the invention is directed to a composition comprising or consisting of a peptide oxalyl-CoA decarboxylase (OXC_{Me}) encoded by a gene from Methylobacterium extorquens or a mutant thereof, wherein in the mutant at least one tyrosine or serine is replaced independently of each other by alanine, phenylalanine, and/or glycine, and a thioesterase YciA encoded by a gene selected from E. coli. Preferably, at least one tyrosine or serine is replaced by alanine. It could be shown that the formation of mandelyl-CoA is higher using said mutant than the wild type OXC_{Me} (example 9, and FIGS. 14A and 14B).

Further, an aspect of the present invention is directed to use of YciA for the hydrolysis of non-natural α-hydroxyacyl-CoA thioester in particular mandelyl-CoA or for the synthesis non-natural α-hydroxy carboxylic acids, or salts thereof in particular mandelic acid and its derivatives.

Another aspect of the present invention is related to use of a composition comprising a peptide oxalyl-CoA decarboxylase (OXC_{Me}) encoded by a gene from Methylobacterium extorquens or a mutant thereof, wherein in the mutant at least one tyrosine or serine is replaced by an amino acid selected independently of each other from the group comprising or consisting of alanine, phenylalanine and glycine and a thioesterase YciA encoded by a gene selected from E. coli for the preparation of an α-hydroxy carboxylic acid, or its salt preferably starting from oxalyl-CoA and an aldehyde. Preferably, at least one tyrosine or serine is replaced by alanine.

A preferred embodiment of the invention is directed to a method for preparation of a compound of the formula (II)

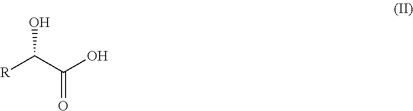

comprising:
A) providing a compound R—CHO (1) and an oxalyl-CoA (2′)

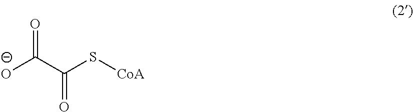

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

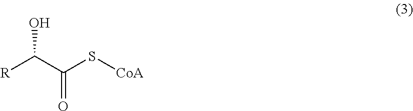

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

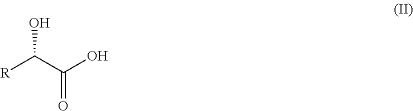

wherein the hydrolase is acyl-CoA thioester hydrolase YciA (4) encoded by a gene selected from E. coli; and

wherein steps A), B) and C) are carried out as cascade reaction in one-pot system.

In the methods according to invention as described herein, the acyl-CoA (2) can be prepared by the following step A′):
A′) reacting a carboxylate (2a) with a free coenzyme A (2b) by acyl-CoA synthetase (2c) in the presence of ATP and a magnesium (II) cation (2e) to produce an acyl-CoA (2).

The carboxylate (2a) can be an oxalate or a formate. As a source for the carboxylate the protonated form of the carboxylate can be used if the carboxylate is generated in solution. In case of the oxalate it is possible to use an oxalic acid (H₂C₂O₄) or a hydrogen oxalate (HC₂O₄⁻) as a source for the oxalate. For the formate, formic acid could be used as a source for the formate.

Thus, in the methods according to invention as described herein, the acyl-CoA (2) can be prepared by the following step A′):
A′) reacting an oxalate (C₂O₄²⁻) (2a′) with a free coenzyme A (2b) by oxalyl-CoA synthetase (2c′) in the presence of ATP (2e) to produce an oxalyl-CoA (2′) or reacting a formate (HCOO⁻) (2a″) with a free coenzyme A (2b) by formyl-CoA synthetase (2c″) optional in the presence of ATP and magnesium(II) cation to produce formyl-CoA (2″).

In the methods according to invention as described herein, the oxalyl-CoA (2′) or the formyl-CoA (2″) can be prepared by the following step A′):
A′) reacting an oxalate (C₂O₄²⁻) (2a′) with a free coenzyme A (2b) by oxalyl-CoA synthetase (2c′) in the presence of ATP (2e) to produce an oxalyl-CoA (2′) or reacting a formate (HCOO⁻) (2a″) with a free coenzyme A (2b) by formyl-CoA synthetase (2c″) to produce formyl-CoA (2″).

The steady-state kinetic parameters are shown in FIG. 18.

Thus, the step A′) is preferably included in the method according to the invention as described herein.

The term “acyl-CoA synthetase” refers to an enzyme capable of catalyzing the formation of acyl-CoA (2) starting from a carboxylate (2a). This includes oxalyl-CoA synthetase, acetyl-CoA synthetase and formyl-CoA synthetase.

The oxalyl-CoA synthetase is preferably encoded by a gene selected from Methylobacterium extorquens preferably having the sequence SEQ ID NO. 5 (FIG. 21).

Therefore, another aspect of the invention is related to the use of an oxalyl-CoA synthetase encoded by a gene selected from Methylobacterium extorquens preferably having the sequence SEQ ID NO. 5 (FIG. 21)) for the synthesis of an α-hydroxycarboxylic acid, salts thereof or an α-hydroxyacyl-CoA thioester.

As a formyl-CoA synthetase, an acyl-CoA-synthetase can be used preferably encoded by a gene selected from Erythrobacter sp. having the sequence SEQ ID NO. 18 (FIG. 23) or mutants thereof can be used. More preferably, the formyl-CoA synthetase is an acyl-CoA synthetase is a mutant of the formyl-CoA synthetase encoded by a gene selected from Erythrobacter sp. Still more preferably, the formyl-CoA synthetase is an acyl-CoA synthetase is a mutant of the formyl-CoA synthetase encoded by a gene selected from Erythrobacter sp., wherein at least one amino acid is replaced by another proteinogenic amino acid. Still more preferably, the formyl-CoA synthetase is an acyl-CoA synthetase is a mutant of the formyl-CoA synthetase encoded by a gene selected from Erythrobacter sp., wherein at position 379 valine is replaced by a proteinogenic amino acid. Most preferably, the formyl-CoA synthetase is an acyl-CoA synthetase is a mutant of the formyl-CoA synthetase encoded by a gene selected from Erythrobacter sp. having the sequence SEQ ID NO. 19 (FIG. 24), i.e. most preferably, the formyl-CoA synthetase is an acyl-CoA synthetase is a mutant of the formyl-CoA synthetase encoded by a gene selected from Erythrobacter sp., wherein at position 379 valine is replaced by isoleucine.

Thus, another aspect of the invention is directed to a mutant of the acyl-CoA synthetase encoded by a gene selected from Erythrobacter sp. having the sequence SEQ ID NO. 19. A further aspect is related to the use of a mutant of the acyl-CoA synthetase encoded by a gene selected from Erythrobacter sp. having the sequence SEQ ID NO. 19 as an oxalyl-CoA synthetase.

An embodiment according to the invention is related to a method for the preparation of α-hydroxyacyl compound of the formula (I)

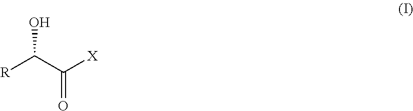

comprising:
A) providing a compound R—CHO (1) and an acyl-CoA (2)

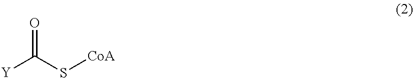

wherein Y represents —H, or —COO⁻,
in a buffer solution with a pH-value in the range of 5.0 to 7.5 and optionally together with at least one co-solvent, and wherein acyl-CoA (2) can be prepared by the following step A′):
A′) reacting an oxalate (C₂O₄²⁻) (2a′) with a free coenzyme A (2b) by oxalyl-CoA synthetase (2c′) in the presence of ATP (2e) to produce an oxalyl-CoA (2′) or reacting a formate (HCOO⁻) (2a″) with a free coenzyme A (2b) by formyl-CoA synthetase to produce formyl-CoA (2″);
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

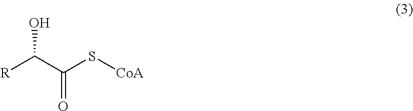

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

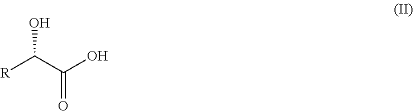

wherein the hydrolase is acyl-CoA thioester hydrolase YciA (4) encoded by a gene selected from E. coli.

An embodiment of the invention is directed to a method for preparation of a compound of the formula (I)

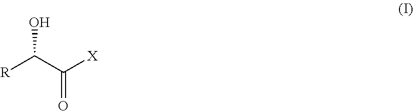

comprising:
A) providing a compound R—CHO (1) and an oxalyl-CoA (2′)

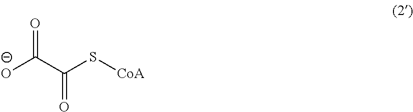

in a buffer solution with a pH-value in the range of 5.0 to 7.5, optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

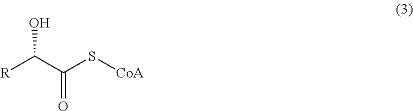

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

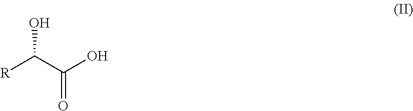

wherein the hydrolase is acyl-CoA thioester hydrolase YciA (4) encoded by a gene selected from E. coli.

A preferred embodiment of the invention is related to a method for preparation of a compound of the formula (II)

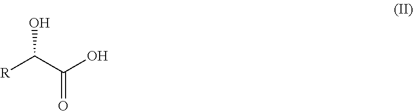

comprising:
A) providing a compound R—CHO (1) and an oxalyl-CoA (2′)

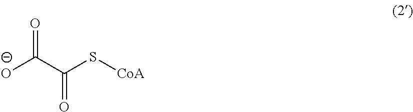

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

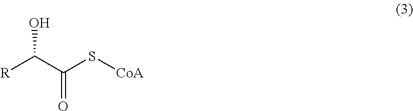

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoAoxalate CoA-transferase and/or α-hydroxyacyl-CoA:formate CoA-transferase to obtain the compound of the formula (II) or a salt thereof

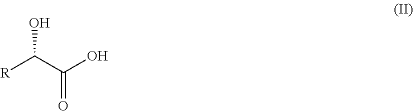

wherein the hydrolase is acyl-CoA thioester hydrolase YciA (4) encoded by a gene selected from E. coli.

A more preferred embodiment of the invention is directed to a method for preparation of a compound of the formula (II)

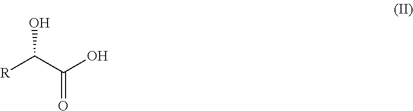

comprising:
A) providing a compound R—CHO (1) and an oxalyl-CoA (2′)

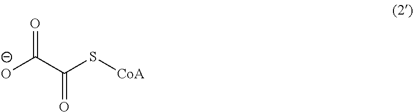

in a buffer solution with a pH-value in the range of 5.0 to 7.5 optionally at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioesteer (3)

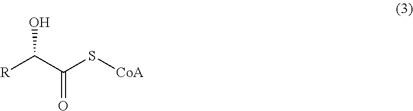

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

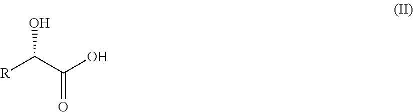

wherein the hydrolase is acyl-CoA thioester hydrolase YciA (4) encoded by a gene selected from E. coli; and

wherein steps A), B) and C) are carried out as cascade reaction in one-pot system.

In general, the compounds generated in step A′) or B) can be isolated, and used in step C). In order to increase the efficiency of the method, the steps A′), A), and B) or A′), A), B), and C) of a method according to the invention can be carried out as cascade reaction in one-pot system. Preferably, A′), A), B), and C) of a method according to the invention can be carried out as cascade reaction in one-pot system.

An embodiment of the present invention is directed to a method for preparation of a compound of the formula (I)

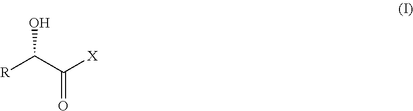

comprising:
A) providing a compound R—CHO (1) and an acyl-CoA (2)

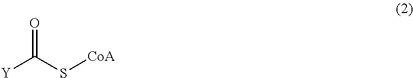

wherein Y represents —H, or —COO⁻,
in a buffer solution with a pH-value in the range of 5.0 to 7.5, optionally together with least one co-solvent, and wherein acyl-CoA (2) can be prepared by the following step A′):
A′) reacting an oxalate (C₂O₄²⁻) (2a′) with a free coenzyme A (2b) by oxalyl-CoA synthetase (2c′) in the presence of ATP (2e) to produce an oxalyl-CoA (2′) or reacting a formate (HCOO⁻) (2a″) with a free coenzyme A (2b) by formyl-CoA synthetase to produce formyl-CoA (2″);
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

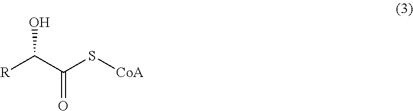

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

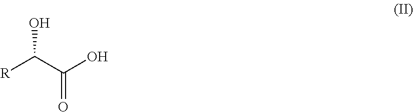

wherein the hydrolase is acyl-CoA thioester hydrolase YciA (4) encoded by a gene selected from E. coli; and

wherein steps A′), A), and B), or steps A′), A), and B), and C) are carried out as cascade reaction in one-pot system.

An embodiment of the invention is directed to a method for preparation of a compound of the formula (I)

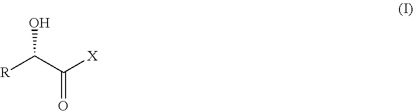

comprising:
A) providing a compound R—CHO (1) and an oxalyl-CoA (2′)

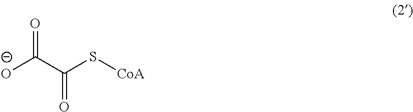

in a buffer solution with a pH-value in the range of 5.0 to 7.5, optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

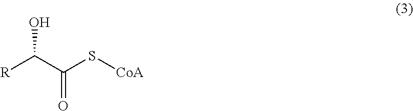

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

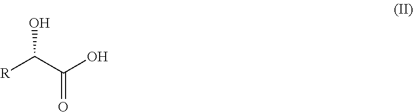

wherein the hydrolase is acyl-CoA thioester hydrolase YciA (4) encoded by a gene selected from E. coli; and

wherein steps A′), A), and B), or steps A′), A), and B), and C) are carried out as cascade reaction in one-pot system.

A preferred embodiment of the invention is related to a method for preparation of a compound of the formula (II)

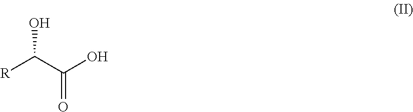

comprising:
A) providing a compound R—CHO (1) and an oxalyl-CoA (2′)

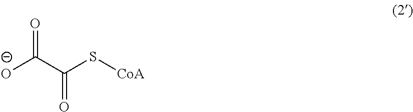

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

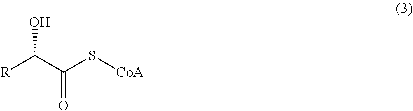

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoAoxalate CoA-transferase and/or α-hydroxyacyl-CoA:formate CoA-transferase to obtain the compound of the formula (II) or a salt thereof

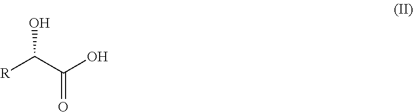

wherein the hydrolase is acyl-CoA thioester hydrolase YciA (4) encoded by a gene selected from E. coli; and

wherein steps A′), A), B), and C) are carried out as cascade reaction in one-pot system.

Since the coenzyme A is cleaved during the hydrolysis of the α-hydroxyacyl-CoA thioester, the free coenzyme A can be used in small amounts (catalytic amounts). Thus, the tree enzymes form a catalytic cycle (FIGS. 3, 4 and 5). Therefore, in a method according to the invention, wherein the steps A′), A), B), and C) are carried out as cascade reaction in one-pot system, a ratio of R—CHO (1) and the free coenzyme A (2b) is 10 to 1000, preferably 20 to 500, and more preferably 40 to 250.

In order to receive a method in which the coenzyme A can be used in small amounts (catalytic amounts) or in other words a catalytic cycle, an enzyme composition has to be provided which is adapted to the various reaction demands such as selectivity, activity or stability of the enzyme, the starting material or the product.

Another aspect of the invention is thus directed to a composition comprising or consisting of a peptide OXC_{Me} encoded by a gene from Methylobacterium extorquens or a mutant thereof, wherein in the mutant at least one tyrosine or serine is replaced by alanine, phenylalanine, and/or glycine, and a thioesterase YciA encoded by a gene selected from E. coli, and an acyl-CoA synthetase in particular oxalyl-CoA synthetase. Preferably, tyrosine or serine is replaced by alanine, and more preferably, the at least one tyrosine or serine is replaced by alanine.

A further aspect of the present invention is related to use of a composition comprising or consisting of a peptide OXC_{Me} encoded by a gene from Methylobacterium extorquens or its mutant, wherein in the mutant at least one tyrosine or serine is replaced by alanine, phenylalanine, and/or glycine, and a thioesterase YciA encoded by a gene selected from E. coli, and an acyl-CoA synthetase in particular oxalyl-CoA synthetase for the preparation of an α-hydroxy carboxylic acid or salts thereof in particular starting from an oxalate, hydrogen oxalate, or oxalic acid and an aldehyde. Preferably, at least one tyrosine or serine is replaced by alanine.

An embodiment according to the invention is related to a method for the preparation of α-hydroxyacyl compound of the formula (I)

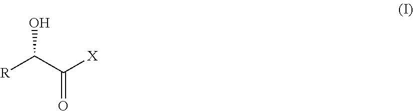

comprising:
A) providing a compound R—CHO (1) and an acyl-CoA (2)

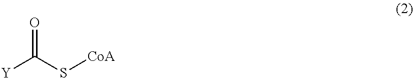

wherein Y represents —H, or —COO⁻,
in a buffer solution with a pH-value in the range of 5.0 to 7.5 optionally together with at least one co-solvent, and wherein acyl-CoA (2) can be prepared by the following step A′):
A′) reacting an oxalate (C₂O₄²⁻) (2a′) with a free coenzyme A (2b) by oxalyl-CoA synthetase (2c′) in the presence of ATP (2e) to produce an oxalyl-CoA (2′) or reacting a formate (HCOO⁻) (2a″) with a free coenzyme A (2b) by formyl-CoA synthetase to produce formyl-CoA (2″);
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

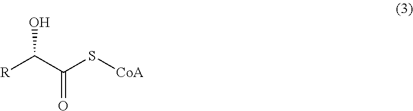

wherein the oxalyl-CoA decarboxylase (3a) is a peptide encoded by a gene selected from Methylobacterium extorquens or a mutant thereof; or additionally comprising the step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

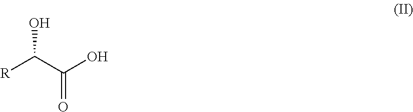

wherein the hydrolase is acyl-CoA thioester hydrolase YciA (4) encoded by a gene selected from E. coli; and

wherein the steps A′), A), and B), and C) are carried out as cascade reaction in one-pot system, and wherein a ratio of R—CHO (1) and the free coenzyme A (2b) is 10 to 1000.

An embodiment of the invention is directed to a method for preparation of a compound of the formula (I)

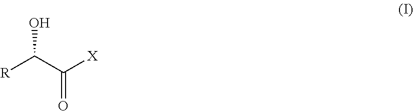

comprising:
A) providing a compound R—CHO (1) and an oxalyl-CoA (2′)

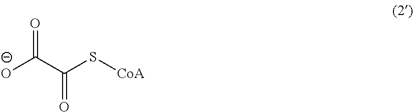

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain (S)-α-hydroxyacyl-CoA thioester (3)

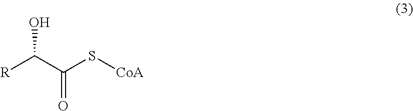

wherein the oxalyl-CoA decarboxylase (3a) is a peptide encoded by a gene selected from Methylobacterium extorquens or a mutant thereof; or additionally comprising the step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

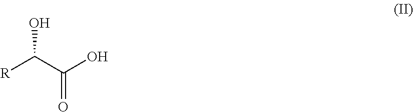

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of R—CHO (1) and the free coenzyme A (2b) is 10 to 1000.

A preferred embodiment of the invention is related to a method for preparation of a compound of the formula (II)

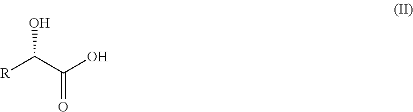

comprising:
A) providing a compound R—CHO (1) and an oxalyl-CoA (2′)

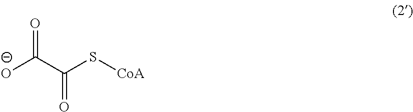

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

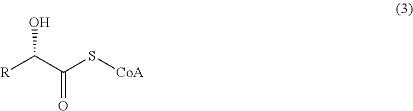

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

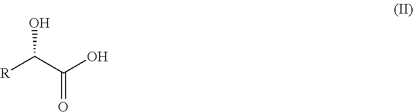

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of R—CHO (1) and the free coenzyme A (2b) is 10 to 1000.

In a method according to the invention wherein the steps A′), A), B), and C) are carried out as cascade reaction in one-pot system a ratio of the carboxylate (2a) and the free coenzyme A (2b) is preferably 10 to 10,000.

An embodiment of the present invention is directed to a method for preparation of a compound of the formula (I)

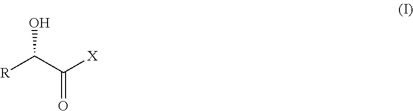

comprising:
A) providing a compound R—CHO (1) and an acyl-CoA (2)

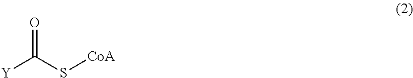

wherein Y represents —H, or —COO⁻,
in a buffer solution with a pH-value in the range of 5.0 to 7.5, optionally together with at least one co-solvent, and wherein acyl-CoA (2) can be prepared by the following step A′):
A′) reacting an carboxylate (2a) with a free coenzyme A (2b) by acyl-CoA synthetase (2c) in the presence of ATP (2e) to produce an acyl-CoA (2);
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

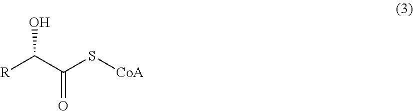

wherein the oxalyl-CoA decarboxylase (3a) is a peptide encoded by a gene selected from Methylobacterium extorquens or a mutant thereof; or additionally comprising the step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

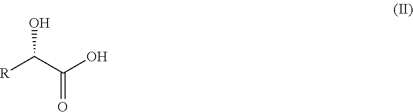

wherein the hydrolase is acyl-CoA thioester hydrolase YciA (4) encoded by a gene selected from E. coli; and

wherein the steps A′), A), and B), and C) are carried out as cascade reaction in one-pot system, and wherein a ratio of the carboxylate (2a) and the free coenzyme A (2b) is preferably 10 to 10,000.

In a method according to the invention, wherein the steps A′), A), B), and C) are carried out as cascade reaction in one-pot system a ratio of the oxalate (2a′) and the free coenzyme A (2b) is preferably 10 to 10,000.

The reduction of the used amount of the free coenzyme A leads to a substrate economic procedure. Moreover, due to the reduction of the amount of starting material solvent can be reduced, and in total a higher amount of the starting material aldehyde can be transformed in an apparatus of the same size.

An embodiment of the invention is directed to a method for preparation of a compound of the formula (I)

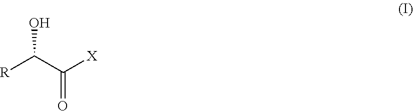

comprising:
A) providing a compound R—CHO (1) and an oxalyl-CoA (2′)

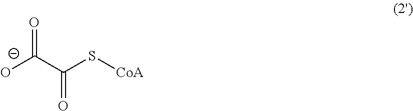

in a buffer solution with a pH-value in the range of 5.0 to 7.5 optionally together with at least one co-solvent wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

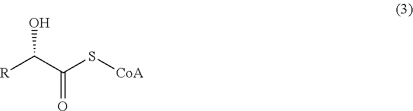

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

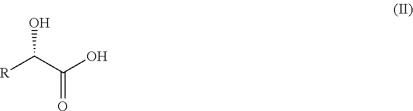

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of the oxalate (2a′) and the free coenzyme A (2b) is 10 to 10,000.

A preferred embodiment of the invention is related to a method for preparation of a compound of the formula (II)

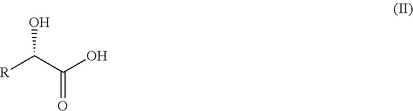

comprising:
A) providing a compound R—CHO (1) and an oxalyl-CoA (2′)

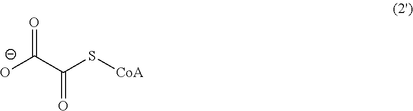

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

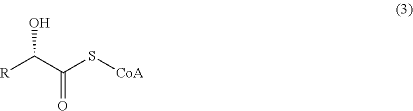

wherein the oxalyl-CoA decarboxylase (3a) is a peptide encoded by a gene selected from Methylobacterium extorquens or a mutant thereof; and
C) cleaving the coenzyme moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

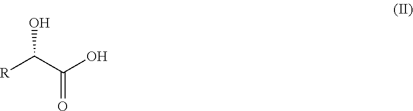

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of the oxalate (2a′) and the free coenzyme A (2b) is 10 to 10,000.

In step A) of a method according to the invention, a concentration of R—CHO (1) can be in the range of 5 mM to 50 mM. Preferably the concentration is in the range of 10 mM to 45 mM, more preferably 15 mM to 40 mM, more preferably 20 mM to 35 mM, more preferably 20 mM to 30 mM, more preferably 22.5 mM to 30 mM, and most preferably 25 mM to 30 mM.

An embodiment of the present invention is directed to a method for preparation of a compound of the formula (I)

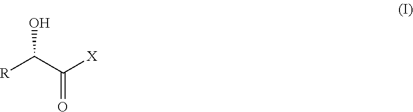

comprising:
A) providing a compound R—CHO (1) and an acyl-CoA (2)

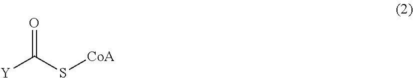

wherein Y represents —H, or —COO⁻, in a buffer solution with a pH-value in the range of 5.0 to 7.5 optionally together with at least one co-solvent, wherein a concentration of R—CHO (1) is in the range of 5 mM to 50 mM (25 mM);
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

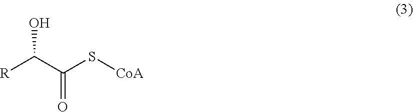

wherein the oxalyl-CoA decarboxylase (3a) is a peptide encoded by a gene selected from Methylobacterium extorquens or a mutant thereof; and optionally comprising the additional step c)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

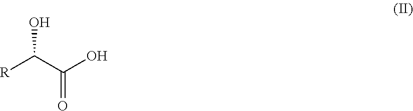

wherein the hydrolase is acyl-CoA thioester hydrolase YciA (4) encoded by a gene selected from E. coli.

An embodiment of the invention is directed to a method for preparation of a compound of the formula (I)

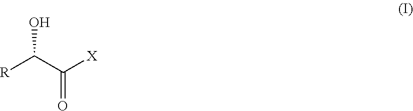

comprising:
A) providing a compound R—CHO (1) and an oxalyl-CoA (2′)

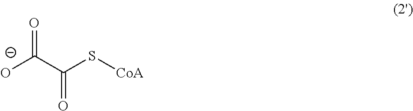

in a buffer solution with a pH-value in the range of 5.0 to 7.5 optionally together with at least one co-solvent, wherein a concentration of R—CHO (1) is in the range of 5 mM to 50 mM (25 mM);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

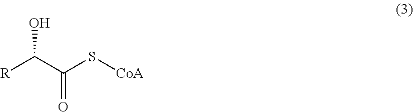

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

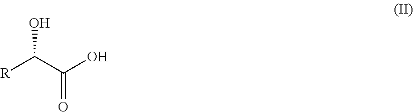

wherein the hydrolase is acyl-CoA thioester hydrolase YciA (4) encoded by a gene selected from E. coli.

Another embodiment of the present invention is related to a method for preparation of a compound of the formula (II)

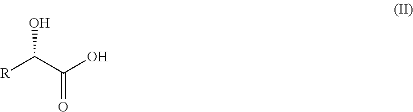

comprising:
A) providing a compound R—CHO (1) and an oxalyl-CoA (2′)

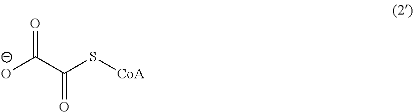

in a buffer solution with a pH-value in the range of 5.0 to 7.5 optionally together with at least one co-solvent, wherein a concentration of R—CHO (1) is in the range of 5 mM to 50 mM (25 mM);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

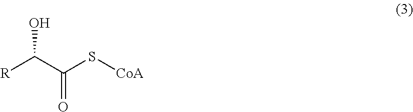

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

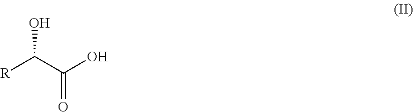

wherein the hydrolase is acyl-CoA thioester hydrolase YciA (4) encoded by a gene selected from E. coli.

A preferred embodiment of the invention is related to a method for preparation of a compound of the formula (II)

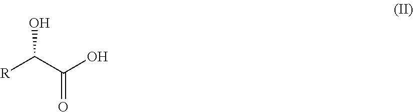

comprising:
A) providing a compound R—CHO (1) and an oxalyl-CoA (2′)

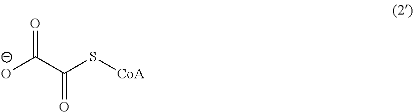

in a buffer solution with a pH-value in the range of 5.0 to 7.5 optionally together with at least one co-solvent, wherein a concentration of R—CHO (1) is in the range of 5 mM to 50 mM (25 mM), wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

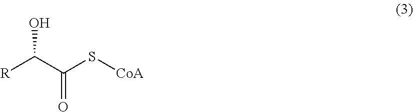

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

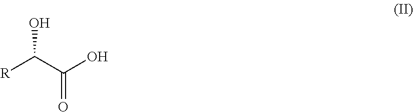

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of R—CHO (1) and the free coenzyme A (2b) is 10 to 10,000.

In step B) of a method according to invention as described herein, a molar ratio of the thiamine diphosphate (3b) compared to R—CHO (1) can be from 0.001 to 0.01, preferably from 0.002 to 0.008, more preferably from 0.003 to 0.006 with a concentration in the range of 0.05 mM to 0.5 mM, and most preferably 0.1 mM to 0.25 mM.

An embodiment of the present invention is directed to a method for preparation of a compound of the formula (I)

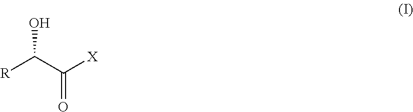

comprising:
A) providing a compound R—CHO (1) and an acyl-CoA (2)

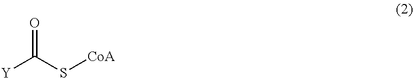

wherein Y represents —H, or —COO⁻, in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

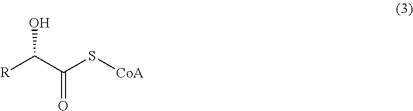

wherein the oxalyl-CoA decarboxylase (3a) is a peptide encoded by a gene selected from Methylobacterium extorquens or a mutant thereof, and wherein a molar ratio of the thiamine diphosphate (3b) compared to R—CHO (1) is 0.001 to 0.01 with a concentration in the range of 0.05 mM to 0.5 mM; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

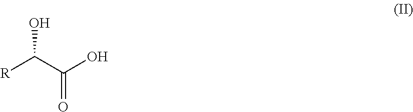

wherein the hydrolase is acyl-CoA thioester hydrolase YciA (4) encoded by a gene selected from E. coli.

An embodiment of the invention is directed to a method for preparation of a compound of the formula (I)

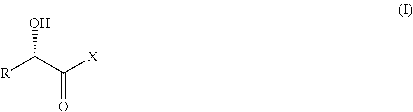

comprising:
A) providing a compound R—CHO (1) and an oxalyl-CoA (2′)

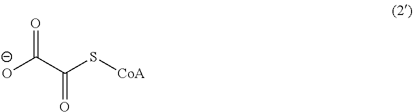

in a buffer solution with a pH-value in the range of 5.0 to 7.5, optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

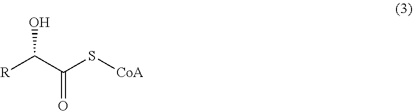

wherein the oxalyl-CoA decarboxylase (3a) is a peptide encoded by a gene selected from Methylobacterium extorquens or a mutant thereof, and wherein a molar ratio of the thiamine diphosphate (3b) compared to R—CHO (1) is 0.001 to 0.01 with a concentration in the range of 0.05 mM to 0.5 mM; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

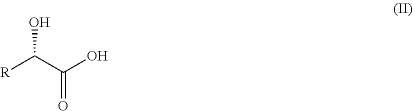

wherein the hydrolase is acyl-CoA thioester hydrolase YciA (4) encoded by a gene selected from E. coli.

Another embodiment of the present invention is related to a method for preparation of a compound of the formula (II)

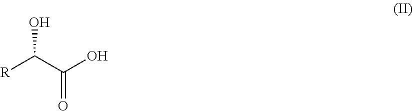

comprising:
A) providing a compound R—CHO (1) and an oxalyl-CoA (2′)

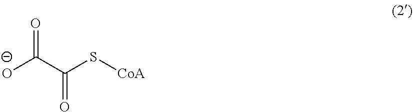

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain (S)-2-hydroxyacyl-CoA thioester (3)

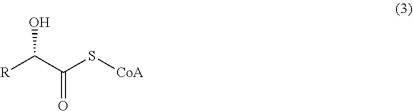

wherein the oxalyl-CoA decarboxylase (3a) is a peptide encoded by a gene selected from Methylobacterium extorquens or a mutant thereof, and wherein a molar ratio of the thiamine diphosphate (3b) compared to R—CHO (1) is 0.001 to 0.01 with a concentration in the range of 0.05 mM to 0.5 mM; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

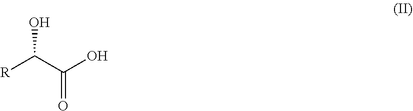

wherein the hydrolase is acyl-CoA thioester hydrolase YciA (4) encoded by a gene selected from E. coli.

A preferred embodiment of the invention is related to a method for preparation of a compound of the formula (II)

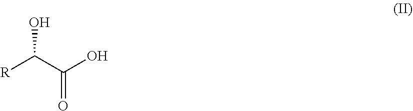

comprising:
A) providing a compound R—CHO (1) and an oxalyl-CoA (2′)

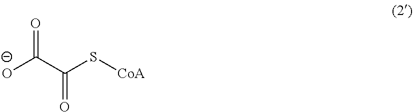

in a buffer solution with a pH-value in the range of 5.0 to 7.5 optionally together with at least one co-solvent, wherein a concentration of R—CHO (1) is in the range of 5 mM to 50 mM, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

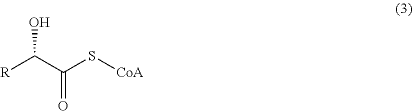

wherein the oxalyl-CoA decarboxylase (3a) is a peptide encoded by a gene selected from Methylobacterium extorquens or a mutant thereof, and wherein a molar ratio of the thiamine diphosphate (3b) compared to R—CHO (1) is 0.001 to 0.01 with a concentration in the range of 0.05 mM to 0.5 mM; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

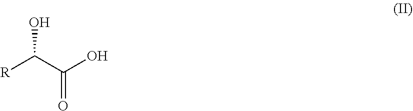

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of R—CHO (1) and the free coenzyme A (2b) is 10 to 1000.

In step A) of a method according to the invention, the pH-value of the buffer solution is preferably in the range of 6.5 to 7.5, and more preferably in the range of 6.7 to 7.0.

In general, in step A) any buffer can be used to adjust the pH value, notably sodium or potassium phosphate buffer. The buffer solution can contain at least one compound selected from the group comprising or consisting of 2-[[1,3-dihydroxy-2-(hydroxymethyl)propan-2-yl]amino]ethanesulfonic acid (TES), 2-(N-morpholino)ethanesulfonic acid (MES), 2-[Bis(2-hydroxyethyl)amino]-2-(hydroxymethyl)propane-1,3-diol (Bis-Tris), 2-[(2-amino-2-oxoethyl)-(carboxymethyl)amino]acetic acid (ADA), 2-(carbamoylmethylamino)ethanesulfonic acid (ACES), 1,4-Piperazinediethanesulfonic acid (PIPES), 3-Morpholino-2-hydroxypropanesulfonic acid (MOPSO), 2,2′-(Propane-1,3-diyldiimino)bis[2-(hydroxymethyl)propane-1,3-diol] (BTP), N,N-Bis(2-hydroxyethyl)-2-aminoethanesulfonic acid (BES), 3-Morpholinopropane-1-sulfonic acid (MOPS), 2-[4-(2-hydroxyethyl)piperazin-1-yl]ethanesulfonic acid (HEPES), potassium phosphate, sodium phosphate, and imidazole, preferably 2-[[1,3-dihydroxy-2-(hydroxymethyl)propan-2-yl]amino]ethanesulfonic acid (TES).

The buffer can be prepared by use of a base such as lithium hydroxide, sodium hydroxide, potassium hydroxide, rubidium hydroxide, and/or caesium hydroxide. Preferably, potassium hydroxide is used.

In step A) of a method according to the invention, the buffer solution can comprise at least one compound selected from the group comprising or consisting of 2-[[1,3-dihydroxy-2-(hydroxymethyl)propan-2-yl]amino]ethanesulfonic acid, 2-(N-morpholino)ethanesulfonic acid (MES), 2-[Bis(2-hydroxyethyl)amino]-2-(hydroxymethyl)propane-1,3-diol (Bis-Tris), 2-[(2-amino-2-oxoethyl)-(carboxymethyl)amino]acetic acid (ADA), 2-(carbamoylmethylamino)ethanesulfonic acid (ACES), 1,4-Piperazinediethanesulfonic acid (PIPES), 3-Morpholino-2-hydroxypropanesulfonic acid (MOPSO), 2,2′-(Propane-1,3-diyldiimino)bis[2-(hydroxymethyl)propane-1,3-diol] (BTP), N,N-Bis(2-hydroxyethyl)-2-aminoethanesulfonic acid (BES), 3-Morpholinopropane-1-sulfonic acid (MOPS), 2-[4-(2-hydroxyethyl)piperazin-1-yl]ethanesulfonic acid (HEPES), potassium phosphate, sodium phosphate, and imidazole in the range of 0.25 wt % to 2.5 wt %, preferably 1 wt % to 2 wt %

Preferably, in step A) of a method according to the invention, the buffer solution can comprise at least one compound selected from the group comprising or consisting 2-[[1,3-dihydroxy-2-(hydroxymethyl)propan-2-yl]amino]ethanesulfonic acid (TES), 2-(N-morpholino)ethanesulfonic acid (MES), 2-[Bis(2-hydroxyethyl)amino]-2-(hydroxymethyl)propane-1,3-diol (Bis-Tris), 2-[(2-amino-2-oxoethyl)-(carboxymethyl)amino]acetic acid (ADA), 2-(carbamoylmethylamino)ethanesulfonic acid (ACES), 1,4-Piperazinediethanesulfonic acid (PIPES), 3-Morpholino-2-hydroxypropanesulfonic acid (MOPSO), 2,2′-(Propane-1,3-diyldiimino)bis[2-(hydroxymethyl)propane-1,3-diol] (BTP), N,N-Bis(2-hydroxyethyl)-2-aminoethanesulfonic acid (BES), 3-Morpholinopropane-1-sulfonic acid (MOPS), 2-[4-(2-hydroxyethyl)piperazin-1-yl]ethanesulfonic acid (HEPES), potassium phosphate, sodium phosphate, and imidazole in the range of 0.25 wt % to 2.5 wt %, preferably 1 wt % to 2 wt %

In step A) of a method according to the invention, the buffer solution can comprise 2-[[1,3-dihydroxy-2-(hydroxymethyl)propan-2-yl]amino]ethanesulfonic acid (TES) in the range of 0.25 wt % to 2.5 wt %, preferably 1 wt % to 2 wt % and the pH-value is in the range of 6.5 to 7.5, and preferably 6.7 to 7.0.

In step A) of a method according to the invention, the buffer solution can comprise 2-[[1,3-dihydroxy-2-(hydroxymethyl)propan-2-yl]amino]ethanesulfonic acid (TES) in the range of 0.25 wt % to 2.5 wt %, preferably 1 wt % to 2 wt % and the pH-value is in the range of 5.0 to 7.5, preferably in the range of 6.5 to 7.5, and more preferably in range of 6.7 to 7.0.

An embodiment of the present invention is directed to a method for preparation of a compound of the formula (I)

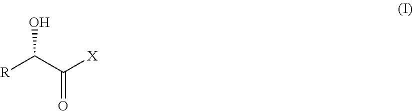

comprising:
A) providing a compound R—CHO (1) and an acyl-CoA (2)

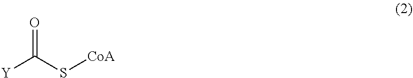

wherein Y represents —H, or —COO⁻,
in a buffer solution with a pH-value in the range of 5.0 to 7.5 optionally together with at least one co-solvent, and wherein the buffer solution comprises at least one compound selected from the group comprising or consisting of 2-[[1,3-dihydroxy-2-(hydroxymethyl)propan-2-yl]amino]ethanesulfonic acid (TES), 2-(N-morpholino)ethanesulfonic acid (MES), 2-[Bis(2-hydroxyethyl)amino]-2-(hydroxymethyl)propane-1,3-diol (Bis-Tris), 2-[(2-amino-2-oxoethyl)-(carboxymethyl)amino]acetic acid (ADA), 2-(carbamoylmethylamino)ethanesulfonic acid (ACES), 1,4-Piperazinediethanesulfonic acid (PIPES), 3-Morpholino-2-hydroxypropanesulfonic acid (MOPSO), 2,2′-(Propane-1,3-diyldiimino)bis[2-(hydroxymethyl)propane-1,3-diol] (BTP), N,N-Bis(2-hydroxyethyl)-2-aminoethanesulfonic acid (BES), 3-Morpholinopropane-1-sulfonic acid (MOPS), 2-[4-(2-hydroxyethyl)piperazin-1-yl]ethanesulfonic acid (HEPES), potassium phosphate, sodium phosphate, and imidazole;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

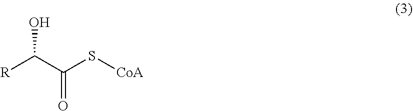

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

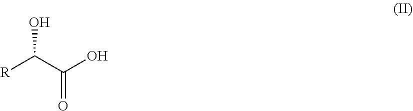

wherein the hydrolase is acyl-CoA thioester hydrolase YciA (4) encoded by a gene selected from E. coli.

A further embodiment of the present invention is directed to a method for preparation of a compound of the formula (I)

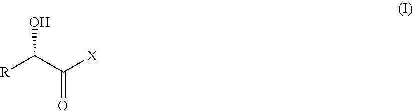

comprising:
A) providing a compound R—CHO (1) and an acyl-CoA (2)

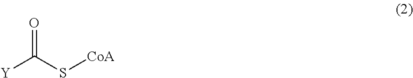

wherein Y represents —H, or —COO⁻,
in a buffer solution with a pH-value in the range of 6.5 to 7.5 optionally together with at least one co-solvent, and wherein the buffer solution comprises at least one compound selected from the group comprising or consisting of 2-[[1,3-dihydroxy-2-(hydroxymethyl)propan-2-yl]amino]ethanesulfonic acid (TES), 2-(N-morpholino)ethanesulfonic acid (MES), 2-[Bis(2-hydroxyethyl)amino]-2-(hydroxymethyl)propane-1,3-diol (Bis-Tris), 2-[(2-amino-2-oxoethyl)-(carboxymethyl)amino]acetic acid (ADA), 2-(carbamoylmethylamino)ethanesulfonic acid (ACES), 1,4-Piperazinediethanesulfonic acid (PIPES), 3-Morpholino-2-hydroxypropanesulfonic acid (MOPSO), 2,2′-(Propane-1,3-diyldiimino)bis[2-(hydroxymethyl)propane-1,3-diol] (BTP), N,N-Bis(2-hydroxyethyl)-2-aminoethanesulfonic acid (BES), 3-Morpholinopropane-1-sulfonic acid (MOPS), 2-[4-(2-hydroxyethyl)piperazin-1-yl]ethanesulfonic acid (HEPES), potassium phosphate, sodium phosphate, and imidazole in the range of 0.25 wt % to 2.5 wt %;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

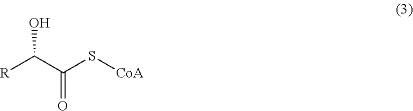

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

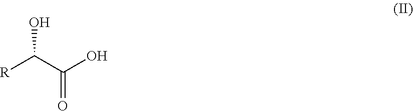

wherein the hydrolase is acyl-CoA thioester hydrolase YciA (4) encoded by a gene selected from E. coli.

An embodiment of the invention is directed to a method for preparation of a compound of the formula (I)

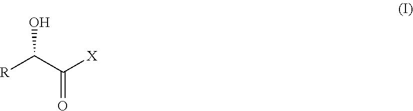

comprising:
A) providing a compound R—CHO (1) and an oxalyl-CoA (2′)

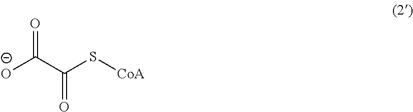

in a buffer solution with a pH-value in the range of 6.5 to 7.5 optionally together with at least one co-solvent, and wherein the buffer solution comprises at least one compound selected from the group comprising or consisting of 2-[[1,3-dihydroxy-2-(hydroxymethyl)propan-2-yl]amino]ethanesulfonic acid (TES), 2-(N-morpholino)ethanesulfonic acid (MES), 2-[Bis(2-hydroxyethyl)amino]-2-(hydroxymethyl)propane-1,3-diol (Bis-Tris), 2-[(2-amino-2-oxoethyl)-(carboxymethyl)amino]acetic acid (ADA), 2-(carbamoylmethylamino)ethanesulfonic acid (ACES), 1,4-Piperazinediethanesulfonic acid (PIPES), 3-Morpholino-2-hydroxypropanesulfonic acid (MOPSO), 2,2′-(Propane-1,3-diyldiimino)bis[2-(hydroxymethyl)propane-1,3-diol] (BTP), N,N-Bis(2-hydroxyethyl)-2-aminoethanesulfonic acid (BES), 3-Morpholinopropane-1-sulfonic acid (MOPS), 2-[4-(2-hydroxyethyl)piperazin-1-yl]ethanesulfonic acid (HEPES), potassium phosphate, sodium phosphate, and imidazole in the range of 0.25 wt % to 2.5 wt %;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

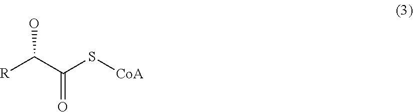

wherein the oxalyl-CoA decarboxylase (3a) is a peptide encoded by a gene selected from Methylobacterium extorquens or a mutant thereof and optionally comprising the additional step C)
C) cleaving coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

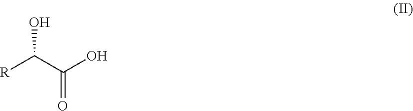

wherein the hydrolase is acyl-CoA thioester hydrolase YciA (4) encoded by a gene selected from E. coli.

Another embodiment of the present invention is related to a method for preparation of a compound of the formula (II)

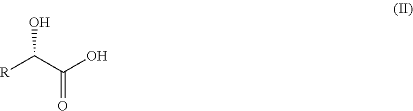

comprising:
A) providing a compound R—CHO (1) and an oxalyl-CoA (2′)

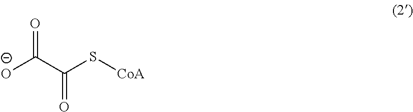

in a buffer solution with a pH-value in the range of 6.5 to 7.5 optionally together with at least one co-solvent, and wherein the buffer solution comprises at least one compound selected from the group comprising or consisting of 2-[[1,3-dihydroxy-2-(hydroxymethyl)propan-2-yl]amino]ethanesulfonic acid (TES), 2-(N-morpholino)ethanesulfonic acid (MES), 2-[Bis(2-hydroxyethyl)amino]-2-(hydroxymethyl)propane-1,3-diol (Bis-Tris), 2-[(2-amino-2-oxoethyl)-(carboxymethyl)amino]acetic acid (ADA), 2-(carbamoylmethylamino)ethanesulfonic acid (ACES), 1,4-Piperazinediethanesulfonic acid (PIPES), 3-Morpholino-2-hydroxypropanesulfonic acid (MOPSO), 2,2′-(Propane-1,3-diyldiimino)bis[2-(hydroxymethyl)propane-1,3-diol] (BTP), N,N-Bis(2-hydroxyethyl)-2-aminoethanesulfonic acid (BES), 3-Morpholinopropane-1-sulfonic acid (MOPS), 2-[4-(2-hydroxyethyl)piperazin-1-yl]ethanesulfonic acid (HEPES), potassium phosphate, sodium phosphate, and imidazole in the range of 0.25 wt % to 2.5 wt %;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

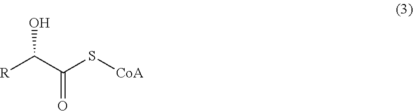

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

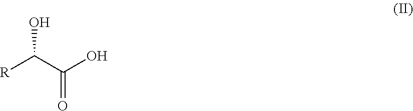

wherein the hydrolase is acyl-CoA thioester hydrolase YciA (4) encoded by a gene selected from E. coli.

A more preferred embodiment of the invention is related to a method for preparation of a compound of the formula (II)

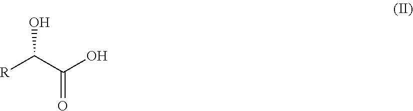

comprising:
A) providing a compound R—CHO (1) and an oxalyl-CoA (2′)

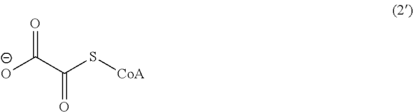

in a buffer solution with a pH-value in the range of 6.5 to 7.5 optionally together with at least one co-solvent, wherein a concentration of R—CHO (1) is in the range of 5 mM to 50 mM, wherein the buffer solution comprises at least one compound selected from the group comprising or consisting of 2-[[1,3-dihydroxy-2-(hydroxymethyl)propan-2-yl]amino]ethanesulfonic acid (TES), 2-(N-morpholino)ethanesulfonic acid (MES), 2-[Bis(2-hydroxyethyl)amino]-2-(hydroxymethyl)propane-1,3-diol (Bis-Tris), 2-[(2-amino-2-oxoethyl)-(carboxymethyl)amino]acetic acid (ADA), 2-(carbamoylmethylamino)ethanesulfonic acid (ACES), 1,4-Piperazinediethanesulfonic acid (PIPES), 3-Morpholino-2-hydroxypropanesulfonic acid (MOPSO), 2,2′-(Propane-1,3-diyldiimino)bis[2-(hydroxymethyl)propane-1,3-diol] (BTP), N,N-Bis(2-hydroxyethyl)-2-aminoethanesulfonic acid (BES), 3-Morpholinopropane-1-sulfonic acid (MOPS), 2-[4-(2-hydroxyethyl)piperazin-1-yl]ethanesulfonic acid (HEPES), potassium phosphate, sodium phosphate, and imidazole in the range of 0.25 wt % to 2.5 wt %, and wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

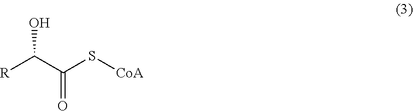

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

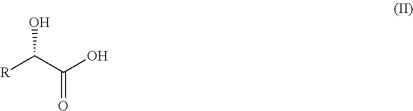

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of R—CHO (1) and the free coenzyme A (2b) is 10 to 1000.

In step B) of a method according to the invention, a concentration of the oxalyl-CoA decarboxylase can be in the range of 1 to 25 μM (5 μM), preferably 5 to 20 and/or a concentration of the magnesium (II) cation is in the range of 2.5 mM to 20 mM, preferably 5 mM to 10 mM.

An embodiment of the present invention is directed to a method for preparation of a compound of the formula (I)

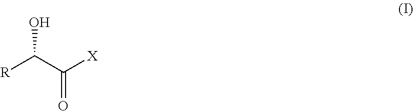

comprising:
A) providing a compound R—CHO (1) and an acyl-CoA (2)

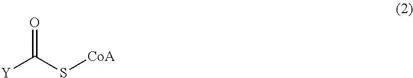

wherein Y represents —H, or —COO⁻,
in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

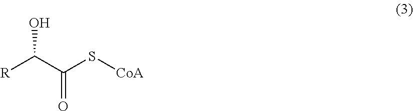

wherein the oxalyl-CoA decarboxylase (3a) is a peptide encoded by a gene selected from Methylobacterium extorquens or a mutant thereof, and wherein a concentration of the oxalyl-CoA decarboxylase is in the range of 1 to 25 μM and/or a concentration of the magnesium (II) cation is in the range of 2.5 mM to 20 mM; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

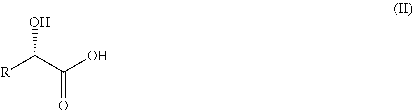

wherein the hydrolase is acyl-CoA thioester hydrolase YciA (4) encoded by a gene selected from E. coli.

An embodiment of the invention is directed to a method for preparation of a compound of the formula (I)

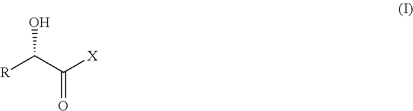

comprising:
A) providing a compound R—CHO (1) and an oxalyl-CoA (2′)

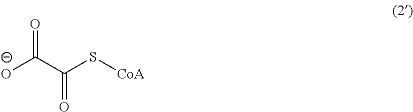

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

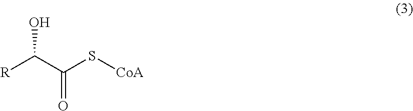

wherein the oxalyl-CoA decarboxylase (3a) is a peptide encoded by a gene selected from Methylobacterium extorquens or a mutant thereof, and wherein a concentration of the oxalyl-CoA decarboxylase is in the range of 1 to 25 μM and/or a concentration of the magnesium (II) cation is in the range of 2.5 mM to 20 mM and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

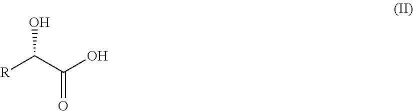

wherein the hydrolase is acyl-CoA thioester hydrolase YciA (4) encoded by a gene selected from E. coli.

Another embodiment of the present invention is related to a method for preparation of a compound of the formula (II)

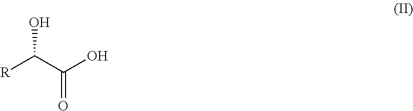

comprising:
A) providing a compound R—CHO (1) and an oxalyl-CoA (2′)

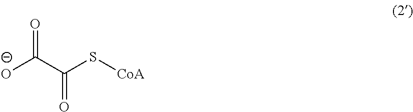

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

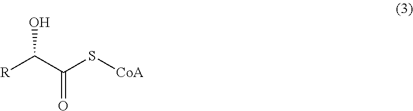

wherein the oxalyl-CoA decarboxylase (3a) is a peptide encoded by a gene selected from Methylobacterium extorquens or a mutant thereof, and wherein a concentration of the oxalyl-CoA decarboxylase is in the range of 1 to 25 μM and/or a concentration of the magnesium (II) cation is in the range of 2.5 mM to 20 mM; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

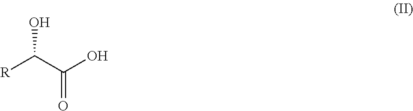

wherein the hydrolase is acyl-CoA thioester hydrolase YciA (4) encoded by a gene selected from E. coli.

A preferred embodiment of the invention is related to a method for preparation of a compound of the formula (II)

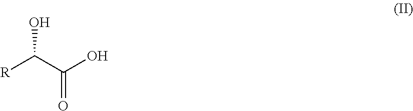

comprising:
A) providing a compound R—CHO (11 and an oxalyl-CoA (2′)

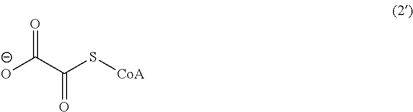

in a buffer solution with a pH-value in the range of 6.5 to 7.5 optionally together with at least one co-solvent, wherein a concentration of R—CHO (1) is in the range of 5 mM to 50 mM, wherein the buffer solution comprises 2-[[1,3-dihydroxy-2-(hydroxymethyl)propan-2-yl]amino]ethanesulfonic acid (TES) in the range of 0.25 wt % to 2.5 wt %, and wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

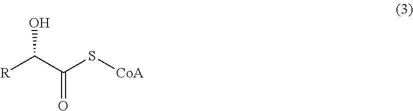

wherein the oxalyl-CoA decarboxylase (3a) is a peptide encoded by a gene selected from Methylobacterium extorquens or a mutant thereof, wherein a molar ratio of the thiamine diphosphate (3b) compared to R—CHO (1) is 0.001 to 0.01 with a concentration in the range of 0.05 mM to 0.5 mM, and wherein a concentration of the oxalyl-CoA decarboxylase is in the range of 1 to 25 μM and/or a concentration of the magnesium (II) cation is in the range of 2.5 mM to 20 mM; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

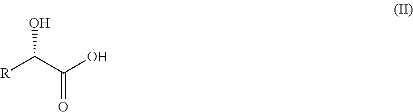

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of R—CHO (1) and the free coenzyme A (2b) is 10 to 1000.

In step B), the mutant can be selected from a group comprising or consisting of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4. Preferably, the used peptide is OXC_{Me-Y497A} having the sequence SEQ ID NO. 2 and/or OXC_{Me-S568A}, and most preferably OXC_{Me-Y497A} having the sequence SEQ ID NO. 2.

An embodiment of the invention is related to a method for the preparation of a-hydroxyacyl compound of the formula (I)

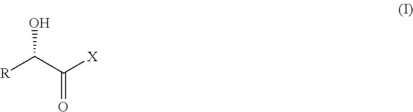

comprising:
A) providing a compound R—CHO (1) and an acyl-CoA (2)

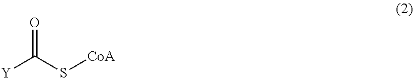

wherein Y represents —H, or —COO⁻,
in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

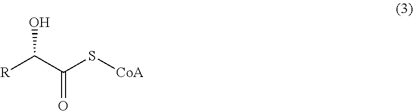

wherein the oxalyl-CoA decarboxylase (3a) is a peptide encoded by a gene selected from Methylobacterium extorquens or a mutant thereof and wherein the mutant preferably is selected from the group comprising or consisting of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

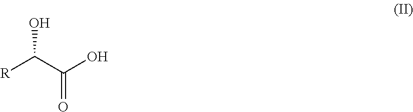

wherein the hydrolase is acyl-CoA thioester hydrolase YciA (4) encoded by a gene selected from E. coli.

An embodiment of the invention is directed to a method for preparation of a compound of the formula (I)

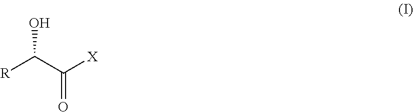

comprising:
A) providing a compound R—CHO (1) and an oxalyl-CoA (2′)

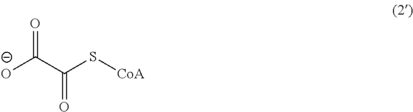

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

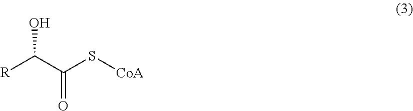

wherein the oxalyl-CoA decarboxylase (3a) is a peptide encoded by a gene selected from Methylobacterium extorquens or a mutant thereof and wherein the mutant preferably is selected from the group comprising or consisting of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

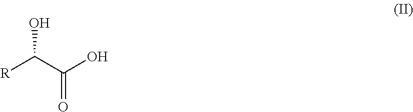

wherein the hydrolase is acyl-CoA thioester hydrolase YciA (4) encoded by a gene selected from E. coli.

Another embodiment of the present invention is related to a method for preparation of a compound of the formula (II)

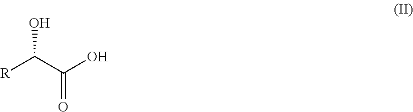

comprising:
A) providing a compound R—CHO (1) and an oxalyl-CoA (2′)

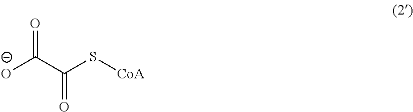

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

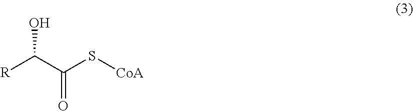

wherein the oxalyl-CoA decarboxylase (3a) is a peptide encoded by a gene selected from Methylobacterium extorquens or a mutant thereof, and wherein the mutant preferably is selected from the group comprising or consisting of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

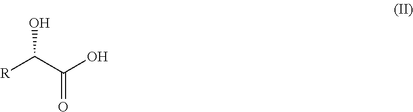

wherein the hydrolase is acyl-CoA thioester hydrolase YciA (4) encoded by a gene selected from E. coli.

A preferred embodiment of the invention is related to a method for preparation of a compound of the formula (II)

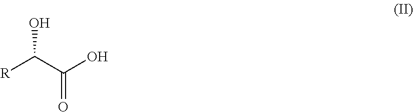

comprising:
A) providing a compound R—CHO (1) and an oxalyl-CoA (2′)

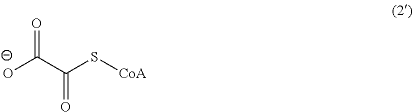

in a buffer solution with a pH-value in the range of 6.5 to 7.5 optionally together with at least one co-solvent, wherein a concentration of R—CHO (1) is in the range of 5 mM to 50 mM, wherein the buffer solution comprises 2-[[1,3-dihydroxy-2-(hydroxymethyl)propan-2-yl]amino]ethanesulfonic acid (TES) in the range of 0.25 wt % to 2.5 wt %, and wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

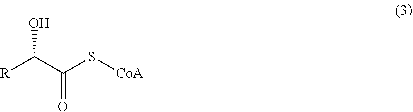

wherein the oxalyl-CoA decarboxylase (3a) is a peptide encoded by a gene selected from Methylobacterium extorquens or a mutant thereof, wherein a molar ratio of the thiamine diphosphate (3b) compared to R—CHO (1) is 0.001 to 0.01 with a concentration in the range of 0.05 mM to 0.5 mM, wherein a concentration of the oxalyl-CoA decarboxylase is in the range of 1 to 25 μM and/or a concentration of the magnesium (II) cation is in the range of 2.5 mM to 20 mM, and wherein the mutant preferably is selected from the group comprising or consisting of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

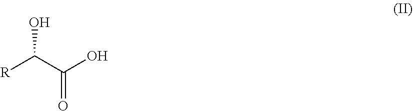

wherein the hydrolase is acyl-CoA thioester hydrolase YciA (4) encoded by a gene selected from E. coli; and

wherein steps A′), A), B), and C) are carried out as cascade reaction in one-pot system, and wherein a ratio of R—CHO (1) and the free coenzyme A (2b) is 10 to 1000.

It is especially preferred, when the oxalyl-CoA decarboxylase (3a) is selected from a group comprising or consisting of OXC_{Me}, OXC_{Me-Y497A}, OXC_{Me-S568A} or OXC_{Me-Y497A-S568A}.

Decarboxylation of oxalyl-CoA proceeds via formation of the thiamine-α-hydroxyl-CoA adduct (see FIG. 3), which can be either protonated and released as formyl-CoA or undergo a nucleophilic attack on benzaldehyde to form α-hydroxyacyl-CoA in particular mandelyl-CoA. Surprisingly, it could be found that mandelyl-CoA formation can be increased by suppressing the unwanted protonation reaction (and subsequent release of formyl-CoA) that competes with carboligation.

In OXC_{Me} variants OXC_{Me-Y497A} and OXC_{Me-S568A}, formyl-CoA formation was decreased 20 to 50-fold (FIG. 14B), while the K_{M} of both variants was largely unaffected (example 6, table 3, and FIG. 13). When starting with oxalyl-CoA and benzaldehyde as an example OXC_{Me-Y497A} showed 5-fold increased mandelyl-CoA production rate (20 min⁻¹; FIG. 14C), suggesting that activity of the enzyme was successfully redirected towards carboligation by suppressing protonation of thiamine-α-hydroxyl-CoA-adduct (see FIG. 3). Moreover, the reaction time starting from formyl-CoA could be reduced remarkably by means of OXC_{Me-Y497A} in comparison to OXC_{Me} (example 9, FIG. 14).

When the steps A′), A), B) and C) are carried out as cascade reaction in one-pot system, the reaction time could also be reduced significantly along with a higher enantiomeric excess by means of OXC_{Me-Y497A} in comparison to OXC_{Me}.

The variant OXC_{Me-Y497A-S568A} is also suitable for the carbon-carbon-bond formation. The kinetic data are shown example 6, table 3, and FIG. 13.

Thus, another aspect of the present invention is directed to a mutant of a peptide OXC_{Me} encoded by a gene from Methylobacterium extorquens having a sequence according to SEQ ID No. 1 (FIG. 8), wherein at least one tyrosine or serine is replaced by an amino acid selected independently of each other from the group comprising or consisting of alanine, phenylalanine, and glycine, preferably tyrosine is replaced by phenylalanine, alanine, and/or glycine, and serine is replaced by alanine and/or glycine, most preferably the tyrosine of SEQ ID NO. 1 at position 497 is replaced by alanine, phenylalanine or glycine, and/or serine of SEQ ID NO. 1 at position 568 is replaced by alanine or glycine.

The present invention is thus also directed to a peptide having a sequence SEQ ID NO. 1 (FIG. 8), wherein at least one tyrosine or serine is replaced independently of each other by an amino acid selected from the group comprising or consisting of phenylalanine, alanine and glycine, and preferably alanine.

A preferred embodiment according to the invention is related to a peptide OXC_{Me-Y497A} having a sequence SEQ ID NO.2, OXC_{Me-S568A} having the sequence SEQ ID NO. 3 or OXC_{Me-Y497A-S568A} having the sequence SEQ ID NO. 4.

Thus, a preferred embodiment according to the invention is directed to a peptide having a sequence SEQ ID NO. 2 (FIG. 9), SEQ ID NO. 3 (FIG. 10) and/or SEQ ID NO. 4 (FIG. 11).

An embodiment of the present invention is related to a mutant of the peptide oxalyl-CoA decarboxylase OXC_{Me} encoded by a gene from Methylobacterium extorquens, wherein preferably the position 497 and/or 568 of OXC_{Me} having the sequence SEQ ID NO. 1 is replaced independently of each other by amino acid selected from the group comprising or consisting of alanine, phenylalanine, and glycine, preferably alanine.

A preferred embodiment according to the present invention is related to the use of peptide having a sequence SEQ ID NO. 2 (FIG. 9), SEQ ID NO. 3 (FIG. 10) and/or SEQ ID NO. 4 (FIG. 11) for the synthesis of an α-hydroxycarboxylic acids, salts thereof, or α-hydroxyacyl-CoA thioester.

A preferred embodiment according to the invention is directed to a composition comprising or consisting of a peptide having a sequence SEQ ID NO. 2 (FIG. 9), SEQ ID NO. 3 (FIG. 10) and/or SEQ ID NO. 4 (FIG. 11), and a thioesterase YciA encoded by a gene selected from E. coli.

A preferred embodiment according to the present invention is related to the use of a composition comprising or consisting of a peptide having a sequence SEQ ID NO. 2 (FIG. 9), SEQ ID NO. 3 (FIG. 10) and/or SEQ ID NO. 4 (FIG. 11), and a thioesterase YciA encoded by a gene selected from E. coli for the preparation of an α-hydroxycarboxylic acid, salts thereof, or α-hydroxyacyl-CoA thioester.

Another preferred embodiment according to the invention is thus directed to a composition comprising or consisting of a peptide having a sequence SEQ ID NO. 2 (FIG. 9), SEQ ID NO. 3 (FIG. 10) and/or SEQ ID NO. 4 (FIG. 11), a thioesterase YciA encoded by a gene selected from E. coli, and an oxalyl-CoA synthetase OXS encoded by a gene selected from Methylobacterium extorquens.

Another preferred embodiment according to the invention is related to the use of a composition comprising or consisting of a peptide having a sequence SEQ ID NO. 2 (FIG. 9), SEQ ID NO. 3 (FIG. 10) and/or SEQ ID NO. 4 (FIG. 11), a thioesterase YciA encoded by a gene selected from E. coli, and an oxalyl-CoA synthetase (OXS) encoded by a gene selected from Methylobacterium extorquens for the synthesis of an α-hydroxycarboxylic acid, or salts thereof.

A further aspect of the present invention is related to a method for preparation of a mutant of oxalyl-CoA decarboxylase from Methylobacterium extorquens (OXC_{Me}), wherein OXC_{Me} have the sequence SEQ ID NO. 1, comprising the following steps:
I) Providing a chromosomal DNA from Methylobacterium extorquens;
II) Providing at least one primer;
III) Polymer chain reaction to obtain a DNA of oxalyl-CoA decarboxylase (OXC_{Me});
IV) Ligation of the DNA of oxalyl-CoA decarboxylase (OXC_{Me}) into a vector to obtain a vector ligated with the DNA of oxalyl-CoA decarboxylase;
V) mutation of ligated vector obtained in step IV) by polymer chain reaction using at least one primer and the ligated vector obtained in step IV);
VI) providing a cell culture containing a host cell;
VII) introducing the ligated vector according to step V) into the host cell and expressing a mutant of oxalyl-CoA decarboxylase from Methylobacterium extorquens; and
VIII) Isolating of the mutant of oxalyl-CoA decarboxylase from Methylobacterium extorquens.

“Vector”, as used herein, refers to a natural or synthetic single or double stranded plasmid or viral nucleic acid molecule, or any other nucleic acid molecule that can be transfected or transformed into cells and replicate independently of, or within, the host cell genome. A nucleic acid can be inserted into a vector by cutting the vector with restriction enzymes and ligating the pieces together. The nucleic acid molecule can be RNA or DNA. Preferably the nucleic acid molecule is DNA. Preferably, the vector is a plasmid. Preferably, the plasmid is pET-16b.

The host cell can be any cell being suitable for the expression of a mutated oxalyl-CoA decarboxylase from Methylobacterium extorquens. Preferably, the host cell is E. coli.

In step VI) or in a step VI″), further ingredients can be added to the cell culture including induction agent for the protein expression and selection agent.

As an induction agent for protein expression L-(+)-Arabinose or Isopropyl β-D-1-thiogalactopyranoside (IPTG) can be used. Preferably, the induction agent for protein expression is IPTG.

As a selection agent an antibiotic can be used. Preferably, the antibiotic is ampicillin or chloramphenicol.

If a selection agent is used, the method contains a step VII″) after step VII) cultivation of the host cells containing the ligated vector.

“Primer”, as used herein, has a nucleotide sequence complementary to a portion of a targeted nucleic acid molecule, i.e., a priming sequence. The term “complementary” used herein refers to “substantially complementary”, which indicates that a primer is sufficiently complementary to a template or targeted nucleic acid sequence to be selectively hybridized with the template or targeted nucleic acid sequence. Thus, the primer may have at least one mismatch with the template as long as it functions as a primer.

Preferably, the at least one primer is selected from the group comprising or consisting of the following sequences:

(SEQ ID NO. 13)
CAACAACGGCATCGCTCGCGGCACCGAC
(SEQ ID NO. 14)
GTCGGTGCCGCGAGCGATGCCGTTGTTG
(SEQ ID NO. 15)
CCGGCAGCGAGGCCGGCAATATCGG
(SEQ ID NO. 16)
CCGATATTGCCGGCCTCGCTGCCGG
“DNA” refers to deoxyribonucleic acid.

Further, in order to accelerate the carbon-carbon formation in step B) or within the cascade reaction in a one-pot system according to a method of invention since the oxalyl-CoA which is partially transformed to formyl-CoA via the protonation of the thiamine adduct during the reaction can be regenerated by means of formyl-CoA:oxalate transferase (FIG. 4).

Moreover, in case of a cascade reaction in a one pot system when using oxalyl-CoA the hydrolysis of the formed formyl-CoA by the thioesterase YciA can be prevented, and thus the consumption of ATP being needed for the hydrolysis by the thioesterase YciA (FIG. 5).

In case of a cascade reaction in a one pot system when using oxalyl-CoA it is further preferred if the acyl-CoA thioester hydrolase YciA (4) encoded by a gene selected from E. coli and/or α-hydroxyacyl-CoA:oxalate CoA-transferase is used.

A “formyl-CoA:oxalate transferase” (“FRC”) transfers the coenzyme A moiety from the formyl-CoA to the oxalate, and thus forms oxalyl-CoA.

Thus, preferably in step B) according to a method of the present invention a formyl-CoA:oxalate transferase (FRC) is used in addition to the oxalyl-CoA decarboxylase. More preferably, the formyl-CoA:oxalate transferase which can be used in step B) according to a method of the present invention is a formyl-CoA:oxalate transferase encoded by a gene selected from Oxalobacter formigenes or a mutant thereof.

In step C) of a method according to the invention, the cleavage of the coenzyme A moiety can be performed by a hydrolase such as the acyl-CoA thioester hydrolase (thioesterase) YciA (4).

Alternative, in step C) of a method according to the invention the cleavage can be performed by a α-hydroxyacyl-CoA:oxalate CoA-transferase or α-hydroxyacyl-CoA:formate CoA-transferase. In this case, a carboxylic acid or a salt thereof is generated by the transfer of the coenzyme A moiety to an oxalate or formate.

Thus, in step C) the coenzyme A moiety can be cleaved from the α-hydroxyacyl-CoA thioester in particular (S)-α-hydroxyacyl-CoA thioester by transferring of said moiety to an oxalate or a formate through α-hydroxyacyl-CoA:oxalate CoA-transferase and/or α-hydroxyacyl-CoA:formate CoA-transferase, or by hydrolysing the α-hydroxyacyl-CoA thioester by a hydrolase, wherein the hydrolase is acyl-CoA thioester hydrolase YciA (4) encoded by a gene selected from E. coli.

Hydrolysing said α-hydroxyacyl-CoA thioester (3′) by a hydrolase to obtain the compound of the formula (II′) or a salt thereof

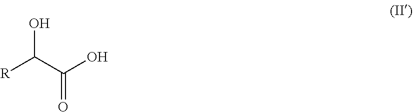

wherein the hydrolase is acyl-CoA thioester hydrolase YciA (4) encoded by a gene selected from E. coli in step C) is preferred.

Hydrolysing said (S)-α-hydroxyacyl-CoA thioester (3) by a hydrolase to obtain the compound of the formula (II) or a salt thereof

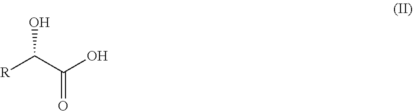

wherein the hydrolase is acyl-CoA thioester hydrolase YciA (4) encoded by a gene selected from E. coli in step C) is more preferred.

“α-hydroxyacyl-CoA:oxalate CoA-transferase” refers a peptide catalyzing the transfer of the coenzyme A of the mandelyl-CoA thioester or a derivative thereof to the oxalate, and thus forms an oxalyl-CoA thioester and a carboxylic acid or or salts thereof.

“α-hydroxyacyl-CoA:formate CoA-transferase” refers a peptide catalyzing the transfer of the coenzyme A of the mandelyl-CoA thioester or a derivative thereof to the formate, and thus forms a formyl-CoA thioester and a carboxylic acid or salts thereof.

In case the method is carried out as a cascade reaction in one-pot system, the catalytic cycle using oxalate and formate is depicted in FIGS. 6 and 7, respectively. It is apparent that stoichiometric amounts of ATP are not required.

“Mandelyl-CoA:oxalate CoA-transferase” refers a peptide catalyzing the transfer of the coenzyme A of the mandelyl-CoA thioester or a derivative thereof to the oxalate, and thus forms an oxalyl-CoA thioester and the mandelic acid or mandelic acid derivative or salts thereof.

“Mandelyl-CoA:formate CoA-transferase” refers a peptide catalyzing the transfer of the coenzyme A of the mandelyl-CoA thioester or a derivative thereof to the formate, and thus forms a formyl-CoA thioester and the mandelic acid or mandelic acid derivative or salts thereof.

The α-hydroxyacyl-CoA:oxalate CoA-transferase or α-hydroxyacyl-CoA:formate CoA-transferase can be a mutant of formyl-CoA:oxalate CoA-transferase in particular encoded by a gene selected from Oxalobacter formigenes, a cinnamoyl-CoA:phenyllactate transferase or a mutant thereof in particular encoded by a gene selected from Clostridium botulinum, isocaprenoyl-CoA:α-hydroxyisocaproate CoA-transferase or a mutant thereof in particular encoded by a gene selected from Clostridium difficile.

The mandelyl-CoA:oxalate transferase or mandelyl-CoA:formate transferase can be a mutant of formyl-CoA:oxalate CoA-transferase in particular encoded by a gene selected from Oxalobacter formigenes, a cinnamoyl-CoA:phenyllactate transferase or a mutant thereof in particular encoded by a gene selected from Clostridium botulinum, isocaprenoyl-CoA:α-hydroxyisocaproate CoA-transferase or a mutant thereof in particular encoded by a gene selected from Clostridium difficile.

A preferred embodiment of the present invention is thus directed to method for the preparation of an α-hydroxyacyl compound of the formula (I′)

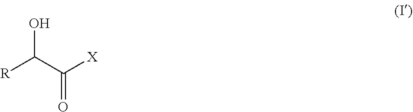

comprising:
A) providing a compound R—CHO (1) and an acyl-CoA (2)

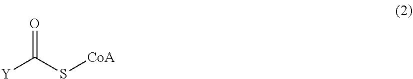

wherein Y represents —H, or —COO⁻;
in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the α-hydroxyacyl-CoA thioester (3′)

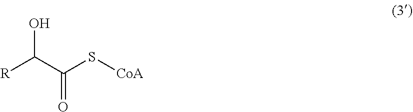

wherein the oxalyl-CoA decarboxylase (3a) is a peptide encoded by a gene selected from Methylobacterium extorquens or a mutant thereof; and optionally comprising the additional step C)
C) hydrolysing said α-hydroxyacyl-CoA thioester (3′) by a hydrolase to obtain the compound of the formula (II′) or a salt thereof

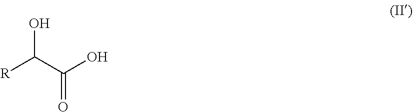

wherein the hydrolase is acyl-CoA thioester hydrolase YciA (4) encoded by a gene selected from E. coli.

Another preferred embodiment of the present invention is related to a method for the preparation of α-hydroxyacyl compound of the formula (I)

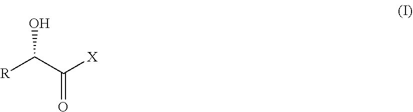

A) providing a compound R—CHO (1) and an acyl-CoA (2)

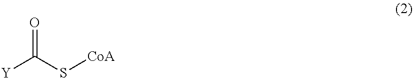

wherein Y represents —H, or —COO⁻,
in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

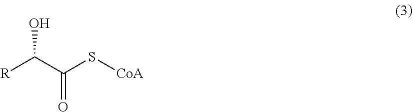

wherein the oxalyl-CoA decarboxylase (3a) is a peptide encoded by a gene selected from Methylobacterium extorquens or a mutant thereof; or additionally comprising the step C)
C) hydrolysing said (S)-α-hydroxyacyl-CoA thioester (3) by a hydrolase to obtain the compound of the formula (II) or a salt thereof

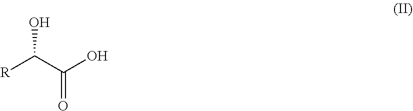

wherein the hydrolase is acyl-CoA thioester hydrolase YciA (4) encoded by a gene selected from E. coli.

A further preferred embodiment of the present invention is directed to a method for preparation of a compound of the formula (I′)

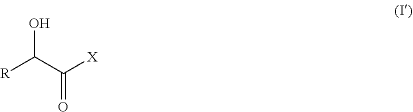

comprising:
A) providing a compound R—CHO (1) and an oxalyl-CoA (2′)

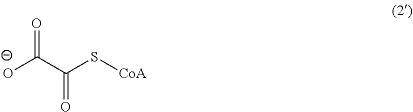

in a buffer solution with a pH-value in the range of 5.0 to 7.5, optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the α-hydroxyacyl-CoA thioester (3′)

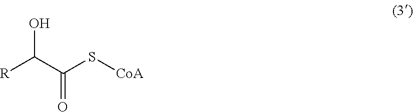

wherein the oxalyl-CoA decarboxylase (3a) is a peptide encoded by a gene selected from Methylobacterium extorquens or a mutant thereof; or additionally comprising the step C)
C) hydrolysing said α-hydroxyacyl-CoA thioester (3′) by a hydrolase to obtain the compound of the formula (II′) or a salt thereof

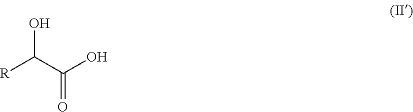

wherein the hydrolase is acyl-CoA thioester hydrolase YciA (4) encoded by a gene selected from E. coli.

Further, a preferred embodiment of the invention is directed to a method for preparation of a compound of the formula (I)

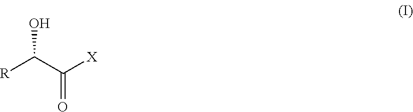

comprising:
A) providing a compound R—CHO (1) and an oxalyl-CoA (2′)

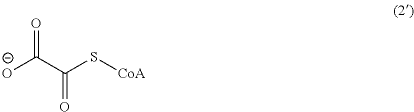

in a buffer solution with a pH-value in the range of 5.0 to 7.5, optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

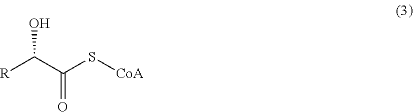

wherein the oxalyl-CoA decarboxylase (3a) is a peptide encoded by a gene selected from Methylobacterium extorquens or a mutant thereof; or additionally comprising the step C)
C) hydrolysing said (S)-α-hydroxyacyl-CoA thioester (3) by a hydrolase to obtain the compound of the formula (II) or a salt thereof

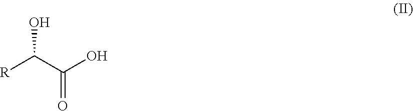

wherein the hydrolase is acyl-CoA thioester hydrolase YciA (4) encoded by a gene selected from E. coli.

Another preferred embodiment of the present invention is related to a method for preparation of a compound of the formula (II′)

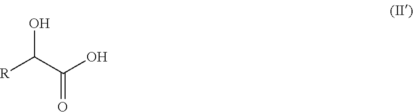

comprising:
A) providing a compound R—CHO (1) and an oxalyl-CoA (2′)

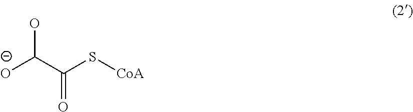

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the α-hydroxyacyl-CoA thioester (3′)

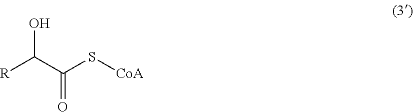

wherein the oxalyl-CoA decarboxylase (3a) is a peptide encoded by a gene selected from Methylobacterium extorquens or a mutant thereof;

and
C) hydrolysing said α-hydroxyacyl-CoA thioester (3′) by a hydrolase to obtain the compound of the formula (II′) or a salt thereof

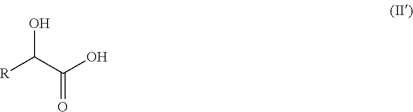

wherein the hydrolase is acyl-CoA thioester hydrolase YciA (4) encoded by a gene selected from E. coli.

In step C) of a method according to the invention as described herein, a concentration of the acyl-CoA thioester hydrolase YciA encoded by a gene selected from E. coli (4) can be in the range of 0.25 to 5 μM, preferably 0.5 to 3, and more preferably 1 to 2.5.

An embodiment of the invention is related to a method for the preparation of α-hydroxyacyl compound of the formula (I)

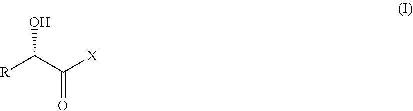

comprising:
A) providing a compound R—CHO (1) and an acyl-CoA (2)

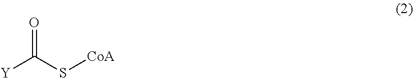

wherein Y represents —H, or —COO⁻,
in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

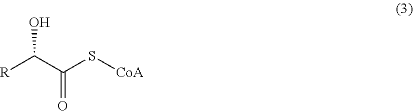

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

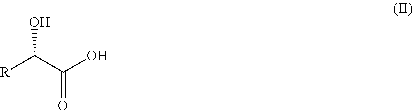

wherein the hydrolase is acyl-CoA thioester hydrolase YciA (4) encoded by a gene selected from E. coli, and wherein a concentration of the acyl-CoA thioester hydrolase YciA (4) is in the range of 0.25 to 5 μM.

An embodiment of the invention is directed to a method for preparation of a compound of the formula (I)

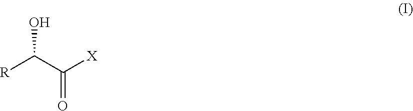

comprising:
A) providing a compound R—CHO (1) and an oxalyl-CoA (2′)

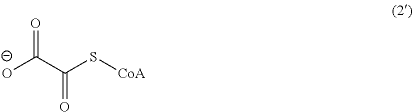

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

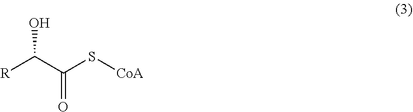

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

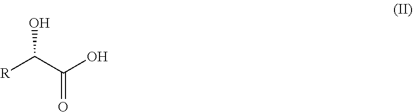

wherein the hydrolase is acyl-CoA thioester hydrolase YciA (4) encoded by a gene selected from E. coli, and wherein a concentration of the acyl-CoA thioester hydrolase YciA (4) is in the range of 0.25 to 5 μM (2 μM).

Another embodiment of the present invention is related to a method for preparation of a compound of the formula (II)

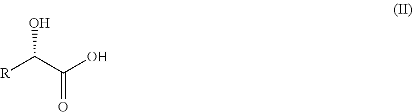

comprising:
A) providing a compound R—CHO (1) and an oxalyl-CoA (2′)

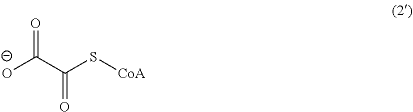

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

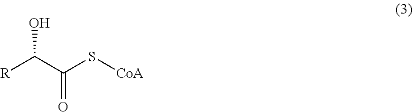

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

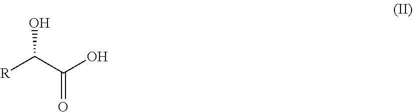

wherein the hydrolase is acyl-CoA thioester hydrolase YciA (4) encoded by a gene selected from E. coli, and wherein a concentration of the acyl-CoA thioester hydrolase YciA (4) is in the range of 0.25 to 5 μM.

A preferred embodiment of the invention is related to a method for preparation of a compound of the formula (II)

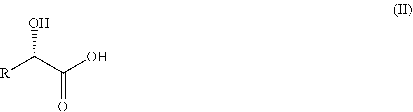

comprising:
A) providing a compound R—CHO (1) and an oxalyl-CoA (2′)

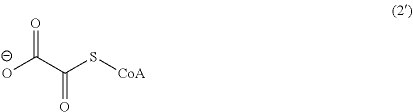

in a buffer solution with a pH-value in the range of 6.5 to 7.5 optionally together with at least one co-solvent, wherein a concentration of R—CHO (1) is in the range of 5 mM to 50 mM, wherein the buffer solution comprises 2-[[1,3-dihydroxy-2-(hydroxymethyl)propan-2-yl]amino]ethanesulfonic acid (TES) in the range of 0.25 wt % to 2.5 wt %, and wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′);
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

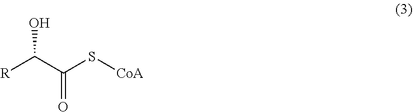

wherein the oxalyl-CoA decarboxylase (3a) is a peptide encoded by a gene selected from Methylobacterium extorquens or a mutant thereof, wherein a molar ratio of the thiamine diphosphate (3b) compared to R—CHO (1) is 0.001 to 0.01 with a concentration in the range of 0.05 mM to 0.5 mM, wherein a concentration of the oxalyl-CoA decarboxylase is in the range of 1 to 25 μM and/or a concentration of the magnesium (II) cation is in the range of 2.5 mM to 20 mM, and wherein the mutant preferably is selected from the group comprises or consists of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

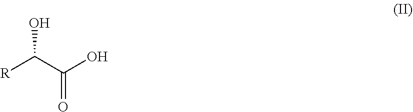

wherein the hydrolase is acyl-CoA thioester hydrolase YciA (4) encoded by a gene selected from E. coli, and wherein a concentration of the acyl-CoA thioester hydrolase YciA (4) is in the range of 0.25 to 5 μM; and

wherein steps A′), A), B), and C) are carried out as cascade reaction in one-pot system, and wherein a ratio of R—CHO (1) and the free coenzyme A (2b) is 10 to 1000.

In the methods according to the invention, in step A′) a concentration of the carboxylate (2a) can be in the range of 1 to 100 mM, preferably 5 mM to 50 mM, more preferably 10 mM to 25 mM, and/or a concentration of acyl-CoA synthetase (2c) can be in the range of 1 to 25 μM, preferably 2 to 15, more preferably 5 to 10, and/or ATP can be in the range of 1 mM to 50 mM preferably 5 to 25, more preferably 10 to 20.

An embodiment of the present invention is directed to a method for preparation of a compound of the formula (I)

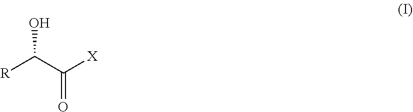

comprising:
A) providing a compound R—CHO (1) and an acyl-CoA (2)

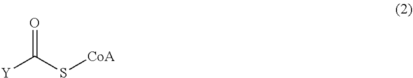

wherein Y represents —H, or —COO⁻, in a buffer solution with a pH-value in the range of 5.0 to 7.5, optionally together with at least one co-solvent, and wherein acyl-CoA (2) can be prepared by the following step A′):
A′) reacting an carboxylate (2a) with a free coenzyme A (2b) by acyl-CoA synthetase (2c) in the presence of ATP (2e) to produce an acyl-CoA (2) wherein a concentration of the carboxylate (2a) is in the range of 1 to 100 mM and/or a concentration of acyl-CoA synthetase (2c) is in the range of 1 to 25 μM (5 μM) and/or ATP is in the range of 1 mM to 50 mM;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

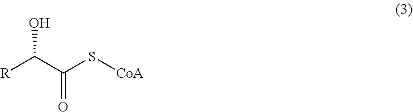

wherein the oxalyl-CoA decarboxylase (3a) is a peptide encoded by a gene selected from Methylobacterium extorquens or a mutant thereof; or additionally comprising the step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

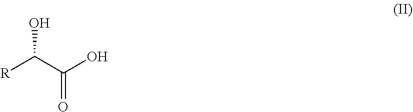

wherein the hydrolase is acyl-CoA thioester hydrolase YciA (4) encoded by a gene selected from E. coli; and

wherein the steps A′), A), and B), and C) are carried out as cascade reaction in one-pot system, and wherein a ratio of the carboxylate (2a) and the free coenzyme A (2b) is preferably 10 to 10,000

In the methods according to the invention, in step A′) a concentration of the oxalate can be in the range of 1 to 100 mM and/or a concentration of oxalyl-CoA synthetase (2c′) can be in the range of 1 to 25 μM (5 μM) and/or ATP can in the range of 1 mM to 50 mM.

An embodiment of the invention is directed to a method for preparation of a compound of the formula (I)

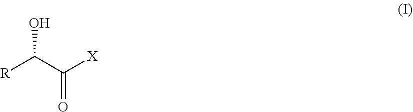

comprising:
A) providing a compound R—CHO (1) and an oxalyl-CoA (2′)

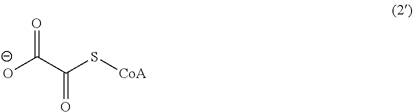

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′), wherein a concentration of the oxalate is in the range of 1 to 100 mM and/or a concentration of oxalyl-CoA synthetase (2c′) is in the range of 1 to 25 μM and/or ATP is in the range of 1 mM to 50 mM;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

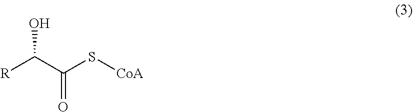

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

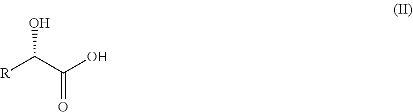

wherein the hydrolase is acyl-CoA thioester hydrolase YciA (4) encoded by a gene selected from E. coli.

Another embodiment of the present invention is related to a method for preparation of a compound of the formula (II)

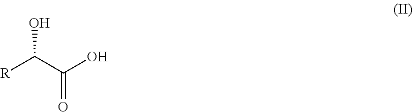

comprising:
A) providing a compound R—CHO (1) and an oxalyl-CoA (2′)

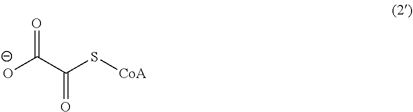

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′), wherein a concentration of the oxalate is in the range of 1 to 100 mM and/or a concentration of oxalyl-CoA synthetase (2c′) is in the range of 1 to 25 μM (5 μM) and/or ATP is in the range of 1 mM to 50 mM;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

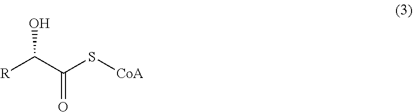

wherein the oxalyl-CoA decarboxylase (3a) is a peptide encoded by a gene selected from Methylobacterium extorquens or a mutant thereof;

and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

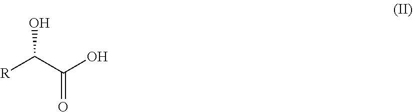

wherein the hydrolase is acyl-CoA thioester hydrolase YciA (4) encoded by a gene selected from E. coli.

A preferred embodiment of the invention is related to a method for preparation of a compound of the formula (II)

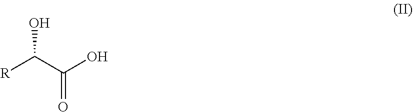

comprising:
A) providing a compound R—CHO (1) and an oxalyl-CoA (2′)

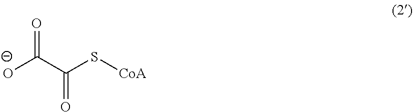

in a buffer solution with a pH-value in the range of 6.5 to 7.5 optionally together with at least one co-solvent, wherein a concentration of R—CHO (1) is in the range of 5 mM to 50 mM, wherein the buffer solution comprises 2-[[1,3-dihydroxy-2-(hydroxymethyl)propan-2-yl]amino]ethanesulfonic acid (TES) in the range of 0.25 wt % to 2.5 wt %, and wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′), wherein a concentration of the oxalate is in the range of 1 to 100 mM and/or a concentration of oxalyl-CoA synthetase (2c′) is in the range of 1 to 25 μM (5 μM) and/or ATP is in the range of 1 mM to 50 mM;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

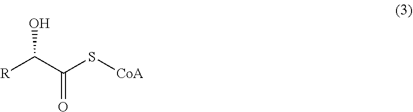

wherein the oxalyl-CoA decarboxylase (3a) is a peptide encoded by a gene selected from Methylobacterium extorquens or a mutant thereof, wherein a molar ratio of the thiamine diphosphate (3b) compared to R—CHO (1) is 0.001 to 0.01 with a concentration in the range of 0.05 mM to 0.5 mM, wherein a concentration of the oxalyl-CoA decarboxylase is in the range of 1 to 25 μM and/or a concentration of the magnesium (II) cation is in the range of 2.5 mM to 20 mM, and wherein the mutant preferably is selected from the group comprises or consists of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

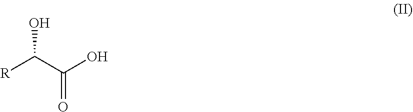

wherein the hydrolase is acyl-CoA thioester hydrolase YciA (4) encoded by a gene selected from E. coli, and wherein a concentration of the acyl-CoA thioester hydrolase YciA (4) is in the range of 0.25 to 5 μM; and

wherein steps A′), A), B), and C) are carried out as cascade reaction in one-pot system, and wherein a ratio of R—CHO (1) and the free coenzyme A (2b) is 10 to 1000.

In step A′) of the method according to the invention, the carboxylate (2a) have a cationic counterion selected from the group comprising or consisting of NH₄⁺, Li⁺, Na⁺, K⁺, Rb⁺, Cs⁺, Mg²⁺, Ca²⁺, Zn²⁺, Fe²⁺, Mn²⁺, Ni²⁺, Cu²⁺, and Co²⁺. Preferably NH₄⁺, Na⁺, K⁺, Mg²⁺, and Ca²⁺.

An embodiment of the present invention is directed to a method for preparation of a compound of the formula (I)

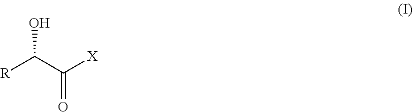

comprising:
A) providing a compound R—CHO (1) and an acyl-CoA (2)

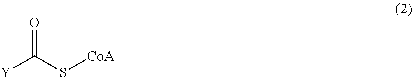

wherein Y represents —H, or —COO⁻, in a buffer solution with a pH-value in the range of 5.0 to 7.5, optionally together with at least one co-solvent, and wherein acyl-CoA (2) can be prepared by the following step A′):
A′) reacting an carboxylate (2a) with a free coenzyme A (2b) by acyl-CoA synthetase (2c) in the presence of ATP (2e) to produce an acyl-CoA (2), and wherein the carboxylate (2a) have a cationic counterion selected from the group consisting of NH₄⁺, Li⁺, Na⁺, K⁺, Rb⁺, Cs⁺, Mg²⁺, Ca²⁺, Zn²⁺, Fe²⁺, Mn²⁺, Ni²⁺, Co²⁺ and Cu²⁺;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

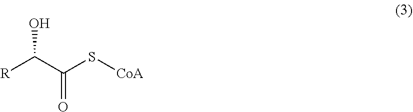

wherein the oxalyl-CoA decarboxylase (3a) is a peptide encoded by a gene selected from Methylobacterium extorquens or a mutant thereof; or additionally comprising the step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

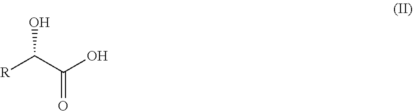

wherein the hydrolase is acyl-CoA thioester hydrolase YciA (4) encoded by a gene selected from E. coli.

In step A′) of the method according to the invention, the oxalate (2a′) can be selected from the group comprising or consisting of (NH₄)₂C₂O₄, Li₂C₂O₄, Na₂C₂O₄, K₂C₂O₄, Rb₂C₂O₄, Cs₂C₂O₄, MgC₂O₄, CaC₂O₄, ZnC₂O₄, FeC₂O₄, MnC₂O₄, NiC₂O₄, CuC₂O₄, and CoC₂O₄.

An embodiment of the invention is directed to a method for preparation of a compound of the formula (I)

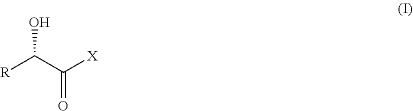

comprising:
A) providing a compound R—CHO (1) and an oxalyl-CoA (2′)

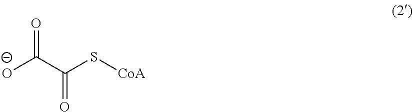

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′), wherein the oxalate is selected from the group comprising or consisting of (NH₄)₂C₂O₄, Li₂C₂O₄, Na₂C₂O₄, K₂C₂O₄, Rb₂C₂O₄, Cs₂C₂O₄, MgC₂O₄, CaC₂O₄, ZnC₂O₄, FeC₂O₄, MnC₂O₄, NiC₂O₄, CuC₂O₄; and CoC₂O₄
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

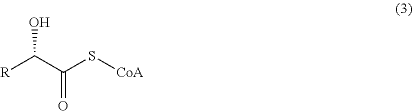

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

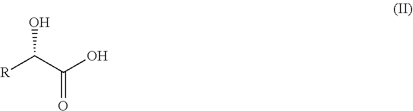

wherein the hydrolase is acyl-CoA thioester hydrolase YciA (4) encoded by a gene selected from E. coli.

Another embodiment of the present invention is related to a method for preparation of a compound of the formula (II)

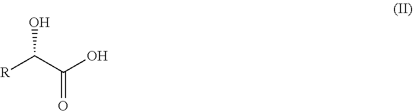

comprising:
A) providing a compound R—CHO (1) and an oxalyl-CoA (2′)

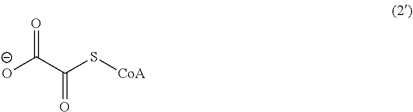

in a buffer solution with a pH-value in the range of 5.0 to 7.5 optionally together with at least one co-solvent, wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′), wherein the oxalate is selected from the group comprising or consisting of (NH₄)₂C₂O₄, Li₂C₂O₄, Na₂C₂O₄, K₂C₂O₄, MgC₂O₄, CaC₂O₄, ZnC₂O₄, FeC₂O₄, MnC₂O₄, NiC₂O₄, CuC₂O₄, Rb₂C₂O₄, Cs₂C₂O₄, and CoC₂O₄
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

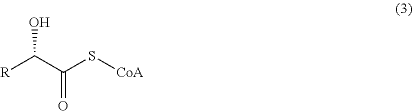

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

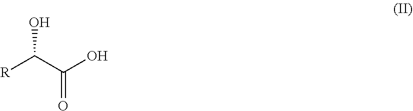

wherein the hydrolase is acyl-CoA thioester hydrolase YciA (4) encoded by a gene selected from E. coli.

A preferred embodiment of the invention is related to a method for preparation of a compound of the formula (II)

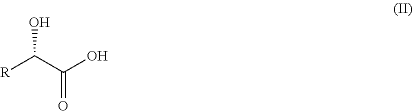

comprising:
A) providing a compound R—CHO (1) and an oxalyl-CoA (2′)

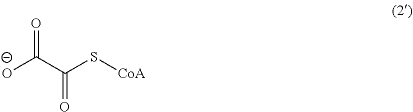

in a buffer solution with a pH-value in the range of 6.5 to 7.5 optionally together with at least one co-solvent, wherein a concentration of R—CHO (1) is in the range of 5 mM to 50 mM, wherein the buffer solution comprises 2-[[1,3-dihydroxy-2-(hydroxymethyl)propan-2-yl]amino]ethanesulfonic acid (TES) in the range of 0.25 wt % to 2.5 wt %, and wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′) wherein a concentration of the oxalate is in the range of 1 to 100 mM and/or a concentration of oxalyl-CoA synthetase (2c′) is in the range of 1 to 25 μM and/or ATP is in the range of 1 mM to 50 mM, and wherein the oxalate is selected from the group comprising or consisting of (NH₄)₂C₂O₄, Li₂C₂O₄, Na₂C₂O₄, K₂C₂O₄, MgC₂O₄, CaC₂O₄, ZnC₂O₄, FeC₂O₄, MnC₂O₄, NiC₂O₄, CuC₂O₄, Rb₂C₂O₄, Cs₂O₂O₄, and CoC₂O₄, preferably (NH₄)₂C₂O₄, Li₂C₂O₄, Na₂C₂O₄, K₂C₂O₄, more preferably Na₂C₂O₄;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

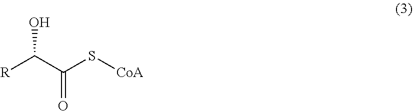

wherein the oxalyl-CoA decarboxylase (3a) is a peptide encoded by a gene selected from Methylobacterium extorquens or a mutant thereof, wherein a molar ratio of the thiamine diphosphate (3b) compared to R—CHO (1) is 0.001 to 0.01 with a concentration in the range of 0.05 mM to 0.5 mM, wherein a concentration of the oxalyl-CoA decarboxylase is in the range of 1 to 25 μM and/or a concentration of the magnesium (II) cation is in the range of 2.5 mM to 20 mM, and wherein the mutant preferably is selected from the group comprises or consists of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

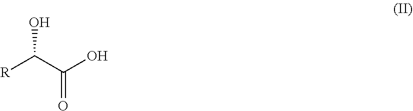

wherein the hydrolase is acyl-CoA thioester hydrolase YciA (4) encoded by a gene selected from E. coli, and wherein a concentration of the acyl-CoA thioester hydrolase YciA (4) is in the range of 0.25 to 5 μM; and wherein steps A′), A), B), and C) are carried out as cascade reaction in one-pot system, and wherein a ratio of R—CHO (1) and the free coenzyme A (2b) is 10 to 1000.

In principal each co-solvent can be used when it does not denaturize the enzyme under the used conditions. A skilled person in the art knows how to choose a suitable co-solvent. A co-solvent can be selected from the group comprising or consisting of dimethyl sulfoxide, acetone, acetonitrile (ACN), 2-butanol, tert-butyl alcohol, 1,2-dimethoxy-ethane (DME), dimethyl-formamide (DMF), 1,4-dioxane, ethanol, methanol, 1-propanol, 2-propanol, tetrahydrofuran (THF), and ethylene glycol (EG). Preferably, one of the at least one co-solvent is dimethyl sulfoxide. Preferably, the at least one co-solvent is dimethyl sulfoxide.

Preferably, R represents

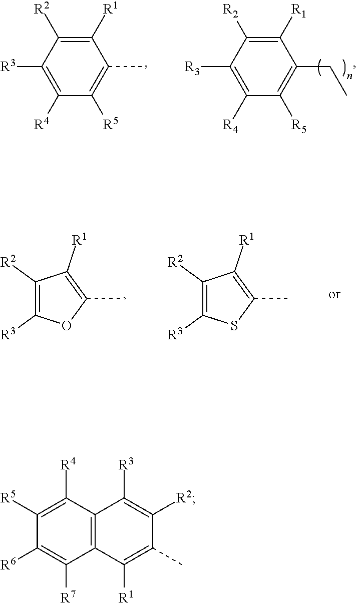

and

R¹, R², R³, R⁴, R⁵, R⁶ and R⁷ represents independently of each other —H, —Cl, —OH, —OCH₃, —NO₂, —N(CH₃)₂, —C≡CH, —CHO, —COCH₃ or —COOH, and n represents an integer 1.

An embodiment of the invention is related to a method for the preparation of α-hydroxyacyl compound of the formula (I)

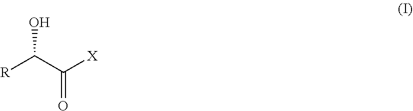

wherein R represents

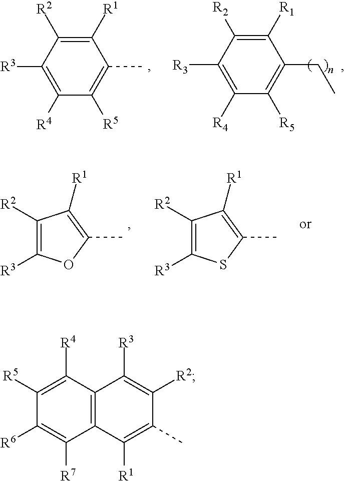

and

R¹, R², R³, R⁴, R⁵, R⁶ and R⁷ represents independently of each other —H, —Cl, —OH, —OCH₃, —NO₂, —N(CH₃)₂, —C≡CH, —CHO, —COCH₃ or —COOH, and represents an integer 1; or a salt thereof.

comprising:
A) providing a compound R—CHO (1) and an acyl-CoA (2)

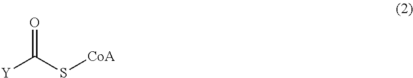

wherein Y represents —H, or —COO⁻,
in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

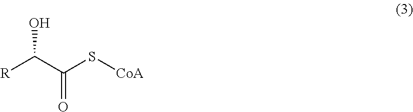

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

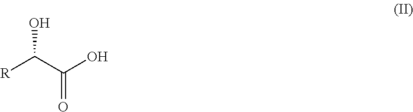

wherein the hydrolase is acyl-CoA thioester hydrolase YciA (4) encoded by a gene selected from E. coli.

A preferred embodiment of the invention is related to a method for the preparation of α-hydroxyacyl compound of the formula (I)

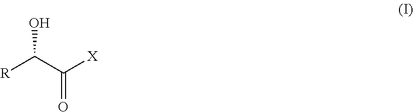

wherein R represents

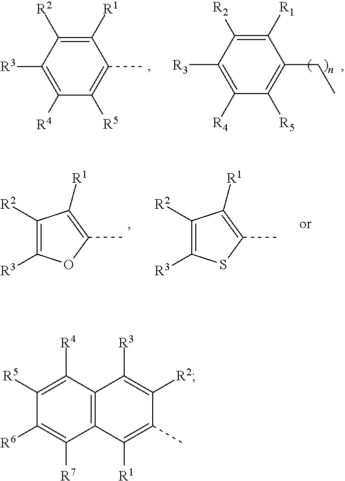

and

R¹, R², R³, R⁴, R⁵, R⁶ and R⁷ represents independently of each other —H, —Cl, —OH, —OCH₃, —NO₂, —N(CH₃)₂, —C≡CH, —CHO, —COCH₃ or —COOH, and represents an integer 1; or a salt thereof.

comprising:
A) providing a compound R—CHO (1) and an acyl-CoA (2)

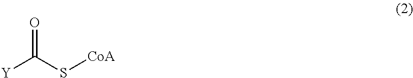

wherein Y represents —H, or —COO⁻, in a buffer solution with a pH-value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the (S)-α-hydroxyacyl-CoA thioester (3)

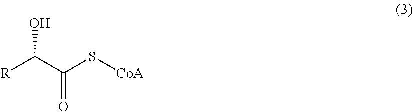

wherein the oxalyl-CoA decarboxylase (3a) is a peptide encoded by a gene selected from Methylobacterium extorquens or a mutant thereof; and optionally comprising the additional step C)
C) hydrolysing said (S)-α-hydroxyacyl-CoA thioester (3) by a hydrolase to obtain the compound of the formula (II) or a salt thereof,

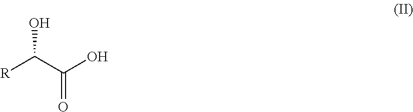

wherein the hydrolase is acyl-CoA thioester hydrolase YciA (4) encoded by a gene selected from E. coli.

An embodiment of the invention is directed to a method for preparation of a compound of the formula (I)

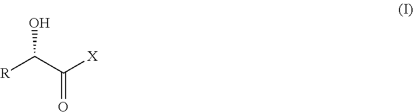

wherein R represents

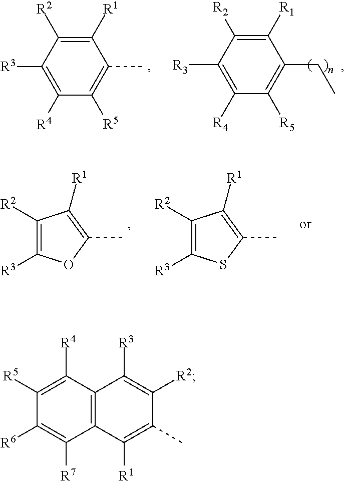

and

R¹, R², R³, R⁴, R⁵, R⁶ and R⁷ represents independently of each other —H, —Cl, —OH, —OCH₃, —NO₂, —N(CH₃)₂, —C≡CH, —CHO, —COCH₃ or —COOH, and represents an integer 1; or a salt thereof.

comprising:
A) providing a compound R—CHO (1) and an oxalyl-CoA (2′)

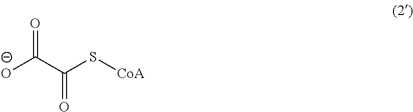

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

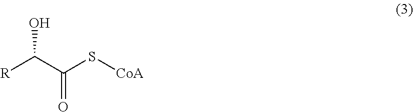

wherein the oxalyl-CoA decarboxylase (3a) is a peptide encoded by a gene selected from Methylobacterium extorquens or a mutant thereof; and optionally comprising the additional step C)
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

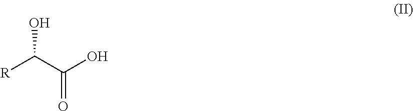

wherein the hydrolase is acyl-CoA thioester hydrolase YciA (4) encoded by a gene selected from E. coli.

Another embodiment of the present invention is related to a method for preparation of a compound of the formula (II)

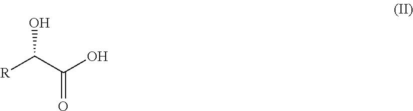

wherein R represents

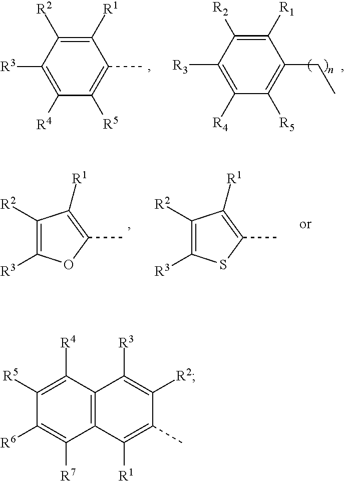

and

R¹, R², R³, R⁴, R⁵, R⁶ and R⁷ represents independently of each other —H, —Cl, —OH, —OCH₃, —NO₂, —N(CH₃)₂, —C≡CH, —CHO, —COCH₃ or —COOH, and represents an integer 1; or a salt thereof.

comprising:
A) providing a compound R—CHO (1) and an oxalyl-CoA (2′)

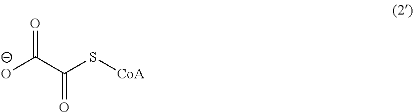

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

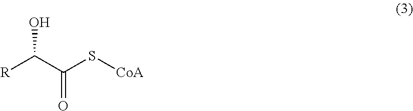

wherein the oxalyl-CoA decarboxylase (3a) is a peptide encoded by a gene selected from Methylobacterium extorquens or a mutant thereof; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

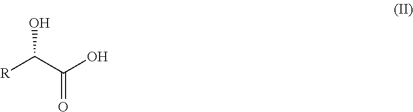

wherein the hydrolase is acyl-CoA thioester hydrolase YciA (4) encoded by a gene selected from E. coli.

A preferred embodiment of the present invention is related to a method for preparation of a compound of the formula (II)

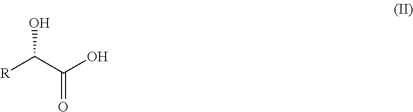

wherein R represents

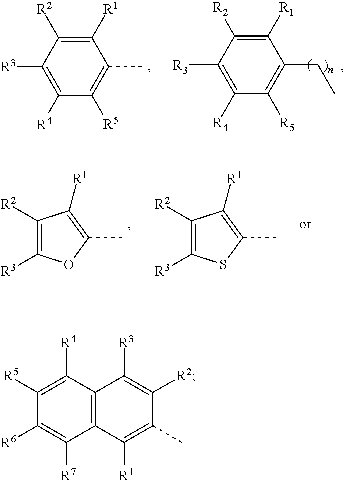

and

R¹, R², R³, R⁴, R⁵, R⁶ and R⁷ represents independently of each other —H, —Cl, —OH, —OCH₃, —NO₂, —N(CH₃)₂, —C≡CH, —CHO, —COCH₃ or —COOH, and represents an integer 1; or a salt thereof.

comprising:
A) providing a compound R—CHO (1) and an oxalyl-CoA (2′)

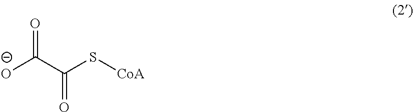

in a buffer solution with a pH-value in the range of 5.0 to 7.5 optionally together with at least one co-solvent;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

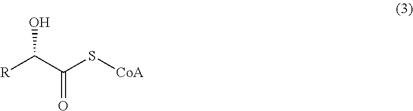

wherein the oxalyl-CoA decarboxylase (3a) is a peptide encoded by a gene selected from Methylobacterium extorquens or a mutant thereof; and
C) hydrolyzing said (S)-α-hydroxyacyl-CoA thioester (3) by a hydrolase to obtain the compound of the formula (II) or a salt thereof

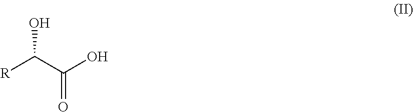

wherein the hydrolase is acyl-CoA thioester hydrolase YciA (4) encoded by a gene selected from E. coli.

A preferred embodiment of the invention is related to a method for preparation of a compound of the formula (II)

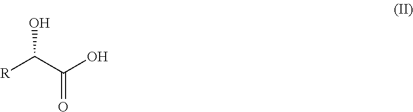

wherein R represents

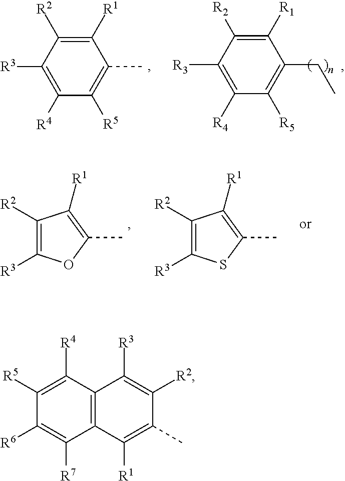

and

wherein R⁻¹, R², R³, R⁴, R⁵, R⁶ and R⁷ represent independently —H, —Cl, —OH, —OCH₃, —NO₂, —N(CH₃)₂, —C≡CH, —CHO, —COCH₃ or —COOH, wherein n represents an integer 1; or a salt thereof.

comprising:
A) providing a compound R—CHO (1) and an oxalyl-CoA (2′)

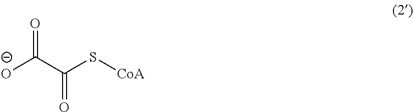

in a buffer solution with a pH-value in the range of 6.5 to 7.5 optionally together with at least one co-solvent, wherein a concentration of R—CHO (1) is in the range of 5 mM to 50 mM, wherein the buffer solution comprises 2-[[1,3-dihydroxy-2-(hydroxymethyl)propan-2-yl]amino]ethanesulfonic acid (TES) in the range of 0.25 wt % to 2.5 wt %, and wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′) wherein a concentration of the oxalate is in the range of 1 to 100 mM (FIG. 1.C) and/or a concentration of oxalyl-CoA synthetase (2c′) is in the range of 1 to 25 μM and/or ATP is in the range of 1 mM to 50 mM, and wherein the oxalate is selected from the group comprising or consisting of (NH₄)₂C₂O₄, Li₂C₂O₄, Na₂C₂O₄, K₂C₂O₄, Rb₂C₂O₄, Cs₂C₂O₄, MgC₂O₄, CaC₂O₄, ZnC₂O₄, FeC₂O₄, MnC₂O₄, NiC₂O₄, CuC₂O₄, and CoC₂O₄, preferably (NH₄)₂C₂O₄, Li₂C₂O₄, Na₂C₂O₄, K₂C₂O₄, more preferably Na₂C₂O₄;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

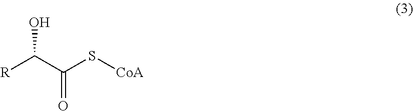

wherein the oxalyl-CoA decarboxylase (3a) is a peptide encoded by a gene selected from Methylobacterium extorquens or a mutant thereof, wherein a molar ratio of the thiamine diphosphate (3b) compared to R—CHO (1) is 0.001 to 0.01 with a concentration in the range of 0.05 mM to 0.5 mM, wherein a concentration of the oxalyl-CoA decarboxylase is in the range of 1 to 25 μM and/or a concentration of the magnesium (II) cation is in the range of 2.5 mM to 20 mM, and wherein the mutant preferably is selected from the group comprises or consists of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and
C) cleaving the coenzyme A moiety of said (S)-α-hydroxyacyl-CoA thioester (3) by a hydrolase, α-hydroxyacyl-CoA:oxalate CoA-transferase and/or α-hydroxyacyl-CoA:formate CoA-transferase to obtain the compound of the formula (II) or a salt thereof

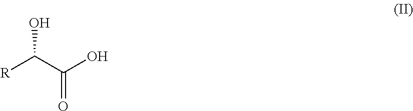

wherein the hydrolase is acyl-CoA thioester hydrolase YciA (4) encoded by a gene selected from E. coli, and wherein a concentration of the acyl-CoA thioester hydrolase YciA (4) is in the range of 0.25 to 5 μM (2 μM); and

wherein steps A′), A), B), and C) are carried out as cascade reaction in one-pot system, and wherein a ratio of R—CHO (1) and the free coenzyme A (2b) is 10 to 1000.

A more preferred embodiment of the invention is related to a method for preparation of a compound of the formula (II)

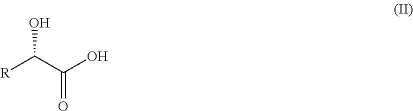

wherein R represents

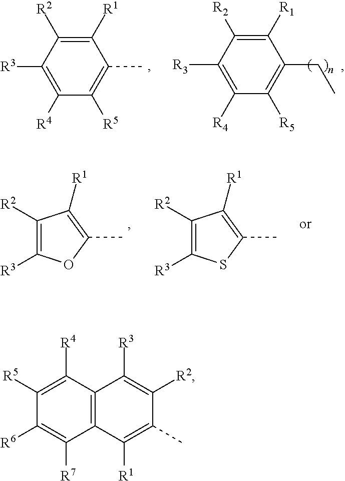

and

wherein R¹, R², R³, R⁴, R⁵, R⁶ and R⁷ represent independently —H, —Cl, —OH, —OCH₃, —NO₂, —N(CH₃)₂, —C≡CH, —CHO, —COCH₃ or —COOH, wherein n represents an integer 1; or a salt thereof.

comprising:
A) providing a compound R—CHO (1) and an oxalyl-CoA (2′)

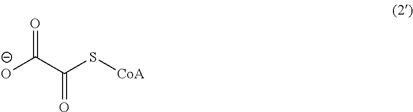

in a buffer solution with a pH-value in the range of 6.5 to 7.5 optionally together with at least one co-solvent, wherein a concentration of R—CHO (1) is in the range of 5 mM to 50 mM, wherein the buffer solution comprises 2-[[1,3-dihydroxy-2-(hydroxymethyl)propan-2-yl]amino]ethanesulfonic acid (TES) in the range of 0.25 wt % to 2.5 wt %, and wherein the oxalyl-CoA (2′) is prepared by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′) wherein a concentration of the oxalate is in the range of 1 to 100 mM and/or a concentration of oxalyl-CoA synthetase (2c′) is in the range of 1 to 25 μM and/or ATP is in the range of 1 mM to 50 mM, and wherein the oxalate is selected from the group comprising or consisting of (NH₄)₂C₂O₄, Li₂C₂O₄, Na₂C₂O₄, K₂C₂O₄, Rb₂C₂O₄, Cs₂C₂O₄, MgC₂O₄, CaC₂O₄, ZnC₂O₄, FeC₂O₄, MnC₂O₄, NiC₂O₄, CuC₂O₄, and CoC₂O₄, preferably (NH₄)₂C₂O₄, Li₂C₂O₄, Na₂C₂O₄, K₂C₂O₄, more preferably Na₂C₂O₄;
B) performing a coupling reaction of R—CHO (1) and oxalyl-CoA (2′) by an oxalyl-CoA decarboxylase (3a) in the presence of thiamine diphosphate (3b) and a magnesium (II) cation to obtain the (S)-α-hydroxyacyl-CoA thioester (3)

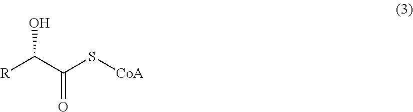

wherein the oxalyl-CoA decarboxylase (3a) is a peptide encoded by a gene selected from Methylobacterium extorquens or a mutant thereof, wherein a molar ratio of the thiamine diphosphate (3b) compared to R—CHO (1) is 0.001 to 0.01 with a concentration in the range of 0.05 mM to 0.5 mM, wherein a concentration of the oxalyl-CoA decarboxylase is in the range of 1 to 25 μM and/or a concentration of the magnesium (II) cation is in the range of 2.5 mM to 20 mM, and wherein the mutant preferably is selected from the group comprises or consists of OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4; and
C) hydrolysing said (S)-α-hydroxyacyl-CoA thioester (3) by a hydrolase to obtain the compound of the formula (II) or a salt thereof

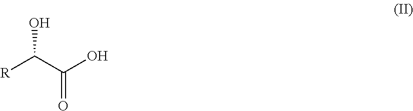

wherein the hydrolase is acyl-CoA thioester hydrolase YciA (4) encoded by a gene selected from E. coli, and wherein a concentration of the acyl-CoA thioester hydrolase YciA (4) is in the range of 0.25 to 5 μM (2 μM); and

wherein steps A′), A), B), and C) are carried out as cascade reaction in one-pot system, and wherein a ratio of R—CHO (1) and the free coenzyme A (2b) is 10 to 1000.

The method according to the invention as described herein can further be improved when employing a whole-cell catalysts which provides not only the cascade enzymes but also ATP regeneration and a CoA pool without addition of purified enzymes and cofactors.

### DESCRIPTION OF THE FIGURES

FIG. 1 shows the reaction of an aldehyde R—CHO with formyl-CoA or oxalyl-CoA to an α-hydroxyacyl-CoA thioester, and a possible subsequent formation of an α-hydroxy carboxylic acid or an α-hydroxy carboxylate.

FIG. 2 shows the hydrolysis of an α-hydroxyacyl-CoA thioester to the α-hydroxy carboxylate (first reaction), and the transfer of the coenzyme A moiety from the α-hydroxyacyl-CoA thioester to a formate (second reaction) resulting in the formation of an α-hydroxy carboxylate and a formyl-CoA thioester as well as the transfer of the coenzyme A moiety from α-hydroxyacyl-CoA thioester to an oxalate resulting in the formation of an α-hydroxy carboxylate and an oxalyl-CoA thioester.

FIG. 3 shows the catalytic cycle of a one-pot system performed as a cascade reaction using a thioesterase YciA in order to hydrolyse an α-hydroxyacyl-CoA thioester to an α-hydroxy carboxylate.

FIG. 4 shows the generation of oxalyl-CoA thioester by a transfer of the coenzyme A moiety from formyl-CoA thioester by means of a formyl-CoA:oxalate CoA transferase (FRC).

FIG. 5 shows the catalytic cycle of a one-pot system performed as a cascade reaction using a thioesterase YciA in order to hydrolyse an α-hydroxyacyl-CoA thioester to an α-hydroxy carboxylate, wherein a formyl-CoA thioester is transferred to a oxalyl-CoA thioester by means of a formyl-CoA:oxalate CoA-transferase (FRC).

FIG. 6 shows the catalytic cycle of a one-pot system performed as a cascade reaction using a transferase in order to transfer the coenzyme A moiety from an α-hydroxyacyl-CoA thioester to an oxalate resulting in the formation of an α-hydroxy carboxylate and oxalyl-CoA thioester.

FIG. 7 shows the catalytic cycle of a one-pot system performed as a cascade reaction using a transferase in order to transfer the coenzyme A moiety from an α-hydroxyacyl-CoA thioester to a formate resulting in the formation of an α-hydroxy carboxylate and formyl-CoA thioester.

FIG. 8 shows the peptide sequence SEQ ID No. 1 of oxalyl-CoA decarboxylase encoded by a gene selected from Methylobacterium extorquens (OXC_{Me}).

FIG. 9 shows the peptide sequence SEQ ID No. 2 (OXC_{Me-Y497A}) of a mutation of oxalyl-CoA decarboxylase encoded by a gene selected from Methylobacterium extorquens.

FIG. 10 shows the peptide sequence SEQ ID No. 3 (OXC_{Me-S568A}) of a mutation of oxalyl-CoA decarboxylase encoded by a gene selected from Methylobacterium extorquens.

FIG. 11 shows the peptide sequence SEQ ID No. 4 (OXC_{Me-Y497A-S568A}) of a mutation of oxalyl-CoA decarboxylase encoded by a gene selected from Methylobacterium extorquens.

FIG. 12 shows the MS/MS spectra of the α-hydroxyacyl-CoA thioester products. Calculated m/z values of the fragmentation products are indicated on top. Spectra on the left show the parent ion. Spectra on the right show the fragmentation products of the parent ion (asterisk). The numbers in the spectrum indicate measured m/z values. Mandelyl-CoA was produced from deuterated benzaldehyde; the deuterium is retained on the α-carbon in the product.

FIG. 13 shows Michaelis-Menten graphs of OXC_{Me} and mutants thereof according to example 6. Error bars show standard deviation of three replicates.

FIG. 14 shows competing reaction pathways of OXC. Assays were carried out at 30° C. in reaction buffer consisting of 50 mM TES-KOH pH 6.8, 10 mM MgCl₂. 0.5 mM ADP, 0.15 mM ThDP and 25 mM benzaldehyde, 1 μM OXC_{Me} or 1 μM OXC_{Me-Y497A}. The reaction was started by adding either 1 μM formyl-CoA (A) or 1 μM oxalyl-CoA (B). Samples were taken after 0, 2, 5, 10 and 20 minutes and analyzed with the LC-MS detection of CoA esters. CoA esters were quantified by comparison to synthetic standards of formyl-CoA, oxalyl-CoA and mandelyl-CoA, respectively. Concentration of CoA-esters was determined by comparison to a standard curve obtained from chemically synthesized formyl-CoA, oxalyl-CoA and mandelyl-CoA, respectively. Error bars show standard deviation of three replicates. C, apparent turnover numbers were determined by linear regression of the mandelyl-CoA formation rate over 20 min. wt=wild type=OXC_{Me}; Y497A=OXC_{Me-Y497A}.

FIG. 15 shows the screen for mandelyl-CoA Thioesterase activity. CoA formation was detected with the Ellman's reagent (5,5′-dithiobis-(2-nitrobenzoic acid)), which reacts with free thiols under release of 2-nitro-5-thiobenzoate (ε₄₁₂ₙₘ=14, 150 M⁻¹ cm⁻¹). Assays were carried out at 30° C. in 50 mM MES-KOH pH 6.8 and contained 1 mM Ellman's reagent and 0.5 μM YciA or 0.5 μM TesB or 0.5 μM Paal. The reaction was started by adding 0.1 mM mandelyl-CoA (time point indicated with the arrow).

FIG. 16 shows the thioesterase activity of YciA towards oxalyl-CoA, formyl-CoA and racemic mandelyl-CoA. Assays were carried out at 30° C. in reaction buffer consisting of 50 mM TES-KOH pH 6.8, 10 mM MgCl₂. 0.5 mM ADP, 0.15 mM ThDP and 25 mM benzaldehyde, 2 μM YciA (A) or no YciA (B). The reaction was started by adding either 0.5 mM formyl-CoA or 0.5 mM oxalyl-CoA or 0.5 mM mandelyl-CoA. Samples were taken after 0, 1, 5 and 30 minutes and analyzed with the LC-MS detection of CoA esters. CoA esters were quantified by comparison to synthetic standards of formyl-CoA, oxalyl-CoA and mandelyl-CoA, respectively. Error bars show standard deviation of two.

FIG. 17 shows calibration curves of commercially obtained rac. mandelic acid, rac. 3-phenyllactic acid, rac. 4-chloromandelic acid and (S)-2-chloromandelic acid. The lines show linear fit to the data.

FIG. 18 shows Michaelis-Menten graph of OXS with oxalate as substrate according to example 5. Oxalyl-CoA production was followed by coupling OXS to purified PanE2, an NADPH-dependent oxalyl-CoA reductase from M. extorquens. An assay containing 50 mM potassium phosphate pH 6.5, 0.3 mM NADPH, 10 mM MgCl₂, 1 mM ATP, 2.5 mM CoA, 1.2 μM PanE2, 176 nM OXS was preincubated for 2 min and the reaction started by adding disodium oxalate to a final concentration of 5, 10, 25, 100, 250, 1,000 μM, respectively. Reaction procedure was monitored by following the oxidation of NADPH at 340 nm. Error bars show standard deviation of three replicates.

FIG. 19 shows mandelic acid formation of the cascade over time. ee of (S)-mandelic acid is indicated for the last time point (22 h) according to example 13. Mandelic acid formation of the OXS-OXC-YciA cascade over time. Enantiomeric excess of (S)-mandelic acid is indicated for the last time point (22 h). Assays were carried out at 30° C. in reaction buffer consisting of 50 mM TES-KOH pH 6.8, 10 mM MgCl₂. 0.5 mM ADP, 0.15 mM ThDP, 0.5 mM CoA, 10 mM ATP, 25 mM benzaldehyde, 5 μM OXS, 5 μM OXC and 2 μM YciA. The reaction was started by adding 10 mM oxalate. As negative controls, YciA, OXS or OXC were omitted in separate reactions. Samples were taken after 0, 0.05, 0.25, 0.66, 1, 2, 3, 22 hours and analyzed with the LC-MS detection of mandelic acid derivatives. Mandelic acid was quantified by comparison to commercially obtained standards of mandelic acid.

FIG. 20 shows the oxalyl-CoA decarboxylation activity of OXC_{Me} and HACL_{Hs}. Assays were carried out at 30° C. in 100 mM MES-KOH pH 6.8 and contained 10 mM MgCl₂. 0.5 mM ADP, 0.15 mM ThDP, 0.05 μM OXC_{Me} or 1 μM HACL_{Hs} or no enzyme. The reaction was started by adding 0.5 mM oxalyl-CoA. Samples were taken after 1, 15, 30, 60 min and analyzed with the LC-MS detection of CoA esters.

FIG. 21 shows the peptide sequence of oxalyl-CoA synthetase OXS encoded by a gene selected by Methylobacterium extorquens (SEQ ID NO. 5).

FIG. 22 shows the peptide sequence of YciA by a gene selected from E. coli (SEQ ID NO.6).

FIG. 23 shows the amino acid sequence SEQ ID NO. 18 of acyl-CoA synthetase (eryACS-1) from Erythrobacter sp. NAP1 used as a formyl-CoA synthetase.

FIG. 24 shows the amino acid sequence SEQ ID NO. 19 of a mutant of acyl-CoA synthetase used as a formyl-CoA synthetase.

FIG. 25 shows the nucleotide sequence of eryACS-1 (ENA|EAQ27819|EAQ27819.1acetyl-CoA synthetase from Erythrobactersp. NAP1 a; herein SEQ ID NO. 20).

FIG. 26 shows the nucleotide sequence of eryACS-1 (ENA|EAQ27819|EAQ27819.1 acetyl-CoA synthetase from Erythrobacter sp. NAP1; herein SEQ ID NO. 21) being codon-optimized for E. coli.

FIG. 27 shows the primers used for site directed mutagenesis of eryACS-1 (from Erythrobacter sp. NAP1)

FIG. 28 shows the DNA sequence SEQ ID NO. 25 of the thioesterase YciA encoded by a gene selected from E. coli.

FIG. 29 shows the one-pot synthesis of mandelyl-CoA from oxalate, CoA and benzaldehyde, by combining OXS and OXC_{Me}. Assays were carried out at 30° C. in reaction buffer containing 50 mM TES-KOH pH 6.8, 10 mM MgCl₂. 0.5 mM ADP, 0.15 mM ThDP, 0.85 mM CoA, 10 mM ATP, 25 mM benzaldehyde, 5 μM OXS and 5 μM OXC_{Me}. The reaction was started by adding 10 mM disodium oxalate. Samples were taken after 0, 10, 60, 240 min and analyzed with the CoA ester method described above. Error bars show standard deviation of two replicates.

FIG. 30 shows Michaelis-Menten graph of eryACS-1 and variants thereof with formate as substrate according to example 6. AMP production was followed at 30° C. by coupling eryACS-1 to purified adenylate kinase (ADK), pyruvate kinase (PK) and lactate dehydrogenase (LDH). An assay containing 200 mM MOPS-KOH pH 7.8, 0.3 mM NADH, 10 mM MgCl₂, 2 mM ATP, 0.5 mM CoA, 2.5 mM PEP, 0.2 mg/mL ADK, 15 U/mL PK from rabbit muscle (Sigma), 23 U/mL LDH from rabbit muscle (Sigma), and an appropriate concentration of eryACS-1 was preincubated for 2 min and the reaction started by adding sodium formate to a final concentration of 10, 25, 50, 100, 150, 200 mM, respectively. Reaction procedure was monitored by following the oxidation of NADH at 340 nm. Error bars show standard deviation of three replicates. eryACS-1 V379I=eryACS-1, wherein at position 379 valine is replaced by isoleucine. eryACS-1 V379I Δpatz=eryACS-1, wherein at position 379 valine is replaced by isoleucine, and the enzyme were expressed by an E. coli strain lacking lysine acetyltransferase patZ.

FIG. 31 shows the one-pot synthesis of mandelyl-CoA from formate, CoA and benzaldehyde, by combining FCS and OXC_{Me} according to example 21. Assays were carried out at 30° C. in reaction buffer containing 50 mM TES-KOH pH 6.8, 10 mM MgCl₂. 0.5 mM ADP, 0.15 mM ThDP, 0.85 mM CoA, 10 mM ATP, 25 mM benzaldehyde, 5 μM FCS and 5 μM OXC_{Me}. The reaction was started by adding 50 mM sodium formate. Samples were taken after 0, 10, 60, 240 min and analyzed with the CoA ester method described above. Error bars show standard deviation of two replicates.

FIG. 32 shows (S)-mandelic acid formation of the cascade over time. (S)-Mandelic acid formation of the FCS-OXC-YciA cascade over time. Assays were carried out at 30° C. in reaction buffer consisting of 50 mM TES-KOH pH 6.8, 10 mM MgCl₂, 0.5 mM ADP, 0.15 mM ThDP, 0.5 mM CoA, 5 mM ATP, 25 mM benzaldehyde, 1 μM FCS, 2 μM OXC and 2 μM YciA. The reaction was started by adding 50 mM sodium formate. Samples were taken after 0, 2, 15, 30, 60, 120, 300 min and analyzed with the LC-MS detection of mandelic acid derivatives. Mandelic acid was quantified by comparison to commercially obtained standards of mandelic acid.

FIG. 33 shows formyl-CoA ‘proofreading’ by FRC. FRC converts formyl-CoA and oxalate into formate and oxalyl-CoA. Because mandelyl-CoA formation rate proceeds faster with oxalyl-CoA than with formyl-CoA as C₁ donor, addition of FRC results in increased mandelyl-CoA formation rate. Assays were carried out at 30° C. in reaction buffer consisting of 50 mM TES-KOH pH 6.8, 10 mM MgCl₂, 0.5 mM ADP, 0.15 mM ThDP and 25 mM benzaldehyde, 1 μM OXC_{Me} (A,C) or 1 μM OXC_{Me-Y497A} (B,D). In B and D the reaction additionally contained 20 μM FRC. The reaction was started by adding 1 mM oxalyl-CoA. Samples were taken after 0, 2, 5, 10 and 20 minutes and analyzed with the LC-MS detection of CoA esters. CoA esters were quantified by comparison to synthetic standards of formyl-CoA, oxalyl-CoA and mandelyl-CoA, respectively. Error bars show standard deviation of three replicates. E, mandelyl-CoA formation rates were determined by linear regression (slope of mandelyl-CoA in the graphs). Final concentration refers to the last time point (20 minutes). wt=wild type=OXC_{Me}; Y497A=OXC_{Me-Y497A}.

FIG. 34 shows formyl-CoA ‘proofreading’ by FRC in the OXS-OXC_{Me}-YciA cascade according to example 27. Addition of FRC results in increased mandelic acid and 3-phenyllactic acid formation rate. Assays were carried out at 30° C. in reaction buffer containing 50 mM TES-KOH pH 6.8, 10 mM MgCl₂. 0.5 mM ADP, 0.15 mM ThDP, 0.5 mM CoA, 10 mM ATP, 7.6 mM benzaldehyde or phenylacetaldehyde, 2 μM FRC, 2 μM YciA, 10 μM OXC_{Me-Y497A} and 5 μM OXS. The reaction was started by adding 10 mM disodium oxalate. Samples were taken after 0, 15, 30, 60 min and analyzed with the mandelic acid derivatives method described above.

FIG. 35 shows the one-pot synthesis of mandelyl-CoA from oxalate, CoA and benzaldehyde, by combining OXS, OXC_{Me} and FRC according to example 29, which acts as formyl-CoA proofreading enzyme. Assays were carried out at 30° C. in reaction buffer containing 50 mM TES-KOH pH 6.8, 10 mM MgCl₂, 0.5 mM ADP, 0.15 mM ThDP, 0.85 mM CoA, 10 mM ATP, 25 mM benzaldehyde, 1 μM OXC_{Me}, 5 μM OXS and 20 μM FRC and were mixed and the reaction was initiated by the addition of 10 mM disodium oxalate. Samples were taken after 0, 2, 5, 10, 30 min and quenched with 4% formic acid and products analyzed with the CoA ester method described above.

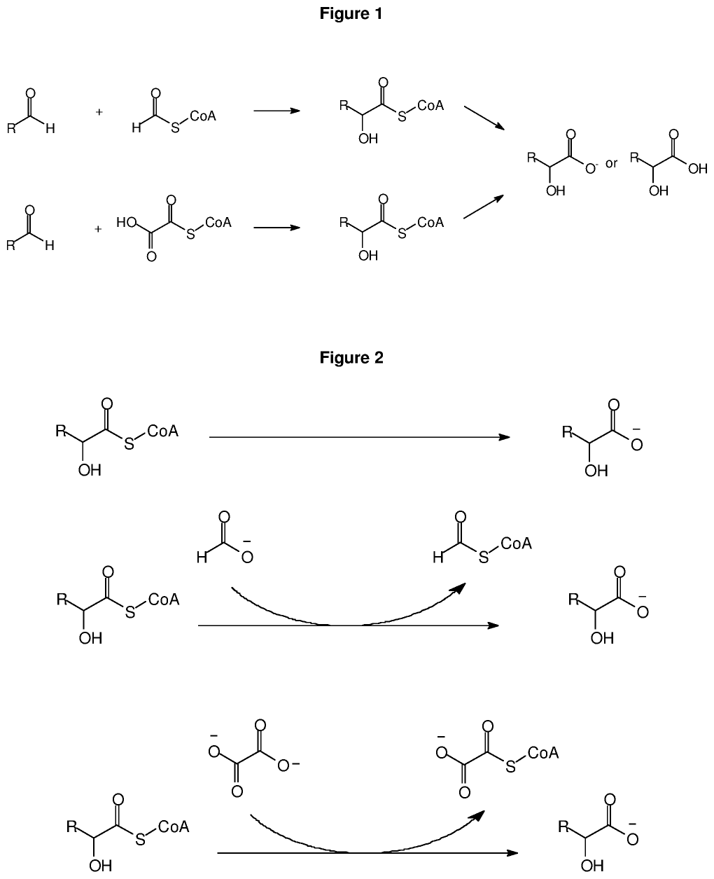

### EXAMPLES

Abbreviations
LC-MS liquid chromatography mass spectrometry
rcf relative centrifugal force
HPLC high performance liquid chromatography
UPLC ultra performance liquid chromatography
TOF time of flight
ESI electron spray ionization
HEPES 2-[4-(2-hydroxyethyl)piperazin-1-yl]ethanesulfonic acid
MES 2-(N-morpholino)ethanesulfonic acid
MOPS 3-Morpholinopropane-1-sulfonic acid
TES 2-[[1,3-dihydroxy-2-(hydroxymethyl)propan-2-yl]amino]ethanesulfonic acid
ThDP thiamine diphosphate
UV ultraviolet
CoA coenzyme A
ATP adenosine triphosphate
dNTP deoxy nucleoside triphosphate
NADH nicotinamide adenine dinucleotide
NADPH nicotinamide adenine dinucleotide phosphate
CDI carbonyldiimidazole
THF tetrahydrofuran
ACN acetonitrile
DMF dimethylformamide
EG ethylene glycol
DMSO dimethyl sulfoxide
FCS formyl-CoA synthetase
OXS oxalyl-CoA synthetase
OXC oxalyl-CoA decarboxylase
OXC_{Me} oxalyl-CoA decarboxylase from Methylobacterium extorquens
HACL hydroxyacyl-CoA lyases
HACL_{Hs} human hydroxyacyl-CoA lyases
HACOT α-hydroxyacyl-CoA:oxalate transferase
HAFT α-hydroxyacyl-CoA:formate transferase
FRC formyl-CoA:oxalate transferase
ADK adenylate kinase
LDH lactate dehydrogenase
PK pyruvate kinase
NdeI endonuclease isolated from Neisseria denitrificans
BamHI endonuclease isolated from Bacillus amyloliquefaciens
DpnI endonuclease isolated from Diplococcus pneumoniae
PCR polymerase chain reaction
LB lysogeny broth
TB terrific broth
IPTG Isopropyl β-D-1-thiogalactopyranoside
OD₆₀₀ optical density at 600 nm
FPLC fast protein liquid chromatography
SDS-PAGE sodium dodecyl sulfate-polyacrylamide gel electrophoresis

### Material and Methods

Chemicals

Unless stated otherwise, standard laboratory reagents were obtained from Sigma-Aldrich® (Steinheim, Germany) or Carl Roth GmbH & Co. KG (Karlsruhe, Germany) with the highest purity available. Propionaldehyde, Vanillin, (R)-mandelic acid were (S)-mandelic acid were obtained from Tokyo Chemical Industry (Zwijndrecht, Belgium). 4-chloromandelic acid was obtained from Alfa Aesar (ThermoFisher (Kandel) GmbH; Kandel, Germany).

Method for Detection of α-Hydroxyacyl-CoA Thioester (that is the Method Described in LC-MS Detection of CoA Esters) or α-Hydroxy Carboxylic Acids (that is the Method Described in LC-MS Detection of Mandelic Acid Derivatives)

LC-MS analyses was used to investigate the formation of the product and side products.

LC-MS Analyses

Samples were prepared for LC-MS analysis by quenching an aliquot of a reaction with formic acid (final concentration 4%) and centrifuging for 10 min at 17,000 rcf, to remove precipitated proteins. LC-MS data were analyzed and quantified using MassHunter Qualitative Navigator and Quantitative Analysis software (Agilent, Waldbronn, Germany).

LC-MS Detection of CoA Esters

Samples were diluted 1:10 in H₂O. UPLC-high resolution MS of CoA esters was performed as described previously.^{[5]} CoA esters were analyzed using an Agilent 6550 iFunnel Q-TOF LC-MS system equipped with an electrospray ionization source set to positive ionization mode. Compounds were separated on a RP-18 column (50 mm×2.1 mm, particle size 1.7 μm, Kinetex EVO C18, Phenomenex) using a mobile phase system comprised of 50 mM ammonium formate pH 8.1 (A) and methanol (B). Chromatographic separation was carried out using the following gradient condition at a flow rate of 250 μl/min: 0 min 2.5% B; 2.5 min 2.5% B; 8 min 23% B; 10 min 80% B; 11 min 80%; 12 min 2.5% B; 12.5 min 0% B. The column oven was set to 40° C. and autosampler was maintained at 10° C. Standard injection volume was 1 μl. Capillary voltage was set at 3.5 kV and nitrogen gas was used as nebulizing (20 psig), drying (13 l/min, 225° C.) and sheath gas (12 l/min, 400° C.). The time of flight (kindly confirm) (TOF) was calibrated using an ESI-L Low Concentration Tuning Mix (Agilent) before measurement (residuals less than 2 ppm for five reference ions) and was recalibrated during a run using 922.0908 m/z as reference mass. The scan range for MS and MS/MS data is 100-1000 m/z and 50-1000 m/z respectively. Collision energy used for MS/MS fragmentation was 35 eV.

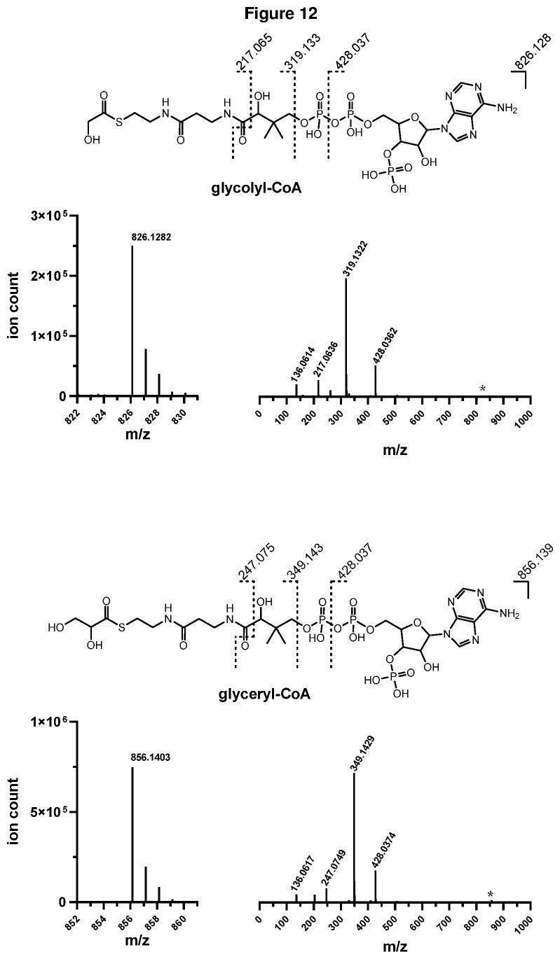

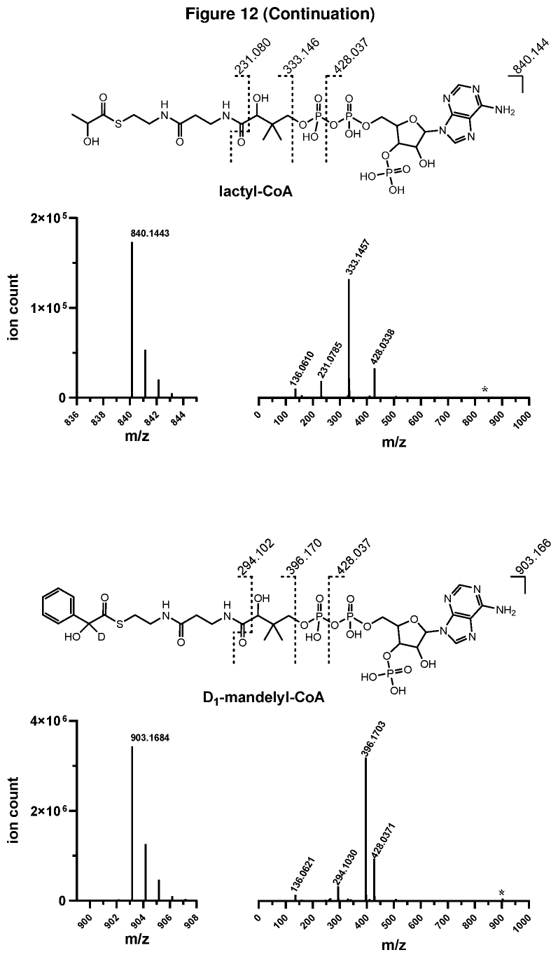

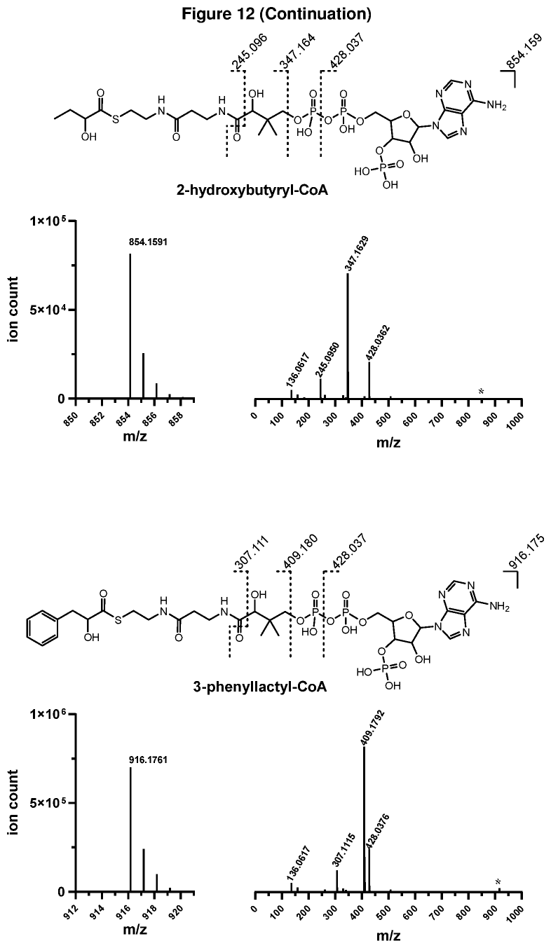

The MS/MS spectra are depicted in FIG. 12.

LC-MS Detection of Mandelic Acid Derivatives

Samples were diluted 1:10 in H₂O. UPLC-high resolution MS analyses were performed on an Agilent 6550 iFunnel QTOF LC/MS system equipped with an electrospray ionization source to negative ionization mode. The analytes were isocratically chromatographed on a chiral column (100 mm×2.1 mm, particle size 2.7 μm, Poroshell 120 Chiral-T, Agilent) kept at ambient temperature using a mobile phase system comprised of 30:70 20 mM ammonium formate pH 4/methanol at a flow rate of 250 μl/min for 10 min. Samples were held at 10° C. and injection volume was 1 μl. Capillary voltage was set at 3.5 kV and nitrogen gas was used as nebulizing (20 psig), drying (13 l/min, 225° C.) and sheath gas (12 l/min, 400° C.). MS data were acquired with a scan range of 100-1100 m/z. For quantification the calculated m/z value of [M-H]⁻ was used to obtain the extracted ion count from the total ion count.

Enzyme Assays with LC-MS Detection

Unless noted otherwise, all LC-MS assays were carried out at 30° C. in reaction buffer consisting of 50 mM TES-KOH pH 6.8, 10 mM MgCl₂. 0.5 mM ADP, 0.15 mM ThDP.

Kinetic Investigations

Spectrophotometric enzyme assays Assays were performed on a Cary-60 UV/Vis spectrophotometer (Agilent) at 30° C. using quartz cuvettes (10 mm path length; Hellma, Müllheim, Germany). For the determination of steady-state kinetic parameters, each concentration was measured in triplicates and the obtained curves were fit using GraphPad Prism 7. Hyperbolic curves were fit to the Michaelis-Menten equation to obtain apparent k_{cat} and K_{M} values.

### Synthesis of the Starting Materials and Reference Samples

### Example 1—Formyl-CoA Synthesis

Formyl-CoA was synthesized as described previously (W. S. Sly, E. R. Stadtman, J. Biol. Chem. 1963, 238, 2632-2638; b) S. Jonsson, S. Ricagno, Y. Lindqvist, N. G. Richards, J. Biol. Chem. 2004, 279, 36003-36012.). After Extraction with diethylether formyl-CoA was purified by preparative HPLC-MS with an acetonitrile gradient in 25 mM ammonium formate pH 4.2. The fractions containing the product were lyophilized and stored at −20° C. Formyl-CoA was dissolved in aq. HCl (pH 4). The concentration was determined by enzymatic depletion with PduP (J. Zarzycki, M. Sutter, N. S. Cortina, T. J. Erb, C. A. Kerfeld, Sci. Rep. 2017, 7, 42757.), following NADH consumption at 340 nm.

### Example 2—Oxalyl-CoA Synthesis

Oxalyl-CoA was synthesized enzymatically with OXS. A 5 mL reaction containing 50 mg mM CoA (0.064 mmol, 1 eq.), 52 mg ATP (0.086 mmol, 1.3 eq.), 10 mg disodium oxalate (0.075 mmol, 1.2 eq.) in buffer (100 mM MES-KOH pH 6.8, 15 mM MgCl₂) was started by adding OXS to a final concentration of 0.4 mg/mL and incubated at 30° C. for 1 hour. The reaction was quenched with 250 μL formic acid and the enzyme removed by centrifugation (4,000×g, 4° C., 10 min). Oxalyl-CoA was purified by preparative HPLC-MS with an acetonitrile gradient in 25 mM ammonium formate pH 4.2. The fractions containing the product were lyophilized and stored at −20° C. Oxalyl-CoA was dissolved in 10 mM acetate buffer pH 4.5. The concentration was determined spectrophotometrically, using the extinction coefficient for saturated CoA esters (ε₂₆₀ₙₘ=16.4 cm⁻¹ mM⁻¹) or by enzymatic depletion with PanE2, following NADPH consumption at 340 nm.

### Example 3—Mandelyl-CoA Synthesis (Reference Sample)

Mandelyl-CoA and (S)-mandelyl-CoA were synthesized chemically with the carbonyldiimidazole (CDI) CoA-acylation method described previously (D. M. Peter, B. Vögeli, N. S. Cortina, T. J. Erb, Molecules 2016, 21, 517.). 21 mg CDI (0.127 mmol, 4 eq.) was dissolved in 1 mL tetrahydrofuran, mandelic acid or (S)-mandelic acid was added (0.127 mmol, 4 eq.) and the mixture stirred at 22° C. for 15 min. 25 mg CoA (0.032 mmol, 1 eq.) was dissolved in 1 mL 0.5 M NaHCO₃ and added to the reaction mixture, followed by stirring at 22° C. for 30 min. THF was removed by applying vacuum (100 mbar) for 5 min. The mixture was then purified by preparative HPLC with a methanol gradient in 25 mM ammonium formate pH 8.0. The fractions containing the product were lyophilized and stored at −20° C. Mandelyl-CoA was dissolved in 10 mM acetate buffer pH 4.5. The concentration was determined spectrophotometrically, using the extinction coefficient for saturated CoA esters (ε₂₆₀ₙₘ=16.4 cm⁻¹ mM⁻¹). The concentration was confirmed by enzymatic depletion with YciA, detecting the liberated CoA with Ellman's reagent (ε₄₁₂ₙₘ=14.15 cm⁻¹ mM⁻¹).

### Example 4—Synthesis of the Peptides

### Example 4a—Cloning and Mutagenesis

Oligonucleotides were obtained from Eurofins Genomics (Ebersbach, Germany). oxc (MexAM1_META1p0990), oxs (MexAM1_META1p2130), and panE2 (MexAM1_META1p3141) were PCR-amplified from M. extorquens chromosomal DNA using the corresponding primers (Table 1). The purified PCR products were digested with NdeI and BamHI and ligated into pET-16b. Correct cloning was confirmed by sequencing (Eurofins Genomics).

The gene encoding eryACS-1 (GenBank: EAQ27819.1) was obtained by gene synthesis performed by the Joint Genome Institute (Walnut Creek, Calif., USA). The gene was codon optimized for E. coli and sub-cloned into pSEVA141.

Human Hacl1 (GenBank: CAB60200.1) was obtained by gene synthesis (Table 2), performed by BaseClear (Leiden, The Netherlands). The gene was codon optimized for E. coli and sub-cloned into pET-16b.

adk, paal, tesB and yciA were obtained from the ASKA collection ([M. Kitagawa, T. Ara, M. Arifuzzaman, T. Ioka-Nakamichi, E. Inamoto, H. Toyonaga, H. Mori, DNA Res. 2005, 12, 291-299).

Point mutations were introduced into oxc by PCR using mismatch primers (Table 1). A 50 μL reaction contained 60 ng of pET-16b_OXC_{Me}, 0.25 μM forward and reverse primer, 200 μM dNTP, 5 μL 10× reaction buffer, 1 μL Phusion polymerase (2 U/μL). Template plasmid was removed by DpnI digest (10 U) at 37° C. immediately after PCR amplification. Mutations were confirmed by sequencing.

TABLE 1
Primers used for cloning of OXC and OXS
and site-directed mutagenesis of OXC.

| | Nucleotide sequence (5′ to 3′) restriction |
|---|---|
| Primer name | sites or mismatches are underlined) |
| oxc_fw_Ndel | GTTCACATATGACCGTCCAGGCCCAG (SEQ ID NO. 7) |
| oxc_rv_BamHI | CGCTGGATCCTCACTTCTTCTTCAAGGTGCTC (SEQ ID NO. |
| | 8) |
| oxs_fw_Ndel | GTTCACATATGACGATGCTTCTGCC (SEQ ID NO. 9) |
| oxs_rv_BamHI | CAATGGATCCTCAGACCAGCCCGAG (SEQ ID NO. 10) |
| panE2_fw_Ndel | GCGCACATATGAGCATCGCGATCGTCG (SEQ ID NO. 11) |
| panE2_rv_BamHI | CAGAGGATCCTCATGCTCCCTGGATCGC (SEQ ID NO. 12) |
| oxc_Y497A_fw | CAACAACGGCATCGCTCGCGGCACCGAC (SEQ ID NO. 13) |
| oxc_Y497A_rv | GTCGGTGCCGCGAGCGATGCCGTTGTTG (SEQ ID NO. 14) |
| oxc_S568A_fw | CCGGCAGCGAGGCCGGCAATATCGG (SEQ ID NO. 15) |
| oxc_S568A_rv | CCGATATTGCCGGCCTCGCTGCCGG (SEQ ID NO. 16) |

TABLE 2
Synthetic gene sequences

| Gene name | DNA sequence |
|---|---|
| hacl1 | ATGCCGGACAGTAACTTCGCAGAGCGCAGCGAGGAGCAGG |
| (Gen Bank: | TGTCTGGTGCTAAAGTCATCGCTCAGGCCCTGAAAACGCAA |
| CAB60200) | GATGTGGAGTACATATTTGGCATCGTAGGCATCCCAGTGAC |
| (SEQ ID NO. | CGAAATCGCCATTGCTGCCCAGCAGCTAGGCATCAAGTACA |
| 24) | TCGGGATGAGGAATGAGCAAGCGGCTTGTTATGCTGCCTCC |
| | GCGATTGGATATCTGACAAGCAGGCCAGGAGTCTGCCTTGT |
| | TGTTTCTGGCCCAGGTCTCATCCATGCCTTGGGCGGTATGG |
| | CAAATGCAAACATGAACTGCTGGCCCTTGCTTGTGATTGGTG |
| | GTTCCTCTGAAAGAAACCAAGAAACAATGGGAGCTTTCCAGG |
| | AGTTTCCTCAGGTTGAAGCTTGTAGATTATATACCAAGTTCTC |
| | TGCCCGCYCAAGCAGCATAGAAGCTATTCCTTTTGTTATTGA |
| | AAAGGCAGTGAGAAGCAGTATCTATGGTCGTCCAGGTGCTT |
| | GCTATGTTGACATACCAGCAGATTTTGTGAACCTTCAGGTGA |
| | ATGTGAATTCTATAAAGTACATGGAACGCTGCATGTCACCTC |
| | CTATTAGCATGGCAGAAACCTCTGCTGTGTGCACGGCGGCT |
| | TCTGTTATTAGGAATGCCAAACAACCCCTTCTTATCATCGGG |
| | AAAGGTGCTGCTTACGCTCATGCAGAAGAGAGTATCAAGAAA |
| | TTGGTGGAGCAATATAAACTGCCATTTTTGCCCACCCCTATG |
| | GGAAAGGGTGTTGTCCCTGACAACCATCCATACTGTGTAGG |
| | TGCAGCCAGATCCAGGGCTTTGCAATTTGCTGATGTAATTGT |
| | GTTATTTGGTGCCAGACTAAATTGGATTTTACATTTTGGACTG |
| | CCTCCAAGATATCAGCCAGATGTGAAGTTTATCCAGGTTGAT |
| | ATCTGTGCAGAAGAATTGGGGAATAATGTAAAGCCCGCTGTT |
| | ACTTTGCTAGGAAACATACATGCTGTCACTAAGCAGCTTTTA |
| | GAGGAACTTGATAAAACACCATGGCAGTATCCTCCAGAGAG |
| | CAAGTGGTGGAAAACTCTGAGAGAAAAAATGAAGAGCAATG |
| | AAGCTGCATCCAAGGAACTAGCTTCTAAAAAATCCCTGCCTA |
| | TGAATTATTACACAGTATTCTACCATGTTCAAGAACAACTACC |
| | TAGAGACTGTTTCGTGGTAAGTGAAGGAGCAAATACTATGGA |
| | CATTGGACGGACTGTGCTTCAGAACTACCTTCCTCGTCACAG |
| | GCTTGATGCTGGTACTTTCGGAACAATGGGAGTTGGTTTGG |
| | GATTTGCTATTGCAGCTGCCGTGGTGGCTAAAGATAGAAGC |
| | CCTGGGCATTGGATCATCTGTGTGGAAGGAGACAGTGCATT |
| | TGGGTTTTCTGGCATGGAGGTAGAAACCATCTGCAGGTACA |
| | ACTTGCCAATCATACTGTTGGTAGTGAATAACAATGGAATTTA |
| | CCAAGGTTTTGATACAGATACTTGGAAAGAAATGTTAAAATTT |
| | CAAGATGCTACTGCAGTGGTCCCTCCAATGTGTTTGCTGCCA |
| | AATTCACATTATGAGCAAGTCATGACTGCATTTGGAGGCAAA |
| | GGGTATTTTGTACAAACACCAGAAGAACTCCAAAAATCCCTG |
| | GAGCAGAGCCTAGCAGACACAACTAAACCTTCTCTTATCAAC |
| | ATCATGATTGAGCCACAAGCCACACGGAAGGCCCAGGATTT |
| | TCATTGGCTGACCCGCTCTAATATGTAA |
| hacl1 codon | ATGCCGGATTCAAACTTTGCTGAACGTTCAGAAGAACAGGTT |
| optimized for | TCTGGCGCAAAAGTGATTGCGCAAGCGCTAAAAACCCAGGA |
| E. coli (SEQ ID | TGTCGAATATATCTTCGGTATAGTGGGTATTCCGGTGACGGA |
| NO. 17) | AATCGCCATCGCGGCTCAGCAACTCGGTATTAAATACATCGG |
| | TATGCGTAATGAGCAGGCGGCTTGTTACGCAGCAAGCGCGA |
| | TTGGCTATCTGACCTCAAGACCGGGGGTATGCCTTGTTGTCA |
| | GCGGCCCGGGCCTGATTCATGCCCTGGGTGGTATGGCGAA |
| | TGCTAACATGAACTGCTGGCCGCTGTTAGTTATAGGCGGCA |
| | GCAGTGAACGCAATCAGGAGACCATGGGCGCATTCCAAGAG |
| | TTTCCTCAGGTGGAAGCCTGCCGCCTGTATACCAAATTTTCT |
| | GCTAGACCTTCGTCAATAGAAGCCATCCCCTTCGTGATTGAA |
| | AAAGCGGTCCGTAGTTCTATCTATGGGCGCCCTGGGGCTTG |
| | CTATGTTGACATTCCCGCCGACTTCGTAAACCTCCAAGTGAA |
| | TGTCAATTCCATTAAATACATGGAACGCTGTATGTCGCCGCC |
| | GATCAGTATGGCCGAGACCAGCGCCGTGTGCACAGCGGCG |
| | AGTGTTATCCGCAACGCCAAACAGCCATTACTAATAATTGGG |
| | AAAGGAGCCGCCTATGCTCACGCTGAAGAGTCCATCAAAAA |
| | ACTGGTGGAACAATACAAGTTGCCCTTTTTACCTACTCCCAT |
| | GGGTAAAGGTGTTGTACCTGATAATCATCCATACTGCGTCGG |
| | TGCAGCCCGTTCCCGCGCACTACAGTTCGCTGACGTGATTG |
| | TGCTGTTTGGTGCGCGTTTAAACTGGATACTCCACTTCGGTT |
| | TACCGCCACGCTACCAGCCGGATGTGAAATTTATACAGGTTG |
| | ACATCTGTGCCGAAGAACTGGGCAATAATGTGAAACCAGCA |
| | GTCACTTTGCTGGGGAACATCCATGCAGTCACCAAACAACTG |
| | CTTGAAGAGCTGGACAAGACGCCGTGGCAATATCCGCCAGA |
| | GTCAAAATGGTGGAAGACGCTACGTGAGAAAATGAAGAGCA |
| | ACGAAGCGGCTTCGAAAGAGTTGGCCTCCAAAAAGTCATTG |
| | CCTATGAATTACTATACCGTCTTTTATCATGTCCAAGAGCAGC |
| | TGCCGCGGGATTGCTTTGTCGTGTCGGAGGGCGCCAACACC |
| | ATGGATATAGGTCGCACTGTCCTTCAGAACTATCTGCCCCGG |
| | CATCGCCTGGACGCTGGTACCTTCGGAACTATGGGCGTAGG |
| | GCTGGGCTTTGCTATTGCGGCGGCAGTGGTAGCAAAGGATC |
| | GCAGCCCGGGGCAGTGGATTATTTGTGTAGAAGGGGACTCC |
| | GCATTTGGATTCTCTGGTATGGAGGTAGAGACAATATGCCGT |
| | TATAATTTGCCAATTATCCTTCTAGTGGTGAATAACAATGGTA |
| | TTTATCAGGGCTTTGATACAGATACGTGGAAGGAAATGCTGA |
| | AATTTCAAGATGCCACAGCCGTGGTACCCCCCATGTGTTTGC |
| | TGCCGAACTCTCATTACGAGCAAGTGATGACAGCATTCGGC |
| | GGCAAGGGTTACTTTGTGCAGACACCGGAAGAACTTCAAAA |
| | GAGCCTTCGCCAGAGCTTGGCGGACACCACGAAACCGAGTC |
| | TGATTAATATAATGATTGAACCACAGGCCACAAGGAAGGCAC |
| | AAGACTTTCACTGGCTCACGCGTTCAAATATGTAA |

### Example 4b—Protein Production and Purification

All proteins except HACL_{Hs} and FCS were heterologously produced in E. coli BL21 (DE3). 500 mL TB containing 100 μg/mL ampicillin (in the case of OXC_{Me}, OXS, FRC, PduP, PanE2, eryACS-1) or 34 μg/mL chloramphenicol (AdK, Paal, TesB and YciA) was inoculated with freshly-transformed cells and incubated at 37° C. After reaching an OD₆₀₀ of 0.8, expression was induced by adding IPTG to a final concentration of 0.25 mM and the incubation temperature was lowered to 25° C. FCS was produced in E. coli BL21-AI (ThermoFisher Scientific) in which the peptidyl-lysine N-acetyltransferase gene patZ had been replaced by a kanamycin resistance cassette kanR. 1 L TB containing 100 μg/mL ampicillin and 50 μg/mL kanamycin was inoculated with freshly-transformed cells and incubated at 37° C. After reaching an OD₆₀₀ of 0.6, L-arabinose was added to a final concentration of 0.2% (w/v) and the incubation temperature was lowered to 25° C. After 30 minutes, expression was induced by IPTG to a final concentration of 0.5 mM. HACL_{Hs} was produced in E. coli ArcticExpress (DE3). 1 L LB containing 100 μg/mL ampicillin and 15 μg/mL gentamycin was inoculated with freshly-transformed cells and incubated at 37° C. After reaching an OD₆₀₀ of 0.8 the culture was cooled on ice for 15 min. Then expression was induced by adding IPTG to a final concentration of 0.1 mM and the incubation temperature was lowered to 15° C. Cells were harvested after 16 h (24 h for HACL_{Hs}) by centrifugation (4500×g, 10 min) and resuspended in buffer A (500 mM KCl, 50 mM HEPES-KOH pH 7.6). If not used immediately, cell pellets were flash-frozen in liquid nitrogen and stored at −20° C. The cell lysate obtained by sonication was clarified by centrifugation 75,000×g at 4° C. for 45 min. The supernatant was filtered through a 0.4 μm syringe tip filter (Sarstedt, Nümbrecht, Germany). Ni-affinity purification was performed with an Äkta FPLC system from GE Healthcare (GE Healthcare, Freiburg, Germany). The filtered soluble lysate was loaded onto a 1 mL Ni-Sepharose Fast Flow column (HisTrap FF, GE Healthcare, Little Chalfont, UK) that had been equilibrated with 10 mL buffer A. After washing with 20 mL 85% buffer A, 15% buffer B (500 mM KCl, 50 mM HEPES-KOH pH 7.6, 500 mM imidazole), the protein was eluted with 100% buffer B. Fractions containing purified protein were pooled and the buffer was exchanged to storage buffer (150 mM KCl, 50 mM HEPES-KOH pH 7.6) with a desalting column (HiTrap, GE Healthcare). Proteins were concentrated by ultrafiltration (Amicon Ultra). Concentration was determined on a NanoDrop 2000 Spectrophotometer (Thermo Scientific, Waltham, Mass., USA) using the extinction coefficient at 280 nm, as calculated by protparam (https://web.expasy.org/protparam/). Enzyme purity was confirmed by SDS-PAGE. The purified proteins were stored in 50 vol % glycerol at −20° C. OXC_{Me} wild-type and mutants were flash-frozen in liquid nitrogen and stored at −80° C.

Kinetic Investigations

### Example 5—Michaelis-Menten Kinetics of OXS

Oxalyl-CoA production was followed by coupling OXS to purified PanE2, an NADPH-dependent oxalyl-CoA reductase from M. extorquens. An assay containing 50 mM potassium phosphate pH 6.5, 0.3 mM NADPH, 10 mM MgCl₂, 1 mM ATP, 2.5 mM CoA, 1.2 μM PanE2, 176 nM OXS was preincubated for 2 min and the reaction started by adding disodium oxalate to a final concentration of 5, 10, 25, 100, 250, 1′000 μM, respectively. Reaction procedure was monitored by following the oxidation of NADPH at 340 nm.

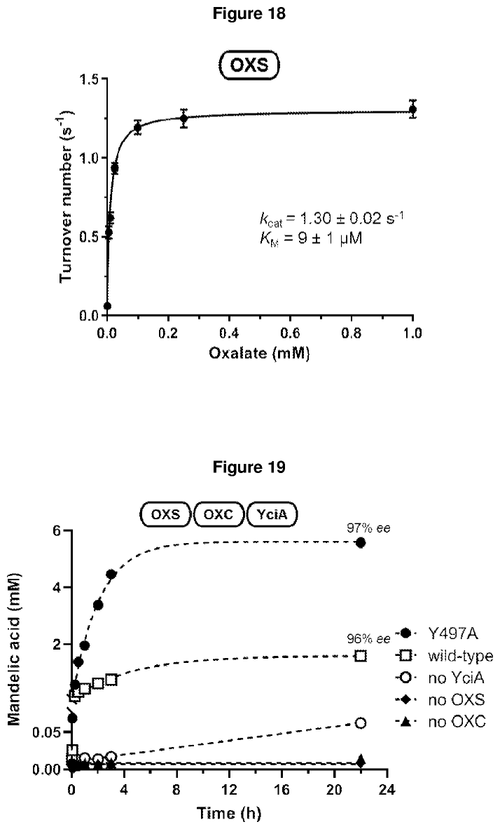

Result: The steady-state kinetic parameters are shown in FIG. 18 (k_{cat}=1.30±0.02 s⁻¹; K_{M}(oxalate)=9±1 μM)

### Example 6—Michaelis-Menten Kinetics OXC_{Me}-Decarboxylation

Formyl-CoA production was followed by coupling OXC to purified PduP, a promiscuous CoA-dependent aldehyde dehydrogenase that reduces formyl-CoA to formaldehyde under NADH consumption. An assay containing 50 mM MES-KOH pH 6.5, 0.3 mM NADH, 10 mM MgCl₂, 0.5 mM ADP, 0.15 mM ThDP, 5 μM PduP, and OXC_{Me} or mutant thereof like OXC_{Me-Y497A}, OXC_{Me-S568A} or OXC_{Me-Y497A-S568A} (concentration depending on the mutant) was preincubated for 2 min and the reaction started by adding oxalyl-CoA (concentrations depending on the mutant). Reaction procedure was monitored by following the oxidation of NADH at 340

The formyl-CoA formed after the decarboxylation of the oxalyl-CoA by ThDP and OXC_{Me} was removed from the reaction mixture in order to investigate the decarboxylation without the influence of the equilibrium.

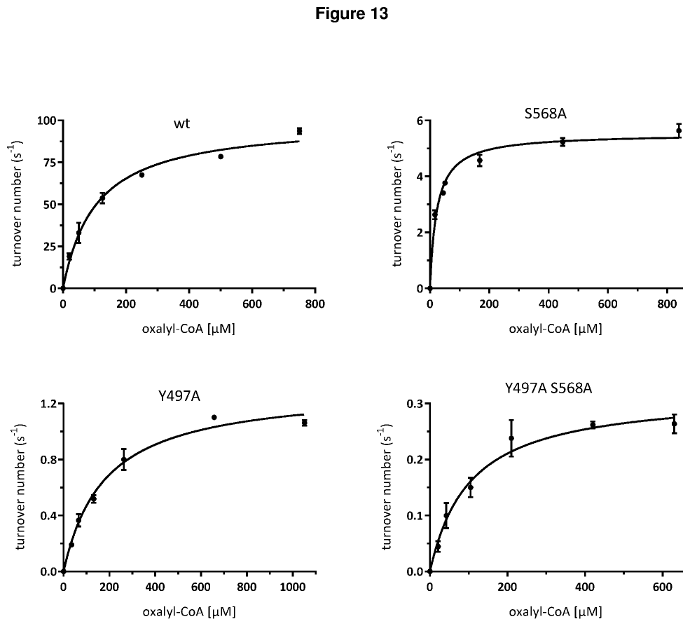

Results: The results are depicted in the following table 3 and in curve of the kinetic investigations in FIG. 13.

TABLE 3
Steady-state kinetic parameters of oxalyl-
CoA decarboxylation catalyzed by OXCMe.^{[a]}

| Mutation | k_{cat} (s⁻¹) | K_{M} (μM) | k_{cat}/K_{M} (s⁻¹ M⁻¹) |
|---|---|---|---|
| wild-type | 98 ± 3 | 105 ± 11 | 9.3 × 10⁵ |
| Y497A | 1.32 ± 0.04 | 180 ± 17 | 7.3 × 10³ |
| (OXC_{Me-Y497A}) | | | |
| S568A | 5.53 ± 0.11 | 23 ± 2 | 1.1 × 10⁵ |
| (OXC_{Me-S568A}) | | | |
| Y497A S568A | 0.32 ± 0.01 | 103 ± 15 | 3.1 × 10³ |
| (OXC_{Me-Y497A-S568A}) | | | |

^{[a]}Michaelis-Menten graphs are shown in FIG. 13.

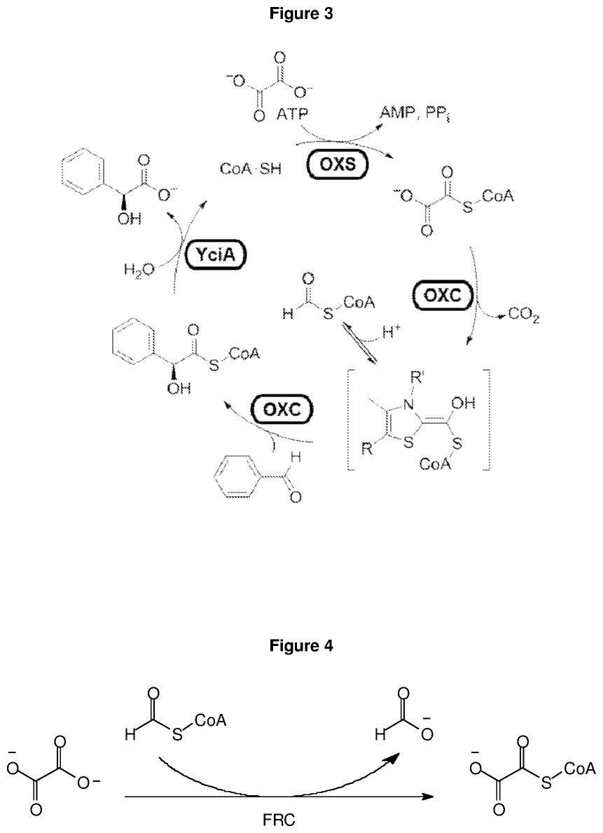

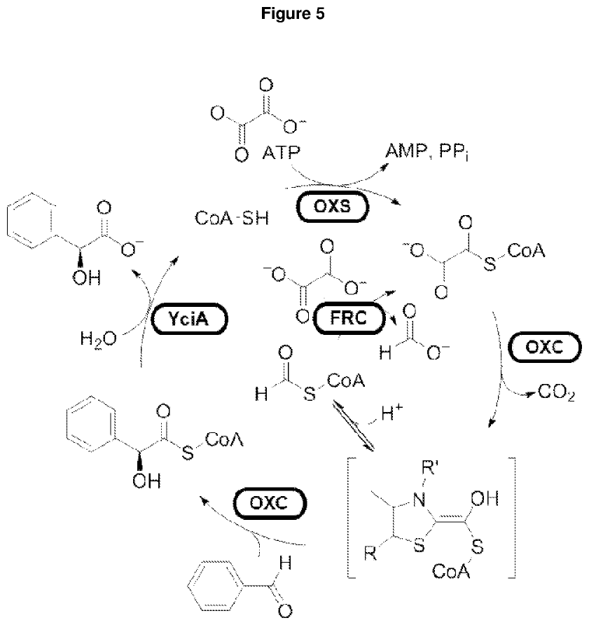

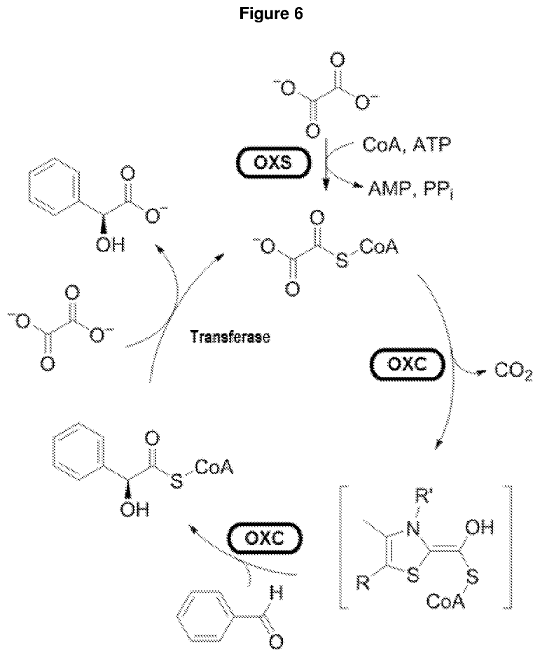

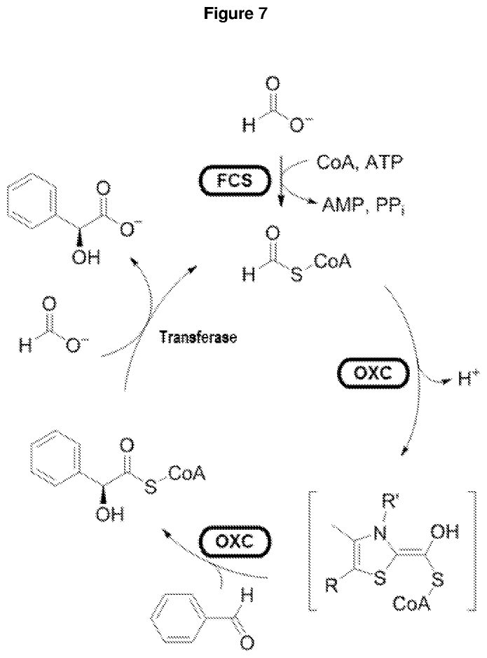

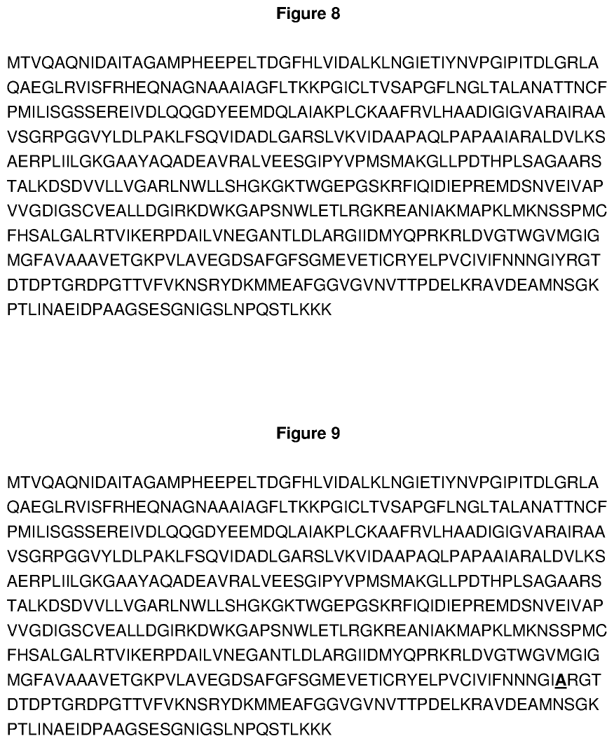

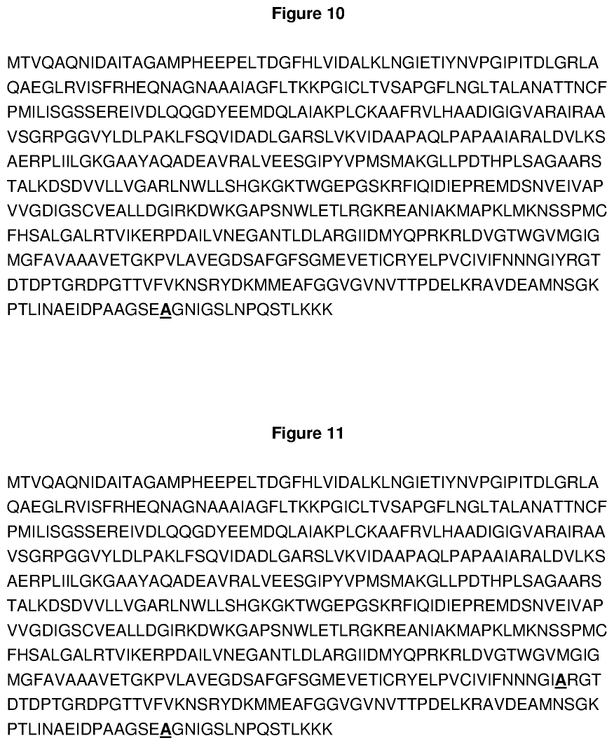

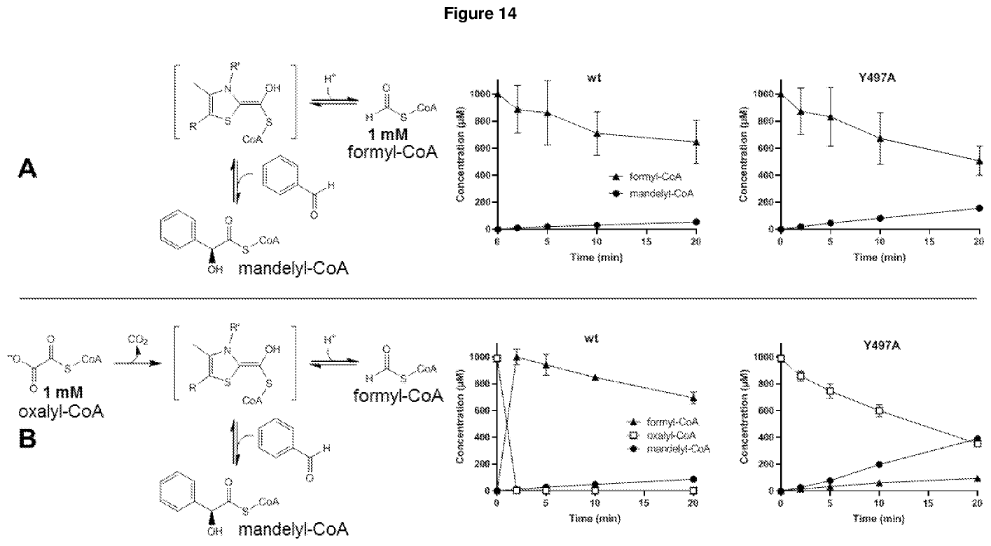

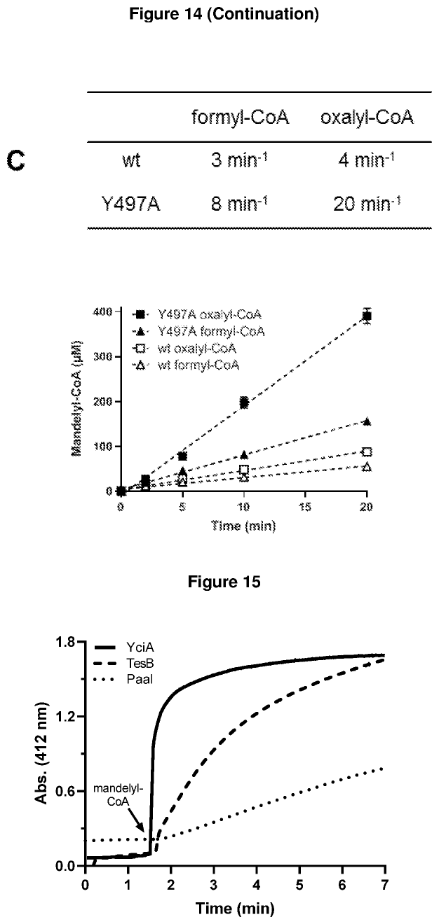

In OXC_{Me} variants OXC_{Me-Y497A} having the sequence SEQ ID NO. 2, and OXC_{Me-S568A} having the sequence SEQ ID NO. 3, formyl-CoA formation was decreased 20 to 50-fold, while the K_{M} of both variants was largely unaffected (Table 3 and FIG. 13). When starting with oxalyl-CoA and benzaldehyde OXC_{Me-Y497A} showed 5-fold increased mandelyl-CoA production rate (20 min⁻¹; FIG. 14C), suggesting that activity of the enzyme was successfully redirected towards carboligation by suppressing protonation of thiamine-α-hydroxyl-CoA-adduct (see FIG. 3).

The mutation Y497A increased the ratio of carboligation to decarboxylation by a factor of approximately 400 compared to the wild-type

### Example 7—OXC_{Me} Aldehyde and Ketone Screen

In reaction buffer, 1 mM formyl-CoA and 10 mM aldehyde (formaldehyde, acetaldehyde, glycolaldehyde, propionaldehyde, glyceraldehyde, glyoxylate, succinic semialdehyde, benzaldehyde, phenylacetaldehyde, and acetone) was mixed with 5 μM OXC_{Me} or HACL_{Hs}. The reaction was stopped after 1 h and products analyzed with the CoA ester method described above.

Result: Glyceraldehyde, glyoxylate and acetone were not accepted by OXC_{Me}, i.e. a carbon-carbon-bond was not formed. The results are listed in table 4:

TABLE 4
Comparison of the aldehyde substrate scope of OXC_{Me}

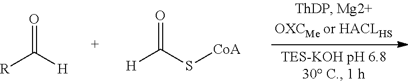

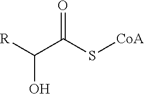

| Entry | R | Product name | OXCMe^{[c]} | HACL_{Hs}^{[c]} |
|---|---|---|---|---|
| 1 | H | glycolyl-CoA | 100 | 11 |
| 2 | CH₃ | lactyl-CoA | 74 | 100 |
| 3 | CH₂CH₃ | 2-hydroxybutyryl- | 5 | 100 |
| | | CoA | | |
| 4 | CH₂OH | glyceryl-CoA | 1 | 100 |
| 5 | CHOHCH₂OH | erythronyl-CoA | n.d.^{[b]} | n.d.^{[b]} |
| 6 | COOH | tartronyl-CoA | n.d. | n.d.^{[b]} |
| 7 | (CH2)2COOH | 2- | 1 | 100 |
| | | hydroxyglutaryl- | | |
| | | CoA | | |
| 8 | Ph | mandelyl-CoA | 100 | 3 |
| 9 | CH2Ph | phenyllactyl-CoA | 22 | 100 |

[a]The reaction contained 2a-2i (10 mM), formyl-CoA (1 mM), OXC_{Me}. Products were analyzed by LC-MS after 1 h reaction time.
^{[b]}Product not detected.
^{[c]}Relative activity in %. Relative activity refers to the comparison of OXC and HACL.

### Example 8—Comparison of the Decarboxylation Activity of OXC_{Me} and HACL_{Hs}

Oxalyl-CoA decarboxylation activity of OXC_{Me} and HACL_{Hs}. Assays were carried out at 30° C. in 100 mM MES-KOH pH 6.8 and contained 10 mM MgCl₂. 0.5 mM ADP, 0.15 mM ThDP, 0.05 μM OXC_{Me} or 1 μM HACL_{Hs} or no enzyme. The reaction was started by adding 0.5 mM oxalyl-CoA. Samples were taken after 1, 15, 30, 60 min and analyzed with the LC-MS detection of CoA esters.

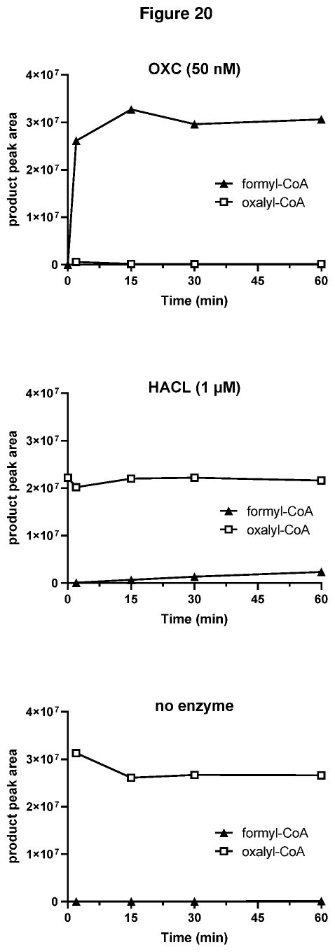

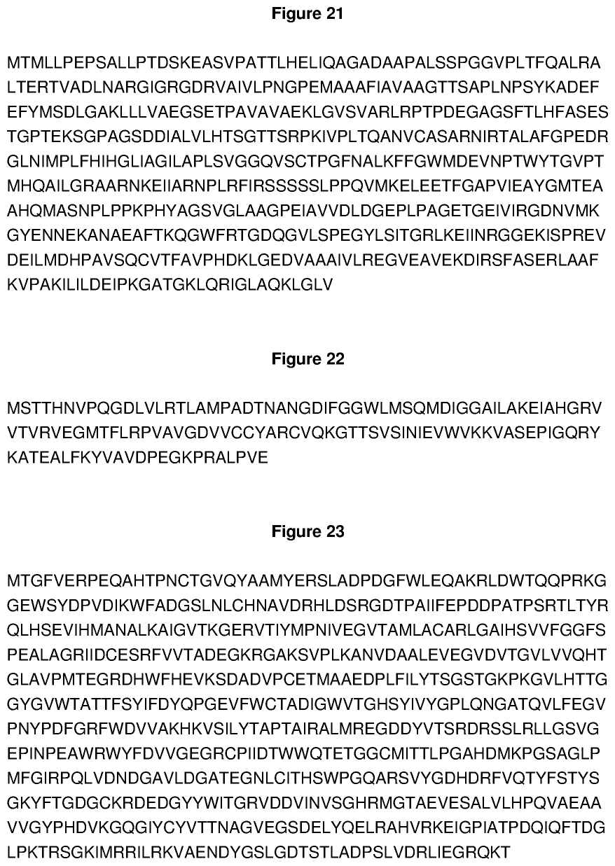

Results: The results are depicted in FIG. 20. In the control experiment without an enzyme the concentration of the oxalyl-CoA does not change, and thus formyl-CoA is not formed.

### Example 9—Comparison OXC Wild Type and OXC_{Me-Y497A} Starting Form Formyl-CoA or Oxalyl-CoA

Assays were carried out at 30° C. in reaction buffer consisting of 50 mM TES-KOH pH 6.8, 10 mM MgCl₂. 0.5 mM ADP, 0.15 mM ThDP and 25 mM benzaldehyde, 1 μM OXC_{Me} or 1 μM OXC_{Me-Y497A}. The reaction was started by adding either 1 mM formyl-CoA or 1 mM oxalyl-CoA. Samples were taken after 0, 2, 5, 10 and 20 minutes and analyzed with the LC-MS detection of CoA esters. CoA esters were quantified by comparison to synthetic standards of formyl-CoA, oxalyl-CoA and mandelyl-CoA, respectively. Concentration of CoA-esters was determined by comparison to a standard curve obtained from chemically synthesized formyl-CoA, oxalyl-CoA and mandelyl-CoA, respectively.

Results: The results are depicted in FIGS. 14A and 14B. Starting from the formyl-CoA the formation is faster by means of OXC_{Me-Y497A} than OXC_{Me} (wild type). When oxalyl-CoA is used as a C₁-source, the mandelyl-CoA is formed much faster in case of the OXC_{Me-Y497A}.

### Example 10—YciA Substrate Screen (Selectivity of YciA Towards Formyl-CoA, Oxalyl-CoA and Mandelyl-CoA)

Thioesterase activity of YciA towards oxalyl-CoA, formyl-CoA and racemic mandelyl-CoA. Assays were carried out at 30° C. in reaction buffer consisting of 50 mM TES-KOH pH 6.8, 10 mM MgCl₂. 0.5 mM ADP, 0.15 mM ThDP and 25 mM benzaldehyde, 2 μM YciA (A) or no YciA (B). The reaction was started by adding either 0.5 mM formyl-CoA or 0.5 mM oxalyl-CoA or 0.5 mM mandelyl-CoA. Samples were taken after 0, 1, 5 and 30 minutes and analyzed with the LC-MS detection of CoA esters. CoA esters were quantified by comparison to synthetic standards of formyl-CoA, oxalyl-CoA and mandelyl-CoA, respectively.

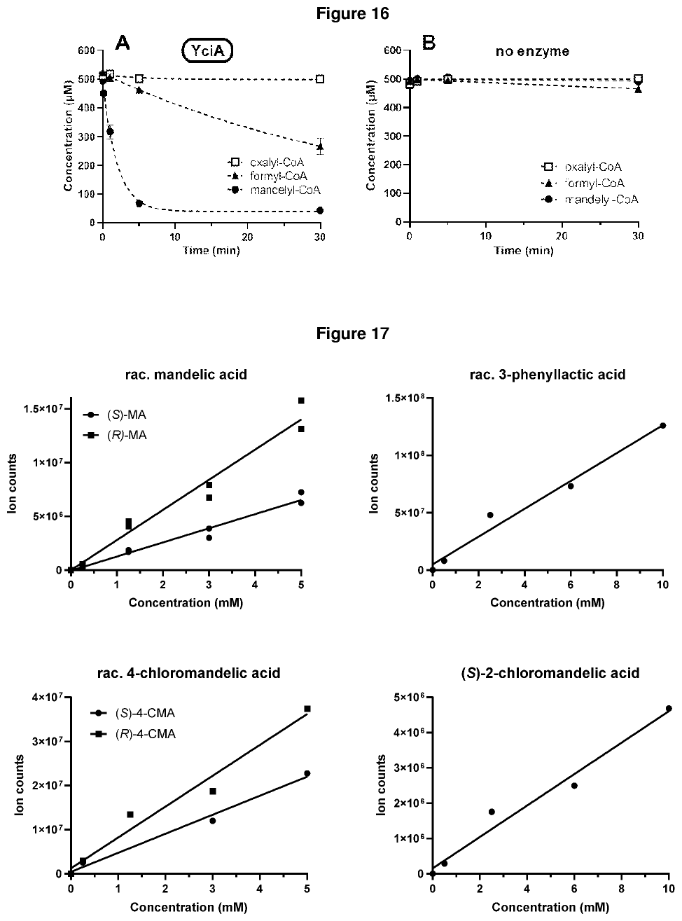

Results: The results are depicted in FIG. 16.

YciA from E. coli has shown a relatively high α-hydroxyacyl-CoA in particular mandelyl-CoA thioesterase activity, low activity with formyl-CoA and no activity with oxalyl-CoA.

### Example 11—Comparison of the Activity of the Thioesterase Activity of YciA, TesB and Paal

Screen for mandelyl-CoA Thioesterase activity. CoA formation was detected with the Ellman's reagent (5,5′-dithiobis-(2-nitrobenzoic acid)), which reacts with free thiols under release of 2-nitro-5-thiobenzoate (ε₄₁₂ₙₘ=14, 150 M⁻¹ cm⁻¹). Assays were carried out at 30° C. in 50 mM MES-KOH pH 6.8 and contained 1 mM Ellman's reagent and 0.5 μM YciA or 0.5 μM TesB or 0.5 μM Paal. The reaction was started by adding 0.1 mM mandelyl-CoA.

Results: The result is depicted in FIG. 15. YciA exhibits a higher thioesterase activity than TesB and Paal.

### Example 12—OXS-OXC_{Me}-YciA Cascade Prototyping

In a 1.5 mL microfuge tube, reaction buffer containing 50 mM TES-KOH pH 6.8, 10 mM MgCl₂. 0.5 mM ADP, 0.15 mM ThDP, 0.5 mM CoA, 10 mM ATP, 25 mM benzaldehyde, 2 μM YciA, 5 μM OXC_{Me} and 5 μM OXS were mixed and the reaction was initiated by the addition of 10 mM disodium oxalate. For the negative controls each enzyme was omitted in a separate reaction. Samples were taken after 0, 3, 15, 40 min, 1, 2, 3 and 22 h and analyzed with the mandelic acid derivatives method described above. For quantification commercial, racemic mandelic acid was diluted in reaction buffer to appropriate concentrations to obtain a calibration curve

### Example 13—Comparison of OXC_{Me} with its Mutant

Assays were carried out at 30° C. in reaction buffer consisting of 50 mM TES-KOH pH 6.8, 10 mM MgCl₂. 0.5 mM ADP, 0.15 mM ThDP, 0.5 mM CoA, 10 mM ATP, 25 mM benzaldehyde, 5 μM OXS, 5 μM OXC and 2 μM YciA. The reaction was started by adding 10 mM oxalate. As negative controls, YciA, OXS or OXC were omitted in separate reactions. Samples were taken after 0, 0.05, 0.25, 0.66, 1, 2, 3, 22 hours and analyzed with the LC-MS detection of mandelic acid derivatives.

Mandelic acid was quantified by comparison to commercially obtained standards of mandelic acid.

Results: The results are depicted in FIG. 19.

When OXC_{Me} is replaced by the OXC_{Me-Y497A} variant having the sequence SEQ ID NO. 2, mandelic acid production rate increased 5-fold and yield increased 4-fold.

### Example 14—OXS-OXC_{Me}-YciA Cascade Aldehyde Scope

The aldehyde substrate screen of the OXS-OXC_{Me-Y497A}-YciA cascade was carried out as described above (example 13), except that the aromatic aldehydes were prepared as 33 mM stocks in 20 vol % DMSO and diluted to final concentration of 25 mM into the assay. Samples were analyzed with the mandelic acid derivatives method described above. Mandelic acid (3a), 4-chloromandelic acid (3c), 2-chloromandelic acid (3d) and 3-phenyllactic acid (3b) were quantified by comparison to commercial standards.

The results are depicted in the following table 5.

TABLE 5
Scope of the OXS-OXC_{Me-Y497A}-YciA cascade for the synthesis of chiral
α-hydroxy acids.^{[a]}

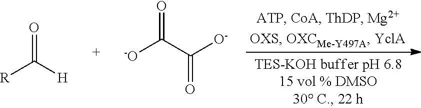

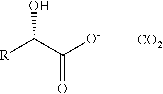

| | | | Conversion | ee |
|---|---|---|---|---|
| Entry | R | Product name | [%]^{[b]} | [%]^{[b]} |
| 1 | Ph | Mandelic acid | 75 | 99 |
| 2 | CH₂Ph | 3-phenyllactic acid | 58 | n.d.^{[c]} |
| 3 | 4-OH—Ph | 4-hydroxymandelic acid | n.d. | 95 |
| 4 | 4-NO₂—Ph | 4-nitromandelic acid | n.d. | 80 |
| 5 | 4-Cl—Ph | 4-chloromandelic acid | 57 | 95 |
| 6 | 3-Cl—Ph | 3-chloromandelic acid | n.d. | 98 |
| 7 | 2-Cl—Ph | 2-chloromandelic acid | 93 | 44 |
| 8 | 4-CHO—Ph | 4-aldehydemandelic acid | n.d. | 46 |
| 9 | 4-COOH—Ph | 4-formylmandelic acid | n.d. | n.d. |
| 10 | 4-COCH₃Ph | 4-acetylmandelic acid | n.d. | 51 |
| 11 | 4-N(CH₃)₂Ph | 4-(dimethylamino)mandelic | n.d. | n.d. |
| | | acid | | |
| 12 | 4-C≡CH—Ph | 4-ethynylmandelic acid | n.d. | 75 |
| 13 | 3-OCH₃-4- | 4-hydroxy-3- | n.d., product | n.d. |
| | OH—Ph | methoxymandelic acid | detected | |
| 14 | 2-furyl | Hydroxy(2-furyl)acetic acid | n.d. | 75 |
| 15 | 2-thiopheneyl | Hydroxy(2- | n.d. | 65 |
| | | thiopheneyl)acetic acid | | |
| 16 | 3-pyridinyl | Hydroxy(3-pyridinyl)acetic | n.d., product | n.d. |
| | | acid | detected | |
| 17 | 2-naphthyl | Hydroxy(2-naphthyl)acetic | n.d. | 80 |
| | | acid | | |

^{[a]}The reaction contained aldehyde (25 mM), disodium oxalate (10 mM), ATP (10 mM), OXS(5 μM), OXC_{Me-y497A} (5 μM), YciA (2 μM).
^{[b]}Determined by chiral HPLC (see Figure S5-7 for detailed quantification).
^{[c]}Not determined.

### Example 14b—OXS-OXC_{Me}-YciA Cascade

Assays were carried out at 30° C. in reaction buffer consisting of 50 mM TES-KOH pH 6.8, 10 mM MgCl₂, 0.5 mM ADP, 0.15 mM ThDP, 0.5 mM CoA, 10 mM ATP, 10 mM oxalate, 5 μM OXS, 5 μM OXC and 2 μM YciA. The reaction was started by adding 25 mM aromatic aldehyde (prepared as 33 mM stock solution containing 20 vol % DMSO). The tested aromatic aldehydes are listed in Table 6. The reactions were quenched by the addition of 4% formic acid after 24 h and analyzed with the LC-MS detection of mandelic acid derivatives.

Results: The results are depicted in Table 6.

TABLE 6
Extended aldehyde scope of the OXS-OXC_{Me-Y497A}-YciA cascade for the
synthesis of chiral α-hydroxy acids.

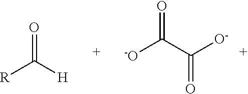

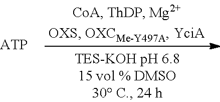

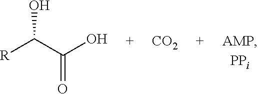

| Entry | R | Product |
|---|---|---|
| 18 | 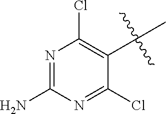 [SMILES: *c1c(Cl)nc(N)nc1Cl] | 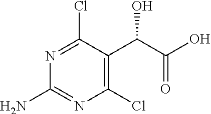 [SMILES: Nc1nc(Cl)c([C@H](O)C(=O)O)c(Cl)n1] |
| 19 | 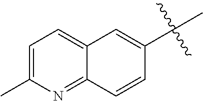 [SMILES: *c1ccc2nc(C)ccc2c1] | 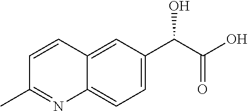 [SMILES: Cc1ccc2cc([C@H](O)C(=O)O)ccc2n1] |
| 20 | 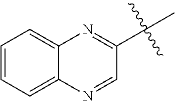 [SMILES: *c1cnc2ccccc2n1] | 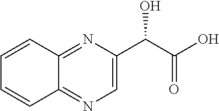 [SMILES: O=C(O)[C@@H](O)c1cnc2ccccc2n1] |
| 21 | 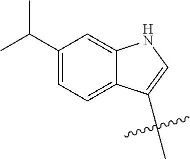 [SMILES: *c1c[nH]c2cc(C(C)C)ccc12] | 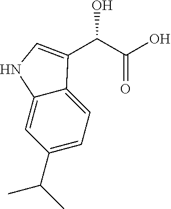 [SMILES: CC(C)c1ccc2c([C@H](O)C(=O)O)c[nH]c2c1] |
| 22 | 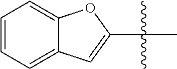 [SMILES: *c1cc2ccccc2o1] | 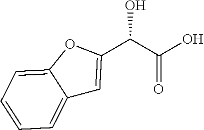 [SMILES: O=C(O)[C@@H](O)c1cc2ccccc2o1] |
| 23 | 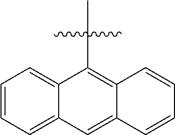 [SMILES: *c1c2ccccc2cc2ccccc12] | 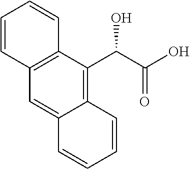 [SMILES: O=C(O)[C@@H](O)c1c2ccccc2cc2ccccc12] |
| 24 | 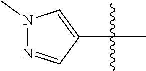 [SMILES: *c1cnn(C)c1] | 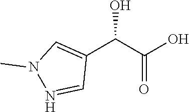 |
| 25 | 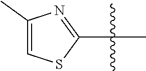 [SMILES: *c1nc(C)cs1] | 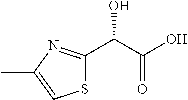 |
| 26 | 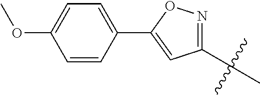 [SMILES: *c1cc(-c2ccc(OC)cc2)on1] | 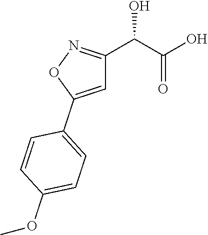 [SMILES: COc1ccc(-c2cc([C@H](O)C(=O)O)no2)cc1] |
| 27 | 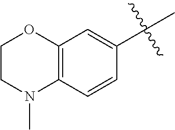 [SMILES: *c1ccc2c(c1)OCCN2C] | 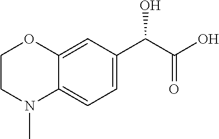 [SMILES: CN1CCOc2cc([C@H](O)C(=O)O)ccc21] |
| 28 | 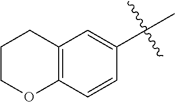 [SMILES: *c1ccc2c(c1)CCCO2] | 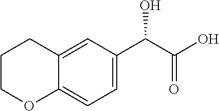 [SMILES: O=C(O)[C@@H](O)c1ccc2c(c1)CCCO2] |
| 29 | 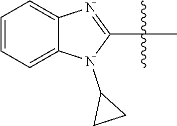 [SMILES: *c1nc2ccccc2n1C1CC1] | 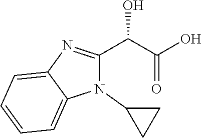 [SMILES: O=C(O)[C@@H](O)c1nc2ccccc2n1C1CC1] |
| 30 | 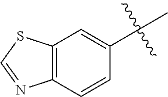 [SMILES: *c1ccc2ncsc2c1] | 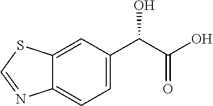 [SMILES: O=C(O)[C@@H](O)c1ccc2ncsc2c1] |
| 31 | 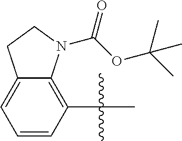 [SMILES: *c1cccc2c1N(C(=O)OC(C)(C)C)CC2] | 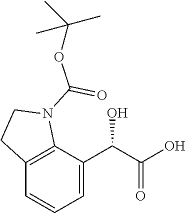 [SMILES: CC(C)(C)OC(=O)N1CCc2cccc([C@H](O)C(=O)O)c21] |
| 32 | 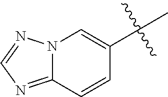 [SMILES: *c1ccc2ncnn2c1] | 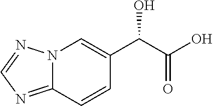 [SMILES: O=C(O)[C@@H](O)c1ccc2ncnn2c1] |
| 33 | 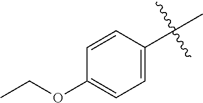 [SMILES: *c1ccc(OCC)cc1] | 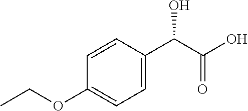 |
| 34 | 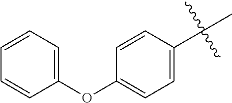 [SMILES: *c1ccc(Oc2ccccc2)cc1] | 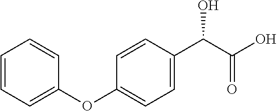 [SMILES: O=C(O)[C@@H](O)c1ccc(Oc2ccccc2)cc1] |
| 35 | 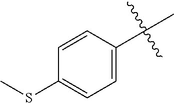 [SMILES: *c1ccc(SC)cc1] | 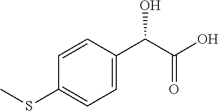 |
| 36 | 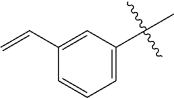 [SMILES: *c1cccc(C=C)c1] | 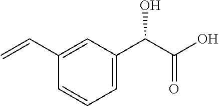 |
| 37 | 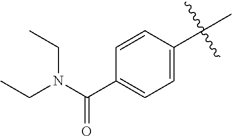 [SMILES: *c1ccc(C(=O)N(CC)CC)cc1] | 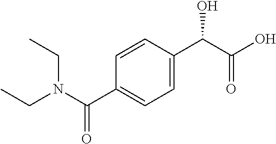 [SMILES: CCN(CC)C(=O)c1ccc([C@H](O)C(=O)O)cc1] |
| 38 | 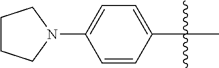 [SMILES: *c1ccc(N2CCCC2)cc1] | 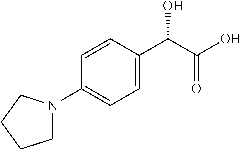 [SMILES: O=C(O)[C@@H](O)c1ccc(N2CCCC2)cc1] |
| 39 | 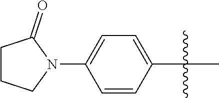 [SMILES: *c1ccc(N2CCCC2=O)cc1] | 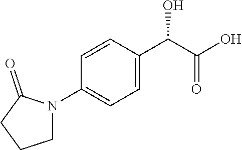 [SMILES: O=C(O)[C@@H](O)c1ccc(N2CCCC2=O)cc1] |
| 40 | 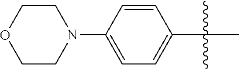 [SMILES: *c1ccc(N2CCOCC2)cc1] | 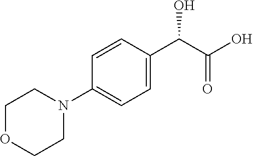 [SMILES: O=C(O)[C@@H](O)c1ccc(N2CCOCC2)cc1] |
| 41 | 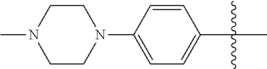 [SMILES: *c1ccc(N2CCN(C)CC2)cc1] | 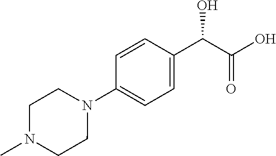 [SMILES: CN1CCN(c2ccc([C@H](O)C(=O)O)cc2)CC1] |
| 42 | 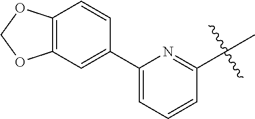 [SMILES: *c1cccc(-c2ccc3c(c2)OCO3)n1] | 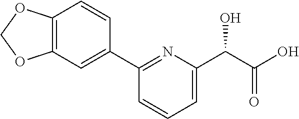 [SMILES: O=C(O)[C@@H](O)c1cccc(-c2ccc3c(c2)OCO3)n1] |
| 43 | 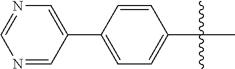 [SMILES: *c1ccc(-c2cncnc2)cc1] | 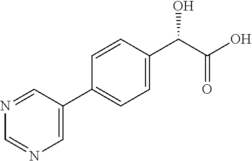 [SMILES: O=C(O)[C@@H](O)c1ccc(-c2cncnc2)cc1] |
| 44 | 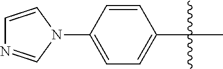 [SMILES: *c1ccc(-n2ccnc2)cc1] | 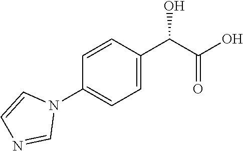 [SMILES: O=C(O)[C@@H](O)c1ccc(-n2ccnc2)cc1] |
| 45 | 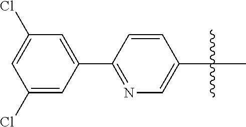 [SMILES: *c1ccc(-c2cc(Cl)cc(Cl)c2)nc1] | 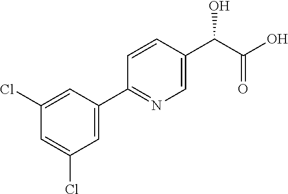 [SMILES: O=C(O)[C@@H](O)c1ccc(-c2cc(Cl)cc(Cl)c2)nc1] |
| 46 | 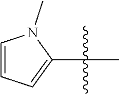 [SMILES: *c1cccn1C] | 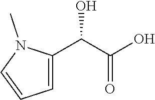 |
| 47 | 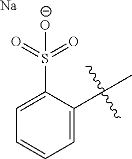 | 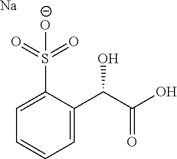 |
| 48 | 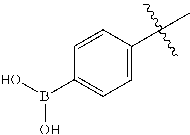 [SMILES: *c1ccc(B(O)O)cc1] | 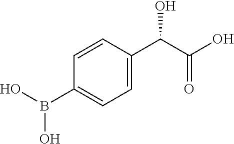 [SMILES: O=C(O)[C@@H](O)c1ccc(B(O)O)cc1] |

### Example 14c: OXS-OXC_{Me}-YciA Cascade on Semi-Preparative Scale

In a glass vial (25 mL reaction volume) 50 mM TES-KOH pH 6.8, 10 mM MgCl₂, 0.5 mM ADP (0.0125 mmol, 5.3 mg), 0.15 mM ThDP (0.00375 mmol, 1.7 mg), 0.5 mM CoA (0.0125 mmol, 9.8 mg), 25 mM disodium oxalate (0.625 mmol, 84 mg), 25 mM aldehyde (0.625 mmol), 2 μM YciA, 10 μM OXC_{Me-Y497A}, 5 μM OXS, 1.3 μM adenylate kinase and 25 units creatine kinase (Roth) were mixed and the reaction was initiated by the addition of 10 mM creatine phosphate. The same amount of creatine phosphate was added after 1, 2, 4 and 6 h to reach a final concentration of 50 mM (1.25 mmol, 409 mg). The vials were incubated without shaking at 30° C. for 24 h. Then the pH was lowered to 3 by adding HCl. The quenched reaction was saturated with NaCl and extracted with 25 mL diethylether. The organic phase was dried over MgSO₄ and filtered. After evaporation of the ether under vacuum, 50 mg (0.331 mmol, 53%) of a white solid remained, which was confirmed to be mandelic acid by UV, HPLC and NMR. ¹H NMR (300 MHz, DMSO-d₆) δ 7.4 (m, 5H); 5.05 (s, 1H). ¹³C NMR (300 MHz, DMSO-d₆) δ 174.5, 140.7, 128.6, 128.1, 127.1, 72.9.

The aldehydes 1-46 were used to prepare α-hydroxy carboxylic acid on the basis of the aforementioned procedure.

### Synthesis of α-Hydroxyacyl-CoA Thioester and α-Hydroxy Carboxylic Acids

### Example 15a—Preparation of α-Hydroxyacyl-CoA Thioester in a One Step Procedure According to the Invention—Starting from Formyl-CoA and Oxalyl-CoA (One Pot)

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 10 mM aldehyde (formaldehyde, acetaldehyde, glycolaldehyde, propionaldehyde, glyceraldehyde, glyoxylate, succinic semialdehyde, benzaldehyde, and phenylacetaldehyde) or 10 mM acetone and 5 μM OXC_{Me} or HACL_{Hs}, respectively. The reaction was started by adding 1 mM formyl-CoA or oxalyl-CoA. The reaction was stopped after 1 h by quenching with 4% formic acid and products analyzed with the CoA ester method described above.

### Example 15b—Preparation of α-Hydroxyacyl-CoA Thioesters a Two Step Procedure Starting from Oxalate by Enzymatic Catalysis (One Pot)

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 0.85 mM CoA, 10 mM ATP, 25 mM benzaldehyde, 5 μM OXC_{Me-Y497A} and 5 μM OXS were mixed and the reaction was initiated by the addition of 10 mM disodium oxalate. Samples were taken after 0, 10, 60, 240 min and quenched with 4% formic acid and products analyzed with the CoA ester method described above.

The aldehydes 1-48 according to example 14 were used to prepare α-hydroxyacyl-CoA thioesters on the basis of the aforementioned procedure.

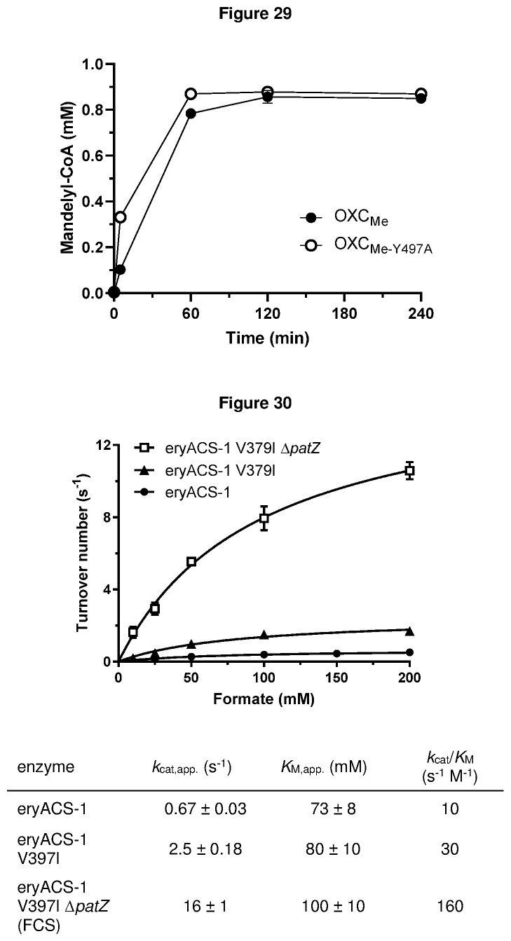

Results: The results for the formation of mandelyl-CoA are depicted in FIG. 29. CoA was converted to mandelyl-CoA. With OXC_{Me-Y497A} the reaction was complete after 60 min, with OXC_{Me} after 120 min.

### Example 16—Preparation of α-Hydroxyacyl-CoA Thioesters in a Two Step Procedure Starting from Formate by Enzymatic Catalysis (One Pot)

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 0.5 mM CoA, 10 mM ATP, 25 mM benzaldehyde, 5 μM OXC_{Me-Y497A} and 5 μM FCS were mixed and the reaction was initiated by the addition of 50 mM sodium formate. Samples were taken after 0, 10, 60, 240 min and quenched with 4% formic acid and products analyzed with the CoA ester method described above.

The aldehydes 1-46 were used to prepare α-hydroxyacyl-CoA thioesters on the basis of the aforementioned procedure.

### Example 17—Preparation of α-Hydroxy Carboxylic Acid Starting from Oxalyl-CoA by Enzymatic Catalysis in a Two Step Procedure According to the Invention (One Pot)

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 10 mM aldehyde, 5 μM OXC_{Me} or OXC_{Me-Y497A}, 2 μM YciA. The reaction was started by adding 1 mM formyl-CoA or oxalyl-CoA. The reaction was stopped after 1 h by quenching with 4% formic acid and products analyzed with the mandelic acid derivatives method described above.

### Example 18—Preparation of α-Hydroxy Carboxylic Acid Starting from Oxalate by Enzymatic Catalysis in a Three Step One Pot Procedure According to the Invention (One Pot)

Assays were carried out at 30° C. in a 1.5 mL microfuge tube. Reactions contained 15 vol % DMSO, 50 mM TES-KOH pH 6.8, 10 mM MgCl₂ 0.5 mM ADP, 0.15 mM ThDP, 0.5 mM CoA, 10 mM ATP, 25 mM aldehyde, 5 μM OXS, 5 μM OXC_{Me} or OXC_{Me-Y497A} and 2 μM YciA. The reaction was started by adding 10 mM oxalate. Samples were taken after 0, 0.05, 0.25, 0.66, 1, 2, 3, 22 hours, quenched with 4% formic acid and analyzed with the LC-MS detection of mandelic acid derivatives.

The aldehydes 1-46 were used to prepare α-hydroxy carboxylic acid on the basis of the aforementioned procedure.

### Example 19—Michaelis-Menten Kinetics of the Formation of Formyl-CoA Catalyzed by Formyl-CoA Synthetase (Acetyl-CoA Synthetase)

The Michaelis-Menten kinetic of eryACS-1 (an acyl-CoA synthetase from Erythrobacter sp.) and variants thereof as formyl-CoA synthetase (FCS) were investigated with formate as substrate according to example 6.

AMP production was followed at 30° C. by coupling eryACS-1 to purified adenylate kinase (ADK), pyruvate kinase (PK) and lactate dehydrogenase (LDH). An assay containing 200 mM MOPS-KOH pH 7.8, 0.3 mM NADH, 10 mM MgCl₂, 2 mM ATP, 0.5 mM CoA, 2.5 mM PEP, 0.2 mg/mL ADK, 15 U/mL PK from rabbit muscle (Sigma), 23 U/mL LDH from rabbit muscle (Sigma), and an appropriate concentration of eryACS-1 was preincubated for 2 min and the reaction started by adding sodium formate to a final concentration of 10, 25, 50, 100, 150, 200 mM, respectively. Reaction procedure was monitored by following the oxidation of NADH at 340 nm.

Results: The results are depicted in FIG. 30.

Acyl-CoA synthetase from Erythrobacter sp. NAP1 (eryACS-1) exhibits a favorable activity towards formate (k_{cat}=0.67±0.03 s⁻¹; K_{M}(formate)=73±8 mM). The enzyme was engineered by replacing valine 379 by isoleucine, which increased catalytic efficiency, mainly through an improved turnover number. Activity of the enzyme was further increased by expressing the gene in an E. coli strain lacking lysine acetyltransferase patZ, which inhibits acetyl-CoA synthetase, to finally obtain a highly active variant (k_{cat}=16±1 s⁻¹; K_{M}(formate)=100±10 mM).

### Example 20—Preparation of Formyl-CoA by FCS

Assays were carried out at 30° C. in a 1.5 mL microfuge tube. Reactions contained 50 mM TES-KOH pH 6.8, 10 mM MgCl₂, 0.5 mM CoA, 10 mM ATP and 0.1 μM FCS were mixed and the reaction was initiated by the addition of 50 mM sodium formate. Samples were taken after 0, 1, 5, 20 min and quenched with 4% formic acid and products analyzed with the CoA ester method described above.

### Example 21—Preparation of α-Hydroxyacyl-CoA Thioesters a Two Step Procedure Starting from Formate by Enzymatic Catalysis (One Pot)

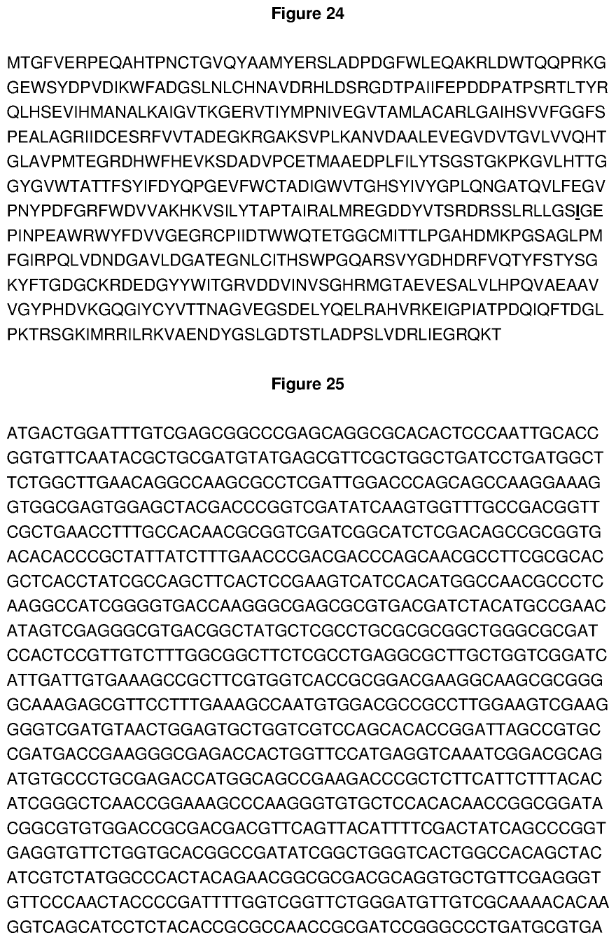

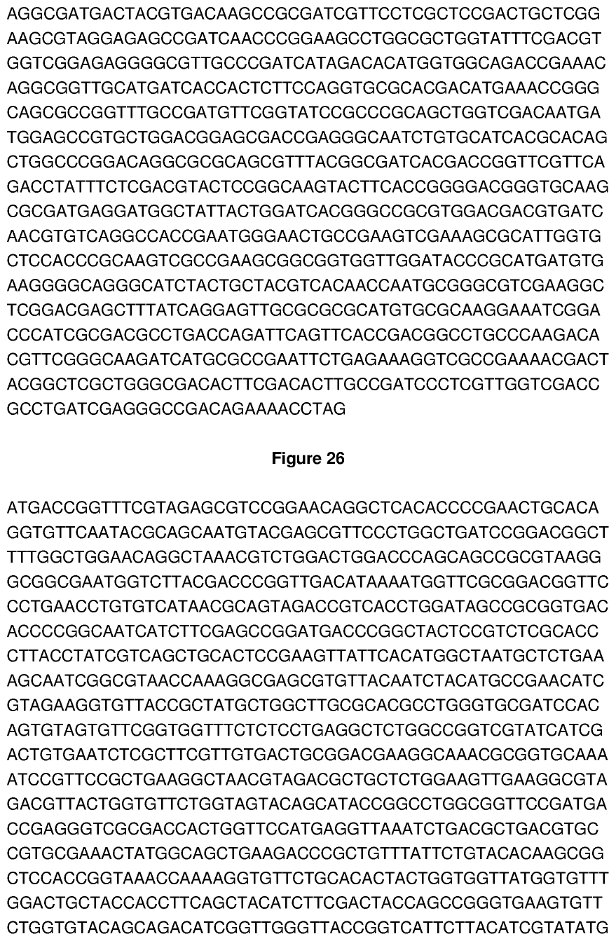

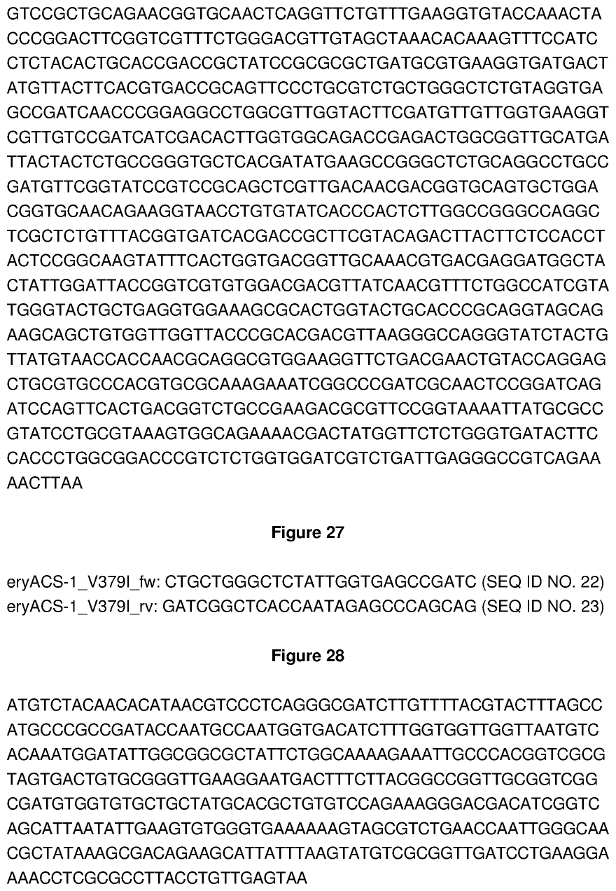

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 0.85 mM CoA, 10 mM ATP, 25 mM benzaldehyde, 5 μM OXC_{Me} and 5 μM FCS (eryACS-1—an acyl-CoA synthetase from Erythrobacter sp., FIG. 24) were mixed and the reaction was initiated by the addition of 50 mM sodium formate. Samples were taken after 0, 10, 60, 240 min and quenched with 4% formic acid and products analyzed with the CoA ester method described above.

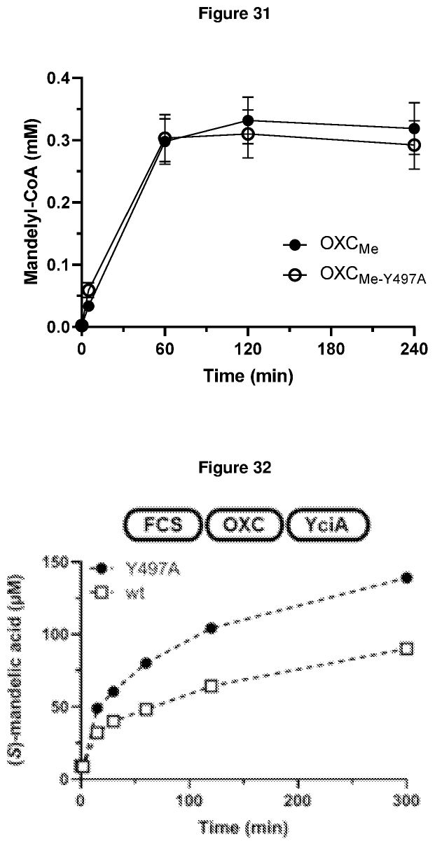

Results: The results are depicted in FIG. 31. CoA was converted to mandelyl-CoA. The reaction proceeded at the same rate for OXC_{Me} and OXC_{Me-Y497A}. 40% conversion was achieved after 240 min.

### Example 22—FCS-OXC_{Me}-YciA Cascade Prototyping

The investigation of (S)-mandelic acid formation of the cascade FCS-OXC_{Me}-YciA using formate as starting material were performed according to example 12.

Assays were carried out at 30° C. in reaction buffer consisting of 50 mM TES-KOH pH 6.8, 10 mM MgCl₂, 0.5 mM ADP, 0.15 mM ThDP, 0.5 mM CoA, 5 mM ATP, 25 mM benzaldehyde, 1 μM FCS, 2 μM OXC and 2 μM YciA. The reaction was started by adding 50 mM sodium formate. Samples were taken after 0, 2, 15, 30, 60, 120, 300 min and analyzed with the LC-MS detection of mandelic acid derivatives. Mandelic acid was quantified by comparison to commercially obtained standards of mandelic acid.

Results: The results are depicted in FIG. 32.

The formyl-CoA synthetase can be used in a cascade reaction in a one-pot system along with a thioesterase YciA and oxalyl-CoA synthetase for obtaining mandelic acid.

The aldehydes 1-46 were used to prepare α-hydroxy carboxylic acid on the basis of the aforementioned procedure.

### Example 23—OXC_{Me-Y497A}, FRC and Formyl-CoA

Formyl-CoA:oxalate CoA-transferase (FRC) from Oxalobacter formigenes (EC 2.8.3.16) catalyzes the reaction formyl-CoA+oxalate=oxalyl-CoA+formate. This reaction is fully reversible. Converting formyl-CoA into oxalyl-CoA in the context of the cascade should have 2 beneficial effects: First, YciA has (low) thioesterase activity towards formyl-CoA, but none towards oxalyl-CoA (FIG. 16). This means that no (or less) ATP equivalents are lost to formyl-CoA hydrolysis, resulting in higher yield. Secondly, OXC_{Me-Y497A} is faster in the carboligation with oxalyl-CoA than with formyl-CoA (FIG. 14). Therefore, turning formyl-CoA into oxalyl-CoA should increase the rate of OXC, and thus the rate of the whole cascade. In order to test these hypothesis FRC was added to a reaction containing OXC, benzaldehyde, oxalate and oxalyl-CoA.

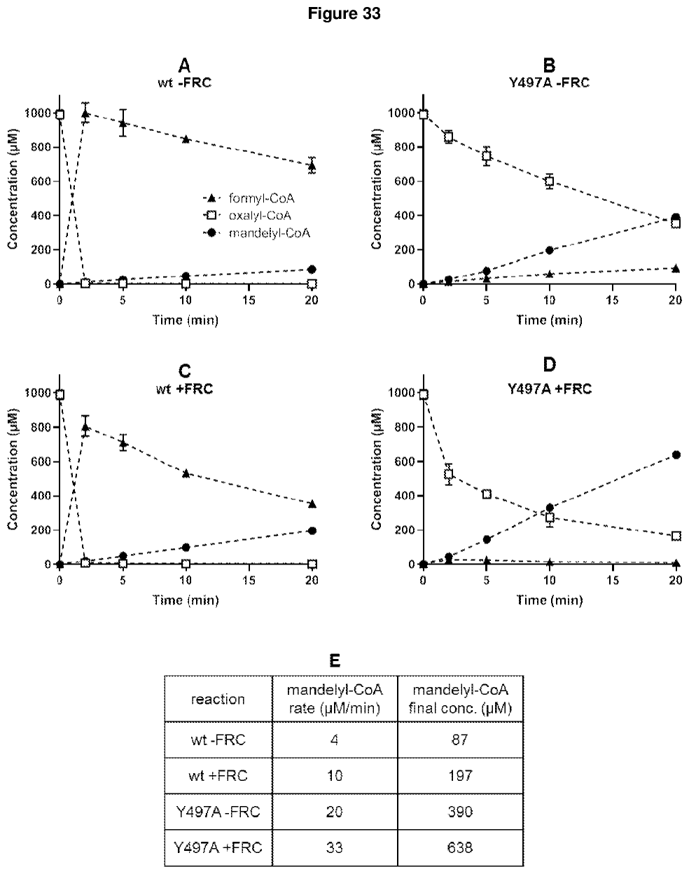

Results: The results are depicted in FIG. 33. OXC_{Me-Y497A} with FRC gave mandelyl-CoA formation rate of 33 μM/min, a 1.65-fold improvement over no FRC. Also note how the formyl-CoA is not accumulating in the reaction with OXC_{Me-Y497A}, whereas it is still formed rapidly in the case of OXC_{Me}. This can be explained by the k_{cat} for decarboxylation (OXC_{Me}, 98 s⁻¹ vs. OXC_{Me-Y497A}, 1.3 s⁻¹).

### Example 24—Preparation of Oxalyl-CoA by FRC

Assays were carried out at 30° C. in a 1.5 mL microfuge tube. Reactions contained 50 mM TES-KOH pH 6.8, 10 mM MgCl₂, 10 mM disodium oxalate and 0.1 μM FRC were mixed and the reaction was initiated by the addition of 1 mM formyl-CoA. Samples were taken after 0, 1, 5, 20 min and quenched with 4% formic acid and products analyzed with the CoA ester method described above.

### Example 25—Preparation of Formyl-CoA by FRC

Assays were carried out at 30° C. in a 1.5 mL microfuge tube. Reactions contained 50 mM TES-KOH pH 6.8, 10 mM MgCl₂, 10 mM sodium formate and 0.1 μM FRC were mixed and the reaction was initiated by the addition of 1 mM oxalyl-CoA. Samples were taken after 0, 1, 5, 20 min and quenched with 4% formic acid and products analyzed with the CoA ester method described above.

### Example 26—Preparation of α-Hydroxy Carboxylic Acid Starting from an Oxalate Using OXS-OXC_{Me}-YciA with FRC (One Pot)

In a 1.5 mL microfuge tube, reaction buffer containing 50 mM TES-KOH pH 6.8, 10 mM MgCl₂. 0.5 mM ADP, 0.15 mM ThDP, 0.5 mM CoA, 10 mM ATP, 25 mM benzaldehyde, 0.5 μM FRC, 2 μM YciA, 5 μM OXC_{Me} and 5 μM OXS were mixed and the reaction was initiated by the addition of 10 mM disodium oxalate. For the negative control FRC was omitted in a separate reaction. Samples were taken after 0, 3, 15, 40 min, 1, 2, 3 and 22 h and analyzed with the mandelic acid derivatives method described above. For quantification commercial, racemic mandelic acid was diluted in reaction buffer to appropriate concentrations to obtain a calibration curve.

The aldehydes 1-46 were used to prepare α-hydroxy carboxylic acid on the basis of the aforementioned procedure.

### Example 27—Preparation of α-Hydroxy Carboxylic Acid Starting from an Oxalate Using OXS-OXC_{Me}-YciA with FRC (One Pot)

In a 1.5 mL microfuge tube, reaction buffer containing 50 mM TES-KOH pH 6.8, 10 mM MgCl₂. 0.5 mM ADP, 0.15 mM ThDP, 0.5 mM CoA, 10 mM ATP, 7.6 mM benzaldehyde or phenylacetaldehyde, 2 μM FRC (Formyl-CoA:oxalate CoA-transferase from Oxalobacter formigenes EC 2.8.3.16), 2 μM YciA, 10 μM OXC_{Me-Y497A} and 5 μM OXS were mixed and the reaction was initiated by the addition of 10 mM disodium oxalate. For the negative control FRC was omitted in a separate reaction. Samples were taken after 0, 15, 30, 60 min and analyzed with the mandelic acid derivatives method described above.

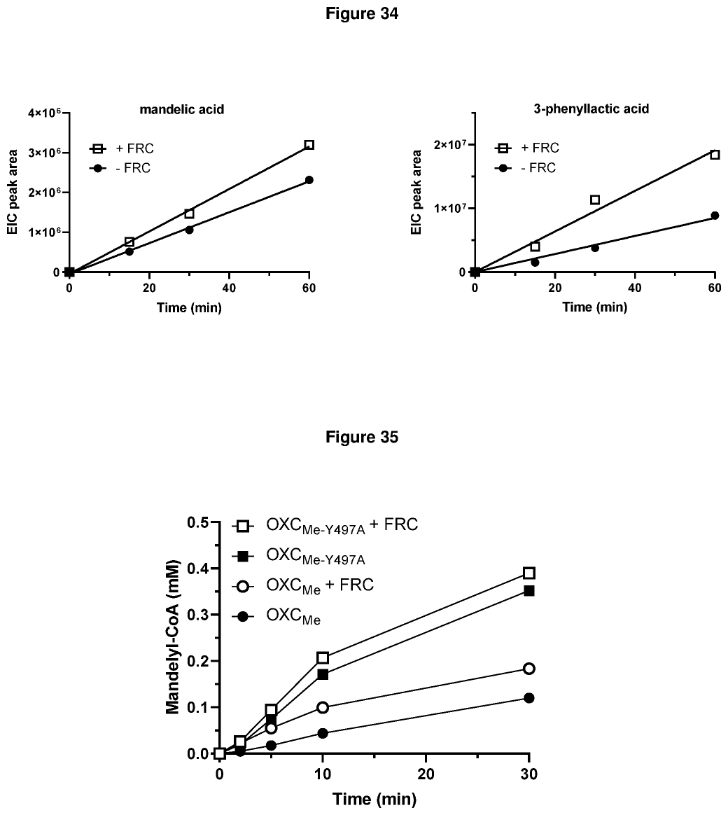

Results: The results are depicted in FIG. 34. Addition of FRC increased product formation rate 1.4-fold and 2.3-fold for mandelic acid and 3-phenyllactic acid, respectively.

### Example 28—Preparation of α-Hydroxy Carboxylic Acid Starting from an Oxalate Using OXS-OXC_{Me}-HACOT without a Hydrolase (One Pot)

In a 1.5 mL microfuge tube, reaction buffer containing 50 mM TES-KOH pH 6.8, 10 mM MgCl₂. 0.5 mM ADP, 0.15 mM ThDP, 0.5 mM CoA, 0.25 mM ATP, 25 mM benzaldehyde, 10 μM HACOT, 5 μM OXC_{Me} and 0.1 μM OXS were mixed and the reaction was initiated by the addition of 10 mM disodium oxalate. For the negative controls FRC was omitted in a separate reaction. Samples were taken after 0, 3, 15, 40 min, 1, 2, 3 and 22 h and analyzed with the mandelic acid derivatives method described above. For quantification commercial, racemic mandelic acid was diluted in reaction buffer to appropriate concentrations to obtain a calibration curve.

The aldehydes 1-46 were used to prepare α-hydroxy carboxylic acid on the basis of the aforementioned procedure.

### Example 29—OXS-OXC_{Me} with Formyl-CoA Proofreading by FRC (One Pot)

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 0.85 mM CoA, 10 mM ATP, 25 mM benzaldehyde, 1 μM OXC_{Me}, 5 μM OXS and 20 μM FRC and were mixed and the reaction was initiated by the addition of 10 mM disodium oxalate. Samples were taken after 0, 2, 5, 10, 30 min and quenched with 4% formic acid and products analyzed with the CoA ester method described above.

Results: The results are depicted in FIG. 35. CoA was converted to mandelyl-CoA. Addition of FRC increased the reaction rate, indicating that formyl-CoA proofreading has a beneficial effect. The effect is greater with the wild-type OXC_{Me}, which is expected from the higher formyl-CoA release rate compared to OXC_{MeY497A}.

### Example 30—Preparation of α-Hydroxy Carboxylic Acid Starting from Oxalyl-CoA Using OXC_{Me}-YciA-FRC

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 25 mM benzaldehyde, 10 mM sodium oxalate, 10 μM OXC_{Me-Y497A}, 2 μM YciA and 2 μM FRC and were mixed and the reaction was initiated by the addition of 5 mM oxalyl-CoA. Samples were taken after 0, 15, 30, 60 min and quenched with 4% formic acid and products analyzed with the mandelic acid derivatives method described above.

### Example 31—Preparation of α-Hydroxy Carboxylic Acid Starting from Oxalyl-CoA Using OXC_{Me}-HACOT without a Hydrolase

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 25 mM benzaldehyde, 10 mM sodium oxalate, 10 μM OXC_{Me-Y497A}, 10 μM HACOT and were mixed and the reaction was initiated by the addition of 5 mM oxalyl-CoA. Samples were taken after 0, 15, 30, 60 min and quenched with 4% formic acid and products analyzed with the mandelic acid derivatives method described above.

### Example 32—Preparation of α-Hydroxy Carboxylic Acid Starting from Formyl-CoA Using OXC_{Me}-YciA

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 25 mM benzaldehyde, 10 μM OXC_{Me-Y497A}, 2 μM YciA and were mixed and the reaction was initiated by the addition of 5 mM formyl-CoA. Samples were taken after 0, 15, 30, 60 min and quenched with 4% formic acid and products analyzed with the mandelic acid derivatives method described above.

### Example 33—Preparation of α-Hydroxy Carboxylic Acid Starting from Formate Using FCS-OXC_{Me}-HAFT (α-Hydroxyacyl-CoA:Formate Transferase) without a Hydrolase

Assays were carried out at 30° C. in a 1.5 mL microfuge tube. Reactions contained 50 mM TES-KOH pH 6.8, 10 mM MgCl₂, 0.5 mM ADP, 0.15 mM ThDP, 0.5 mM CoA, 0.25 mM ATP, 25 mM benzaldehyde, 0.1 μM FCS, 10 μM OXC_{Me-Y497A}, 10 μM HAFT and were mixed and the reaction was initiated by the addition of 50 mM sodium formate. Samples were taken after 0, 30, 60, 120 min and quenched with 4% formic acid and products analyzed with the mandelic acid derivatives method described above.

What is claimed is:

1. A method for preparation of a compound of the formula (I′) 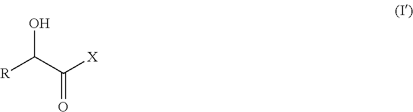
wherein R represents 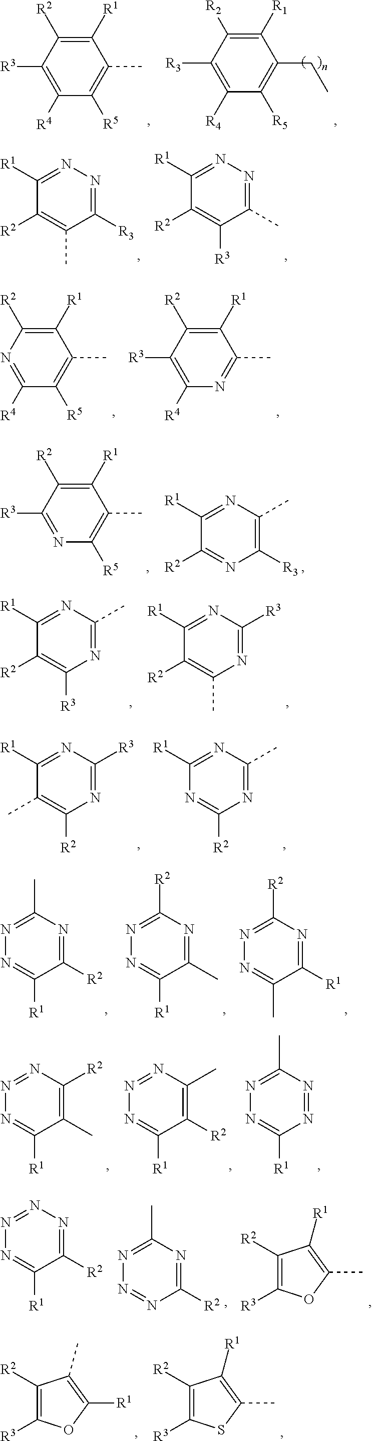 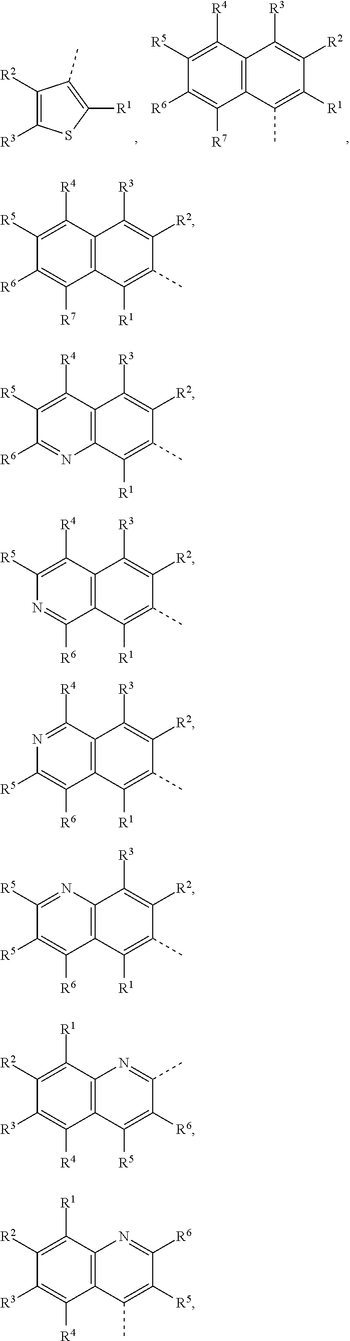 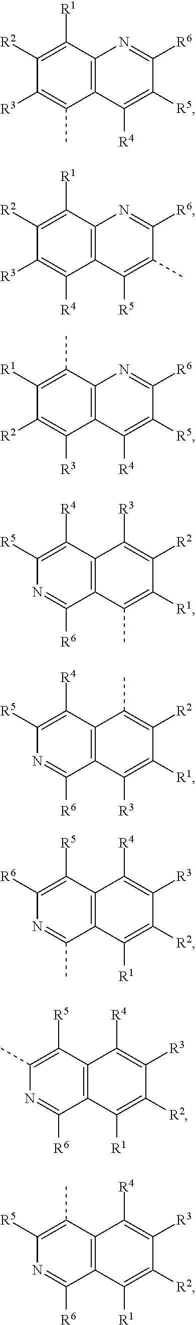 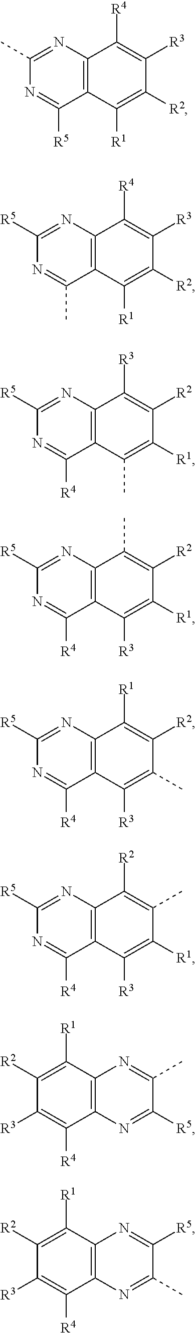 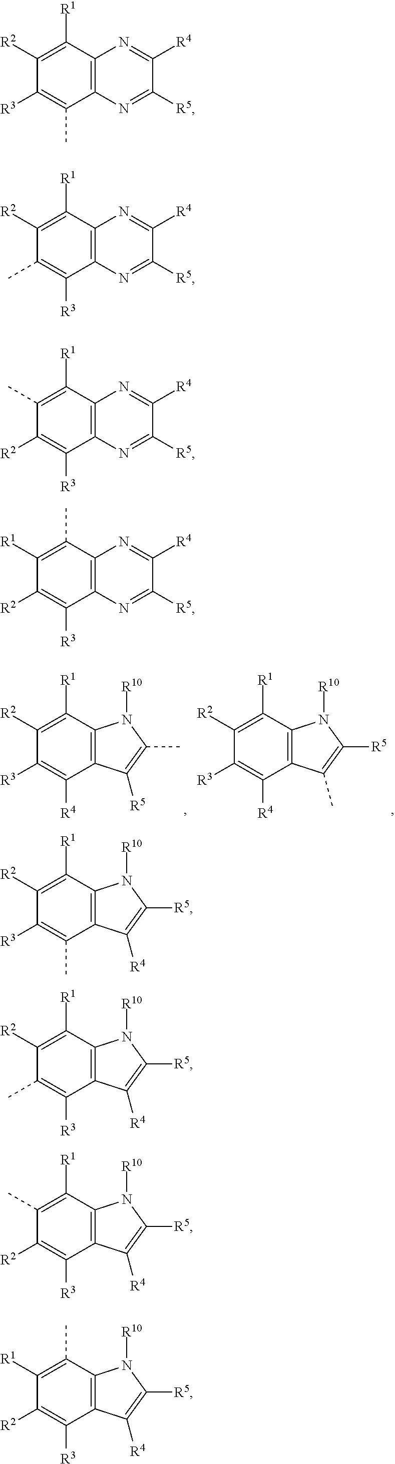 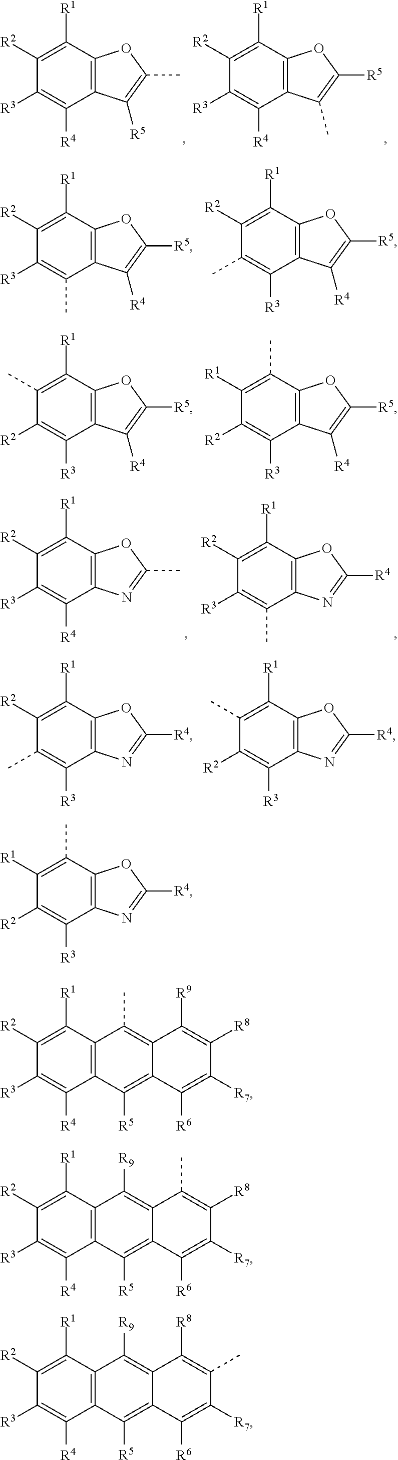 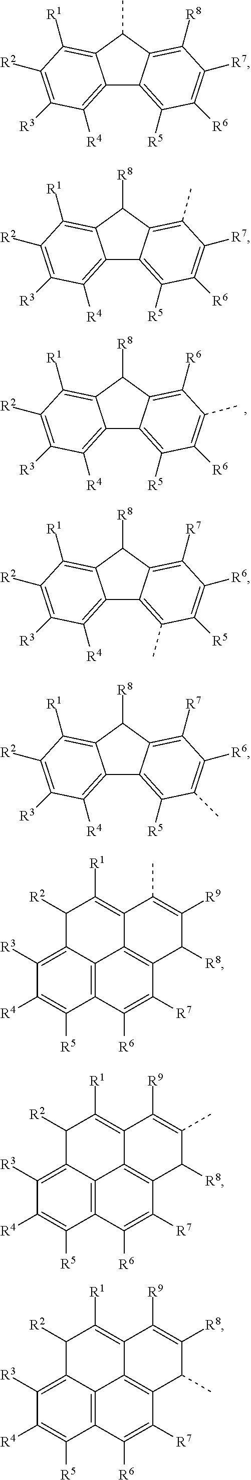 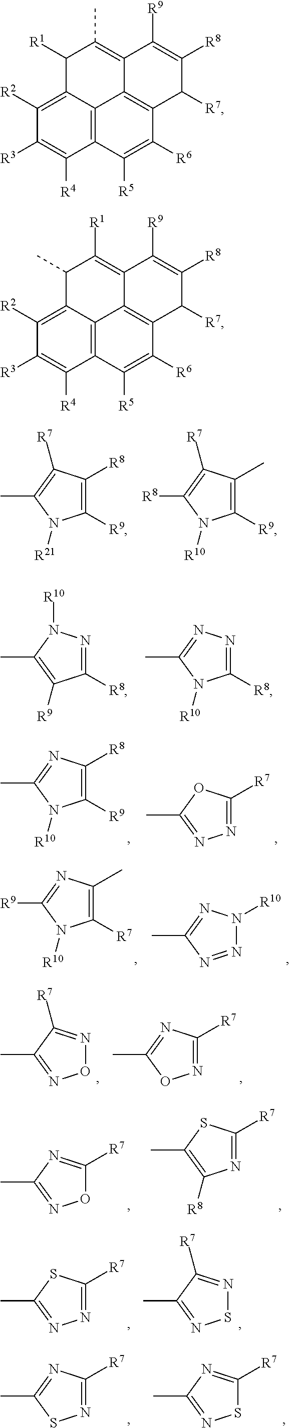 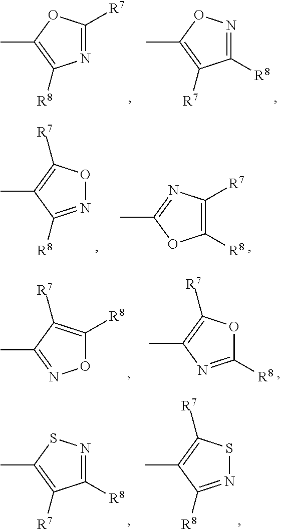 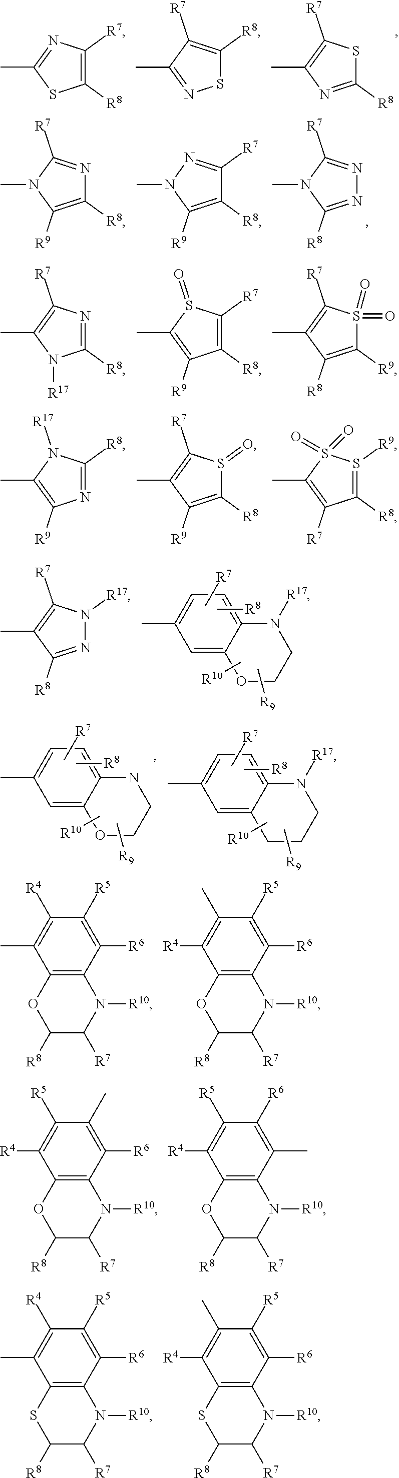 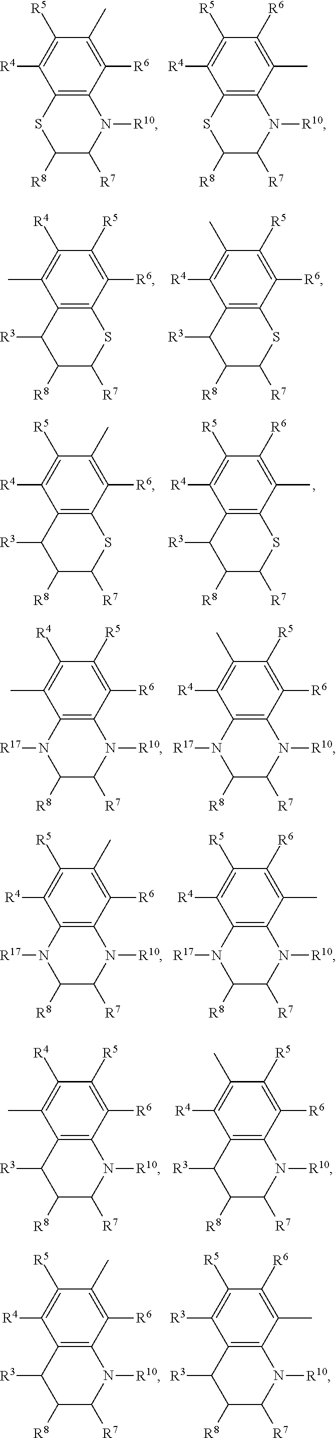 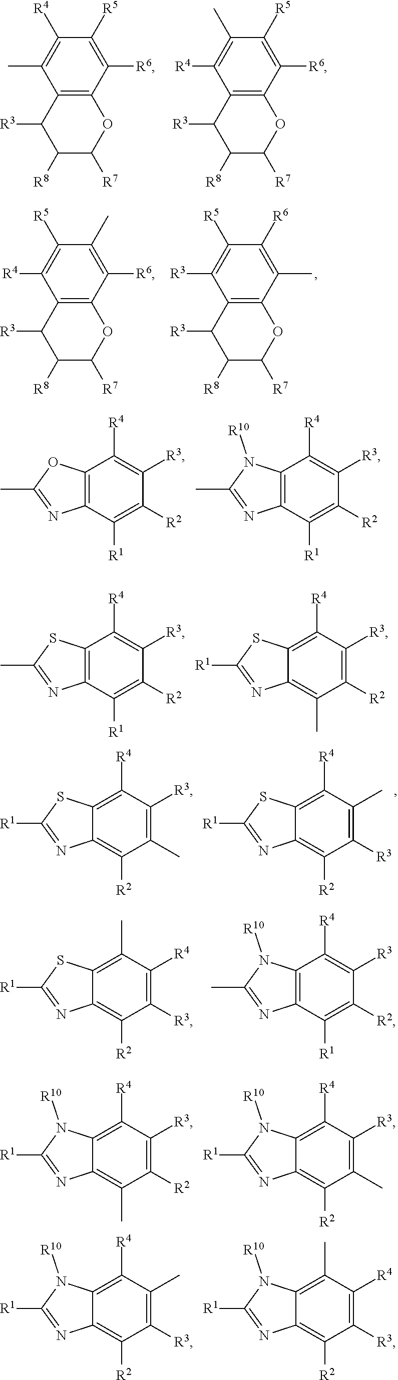 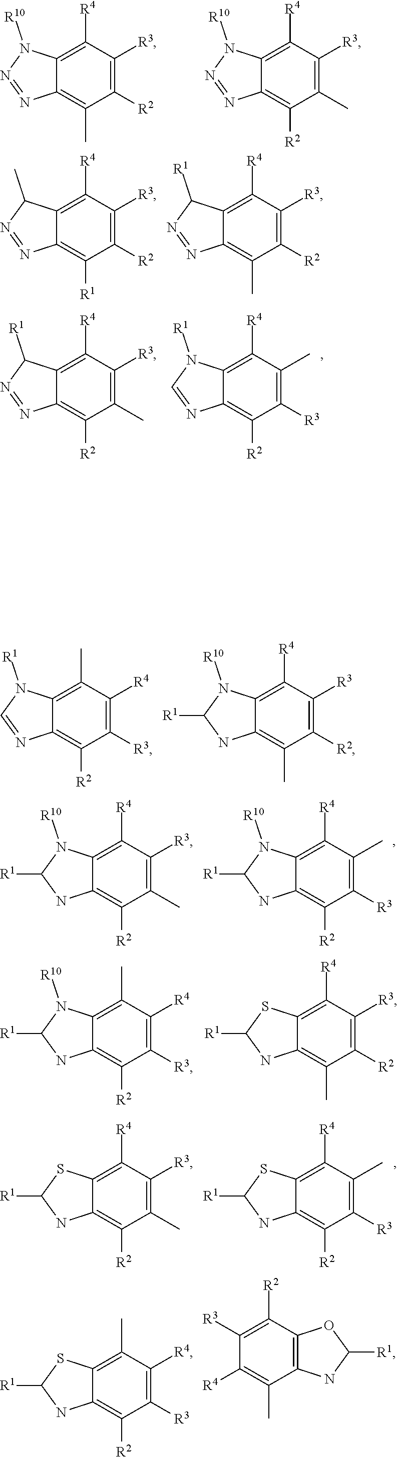 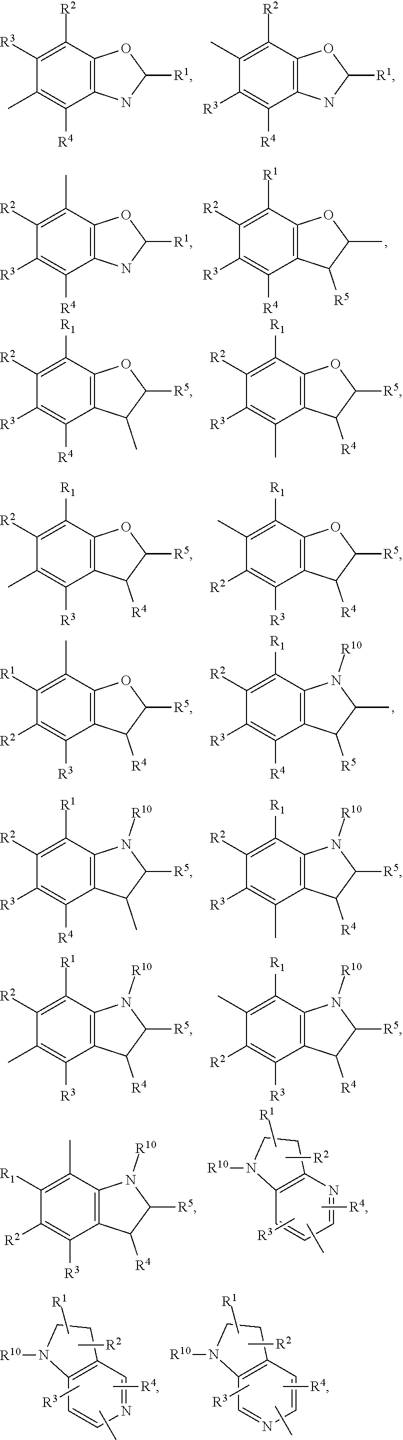 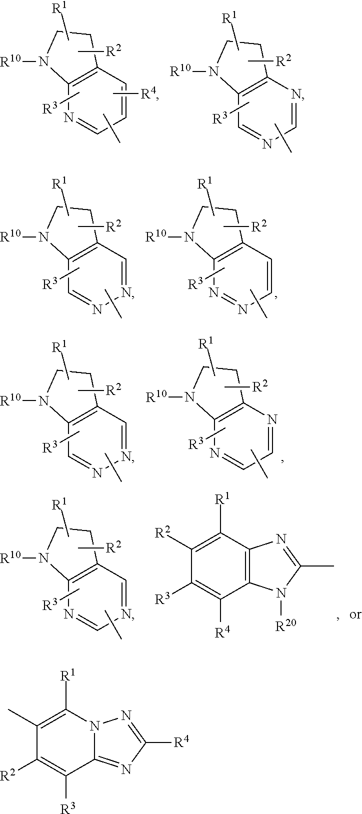
wherein R¹, R², R³, R⁴, R⁵, R⁶, R⁷, R⁸, and R⁹ represent independently of each other —H, —F, —Cl, —Br, —I, -Ph, —OH, —OCH₃, —OC₂H₅, —OC₃H₇, —OCH(CH₃)₂, —OC₄H₉, —OCH(CH₃)CH₂CH₃, —OCH₂CH(CH₃)₂, —OC(CH₃)₃, —O-cyclo-C₃H₅, —O-cyclo-C₄H₇, —O-cyclo-C₅H₉, —O-cyclo-C₆H₁₁, —OPh, —OCH₂-Ph, —OCH₂CH₂-Ph —OCH═CH₂, —OCH₂—CH═CH₂, —OCH₂CH₂—CH═CH₂, —OCF₃, —OC₂F₅, —SCH₃, —SC₂H₅, —SC₃H₇, —SCH(CH₃)₂, —SC₄H₉, —SCH(CH₃)CH₂CH₃, —SCH₂CH(CH₃)₂, —SC(CH₃)₃, —NO₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(C₄H₉)₂, —N(cyclo-C₃H₅)₂, —N[CH(CH₃)₂]₂, —N[C(CH₃)₃]₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH₂—CH₂—CH(CH₃)₂, —CH(CH₃)—CH(CH₃)—CH₃, —C(CH₃)₂—C₂H₅, —C₆H₁₃, —CH(CH₃)—C₄H₉, —CH₂—CH(CH₃)—C₃H₇, —CH₂—CH₂—CH₂—CH(CH₃)₂, —CH(CH₃)—CH(CH₃)—C₂H₅, —C(CH₃)₂—C₃H₇, —C₇H₁₅, —CH(CH₃)—C₅H₁₁, —CH₂—CH(CH₃)—C₄H₉, —CH₂—CH₂—CH₂—CH₂—CH(CH₃)₂, —C₈H₁₇, —C₉H₁₉, —C₁₀H₂₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —CH₂F, —CF₂I, —CHF₂, —CF₃, —CH₂I, —CH₂Br, —CH₂I, —CH₂—CH₂F, —CH₂—CHF₂, —CH₂—CF₃, —CH₂—CH₂I, —CH₂—CH₂Br, —CH₂—CH₂I, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COO-cyclo-C₄H₇, —COO-cyclo-C₅H₉, —COO-cyclo-C₆H₁₁, —COOCH(CH₃)₂, —COOC(CH₃)₃, —O—COOCH₃, —O—COOC₂H₅, —O—COOC₃H₇, —O—COO-cyclo-C₃H₅, —O—COOCH(CH₃)₂, —O—COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃Na, —SO₃K, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —B(OH)₂, —B(OR²²), 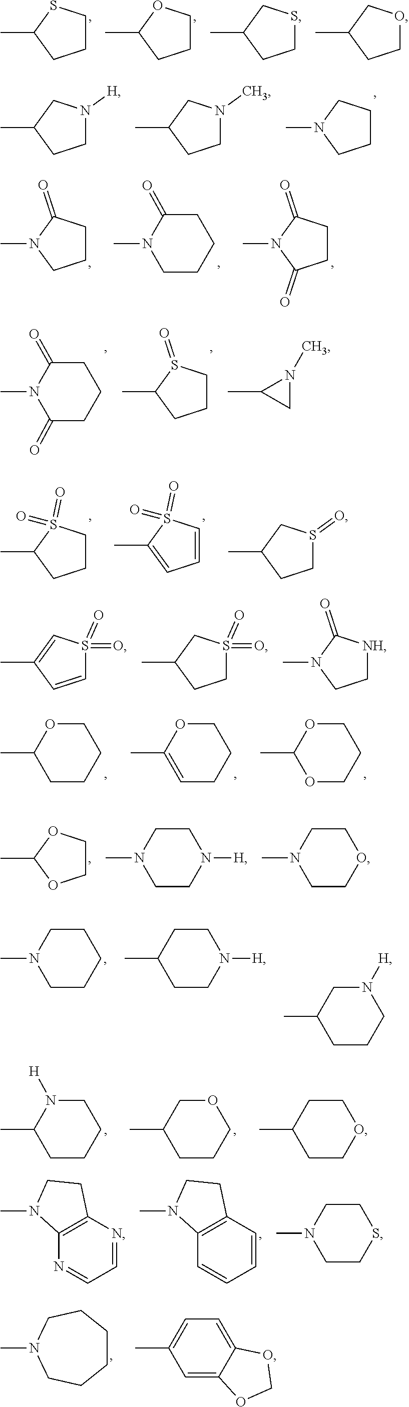 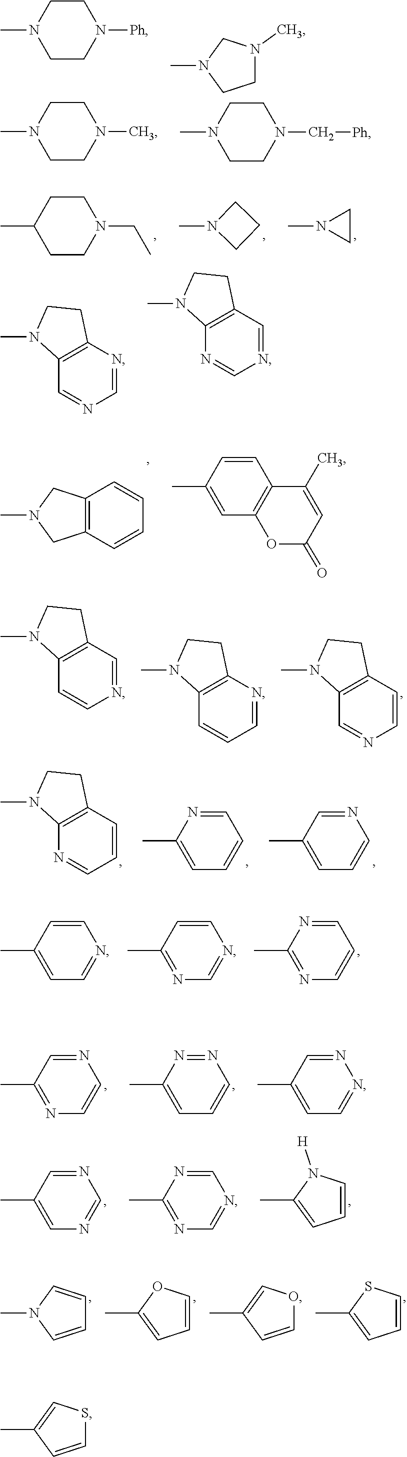 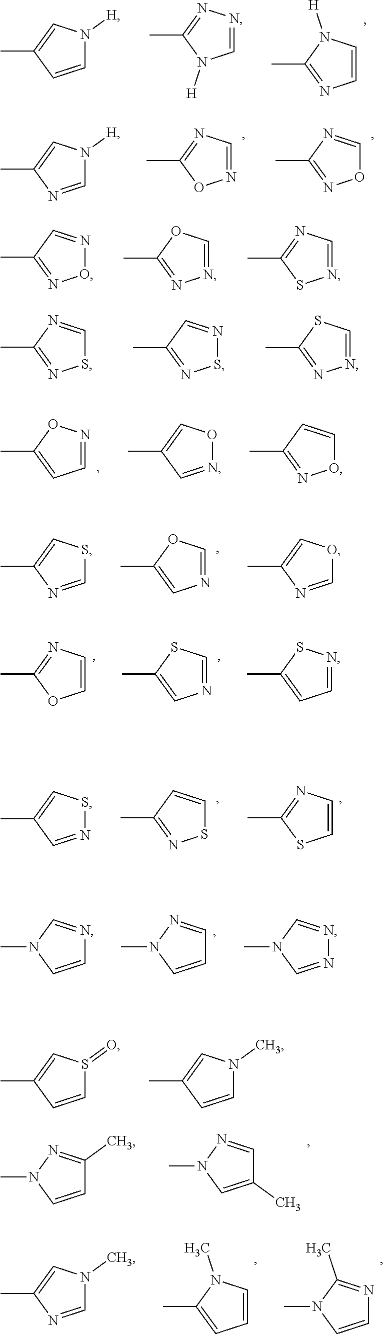 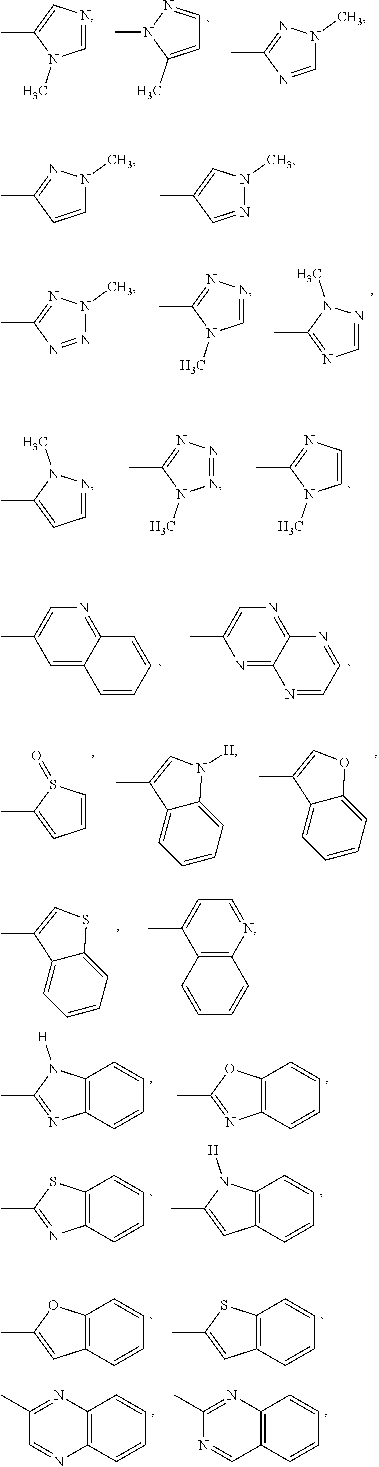 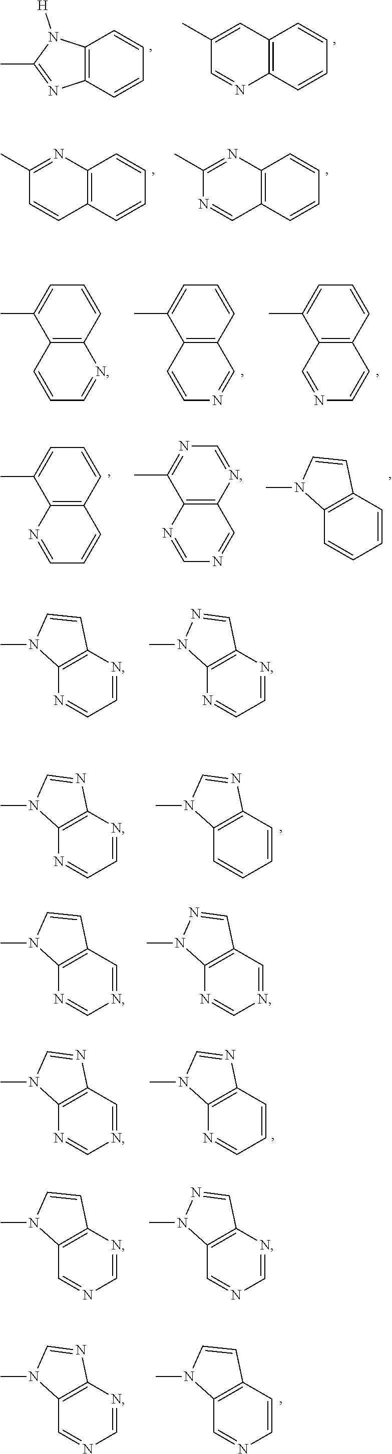 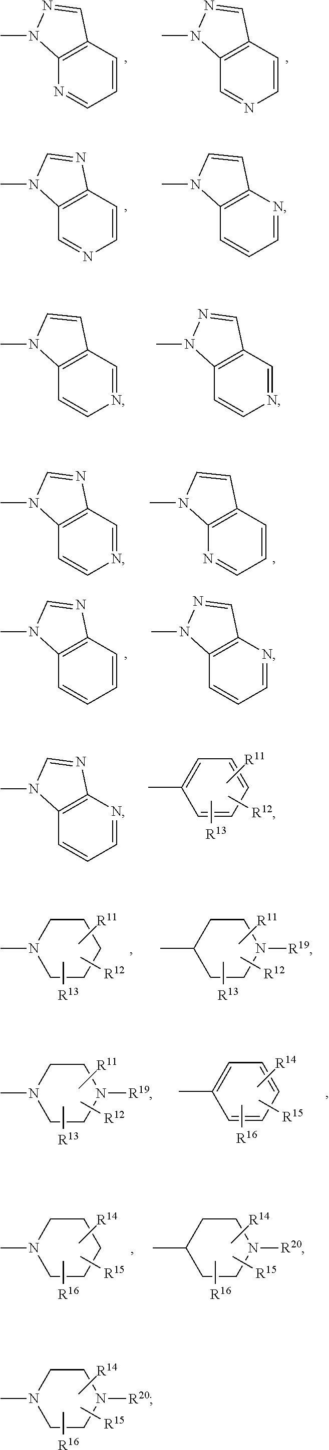
R¹¹ to R¹⁶ represents independently of each other:
—H, —NH₂, —OH, —OCH₃, —OC₂H₅, —OC₃H₇, —OCF₃, —CF₃, —F, —Cl, —Br, —I, —CH₃, —C₂H₅, —C₃H₇, —CH(CH₃)₂, -Ph, or —CN;
R¹⁰, R¹⁷, R¹⁸, R¹⁹, R²⁰, and R²¹ represents independently of each other —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, CH₂—CH₂F, —CH₂—CHF₂, —CH₂—CF₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, -cyclo-C₃H₅, -cyclo-C₄H₇, -cyclo-C₅H₉, -cyclo-C₆H₁₁, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, preferably R²¹ cannot be —H;
R²² represents —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, CH₂—CH₂F, —CH₂—CHF₂, —CH₂—CF₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, -cyclo-C₃H₅,
X represents —OH, —O⁻ or —SCoA,
wherein n is an integer 1, 2, or 3; or a salt thereof, comprising:
A) providing a compound R—CHO (1) and an acyl-CoA (2) 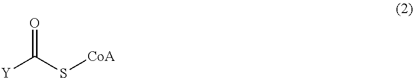
wherein Y represents —H or —COO⁻,
in a buffer solution with a pH value in the range of 5.0 to 7.5 optionally together with at least one co-solvent;
B) performing a coupling reaction of R—CHO (1) and acyl-CoA (2) by an oxalyl-CoA decarboxylase (3a) in the presence of thiamine diphosphate (3b) and a magnesium (II) cation to obtain the α-hydroxyacyl-CoA thioester (I′) 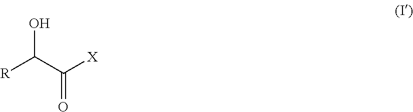
wherein X represents —S—CoA,
and the oxalyl-CoA decarboxylase (3a) is a peptide encoded by a gene selected from Methylobacterium extorquens or a mutant thereof; and optionally comprising the additional step C)
C) cleaving the coenzyme A moiety of said α-hydroxyacyl-CoA thioester (I′) by a hydrolase, α-hydroxyacyl-CoA:oxalate CoA-transferase and/or α-hydroxyacyl-CoA:formate CoA-transferase to obtain the compound of the formula (I′) or a salt thereof 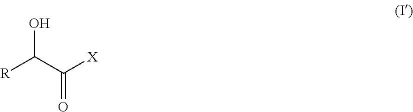
wherein X represents —OH or O⁻,
and the hydrolase is acyl-CoA thioester hydrolase YciA (4) encoded by a gene selected from E. coli.

2. The method for the preparation of the compounds according to claim 1 represented by formula (I) 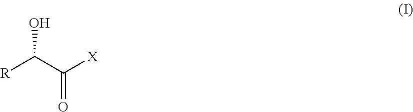
wherein in step B) a (S)-α-hydroxyacyl-CoA thioester (I) 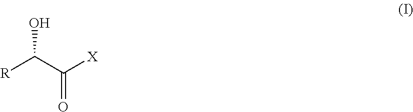
wherein X represents —S—CoA is obtained, or in step C)
the compound of the formula (I) or a salt thereof 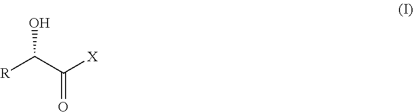
wherein X represents —OH or O⁻ is obtained.

3. The method for preparation of the compounds according to claim 2, wherein the acyl-CoA (2) in step A) and step B) has the following structure 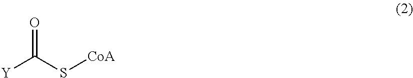
wherein Y represents —COO⁻;
and wherein in step C) the coenzyme A moiety is cleaved by a hydrolase, α-hydroxyacyl-CoA:oxalate CoA-transferase and/or α-hydroxyacyl-CoA:formate CoA-transferase.

4. The method for the preparation of the compounds according to claim 2 represented by formula (II)

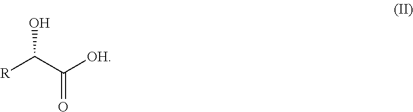

5. The method according to claim 4, wherein steps A), B) and C) are carried out as cascade reaction in one-pot system.

6. The method according to claim 3, further comprising preparing the oxalyl-CoA (2′) by the following step A′)
A′) reacting an oxalate (C₂O₄²⁻) (2a′) with a free coenzyme A (2b) by oxalyl-CoA synthetase (2c′) in the presence of ATP (2e) to produce an oxalyl-CoA (2′).

7. The method according to claim 6, wherein steps A′), A), B), and C) are carried out as cascade reaction in one-pot system.

8. The method according to claim 7, wherein in step A′) a ratio of the oxalate (2a′) and the free coenzyme A (2b) is 10 to 10,000.

9. The method according to claim 1, wherein in step B), a concentration of the oxalyl-CoA decarboxylase is in the range of 1 to 25 μM and/or a concentration of the magnesium (II) cation is in the range of 2.5 mM to 20 mM.

10. The method according to claim 1, wherein in step B), the mutant comprises OXC_{Me-Y497A}, OXC_{Me-S568A}, or OXC_{Me-Y497A-S568A}.

11. The method according to claim 6, wherein in step A′) the oxalate is selected from the group consisting of (NH₄⁺)₂C₂O₄, Li₂C₂O₄, Na₂C₂O₄, K₂C₂O₄, Rb₂C₂O₄, Cs₂C₂O₄, MgC₂O₄, CaC₂O₄, ZnC₂O₄, FeC₂O₄, MnC₂O₄, NiC₂O₄, CuC₂O₄, and CoC₂O₄.

12. The method according to claim 1, wherein
R represents 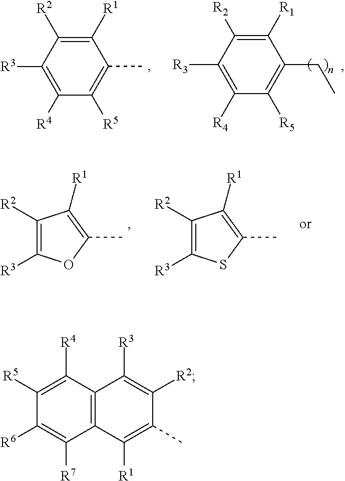
and
R¹, R², R³, R⁴, R⁵, R⁶ and R⁷ represents independently of each other —H, —Cl, —OH, —OCH₃, —NO₂, —N(CH₃)₂, —C≡CH, —CHO, —COCH₃ or —COOH, wherein n represents an integer 1.

13. A peptide OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4.

14. A composition comprising a peptide OXC_{Me} having the sequence SEQ ID NO. 1, OXC_{Me-Y497A} having the sequence SEQ ID NO. 2, OXC_{Me-S568A} having the sequence SEQ ID NO. 3 or OXC_{Me-Y497A-S568A} having the sequence SEQ ID NO. 4 and a thioesterase YciA encoded by a gene selected from E. coli.

15. The composition according to claim 14 further comprising an oxalyl-CoA synthetase.


SEQUENCE LISTING (25 sequences)

SEQ ID NO: 1 - protein, 583 aa, Methylobacterium extorquens
MTVQAQNIDAITAGAMPHEEPELTDGFHLVIDALKLNGIETIYNVPGIPITDLGRLAQAEGLRVISFRHEQNAGNAAAIAGFLTKKPGICLTVSAPGFLNGLTALANATTNCFPMILISGSSEREIVDLQQGDYEEMDQLAIAKPLCKAAFRVLHAADIGIGVARAIRAAVSGRPGGVYLDLPAKLFSQVIDADLGARSLVKVIDAAPAQLPAPAAIARALDVLKSAERPLIILGKGAAYAQADEAVRALVEESGIPYVPMSMAKGLLPDTHPLSAGAARSTALKDSDVVLLVGARLNWLLSHGKGKTWGEPGSKRFIQIDIEPREMDSNVEIVAPVVGDIGSCVEALLDGIRKDWKGAPSNWLETLRGKREANIAKMAPKLMKNSSPMCFHSALGALRTVIKERPDAILVNEGANTLDLARGIIDMYQPRKRLDVGTWGVMGIGMGFAVAAAVETGKPVLAVEGDSAFGFSGMEVETICRYELPVCIVIFNNNGIYRGTDTDPTGRDPGTTVFVKNSRYDKMMEAFGGVGVNVTTPDELKRAVDEAMNSGKPTLINAEIDPAAGSESGNIGSLNPQSTLKKK

SEQ ID NO: 2 - protein, 583 aa, Artificial
  Mutant of oxalyl-CoA decarboxylase from Methylobacterium extorquens
MTVQAQNIDAITAGAMPHEEPELTDGFHLVIDALKLNGIETIYNVPGIPITDLGRLAQAEGLRVISFRHEQNAGNAAAIAGFLTKKPGICLTVSAPGFLNGLTALANATTNCFPMILISGSSEREIVDLQQGDYEEMDQLAIAKPLCKAAFRVLHAADIGIGVARAIRAAVSGRPGGVYLDLPAKLFSQVIDADLGARSLVKVIDAAPAQLPAPAAIARALDVLKSAERPLIILGKGAAYAQADEAVRALVEESGIPYVPMSMAKGLLPDTHPLSAGAARSTALKDSDVVLLVGARLNWLLSHGKGKTWGEPGSKRFIQIDIEPREMDSNVEIVAPVVGDIGSCVEALLDGIRKDWKGAPSNWLETLRGKREANIAKMAPKLMKNSSPMCFHSALGALRTVIKERPDAILVNEGANTLDLARGIIDMYQPRKRLDVGTWGVMGIGMGFAVAAAVETGKPVLAVEGDSAFGFSGMEVETICRYELPVCIVIFNNNGIARGTDTDPTGRDPGTTVFVKNSRYDKMMEAFGGVGVNVTTPDELKRAVDEAMNSGKPTLINAEIDPAAGSESGNIGSLNPQSTLKKK

SEQ ID NO: 3 - protein, 583 aa, Artificial
  Mutant of oxalyl-CoA decarboxylase from Methylobacterium extorquens
MTVQAQNIDAITAGAMPHEEPELTDGFHLVIDALKLNGIETIYNVPGIPITDLGRLAQAEGLRVISFRHEQNAGNAAAIAGFLTKKPGICLTVSAPGFLNGLTALANATTNCFPMILISGSSEREIVDLQQGDYEEMDQLAIAKPLCKAAFRVLHAADIGIGVARAIRAAVSGRPGGVYLDLPAKLFSQVIDADLGARSLVKVIDAAPAQLPAPAAIARALDVLKSAERPLIILGKGAAYAQADEAVRALVEESGIPYVPMSMAKGLLPDTHPLSAGAARSTALKDSDVVLLVGARLNWLLSHGKGKTWGEPGSKRFIQIDIEPREMDSNVEIVAPVVGDIGSCVEALLDGIRKDWKGAPSNWLETLRGKREANIAKMAPKLMKNSSPMCFHSALGALRTVIKERPDAILVNEGANTLDLARGIIDMYQPRKRLDVGTWGVMGIGMGFAVAAAVETGKPVLAVEGDSAFGFSGMEVETICRYELPVCIVIFNNNGIYRGTDTDPTGRDPGTTVFVKNSRYDKMMEAFGGVGVNVTTPDELKRAVDEAMNSGKPTLINAEIDPAAGSEAGNIGSLNPQSTLKKK

SEQ ID NO: 4 - protein, 583 aa, Artificial
  Mutant of oxalyl-CoA decarboxylase from Methylobacterium extorquens
MTVQAQNIDAITAGAMPHEEPELTDGFHLVIDALKLNGIETIYNVPGIPITDLGRLAQAEGLRVISFRHEQNAGNAAAIAGFLTKKPGICLTVSAPGFLNGLTALANATTNCFPMILISGSSEREIVDLQQGDYEEMDQLAIAKPLCKAAFRVLHAADIGIGVARAIRAAVSGRPGGVYLDLPAKLFSQVIDADLGARSLVKVIDAAPAQLPAPAAIARALDVLKSAERPLIILGKGAAYAQADEAVRALVEESGIPYVPMSMAKGLLPDTHPLSAGAARSTALKDSDVVLLVGARLNWLLSHGKGKTWGEPGSKRFIQIDIEPREMDSNVEIVAPVVGDIGSCVEALLDGIRKDWKGAPSNWLETLRGKREANIAKMAPKLMKNSSPMCFHSALGALRTVIKERPDAILVNEGANTLDLARGIIDMYQPRKRLDVGTWGVMGIGMGFAVAAAVETGKPVLAVEGDSAFGFSGMEVETICRYELPVCIVIFNNNGIARGTDTDPTGRDPGTTVFVKNSRYDKMMEAFGGVGVNVTTPDELKRAVDEAMNSGKPTLINAEIDPAAGSEAGNIGSLNPQSTLKKK

SEQ ID NO: 5 - protein, 528 aa, Methylobacterium extorquens
MTMLLPEPSALLPTDSKEASVPATTLHELIQAGADAAPALSSPGGVPLTFQALRALTERTVADLNARGIGRGDRVAIVLPNGPEMAAAFIAVAAGTTSAPLNPSYKADEFEFYMSDLGAKLLLVAEGSETPAVAVAEKLGVSVARLRPTPDEGAGSFTLHFASESTGPTEKSGPAGSDDIALVLHTSGTTSRPKIVPLTQANVCASARNIRTALAFGPEDRGLNIMPLFHIHGLIAGILAPLSVGGQVSCTPGFNALKFFGWMDEVNPTWYTGVPTMHQAILGRAARNKEIIARNPLRFIRSSSSSLPPQVMKELEETFGAPVIEAYGMTEAAHQMASNPLPPKPHYAGSVGLAAGPEIAVVDLDGEPLPAGETGEIVIRGDNVMKGYENNEKANAEAFTKQGWFRTGDQGVLSPEGYLSITGRLKEIINRGGEKISPREVDEILMDHPAVSQCVTFAVPHDKLGEDVAAAIVLREGVEAVEKDIRSFASERLAAFKVPAKILILDEIPKGATGKLQRIGLAQKLGLV

SEQ ID NO: 6 - protein, 132 aa, E. coli
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 7 - DNA, 26 bp, Artificial
  Primer for oxc (DNA of the oxalyl-CoA decarboxylase; oxc_fw_NDEI)
gttcacatatgaccgtccaggcccag

SEQ ID NO: 8 - DNA, 32 bp, Artificial
  Primer for oxc (DNA of the oxalyl-CoA decarboxylase; oxc_rv_BamHI)
cgctggatcctcacttcttcttcaaggtgctc

SEQ ID NO: 9 - DNA, 25 bp, Artificial
  Primer for oxs (DNA of the acetyl-CoA synthetase; oxs_fw_NdeI)
gttcacatatgacgatgcttctgcc

SEQ ID NO: 10 - DNA, 25 bp, Artificial
  Primer for oxs (DNA of the acetyl-CoA synthetase; oxs_rv_BamHI)
caatggatcctcagaccagcccgag

SEQ ID NO: 11 - DNA, 27 bp, Artificial
  Primer for panE2 (DNA of the oxalyl-CoA reductase; panE2_fw_NdeI)
gcgcacatatgagcatcgcgatcgtcg

SEQ ID NO: 12 - DNA, 28 bp, Artificial
  Primer for panE2 (DNA of the oxalyl-CoA reductase; panE2_rv_BamHI)
cagaggatcctcatgctccctggatcgc

SEQ ID NO: 13 - DNA, 28 bp, Artificial
  Primer for oxcY497A (DNA of a mutant of the oxalyl-CoA decarboxylase; oxc_Y497A_fw)
caacaacggcatcgctcgcggcaccgac

SEQ ID NO: 14 - DNA, 28 bp, Artificial
  Primer for oxcY497A (DNA of a mutant of the oxalyl-CoA decarboxylase; oxc_Y497A_rv)
gtcggtgccgcgagcgatgccgttgttg

SEQ ID NO: 15 - DNA, 25 bp, Artificial
  Primer for oxc S568A (DNA of a mutant of the oxalyl-CoA decarboxylase; oxc_S568A_fw)
ccggcagcgaggccggcaatatcgg

SEQ ID NO: 16 - DNA, 25 bp, Artificial
  Primer for oxc S568A (DNA of a mutant of the oxalyl-CoA decarboxylase; oxc_S568A_rv)
ccgatattgccggcctcgctgccgg

SEQ ID NO: 17 - DNA, 1737 bp, Artificial
  Hacl1 codon optimized DNA for E. coli
atgccggattcaaactttgctgaacgttcagaagaacaggtttctggcgcaaaagtgattgcgcaagcgctaaaaacccaggatgtcgaatatatcttcggtatagtgggtattccggtgacggaaatcgccatcgcggctcagcaactcggtattaaatacatcggtatgcgtaatgagcaggcggcttgttacgcagcaagcgcgattggctatctgacctcaagaccgggggtatgccttgttgtcagcggcccgggcctgattcatgccctgggtggtatggcgaatgctaacatgaactgctggccgctgttagttataggcggcagcagtgaacgcaatcaggagaccatgggcgcattccaagagtttcctcaggtggaagcctgccgcctgtataccaaattttctgctagaccttcgtcaatagaagccatccccttcgtgattgaaaaagcggtccgtagttctatctatgggcgccctggggcttgctatgttgacattcccgccgacttcgtaaacctccaagtgaatgtcaattccattaaatacatggaacgctgtatgtcgccgccgatcagtatggccgagaccagcgccgtgtgcacagcggcgagtgttatccgcaacgccaaacagccattactaataattgggaaaggagccgcctatgctcacgctgaagagtccatcaaaaaactggtggaacaatacaagttgccctttttacctactcccatgggtaaaggtgttgtacctgataatcatccatactgcgtcggtgcagcccgttcccgcgcactacagttcgctgacgtgattgtgctgtttggtgcgcgtttaaactggatactccacttcggtttaccgccacgctaccagccggatgtgaaatttatacaggttgacatctgtgccgaagaactgggcaataatgtgaaaccagcagtcactttgctggggaacatccatgcagtcaccaaacaactgcttgaagagctggacaagacgccgtggcaatatccgccagagtcaaaatggtggaagacgctacgtgagaaaatgaagagcaacgaagcggcttcgaaagagttggcctccaaaaagtcattgcctatgaattactataccgtcttttatcatgtccaagagcagctgccgcgggattgctttgtcgtgtcggagggcgccaacaccatggatataggtcgcactgtccttcagaactatctgccccggcatcgcctggacgctggtaccttcggaactatgggcgtagggctgggctttgctattgcggcggcagtggtagcaaaggatcgcagcccggggcagtggattatttgtgtagaaggggactccgcatttggattctctggtatggaggtagagacaatatgccgttataatttgccaattatccttctagtggtgaataacaatggtatttatcagggctttgatacagatacgtggaaggaaatgctgaaatttcaagatgccacagccgtggtaccccccatgtgtttgctgccgaactctcattacgagcaagtgatgacagcattcggcggcaagggttactttgtgcagacaccggaagaacttcaaaagagccttcgccagagcttggcggacaccacgaaaccgagtctgattaatataatgattgaaccacaggccacaaggaaggcacaagactttcactggctcacgcgttcaaatatgtaa

SEQ ID NO: 18 - protein, 641 aa, Erythrobacter sp. NAP1
MTGFVERPEQAHTPNCTGVQYAAMYERSLADPDGFWLEQAKRLDWTQQPRKGGEWSYDPVDIKWFADGSLNLCHNAVDRHLDSRGDTPAIIFEPDDPATPSRTLTYRQLHSEVIHMANALKAIGVTKGERVTIYMPNIVEGVTAMLACARLGAIHSVVFGGFSPEALAGRIIDCESRFVVTADEGKRGAKSVPLKANVDAALEVEGVDVTGVLVVQHTGLAVPMTEGRDHWFHEVKSDADVPCETMAAEDPLFILYTSGSTGKPKGVLHTTGGYGVWTATTFSYIFDYQPGEVFWCTADIGWVTGHSYIVYGPLQNGATQVLFEGVPNYPDFGRFWDVVAKHKVSILYTAPTAIRALMREGDDYVTSRDRSSLRLLGSVGEPINPEAWRWYFDVVGEGRCPIIDTWWQTETGGCMITTLPGAHDMKPGSAGLPMFGIRPQLVDNDGAVLDGATEGNLCITHSWPGQARSVYGDHDRFVQTYFSTYSGKYFTGDGCKRDEDGYYWITGRVDDVINVSGHRMGTAEVESALVLHPQVAEAAVVGYPHDVKGQGIYCYVTTNAGVEGSDELYQELRAHVRKEIGPIATPDQIQFTDGLPKTRSGKIMRRILRKVAENDYGSLGDTSTLADPSLVDRLIEGRQKT

SEQ ID NO: 19 - protein, 641 aa, Artificial
  Mutant of acyl-CoA synthetase from Erythrobacter sp. NAP1
MTGFVERPEQAHTPNCTGVQYAAMYERSLADPDGFWLEQAKRLDWTQQPRKGGEWSYDPVDIKWFADGSLNLCHNAVDRHLDSRGDTPAIIFEPDDPATPSRTLTYRQLHSEVIHMANALKAIGVTKGERVTIYMPNIVEGVTAMLACARLGAIHSVVFGGFSPEALAGRIIDCESRFVVTADEGKRGAKSVPLKANVDAALEVEGVDVTGVLVVQHTGLAVPMTEGRDHWFHEVKSDADVPCETMAAEDPLFILYTSGSTGKPKGVLHTTGGYGVWTATTFSYIFDYQPGEVFWCTADIGWVTGHSYIVYGPLQNGATQVLFEGVPNYPDFGRFWDVVAKHKVSILYTAPTAIRALMREGDDYVTSRDRSSLRLLGSIGEPINPEAWRWYFDVVGEGRCPIIDTWWQTETGGCMITTLPGAHDMKPGSAGLPMFGIRPQLVDNDGAVLDGATEGNLCITHSWPGQARSVYGDHDRFVQTYFSTYSGKYFTGDGCKRDEDGYYWITGRVDDVINVSGHRMGTAEVESALVLHPQVAEAAVVGYPHDVKGQGIYCYVTTNAGVEGSDELYQELRAHVRKEIGPIATPDQIQFTDGLPKTRSGKIMRRILRKVAENDYGSLGDTSTLADPSLVDRLIEGRQKT

SEQ ID NO: 20 - DNA, 1926 bp, Erythrobacter sp. NAP1
atgactggatttgtcgagcggcccgagcaggcgcacactcccaattgcaccggtgttcaatacgctgcgatgtatgagcgttcgctggctgatcctgatggcttctggcttgaacaggccaagcgcctcgattggacccagcagccaaggaaaggtggcgagtggagctacgacccggtcgatatcaagtggtttgccgacggttcgctgaacctttgccacaacgcggtcgatcggcatctcgacagccgcggtgacacacccgctattatctttgaacccgacgacccagcaacgccttcgcgcacgctcacctatcgccagcttcactccgaagtcatccacatggccaacgccctcaaggccatcggggtgaccaagggcgagcgcgtgacgatctacatgccgaacatagtcgagggcgtgacggctatgctcgcctgcgcgcggctgggcgcgatccactccgttgtctttggcggcttctcgcctgaggcgcttgctggtcggatcattgattgtgaaagccgcttcgtggtcaccgcggacgaaggcaagcgcggggcaaagagcgttcctttgaaagccaatgtggacgccgccttggaagtcgaaggggtcgatgtaactggagtgctggtcgtccagcacaccggattagccgtgccgatgaccgaagggcgagaccactggttccatgaggtcaaatcggacgcagatgtgccctgcgagaccatggcagccgaagacccgctcttcattctttacacatcgggctcaaccggaaagcccaagggtgtgctccacacaaccggcggatacggcgtgtggaccgcgacgacgttcagttacattttcgactatcagcccggtgaggtgttctggtgcacggccgatatcggctgggtcactggccacagctacatcgtctatggcccactacagaacggcgcgacgcaggtgctgttcgagggtgttcccaactaccccgattttggtcggttctgggatgttgtcgcaaaacacaaggtcagcatcctctacaccgcgccaaccgcgatccgggccctgatgcgtgaaggcgatgactacgtgacaagccgcgatcgttcctcgctccgactgctcggaagcgtaggagagccgatcaacccggaagcctggcgctggtatttcgacgtggtcggagaggggcgttgcccgatcatagacacatggtggcagaccgaaacaggcggttgcatgatcaccactcttccaggtgcgcacgacatgaaaccgggcagcgccggtttgccgatgttcggtatccgcccgcagctggtcgacaatgatggagccgtgctggacggagcgaccgagggcaatctgtgcatcacgcacagctggcccggacaggcgcgcagcgtttacggcgatcacgaccggttcgttcagacctatttctcgacgtactccggcaagtacttcaccggggacgggtgcaagcgcgatgaggatggctattactggatcacgggccgcgtggacgacgtgatcaacgtgtcaggccaccgaatgggaactgccgaagtcgaaagcgcattggtgctccacccgcaagtcgccgaagcggcggtggttggatacccgcatgatgtgaaggggcagggcatctactgctacgtcacaaccaatgcgggcgtcgaaggctcggacgagctttatcaggagttgcgcgcgcatgtgcgcaaggaaatcggacccatcgcgacgcctgaccagattcagttcaccgacggcctgcccaagacacgttcgggcaagatcatgcgccgaattctgagaaaggtcgccgaaaacgactacggctcgctgggcgacacttcgacacttgccgatccctcgttggtcgaccgcctgatcgagggccgacagaaaacctag

SEQ ID NO: 21 - DNA, 1926 bp, Artificial
  Codon-optimized nucleotide sequence encoding acyl-CoA synthetase from Erythrobacter sp. NAP1 for E. coli
atgaccggtttcgtagagcgtccggaacaggctcacaccccgaactgcacaggtgttcaatacgcagcaatgtacgagcgttccctggctgatccggacggcttttggctggaacaggctaaacgtctggactggacccagcagccgcgtaagggcggcgaatggtcttacgacccggttgacataaaatggttcgcggacggttccctgaacctgtgtcataacgcagtagaccgtcacctggatagccgcggtgacaccccggcaatcatcttcgagccggatgacccggctactccgtctcgcacccttacctatcgtcagctgcactccgaagttattcacatggctaatgctctgaaagcaatcggcgtaaccaaaggcgagcgtgttacaatctacatgccgaacatcgtagaaggtgttaccgctatgctggcttgcgcacgcctgggtgcgatccacagtgtagtgttcggtggtttctctcctgaggctctggccggtcgtatcatcgactgtgaatctcgcttcgttgtgactgcggacgaaggcaaacgcggtgcaaaatccgttccgctgaaggctaacgtagacgctgctctggaagttgaaggcgtagacgttactggtgttctggtagtacagcataccggcctggcggttccgatgaccgagggtcgcgaccactggttccatgaggttaaatctgacgctgacgtgccgtgcgaaactatggcagctgaagacccgctgtttattctgtacacaagcggctccaccggtaaaccaaaaggtgttctgcacactactggtggttatggtgtttggactgctaccaccttcagctacatcttcgactaccagccgggtgaagtgttctggtgtacagcagacatcggttgggttaccggtcattcttacatcgtatatggtccgctgcagaacggtgcaactcaggttctgtttgaaggtgtaccaaactacccggacttcggtcgtttctgggacgttgtagctaaacacaaagtttccatcctctacactgcaccgaccgctatccgcgcgctgatgcgtgaaggtgatgactatgttacttcacgtgaccgcagttccctgcgtctgctgggctctgtaggtgagccgatcaacccggaggcctggcgttggtacttcgatgttgttggtgaaggtcgttgtccgatcatcgacacttggtggcagaccgagactggcggttgcatgattactactctgccgggtgctcacgatatgaagccgggctctgcaggcctgccgatgttcggtatccgtccgcagctcgttgacaacgacggtgcagtgctggacggtgcaacagaaggtaacctgtgtatcacccactcttggccgggccaggctcgctctgtttacggtgatcacgaccgcttcgtacagacttacttctccacctactccggcaagtatttcactggtgacggttgcaaacgtgacgaggatggctactattggattaccggtcgtgtggacgacgttatcaacgtttctggccatcgtatgggtactgctgaggtggaaagcgcactggtactgcacccgcaggtagcagaagcagctgtggttggttacccgcacgacgttaagggccagggtatctactgttatgtaaccaccaacgcaggcgtggaaggttctgacgaactgtaccaggagctgcgtgcccacgtgcgcaaagaaatcggcccgatcgcaactccggatcagatccagttcactgacggtctgccgaagacgcgttccggtaaaattatgcgccgtatcctgcgtaaagtggcagaaaacgactatggttctctgggtgatacttccaccctggcggacccgtctctggtggatcgtctgattgagggccgtcagaaaacttaa

SEQ ID NO: 22 - DNA, 27 bp, Artificial
  Primer for the preparation of a mutant of the gene from Erythrobacter sp. NAP1 encoding acyl-CoA synthetase (GATCGGCTCACCAATAGAGCCCAGCAG)
ctgctgggctctattggtgagccgatc

SEQ ID NO: 23 - DNA, 27 bp, Artificial
  Primer for the preparation of a mutant of the gene from Erythrobacter sp. NAP1 encoding acyl-CoA synthetase (eryACS-1_V379I_rv)
gatcggctcaccaatagagcccagcag

SEQ ID NO: 24 - DNA, 1737 bp, Humane
atgccggacagtaacttcgcagagcgcagcgaggagcaggtgtctggtgctaaagtcatcgctcaggccctgaaaacgcaagatgtggagtacatatttggcatcgtaggcatcccagtgaccgaaatcgccattgctgcccagcagctaggcatcaagtacatcgggatgaggaatgagcaagcggcttgttatgctgcctccgcgattggatatctgacaagcaggccaggagtctgccttgttgtttctggcccaggtctcatccatgccttgggcggtatggcaaatgcaaacatgaactgctggcccttgcttgtgattggtggttcctctgaaagaaaccaagaaacaatgggagctttccaggagtttcctcaggttgaagcttgtagattatataccaagttctctgcccgcycaagcagcatagaagctattccttttgttattgaaaaggcagtgagaagcagtatctatggtcgtccaggtgcttgctatgttgacataccagcagattttgtgaaccttcaggtgaatgtgaattctataaagtacatggaacgctgcatgtcacctcctattagcatggcagaaacctctgctgtgtgcacggcggcttctgttattaggaatgccaaacaaccccttcttatcatcgggaaaggtgctgcttacgctcatgcagaagagagtatcaagaaattggtggagcaatataaactgccatttttgcccacccctatgggaaagggtgttgtccctgacaaccatccatactgtgtaggtgcagccagatccagggctttgcaatttgctgatgtaattgtgttatttggtgccagactaaattggattttacattttggactgcctccaagatatcagccagatgtgaagtttatccaggttgatatctgtgcagaagaattggggaataatgtaaagcccgctgttactttgctaggaaacatacatgctgtcactaagcagcttttagaggaacttgataaaacaccatggcagtatcctccagagagcaagtggtggaaaactctgagagaaaaaatgaagagcaatgaagctgcatccaaggaactagcttctaaaaaatccctgcctatgaattattacacagtattctaccatgttcaagaacaactacctagagactgtttcgtggtaagtgaaggagcaaatactatggacattggacggactgtgcttcagaactaccttcctcgtcacaggcttgatgctggtactttcggaacaatgggagttggtttgggatttgctattgcagctgccgtggtggctaaagatagaagccctgggcattggatcatctgtgtggaaggagacagtgcatttgggttttctggcatggaggtagaaaccatctgcaggtacaacttgccaatcatactgttggtagtgaataacaatggaatttaccaaggttttgatacagatacttggaaagaaatgttaaaatttcaagatgctactgcagtggtccctccaatgtgtttgctgccaaattcacattatgagcaagtcatgactgcatttggaggcaaagggtattttgtacaaacaccagaagaactccaaaaatccctggagcagagcctagcagacacaactaaaccttctcttatcaacatcatgattgagccacaagccacacggaaggcccaggattttcattggctgacccgctctaatatgtaa

SEQ ID NO: 25 - DNA, 399 bp, E. coli
atgtctacaacacataacgtccctcagggcgatcttgttttacgtactttagccatgcccgccgataccaatgccaatggtgacatctttggtggttggttaatgtcacaaatggatattggcggcgctattctggcaaaagaaattgcccacggtcgcgtagtgactgtgcgggttgaaggaatgactttcttacggccggttgcggtcggcgatgtggtgtgctgctatgcacgctgtgtccagaaagggacgacatcggtcagcattaatattgaagtgtgggtgaaaaaagtagcgtctgaaccaattgggcaacgctataaagcgacagaagcattatttaagtatgtcgcggttgatcctgaaggaaaacctcgcgccttacctgttgagtaa